(19) United States
(12) Patent Application Publication
(10) Pub. No.: US 2016/0309672 A1
(43) Pub. Date: Oct. 27, 2016

(54) ALLELES MODIFYING BRASSICA PLANT TOTAL SATURATED FATTY ACID CONTENT

(71) Applicant: CARGILL, INCORPORATED, Wayzata, MN (US)

(72) Inventors: Richard FLETCHER, Windsor, CO (US); David HERRMANN, Fort Collins, CO (US); Honggang ZHENG, Fort Collins, CO (US)

(21) Appl. No.: 15/037,887

(22) PCT Filed: Nov. 21, 2014
(86) PCT No.: PCT/US2014/066973

§ 371 Date: May 19, 2016

Related U.S. Application Data
(60) Provisional application No. 61/907,025, filed on Nov. 21, 2013.

(51) Int. Cl. A01H 5/10; C12N 9/02; C12N 9/16

(52) U.S. Cl. CPC A01H 5/10; C12N 9/16; C12Y 301/02014; C12N 9/0071; C12Y 114/19003

(57) ABSTRACT

The present disclosure sets forth alleles at two genetic loci whose presence reduces the saturated fatty acid content of Brassica seeds. Methods for producing plants containing those alleles, and which produce seeds having low saturated fatty acid content, are also described.

This application claims the benefit of U.S. Provisional Application 61/907,025 filed on Nov. 21, 2013, which is incorporated by reference in its entirety.

### TECHNICAL FIELD

This application contains a sequence listing submitted electronically via EFS-web, which serves as both the paper copy and the computer readable form (CRF) and consists of a file entitled “SequenceListing_033449_8089_WO00.txt”, which was created on Nov. 21, 2014, which is 73,728 bytes in size, and which is herein incorporated by reference in its entirety.

This invention relates to Brassica plants and, more particularly, Brassica plants having modified alleles at two quantitative trait loci (QTLs) that modify the total fatty acid content of oil in their seed. The plants may optionally contain modified fatty acyl-acyl carrier protein thioesterase A2 (FATA2) loci and/or fatty acyl-acyl carrier protein thioesterase B (FATB) loci, which may further contribute to a low total saturated fatty acid content phenotype in combination with a typical, mid, or high oleic acid content.

### BACKGROUND

In recent years, diets high in saturated fats have been associated with increased levels of cholesterol and increased risk of coronary heart disease. As such, current dietary guidelines indicate that saturated fat intake should be no more than 10 percent of total calories. Based on a 2,000-calorie-a-day diet, this is about 20 grams of saturated fat a day. While canola oil typically contains only about 7% to 8% saturated fatty acids, a decrease in its saturated fatty acid content would improve the nutritional profile of the oil.

### SUMMARY

Mutations in FATA2 and FATB alleles in Brassica plants have previously been described as useful in controlling the total saturated fatty acid content oil in the seed of plants of the Brassicaceae, see e.g., WO 2011/075716. The present disclosure describes two additional quantitative trait loci, or QTLs, identified in plants described in WO 2011/075716. Those loci are defined by their contribution to the low, or very low, saturated fatty acid content in their seed oil, and the SNP markers identified herein. The first locus, QTL1, is believed to reside upon Brassica napus chromosome N1, and the second locus, QTL2, is believed to reside upon Brassica napus chromosome N19 in the mapping populations described herein. Although the loci may be referred to or described as residing on chromosome N1 or N19, it is understood that the loci are defined by their SNP alleles and contribution to the fatty acid content of their seed oil and that those loci may appear on other chromosomes, particularly in progeny.

The newly identified QTL1 (N1) and/or QTL2 (N19) may be employed individually or in combination with either or both of FATA2 and/or FATB mutations to produce Brassica plants producing oils with a low total saturated fatty acid content (i.e., 6% or less total saturates) or oils having very low saturates (i.e., having 3.6% or less total saturates). In addition to the mutations present in QTL1, QTL2, and those in FATA2 and/or FATB, Brassica plants also may include mutant fatty acid desaturase (FAD) alleles to tailor the oleic acid and α-linolenic acid content to the desired end use of the oil. Brassica plants described herein are particularly useful for producing canola oils for certain food applications as the plants are not genetically modified, that is to say non-transgenic.

In one embodiment, this document describes Brassica plants (e.g., Brassica napus, Brassica juncea, or Brassica rapa plants) and progeny thereof (e.g., seeds) that include modified alleles at one or more of the QTLs described on chromosomes N1 and N19. Such plants may also have mutations at one or more of the different fatty acyl-acyl carrier protein thioesterase B (FATB) loci (e.g., three or four different loci), wherein each modified allele results in the production of a FATB polypeptide having reduced thioesterase activity relative to a corresponding wild-type FATB polypeptide. The plants bearing modifications at QTL1 and/or QTL2 can be F₁ hybrids.

Modified alleles can include alleles giving rise to a nucleic acid encoding a truncated protein (e.g., a truncated FATB polypeptide). A modified allele can also include a nucleic acid encoding a deletion or frame shift mutation (e.g., a FATB polypeptide having a deletion of a helix/4-stranded sheet (4HBT) domain or a portion thereof). A modified allele can include a nucleic acid encoding a FATB polypeptide having a non-conservative substitution of a residue affecting substrate specificity. A modified allele can include a nucleic acid encoding a polypeptide having a non-conservative substitution of a residue affecting catalytic activity. Any of the modified alleles can be a mutant allele.

In some embodiments, plants comprising QTL1 or QTL2 also may comprise a nucleic coding for a truncated FATB polypeptide having a nucleotide sequence selected from the group consisting of: SEQ ID NO:1, SEQ ID NO:2, SEQ ID NO:3, and SEQ ID NO:4. In some embodiments, the plant contains nucleic acids having the nucleotide sequences set forth in SEQ ID NO:1 and SEQ ID NO:2; SEQ ID NO:1 and SEQ ID NO:3; SEQ ID NO:1 and SEQ ID NO:4; SEQ ID NO:1, SEQ ID NO:2, and SEQ ID NO:3; SEQ ID NO:1, SEQ ID NO:2, and SEQ ID NO:4; SEQ ID NO:1, SEQ ID NO:3, and SEQ ID NO:4; or SEQ ID NO:1, SEQ ID NO:2, SEQ ID NO:3, and SEQ ID NO:4.

A plant can produce seeds yielding an oil having a total saturates content of about 2.5 to about 5.5%. The palmitic acid content of the oil can be about 1.5 to about 3.5%. The stearic acid content of the oil can be about 0.5 to about 2.5%. The oil can have an oleic acid content of about 62 to about 85% (e.g., about 62 to about 65%, about 65 to about 72%, about 72 to about 75%, about 75 to about 80%, about 80 to about 84% or about 82 to about 85%), and/or a linoleic acid content of about 8 to about 10%, and an α-linolenic acid content of no more than about 4% (e.g., about 2 to about 4%).

In another embodiment, the Brassica plants comprising QTL1 and/or QTL2 (e.g., B. napus, B. juncea, or B. rapa plants) and progeny thereof (e.g., seeds) include a modified allele at a fatty acyl-ACP thioesterase A2 (FATA2) locus, wherein the modified allele results in the production of a FATA2 polypeptide (e.g., FATA2b polypeptide) having reduced thioesterase activity relative to a corresponding wild-type FATA2 polypeptide. The modified allele can include a nucleic acid encoding a FATA2 polypeptide having a mutation in a region (SEQ ID NO:29) corresponding to amino acids 242 to 277 of an Arabidopsis FATA2 polypeptide. The FATA2 polypeptide can include a substitution of a leucine residue for proline at position 255. The plant can be an F₁ hybrid. Any of the modified alleles can be a mutant allele.

Any of the plants described herein further can include one or more modified (e.g., mutant) alleles at FAD2 loci. For example, a mutant allele at a FAD2 locus can include a nucleic acid encoding a FAD2 polypeptide having a lysine substituted for glutamic acid in a HECGH (SEQ ID NO:5) motif. A mutant allele at a FAD2 locus can include a nucleic acid encoding a FAD2 polypeptide having a glutamic acid substituted for glycine in a DRDYGILNKV (SEQ ID NO:7) motif or a histidine substituted for leucine in a KYLNNP (SEQ ID NO:6) motif. In some embodiments, the plant contains a mutant allele at two different FAD2 loci: a mutant allele including a nucleic acid encoding a FAD2 polypeptide having a lysine substituted for glutamic acid in a HECGH motif and a mutant allele including a nucleic acid encoding a FAD2 polypeptide having a glutamic acid substituted for glycine in a DRDYGILNKV motif or a histidine substituted for leucine in a KYLNNP motif.

Any of the plants described herein further can include modified alleles (e.g., mutant alleles) at two different FAD3 loci, wherein one of the modified alleles includes a nucleic acid encoding a FAD3A polypeptide having a cysteine substituted for arginine at position 275, and wherein one of the modified alleles includes a FAD3B nucleic acid sequence having a mutation in an exon-intron splice site recognition sequence.

In another aspect, this disclosure features Brassica plants (e.g., B. napus, B. juncea, or B. rapa plants) and progeny thereof (e.g., seeds) that include modified alleles at two or more different FATB loci (e.g., 3 or 4 different FATB loci), wherein each modified allele results in production of a FATB polypeptide having reduced thioesterase activity relative to a corresponding wild-type FATB polypeptide, and further includes a modified allele at a FAD2 locus, wherein the modified allele includes a nucleic acid encoding a FAD2 polypeptide having a lysine substituted for glutamic acid in a HECGH motif. The plant further can include a modified allele at a different FAD2 locus, the modified allele including a nucleic acid encoding a FAD2 polypeptide having a glutamic acid substituted for glycine in a DRDYGILNKV motif or a histidine substituted for leucine in a KYLNNP motif. The FATB modified allele can include a nucleic acid encoding a truncated FATB polypeptide. The nucleic acid encoding the truncated FATB polypeptide can include a nucleotide sequence selected from the group consisting of: SEQ ID NO:1, SEQ ID NO:2, SEQ ID NO:3, and SEQ ID NO:4. For example, the plant can contain nucleic acids having the nucleotide sequences set forth in SEQ ID NO:1, SEQ ID NO:2, SEQ ID NO:3, and SEQ ID NO:4. The plant can be an F₁ hybrid. Any of the modified alleles can be a mutant allele.

In another aspect, this disclosure features a method of producing an oil. The method includes crushing seeds produced from at least one Brassica plant described herein; and extracting the oil from the crushed seeds, the oil having, after refining, bleaching, and deodorizing, a total saturates content of about 2.5 to about 5.5%. The oil further can include an eicosenoic acid content of about 1.6 to about 2.3%. The oil further can include an oleic acid content of about 78 to about 80%, a linoleic acid content of about 8 to 10%, and an α-linolenic acid content of about 2 to about 4%.

This disclosure also features a method for preparing a Brassica plant. The method including the steps of:
a) crossing one or more first Brassica parent plants with one or more second Brassica parent plants,
   wherein said first Brassica parent plants comprise a nucleic acid sequence having greater than 90%, (e.g., 95%, 97.5%, 98%, 99%, 99.9%, 99.99%, 99.999%) identity or having 100% identity to all or part of the genomic sequences between the chromosome N1 (QTL1) SNP markers at positions 20772548 and 22780181 (e.g., between 20843387 and 21080816, or between 20874571 and 20979545) and/or all or part of the genomic sequence between chromosome N19 (QTL2) SNP markers at positions 11538807 and 18172630 (e.g., 12010676 and 13207412, 12378335 and 12979251)
   of the B. napus Salomon line, ATCC (American Type Culture Collection) deposit designation PTA-11453, and wherein either or both of those sequences can give rise to a reduction in the 16:0 fatty acid content of oils found in the seeds of the parent or progeny plants; wherein said first Brassica parent plant is not a plant of the B. napus Salomon line, the 1764 line, the 15.24 line, or any other plant in WO2011/075716 comprising QTL1 and/or QTL2 of the Salomon line; and/or, wherein said first parent plant optionally comprises a mutant allele at one, two, three, four or more FATA2, FATB, and/or FAD2 loci,
   wherein said one or more second Brassica parent plants optionally comprise a mutant allele at one, two three, four or more FATA2, FATB, and/or FAD2 loci that are different from the FATA2, FATB and/or FAD2 loci of said first Brassica parent, and
   wherein each said mutant FATA2 allele, if present, comprises a nucleic acid encoding a FATA2 polypeptide having a mutation in a region corresponding to amino acids 242 to 277 of the polypeptide, each said mutant FATB allele results in the production of a FATB polypeptide having reduced thioesterase activity relative to a corresponding wild-type FATB polypeptide, and each said mutant FAD2 allele at said FAD2 loci comprises a nucleic acid encoding a FAD2 polypeptide having a lysine substituted for glycine in a His-Glu-Cys-Gly-His motif;
   and
b) selecting, for one, two, three, four, five or more generations, for progeny plants having
   (i) all or part of the genomic sequences between the chromosome N1 (QTL1) SNP markers at positions 20772548 and 22780181 (e.g., between 20843387 and 21080816, or between 20874571 and 20979545) and/or all or part of the genomic sequence between chromosome N19 (QTL2) SNP markers at positions 11538807 and 18172630 (e.g., 12010676 and 13207412, 12378335 and 12979251)
   and
   (ii) said mutant alleles at one, two, three, four or more different FATA2, FATB and/or FAD2 loci present in said first and/or second Brassica parent if present in said first or second parent,
      thereby obtaining the Brassica plant. In such an process, the method of selection may include the use of a variety of molecular techniques useful for, among other things, identifying the presences of specific nucleic acid sequences (e.g., hybridization assays, PCR, LCR and nucleic acid sequencing).

The present disclosure includes and provides for methods of selecting Brassica plants for the presence or absence of all or part of QTL1 and/or QTL2 of Salomon (ATCC deposit ATCC PTA-11453); which may be used, for example, to guide breeding programs. Such methods of selecting or breeding Brassica plants comprise obtaining one or more Brassica plants and assessing their DNA to determine the presence or absence of QTL1 (on chromosome N1) and/or all or part of QTL2 (on chromosome N19). Based upon the results of the assessment, plants are selected for the presence or absence of all or part of QTLland/or QTL2 to produce one or more selected plants.

In one embodiment, this disclosure includes and provides for a canola oil having an oleic acid content of about 78 to about 80%, a linoleic acid content of about 8 to about 10%, an α-linolenic acid content of no more than about 4%, and an eicosenoic acid content of about 1.6 to about 2.3%. The palmitic acid content can be about 1.5 to about 3.5%. The stearic acid content can be about 0.5 to about 2.5%. The eicosenoic acid content can be about 1.9 to about 2.2%. The α-linolenic acid content can be about 2 to about 4%. In another embodiment, this disclosure includes and provides for an oil having a total saturated fatty acid content of no more than about 3.7% and an oleic acid content of about 62 to about 85% (e.g., about 62 to about 65%, about 65 to about 72%, about 72 to about 75%, about 75 to about 80%, about 80 to about 84% and/or about 82 to about 85%). The oil can have a palmitic acid content of about 2.2 to about 2.4%. The oil can have a stearic acid content of about 0.5 to about 0.8%. The oil can have an eicosenoic acid content of about 1.6 to about 1.9%. The total saturated fatty acid content can be about 3.4 to about 3.7%.

This disclosure also features plant cells and/or seeds of a Brassica plant that may be non-transgenic that comprise a nucleic acid sequence having greater than 80% identity to all or part of the genomic sequences between the chromosome N1 (QTL1) SNP markers at positions 20772548 and 22780181 and/or all or part of the genomic sequence between chromosome N19 (QTL2) SNP markers at positions 11538807 and 18172630 of the B. napus Salomon line, with the proviso that said plant is not a plant of the B. napus Salomon line, the 1764 line, the 15.24 line, or any other plant in WO2011/075716 comprising QTL1 and/or QTL2 of the Salomon line; and/or, or with the proviso that the plant comprises only one of QTL1 and QTL2, or with the proviso that the plant comprises no more than 2 of QTL1, QTL2 and the QTL on N4 for FATA2 identified in Salomon. Such plant cells and/or seeds may also comprise a modified allele (e.g., mutant allele) at a FATA2 locus, the modified allele containing a nucleic acid encoding a FATA2 polypeptide having a mutation in a region (SEQ ID NO:29) corresponding to amino acids 242 to 277 of the polypeptide, the seeds yielding an oil having an oleic acid content of about 78 to about 80%, a linoleic acid content of about 8 to about 10%, an α-linolenic acid content of no more than about 4%, and an eicosenoic acid content of 1.6 to about 2.3%. The plant cells or seeds can be F₂ generation plant cells or seeds. The plant cells and/or seeds also can comprise modified alleles at four different FATB loci and/or a modified allele at a FAD2 locus and modified alleles at two different FAD3 loci, the FAD2 modified allele can include a nucleic acid encoding a FAD2 polypeptide having a lysine substituted for glutamic acid in a HECGH motif, one of the FAD3 modified alleles can include a nucleic acid encoding a FAD3A polypeptide having a cysteine substituted for arginine at position 275, and one of the FAD3 modified alleles can include a FAD3B nucleic acid sequence having a mutation in an exon-intron splice site recognition sequence.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is an alignment of the nucleotide sequences of Brassica rapa FatA1 (“Brapa FatA1,” SEQ ID NO:33; Genbank Accession No. U17098), Arabidopsis thaliana FatA1 (“AtFatA1,” SEQ ID NO:34); At3g25110; Genbank Accession No. NM_113415), B. napus FatA1 from B. napus line 15.24 (“BnFatA1 1524,” SEQ ID NO:35), B. napus FatA2 from 15.24 (“BnFatA2 1524,” SEQ ID NO:39), A. thaliana FatA2 ((“AtFatA2,” SEQ ID NO:36; At4g13050; Genbank Accession No. NM_117374), and B. napus pNL2 ((“Bnapus pNL2,” SEQ ID NO:37; Genbank Accession No. X73849). The black boxes indicate sequence differences compared to the consensus sequence developed from the alignment; the position marked “1” highlights the SNP unique to 15.24 in the B. napus FatA2b isoform and shows the C to T mutation (Pro to Leu) of 15.24. The position marked as “2” highlights a SNP which distinguishes the B. napus FatA2a and B. napus FatA2b isoforms from each other (see FIG. 4).

FIG. 2 is an alignment of a portion of the FatA2 nucleotide sequences from A. thaliana (“AtFatA2,” SEQ ID NO:41), 15.24 (“15.24FatA2 (1),” SEQ ID NO:40; “15.24FatA2 (2),” SEQ ID NO:38), and the 01OB240 parent (“OB240FatA2 (1),” SEQ ID NO:42; “OB240FatA2 (2),” SEQ ID NO:43). At the position labeled “1,” the “C” to “T” SNP is unique to BnFatA2b sequence in 15.24 germplasm (labeled 15.24FatA2(1)). At the position labeled “2,” the isoform differences between B. napus FatA2a and B. napus FatA2b are apparent (15.24FatA2(2) and OB240FatA2(1) are B. napus FatA2a isoforms, while 15.24FatA2(1) and OB240FatA2(2) are B. napus FatA2b isoforms). Differences in sequence are highlighted in black.

FIG. 3 is an alignment of the amino acid sequence of residues 242 to 277 of the A. thaliana FatA2 (“AtFatA2,” SEQ ID NO:48; GenBank Accession No. NP_193041.1) with the B. napus FatA2 from 15.24 (“15.24FatA2 (1),” SEQ ID NO:49; “15.24FatA2 (2),” SEQ ID NO:50) and 01OB240 (“OB240FatA2 (1),” SEQ ID NO:51; “OB240FatA2 (2),” SEQ ID NO:52). The FatA2 SNP in position “1” (C to T mutation) in 15.24 causes a Pro to Leu change, while the isoform difference at position “2” does not result in an amino acid change in isoforms BnFatA2a and BnFatA2b.

FIG. 4 is an alignment of the BnFatA2a and BnFatA2b sequences from the 01OB240 (SEQ ID NOs:44 and 45, respectively) and 15.24 germplasm (SEQ ID NOs:46 and 47, respectively). Position “1” refers to the “C” to “T” SNP unique to 15.24 in the BnFatA2b sequences that correlate with the low saturate phenotype. See also FIGS. 1-3. Position “2” refers to the “2” positions in FIGS. 1, 2, and 3, and highlights a difference in sequence between the BnFatA2a and BnFatA2b isoforms. Black boxes represent mismatches compared to the 01OB240 BnFatA2b.

FIG. 5 shows the breeding scheme used to develop markers and Near Isogenic Lines (NILs). “RP” refers to Recurrent Parent, and the ellipse struck through with an “x” indicates self-pollination.

FIG. 6 is a genetic linkage map demonstrating the QTL1 interval for C16:0 on Chromosome N1.

FIG. 7 is a genetic linkage map demonstrating the QTL2 interval for C16:0 on chromosome N19.

FIG. 8 is a genetic linkage map demonstrating the QTL interval encompassing FATA2 for the reduction in C18:0 found on chromosome N4.

FIG. 9, Panel A, is the genomic sequence of KASIII or FabH (β-ketoacyl-ACP synthase III) showing the mutation identified in Salomon at position 128475124 on chromosome N19. Panel B of FIG. 9 shows the cDNA sequence. The sequences shown in FIG. 9 are the complement of the marker sequence shown in Table 28. The mutation identified in KASIII relative to Surpass 400 (wild type) is a transition from a “G” in the wild type to an “A” in Salomon.

FIG. 10 is the predicted amino acid sequence of KASIII translated from the cDNA sequence set forth in FIG. 9B for the wild type in Surpass 400 and mutant type in Salomon that produces low amounts of saturated fatty acids in its seeds. The wild type comprises a glycine at position 252, whereas the KASIII of Salomon comprises a glutamic acid at that position.

Unless specifically indicated otherwise, like reference symbols in the various drawings indicate like elements.

### DETAILED DESCRIPTION

In general, this disclosure provides Brassica plants, including B. napus, B. juncea, and B. rapa, that yield seeds producing oils with a low total saturated fatty acid content (i.e., 6% or less) or having very low saturated fatty acid content (i.e., having 3.6% or less) that comprise either or both of two loci termed QTL1 and QTL2. Those loci are defined by their contribution to the low, or very low, saturated fatty acid content of seed oil and the SNP markers identified herein. The first locus, QTL1, is believed to reside upon B. napus chromosome N1, and the second locus, QTL2, is believed to reside upon B. napus chromosome N19 based upon the mapping populations described herein. Although the QTL1 and QTL2 loci may be referred to or described as residing on chromosome N1 or N19 herein, it is understood that the loci are defined by their SNP and contribution to the fatty acids content of their seed oil and that those loci may appear on other chromosomes, particularly in progeny. The appearance of QTL1 and/or QTL2 on other chromosomes may result from a variety of events including, but not limited to, homologous chromosomal crossover events. The occurrence of crossover events may be higher in plants such as B. napus, which is an allopolyploid species.

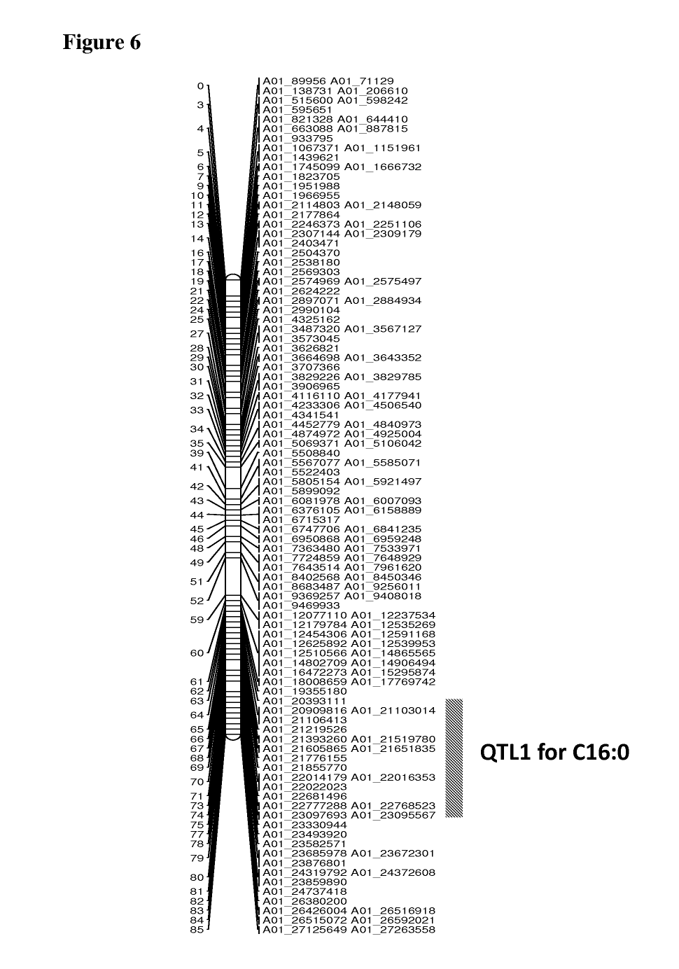

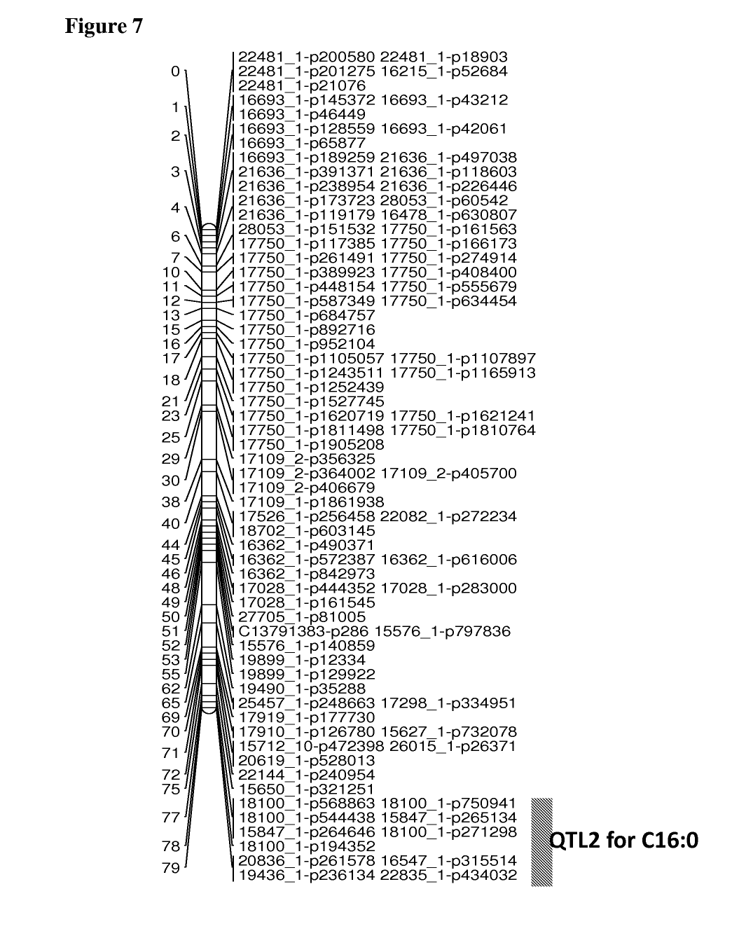

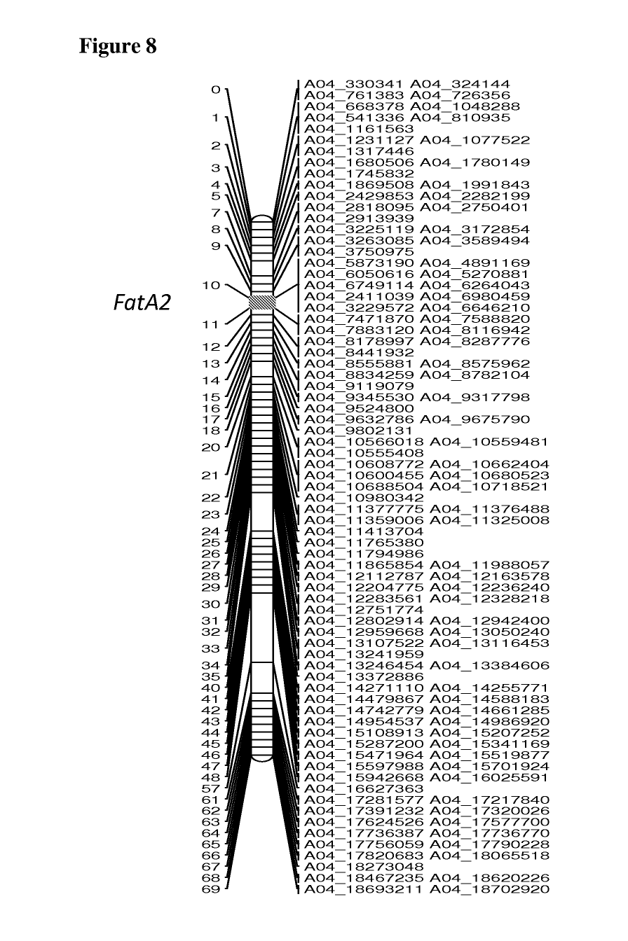

Mapping of QTL1 and QTL2 is accomplished using the Sockeye Red doubled haploid (DH) population derived from a cross between the Salomon line and Surpass 400 (see FIG. 5). Mapping in that population permits localization of QTL1 associated with C16:0 and total saturated fatty acid between the SNP markers A01_20393111 and A01_23097693, which are located at 20280290 and 22599580, respectively, of the B. napus DH12075 reference genome sequence and provided in Table 24 and FIG. 6. The mapping analysis also indicates that QTL2, associated with C16:0, is localized between SNP markers 19436_1-p236134 and 18100_1-p750941, which are located at 14188467 and 18167872, respectively, of the B. napus DH12075 reference genome sequence and provided in Table 25 and FIG. 7. QTL mapping also identifies one QTL interval on the chromosome N4 which encompasses FATA2 gene. This QTL interval is located between the SNP markers, A04_3263085 and A04_8116942, at positions 3170762 and 9985687, respectively, of the B. napus DH12075 reference genome sequence provided in Table 26 and FIG. 8. Analysis of near-isogenic lines (NILs) determines the N1 interval to be between the SNP marker A01_20990218 and the SSR sN2087 at positions 20874310 and 22782616, respectively, of the DH12075 reference genome. Marker analysis of NIL population determines the N19 interval to be between the SNP markers 19436_1-p236134 and 22835_1-p327368, at positions 14188467 and 14907742, respectively, of the DH12075 reference genome. Furthermore, mapping of the genome sequencing data from Salomon, Surpass 400 and IMC201 to a B. napus DH12075 reference genome identifies 52 SNPs spanning from 207725481 to 22780181 of the QTL1 interval on N1 (Table 27) and 58 SNPs spanning from 11538807 to 18172630 of the QTL2 interval on N19 (Table 28). All of the reported SNPs are selected based upon nucleotide variation between Salomon and the lines Surpass 400 and IMC201. Fine mapping of the QTL1 interval on N1 (Tables 29 and 30) and the QTL2 interval on N19 (Tables 31 and 32) further refine the portions of those intervals correlating with reductions in the fraction of 16:0 fatty acid in the oil of Brassica seeds.

In each instance, where map positions are given relative to B. rapa (Chiifu-401) (e.g., in Tables 24 and 26), those positions refer to Version 1.2 of the Chiifu-401 sequence found on CANSEQ consortium web site at http://aafc-aac.usask.ca/canseq/. Where map positions are given relative to B. oleracea (TO1000) (e.g. in Table 25), those positions refer to Version 4 of the TO1000 sequence found at the CANSEQ consortium website at http://aafc-aac.usask.ca/canseq/. For map positions given relative to B. napus (DH12075) (e.g., in Tables 24, 25 and 26), those sequences refer to Version 1.0 of the DH12075 sequence found at the CANSEQ consortium website at http://aafc-aac.usask.ca/canseq/.

Accordingly, in one embodiment, the present disclosure provides for a non-transgenic Brassica plant, or a part thereof, comprising a nucleic acid sequence having greater than 80% (e.g., greater than 90%, 95%, 97.5%, 98%, 99%, 99.9%, 99.99%, or 99.999%) identity to all or part of the genomic sequences within the segments defined by:

the chromosome N1 (QTL1) SNP markers at positions 20772548 and 22780181 (e.g., between 20843387 and 21080816, or between 20874571 and 20979545); and/or

the chromosome N19 (QTL2) SNP markers at positions 11538807 and 18172630 (e.g., 12010676 and 13207412, 12378335 and 12979251) of the B. napus Salomon line, ATCC deposit designation PTA-11453, with the proviso that said plant is not a plant of the B. napus Salomon line, the 1764 line, the 15.24 line, or any other plant in WO2011/075716 comprising QTL1 and/or QTL2 of the Salomon line; and/or; or with the proviso that the plant comprises only one of QTL1 and QTL2 of the Salomon line described herein, or with the proviso that the plant comprises no more than 2 of QTL1, QTL2 and the QTL on N4 for FATA2 of the Salomon line described herein.

In another embodiment the disclosure describes and provides for a non-transgenic Brassica plant, or a part thereof, comprising a nucleic acid sequence having greater than 80% (e.g., greater than 90%, 95%, 97.5%, 98%, 99%, 99.9%, 99.99%, or 99.999%) identity to all or part of the genomic sequence of the B. napus Salomon line, ATCC deposit designation PTA-11453, between the chromosome N1 (QTL1) SNP markers at positions 20772548 and 22780181 with the proviso that the plant lacks 1, 2, 3, 4, 5, 6, 7, 8, 9, 10, 15, 20, 30, 40 or more of the QTL2 SNP markers on chromosome N19 at positions 11538807, 11763228, 11855685, 12010676, 12205222, 12219881, 12355162, 12378335, 12507143, 12615691, 12847514, 12979251, 13003942, 13008581, 13207412, 13364132, 13429175, 13429687, 13460532, 13475876, 13504886, 13704881, 13925427, 14046125, 14135213, 14377562, 14776751, 14801661, 15173478, 15235513, 15387929, 15399385, 15547466, 15623646, 15629066, 15684032, 15741164, 15768411, 15898184, 15943625, 15988083, 16211916, 16238183, 16293509, 16468313, 16698792, 16765722, 16787306, 17041989, 17052864, 17111885, 17219357, 17443797, 17636667, 17893475, 17924151, 18164787, or 18172630.

The disclosure, in another embodiment, also describes and provides for a non-transgenic Brassica plant, or a part thereof, comprising a nucleic acid sequence having greater than 80% (e.g., greater than 90%, 95%, 97.5%, 98%, 99%, 99.9%, 99.99%, or 99.999%) identity to all or part of the genomic sequence of the B. napus Salomon line, ATCC deposit designation PTA-11453, between the chromosome N19 (QTL2) SNP markers at positions 12847514 and 18172630 with the proviso that the plant lacks 1, 2, 3, 4, 5, 6, 7, 8, 9, 10, 15, 20, 30, 40 or more of the SNP markers of Salomon on chromosome N1 (QTL1) at positions 20772548, 20780679, 20843387, 20874199, 20874571, 20924967, 20979545, 21000713, 21057761, 21080816, 21126589, 21175577, 21244175, 21273898, 21301953, 21342623, 21378815, 21425310, 21491979, 21549878, 21597845, 21621627, 21648874, 21700869, 21740913, 21793927, 21825553, 21856527, 21899956, 21938801, 21980398, 22001149, 22060515, 22100267, 22144311, 22180149, 22217506, 22258914, 22260507, 22299725, 22347689, 22379370, 22420077, 22456310, 22498876, 22543194, 22580394, 22621466, 22659331, 22702378, 22739470, or 22780181.

A number of candidate genes that may contribute to the low saturated fatty acid profile of plants are present in or tightly linked to the regions in which QTL1 and QTL2 have been mapped. The genes encoding (1) FATA1 (acyl-ACP thioesterase; AT3G25110 in Arabidopsis), (2) TGD2 (trigalactosyldiacylglycerol 2, a permease-like component of an ABC transporter involved in lipid transfer from endoplasmic reticulum (ER) to chloroplast; AT3G20320 in Arabidopsis), (3) LPAT5 (acyltransferase; AT3G18850 in Arabidopsis), and (4) RFC3 (regulator of fatty acid composition 3; the mutation in Arabidopsis altered composition of fatty acids in roots and seeds; AT3G17170 in Arabidopsis) are among the genes present in the interval, which is believed to be on chromosome N1, onto which QTL1 has been mapped in the Sockeye Red DH population. A number of candidate genes that may contribute to the low saturated fatty acid profile of plants are present in or tightly linked to the regions in which QTL1 and QTL2 have been mapped. FATA1, LPAT5 and RFC3 are among the genes present in the interval, which is believed to be on chromosome N1, onto which QTL1 has been mapped in the Sockeye Red DH population. Several candidate genes have also been identified in the interval, believed to be on chromosome N19, onto which QTL2 has been mapped in the same population. Among the candidate genes in the QTL2 interval on chromosome N19 interval are KAS III/FabH (corresponding to A. thaliana β-ketoacyl-acyl carrier protein synthase III, At1g62640), two genes encoding fatty acid oxidation complex subunit alpha (corresponding to At1g60550 and At5g43280), Fad7/Fad8 encoding omega-3 fatty acid desaturase (corresponding to At3g11170 and At5g05580), and KAS I/FabB (corresponding to A. thaliana β-ketoacyl-[acyl carrier protein] synthase I, At5g46290).

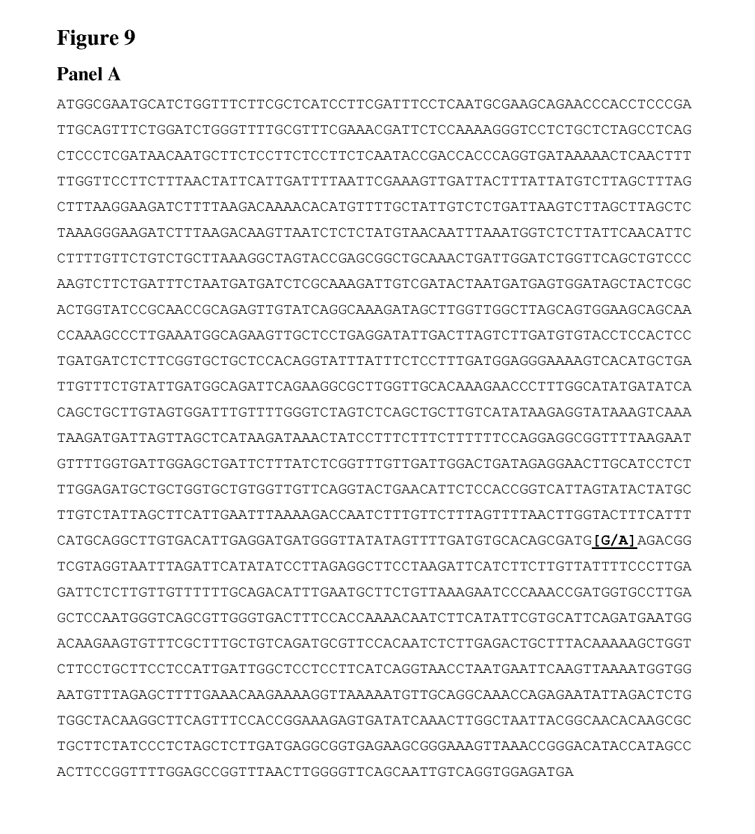

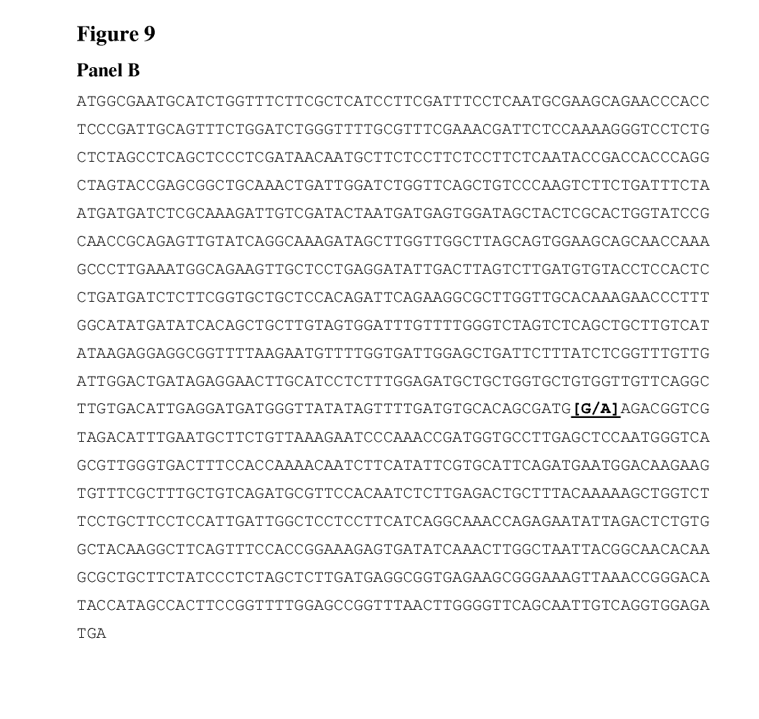

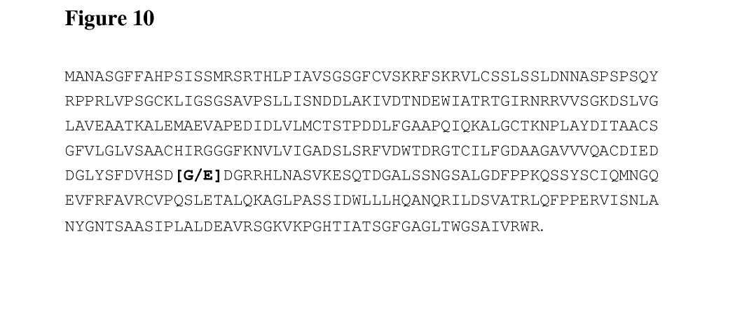

Mapping of the NextGen (Illumina, SanDiego, Calif.) sequencing data from Salomon and Surpass 400 in the QTL2 interval to a B. napus DH12075 reference genome indicates the presence of a single nucleotide mutation in the KAS III gene coding sequence in Salomon relative to Surpass 400, wherein a “G” in that sequence has undergone a transition to an “A” in Salomon [G/A] (see FIG. 9). A survey of 21 B, napus lines shows that transition to be unique to the plants described herein. The change in the amino acid sequence of KAS III translated from the cDNA set forth in FIG. 9, Panel B, shows a replacement of glycine at position 252 by a glutamic acid residue (FIG. 10). Accordingly, in one embodiment the Brassica plants described herein as comprising one or more alleles coding for a KAS III protein have one or more changes from the sequence of that protein in the Surpass 400 line (see FIGS. 9 and 10). In one embodiment, the plants comprise one or more alleles coding for the KAS III protein expressed in Salomon. Such plants may, or may not, contain a nucleic acid coding for the FATA2 mutation found Salomon; such plants may, or may not further comprise a genomic sequence of QTL1 as described in this disclosure.

Analysis of the fatty acid content of plants from the Sockeye Red population indicates that there is a weak or very weak correlation between C16:0 and C18:0, C18:1, C18:2 and C18:3. There is a moderate correlation between C16:0 and total saturated fatty acids. In addition, there is a strong correlation between C16:0 and C14:0, between C18:0 and C20:0 and between C18:0 and total saturated fatty acid content. The results of that correlation analysis shown in Table 23b indicate independent pathways for C16:0/C14:0 (fatty acid synthesis including KAS III and KAS I), C18:0/C20:0 (elongation including KAS II), unsaturated fatty acids, C18:1, C18:2 and C18:3 (desaturation including FAD2 and FADS). KAS III (FabH; β-ketoacyl-ACP synthase III) is an essential enzyme that catalyzes the initiation of fatty acid elongation by condensing malonyl-ACP with acetyl-CoA. KAS I (FabB or FabF1) is responsible for chain elongation up to the 14-carbon fatty acid. KAS II (Fab1) condenses palmitoyl-ACP with malonyl-ACP to form stearoyl-ACP. Note that, while KAS I and KAS II use β-ketoacyl-ACP as the priming unit, KAS III uses acetyl-CoA. The mitochondrion of Arabidopsis is also capable of fatty acid synthesis; however, the mitochondrial KAS performs all of the condensation reactions performed by chloroplasts KAS I, KAS II, and KAS III.

As used herein, total saturated fatty acid content (abbreviated as “Total Sats”) refers to the total of myristic acid (C14:0), palmitic acid (C16:0), stearic acid (C18:0), arachidic acid (C20:0), behenic acid (C22:0), and lignoceric acid (C24:0). For example, Brassica plants described herein can produce oils having a total saturated fatty acid content of about 2.5 to about 5.5%, about 3 to about 5%, about 3 to about 4.5%, about 3.25 to about 3.75%, about 3 to about 3.5%, about 3.6 to about 5%, about 4 to about 5.5%, or about 4 to about 5%. Oils having low or no total saturated fatty acid content have improved nutritional quality and can help consumers reduce their intake of saturated fatty acids.

As described herein, Brassica plants can be made that yield seed oils having a low total saturated fatty acid content in combination with a typical (60%-70%), mid (71%-80%), or high (>80%) oleic acid content. Such Brassica plants can produce seed oils having a fatty acid content tailored to the desired end use of the oil (e.g., frying or food applications). For example, Brassica plants can be produced that yield seeds having a low total saturated fatty acid content, an oleic acid content of about 60 to about 70%, and an α-linolenic acid content of about 2 to about 5%. Total polyunsaturates (i.e., total of linoleic acid and α-linolenic acid) in such seeds typically is <35%. Canola oils having such fatty acid contents are particularly useful for frying applications due to the polyunsaturated content, which is low enough to have improved oxidative stability for frying yet high enough to impart the desired fried flavor to the food being fried, and are an improvement over commodity type canola oils. The fatty acid content of commodity type canola oils typically is about 6 to about 8% total saturated fatty acids, about 55 to about 65% oleic acid, about 22 to about 30% linoleic acid, and about 7 to about 10% α-linolenic acid.

Brassica plants also can be produced that yield seeds having a low total saturated fatty acid content (e.g., about 1.6 to about 3%, about 2 to about 4%, and/or about 3 to about 6%), mid oleic acid content (e.g., about 71 to about 80%) and a low α-linolenic acid content (e.g., about 2 to about 5.0%). Canola oils having such fatty acid contents have an oxidative stability that is higher than oils with a lower oleic acid content or commodity type canola oils, and are useful for coating applications (e.g., spray-coatings), formulating food products, or other applications where shelf-life stability is desired. In addition, Brassica plants can be produced that yield seeds having a low total saturated fatty acid content, high oleic acid content (e.g., about 81 to about 90% oleic acid) and an α-linolenic acid content of about 2 to about 5%. Canola oils having a low total saturated fatty acid content, high oleic acid, and low α-linolenic acid content are particularly useful for food applications requiring high oxidative stability and a reduced saturated fatty acid content.

### Brassica Plants

Brassica plants described herein comprise either or both of QTL1 or QTL2, which contribute to the fatty acid profile of their seed oil. Such plants include those having either of QTL1 or QTL2 and a reduced activity of fatty acyl-ACP thioesterase A2 (FATA2) and/or reduced activity of fatty acyl-ACP thioesterase B (FATB). It is understood that, throughout the disclosure, reference to “plant” or “plants” includes progeny, i.e., descendants of a particular plant or plant line, as well as cells or tissues from the plant unless stated otherwise. Progeny of an instant plant include seeds formed on F₁, F₂, F₃, F₄ and subsequent generation plants, or seeds formed on BC₁, BC₂, BC₃, and subsequent generation plants. Seeds produced by a plant can be grown and then selfed (or outcrossed and selfed, or doubled through formation of double haploids (“DH”)) to obtain seeds homozygous for a mutant allele. The term “allele” or “alleles” refers to one or more alternative forms of a locus. As used herein, a “line” is a group of plants that display little or no genetic variation between individuals for at least one trait. Such lines may be created by several generations of self-pollination and selection, or vegetative propagation from a single parent using tissue or cell culture techniques. As used herein, the term “variety” refers to a line which is used for commercial production, and includes hybrid varieties and open-pollinated varieties.

The present disclosure includes and provides for methods of selecting or breeding Brassica plants for the presence or absence of all or part of QTL1 and/or QTL2 of Salomon (ATCC deposit ATCC PTA-11453) that may be employed, for example, as molecular guided breeding programs. Such methods of selecting or breeding Brassica plants comprise obtaining one or more Brassica plants and assessing their DNA to determine the presence or absence of all or part of QTL1 (on chromosome N1) and/or all or part of QTL2 (on chromosome N19). Based upon the results of the assessment, plants are selected for the presence or absence of all or part of QTL1 and/or QTL2 to produce one or more selected plants. Such methods may be used, for example, to determine which progeny resulting from a cross have all or part of QTL1 and/or QTL2, and accordingly to guide preparation of plants having one or both of those QTLs in combination with other desirable genes/traits.

In one embodiment, determining the presence of all or part of QTL1 in plants comprises determining the presence of mutations appearing in Salomon in the QTL 1 region that do not appear in its parent, line 15.24. In another embodiment, determining the presence of all or part of QTL2 in plants comprises determining the presence of mutations in the QTL2 region appearing in Salomon that do not appear in its parent, 1764. Accordingly, plants can be selected by assessing them for the presence of one or more individual SNPs appearing in Table 27 for QTL1 or Tabe 28 for QTL2. Plants may also be assessed for larger portions of those QTL regions (e.g., regions encompassing one or more SNPs in Tables 27 and/or 28).

In one embodiment, plants may be selected by determining the presence of one, two, three, four, five, ten, fifteen or more QTL1 markers selected from the group consisting of:

20772548, 20780679, 20843387, 20874199, 20874571, 20924967, 20979545, 21000713, 21057761, 21080816, 21126589, 21175577, 21244175, 21273898, 21301953, 21342623, 21378815, 21425310, 21491979, and 21549878.

In one embodiment, plants may be selected by determining the presence of one, two, three, four, five, ten, fifteen or more QTL2 markers selected from the group consisting of:

11538807, 11763228, 11855685, 12010676, 12205222, 12219881, 12355162, 12378335, 12507143, 12615691, 12847514, 12979251, 13003942, 13008581, 13207412, 13364132, 13429175, 13429687, 13460532, 13475876, 13504886, and 13704881.

In one embodiment, plants may be assessed to determine the presence or absence of QTL1 chromosomal segments including a segment selected from the chromosomal regions: beginning with SNP 20772548 and ending with SNP 22780181; beginning with SNP 20772548 and ending with SNP 21342623; beginning with SNP 20772548 and ending with SNP 21126589: beginning with SNP 20772548 and ending with SNP 21000713; beginning with SNP 20772548 and ending with SNP 20874571; and beginning with SNP 20772548 and ending with SNP 21000713.

In one embodiment, plants may be assessed to determine the presence or absence of QTL2 chromosomal segments including a segment selected from the chromosomal regions: beginning with SNP 11538807 and ending with SNP 18172630; beginning with SNP 11538807 and ending with SNP15988083; beginning with SNP 11538807 and ending with SNP 13704881: beginning with SNP 11538807 and ending with SNP 13008581; beginning with SNP 11538807 and ending with SNP 12847514; and beginning with SNP 12219881 and ending with SNP 13008581,

Any suitable method known in the art may be used to assess plants to determine if they comprise all or part of QTL1 and or QTL2. Some suitable methods include, but are not limited to, sequencing, hybridization assays, polymerase chain reaction (PCR), ligase chain reaction (LCR), and genotyping-by-sequencing (GBS).

In addition to selecting plants based upon the presence or absence of all or part of QTL1 or QTL2, the plants may be assessed for their fatty acid content. More specifically, plants may be assessed for their fatty acid profile (i.e., the types and/or relative amount of fatty acids they produce, typically in their seed) and their total fatty acid production. Among the fatty acids that can be examined are saturated fats (e.g., 16:0 and 18:0), monounsaturated fats, and poly unsaturated fats. Analysis of fatty acid profile and/or content may be directed to one or more selected plants (or their seed) selected and/or the progeny of such plants. In some embodiments, the Brassica plants described herein comprise as one or more alleles QTL1 and/or QTL2 of the Salomon line and further comprise a mutant allele for a fatty acyl-ACP thioesterase. Fatty acyl-ACP thioesterases hydrolyze acyl-ACPs in the chloroplast to release the newly synthesized fatty acid from ACP, effectively removing it from further chain elongation in the plastid. The free fatty acid can then leave the plastid, become bound to CoenzymeA (CoA) and enter the Kennedy pathway in the endoplasmic reticulum (ER) for triacylglycerol (TAG) biosynthesis. Members of the FATA family prefer oleoyl (C18:1) ACP substrates with minor activity towards 18:0 and 16:0 ACPs, while members of the FATB family hydrolyze primarily saturated acyl-ACPs between 8 and 18 carbons in length. See Jones et al., Plant Cell 7:359-371 (1995); Ginalski and Rychlewski, Nucleic Acids Res 31:3291-3292 (2003); and Voelker T in Genetic Engineering (Setlow, J K, ed) Vol 18, 111-133, Plenum Publishing Corp., New York (2003).

Reduced activity, including absence of detectable activity, of FATA2 or FATB can be achieved by modifying an endogenous fatA2 or fatB alleles. An endogenous fatA2 or fat3B alleles can be modified by, for example, mutagenesis or by using homologous recombination to replace an endogenous plant gene with a variant containing one or more mutations (e.g., produced using site-directed mutagenesis). See, e.g., Townsend et al., Nature 459:442-445 (2009); Tovkach et al., Plant J., 57:747-757 (2009); and Lloyd et al., Proc. Natl. Acad. Sci. USA, 102:2232-2237 (2005). Similarly, for other genes discussed herein, the endogenous allele can be modified by mutagenesis or by using homologous recombination to replace an endogenous gene with a variant. Modified alleles obtained through mutagenesis are referred to herein as mutant alleles.

Reduced activity, including absence of detectable activity, can be inferred from the decreased level of saturated fatty acids in the seed oil compared with seed oil from a corresponding control plant. In one embodiment, the Brassica line Topas, transmitted to the ATCC on Nov. 20, 2013, Accession No. PTA-120738 can be used as a control plant. Alternatively, reduced activity can be assessed in plant extracts using assays for fatty acyl-ACP hydrolysis. See, for example, Bonaventure et al., Plant Cell 15:1020-1033 (2003); and Eccleston and Ohlrogge, Plant Cell 10:613-622 (1998).

Genetic mutations can be introduced within a population of seeds or regenerable plant tissue using one or more mutagenic agents. Suitable mutagenic agents include, for example, ethyl methane sulfonate (EMS), methyl N-nitrosoguanidine (MNNG), ethidium bromide, diepoxybutane, ionizing radiation, x-rays, UV rays and other mutagens known in the art. In some embodiments, a combination of mutagens, such as EMS and MNNG, can be used to induce mutagenesis. The treated population, or a subsequent generation of that population, can be screened for reduced thioesterase activity that results from the mutation, e.g., by determining the fatty acid profile of the population and comparing it to a corresponding non-mutagenized population. Mutations can be in any portion of a gene, including coding sequence, intron sequence and regulatory elements, that renders the resulting gene product non-functional or with reduced activity. Suitable types of mutations include, for example, insertions or deletions of nucleotides, and transitions or transversions in the wild-type coding sequence. Such mutations can lead to deletion or insertion of amino acids, and conservative or non-conservative amino acid substitutions in the corresponding gene product. In some embodiments, the mutation is a nonsense mutation, which results in the introduction of a stop codon (TGA, TAA, or TAG) and production of a truncated polypeptide. In some embodiments, the mutation is a splice site mutation which alters or abolishes the correct splicing of the pre-mRNA sequence, resulting in a protein of different amino acid sequence than the wild type. For example, one or more exons may be skipped during RNA splicing, resulting in a protein lacking the amino acids encoded by the skipped exons. Alternatively, the reading frame may be altered by incorrect splicing, one or more introns may be retained, alternate splice donors or acceptors may be generated, splicing may be initiated at an alternate position, or alternative polyadenylation signals may be generated. In some embodiments, more than one mutation or more than one type of mutation is introduced.

Insertions, deletions, or substitutions of amino acids in a coding sequence may, for example, disrupt the conformation of essential alpha-helical or beta-pleated sheet regions of the resulting gene product Amino acid insertions, deletions, or substitutions also can disrupt binding, alter substrate specificity, or disrupt catalytic sites important for gene product activity. It is known in the art that the insertion or deletion of a larger number of contiguous amino acids is more likely to render the gene product non-functional, compared to a smaller number of inserted or deleted amino acids. Non-conservative amino acid substitutions may replace an amino acid of one class with an amino acid of a different class. Non-conservative substitutions may make a substantial change in the charge or hydrophobicity of the gene product. Non-conservative amino acid substitutions may also make a substantial change in the bulk of the residue side chain, e.g., substituting an alanine residue for an isoleucine residue.

Examples of non-conservative substitutions include the substitution of a basic amino acid for a non-polar amino acid, or a polar amino acid for an acidic amino acid. Because there are only 20 amino acids encoded in a gene, substitutions that result in reduced activity may be determined by routine experimentation, incorporating amino acids of a different class in the region of the gene product targeted for mutation.

In some embodiments, the Brassica plants described herein comprise as one or more alleles QTL1 and/or QTL2 of the Salomon line and further comprise a mutant allele at a FATA2 locus, wherein the mutant allele results in the production of a FATA2 polypeptide having reduced thioesterase activity relative to a corresponding wild-type FATA2 polypeptide. In such embodiments, the plants are not plants of the B. napus Salomon line, the 1764 line, the 15.24 line, or any other plants comprising QTL1 and/or QTL2 of the Salomon line set forth in WO 2011/075716. Plants of such an embodiment may comprise as an allele QTL1 with the proviso they do not comprise as an allele QTL2 of Salomon; or alternatively, such plants may comprise as an allele QTL2 of Salomon with the proviso they do not comprise as an allele QTL1.

Where a mutant allele at a FATA2 locus is present, the mutant allele can include a nucleic acid that encodes a FATA2 polypeptide having a non-conservative substitution within a helix/4-stranded sheet (4HBT) domain (also referred to as a hot-dog domain) or a non-conservative substitution of a residue affecting catalytic activity or substrate specificity. For example, a Brassica plant can contain a mutant allele that includes a nucleic acid encoding a FATA2b polypeptide having a substitution in a region (SEQ ID NO:29) of the polypeptide corresponding to residues 242 to 277 of the FATA2 polypeptide (as numbered based on the alignment to the Arabidopsis thaliana FATA2 polypeptide set forth in GenBank Accession No. NP_193041.1, protein (SEQ ID NO:30); GenBank Accession No. NM_117374, mRNA). This region of FATA2 is highly conserved in Arabidopsis and Brassica. In addition, many residues in this region are conserved between FATA and FATB, including the aspartic acid at position 259, asparagine at position 263, histidine at position 265, valine at position 266, asparagine at position 268, and tyrosine at position 271 (as numbered based on the alignment to SEQ ID NO:30). See also FIG. 3. The asparagine at position 263 and histidine at position 265 are part of the catalytic triad, and the arginine at position 256 is involved in determining substrate specificity. See also Mayer and Shanklin, BMC Plant Biology 7:1-11 (2007). SEQ ID NO:31 sets forth the predicted amino acid sequence of the Brassica FATA2b polypeptide encoded by exons 2-6 and corresponding to residues 121 to 343 of the A. thaliana sequence set forth in SEQ ID NO:30. For example, the FATA2 polypeptide can have a substitution of a leucine residue for proline at the position corresponding to position 255 of the Arabidopsis FATA2 polypeptide (i.e., position 14 of SEQ ID NO:29 or position 135 of SEQ ID NO:31). The proline in the B. napus sequence corresponding to position 255 in Arabidopsis is conserved among B. napus, B. rapa, B. juncea, Zea mays, Sorghum bicolor, Oryza sativa Indica (rice), Triticum aestivum, Glycine max, Jatropha (tree species), Carthamus tinctorius, Cuphea hookeriana, Iris tectorum, Perilla frutescens, Helianthus annuus, Garcinia mangostana, Picea sitchensis, Physcomitrella patens subsp. Patens, Elaeis guineensis, Vitis vinifera, Elaeis oleifera, Camellia oleifera, Arachis hypogaea, Capsicum annuum, Populus trichocarpa, and Diploknema butyracea. As described in Example 2, the mutation at position 255 is associated with a low total saturated fatty acid phenotype, low stearic acid phenotype, low arachidic acid phenotype, and an increased eicosenoic acid phenotype. The stearic acid content phenotype is negatively correlated with the eicosenoic acid phenotype.

In some embodiments, where a mutant allele at a FATA2 locus is present, the locus has at least 90% (e.g., at least 91, 92, 93, 94, 95, 96, 97, 98, or 99%) sequence identity to the nucleotide sequence set forth in SEQ ID NO:28 or SEQ ID NO:32. The nucleotide sequences set forth in SEQ ID NOs:28 and 32 are representative nucleotide sequences from the fatA2b gene from B. napus line 15.24. As used herein, the term “sequence identity” refers to the degree of similarity between any given nucleic acid sequence and a target nucleic acid sequence. The degree of similarity is represented as percent sequence identity. Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (B12seq) program from the stand-alone version of BLASTZ containing BLASTN and BLASTP. This stand-alone version of BLASTZ can be obtained from Fish & Richardson's web site (World Wide Web at fr.com/blast) or the U.S. government's National Center for Biotechnology Information web site (World Wide Web at ncbi.nlm nih gov). Instructions explaining how to use the B12seq program can be found in the readme file accompanying BLASTZ.

B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default settings. The following command will generate an output file containing a comparison between two sequences: C:\B12seq -i c:\seq1.txt -j c:\seq2.txt -p blastn -o c:\output.txt -q -1-r 2. If the target sequence shares homology with any portion of the identified sequence, then the designated output file will present those regions of homology as aligned sequences. If the target sequence does not share homology with any portion of the identified sequence, then the designated output file will not present aligned sequences.

Once aligned, a length is determined by counting the number of consecutive nucleotides from the target sequence presented in alignment with a portion of the identified sequence, starting with any matched position and ending with any other matched position. A matched position is any position where an identical nucleotide is presented in both the target and identified sequences. Gaps presented in the target sequence are not counted since gaps are not nucleotides. Likewise, gaps presented in the identified sequence are not counted since target sequence nucleotides are counted, not nucleotides from the identified sequence.

The percent identity over a particular length is determined by counting the number of matched positions over that length and dividing that number by the length followed by multiplying the resulting value by 100. For example, if (i) a 500-base nucleic acid target sequence is compared to a subject nucleic acid sequence, (ii) the B12seq program presents 200 bases from the target sequence aligned with a region of the subject sequence where the first and last bases of that 200-base region are matches, and (iii) the number of matches over those 200 aligned bases is 180, then the 500-base nucleic acid target sequence contains a length of 200 and a sequence identity over that length of 90% (i.e., 180/200×100=90).

It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

In some embodiments, the Brassica plants described herein comprise as one or more alleles QTL1 and/or QTL2 of the Salomon line and further comprise a mutant allele at a FATB locus, wherein the mutant allele results in the production of a FATB polypeptide having reduced thioesterase activity relative to a corresponding wild-type FATB polypeptide. In some embodiments, a Brassica plant contains mutant alleles at two or more different FATB loci. In some embodiments, a Brassica plant contains mutant alleles at three different FATB loci or contains mutant alleles at four different FATB loci. B. napus contains 6 different FATB isoforms (i.e., different forms of the FATB polypeptide at different loci), which are called isoforms 1-6 herein. SEQ ID NOs:18-21 and 26-27 set forth the nucleotide sequences encoding FATB isoforms 1-6, respectively, of B. napus. The nucleotide sequences set forth in SEQ ID NOs:18-21 and 26-27 have 82% to 95% sequence identity as measured by the ClustalW algorithm. In such embodiments, the plants are not plants of the B. napus Salomon line, the 1764 line, the 15.24 line, or any other plants comprising QTL1 and/or QTL2 of the Salomon line set forth in WO2011/075716. Plants of such an embodiment may comprise as an allele QTL1 with the proviso they do not comprise as an allele QTL2 of Salomon; or alternatively, such plants may comprise as an allele QTL2 of Salomon with the proviso they do not comprise as an allele QTL1.

For example, a Brassica plant comprising a FATB mutation can have a mutation in a nucleotide sequence encoding FATB isoform 1, isoform 2, isoform 3, isoform 4, isoform 5, or isoform 6. In some embodiments, a plant can have a mutation in a nucleotide sequence encoding isoforms 1 and 2; 1 and 3; 1 and 4; 1 and 5; 1 and 6; 2 and 3; 2 and 4; 2 and 5; 2 and 6; 3 and 4; 3 and 5; 3 and 6; 4 and 5; 4 and 6; 5 and 6; 1, 2, and 3; 1, 2, and 4; 1, 2, and 5; 1, 2, and 6; 2, 3, and 4; 2, 3, and 5; 2, 3, and 6; 3, 4, and 5; 3, 4, and 6; 3, 5, and 6; 4, 5, and 6; 1, 2, 3, and 4; 1, 2, 3, and 5; 1, 2, 3, and 6; 1, 2, 4, and 5; 1, 2, 4, and 6; 1, 3, 4 and 5; 1, 3, 4, and 6; 1, 4, 5, and 6; 2, 3, 4, and 5; 2, 3, 4 and 6; or 3, 4, 5, and 6. In some embodiments, a Brassica plant can have a mutation in nucleotide sequences encoding FATB isoforms 1, 2, and 3; 1, 2, and 4; 2, 3, and 4; or 1, 2, 3, and 4. In some embodiments, a mutation results in deletion of a 4HBT domain or a portion thereof of a FATB polypeptide. FATB polypeptides typically contain a tandem repeat of the 4HBT domain, where the N-terminal 4HBT domain contains residues affecting substrate specificity (e.g., two conserved methionines, a conserved lysine, a conserved valine, and a conserved serine) and the C-terminal 4HBT domain contains residues affecting catalytic activity (e.g., a catalytic triad of a conserved asparagine, a conserved histidine, and a conserved cysteine) and substrate specificity (e.g., a conserved tryptophan). See Mayer and Shanklin, J. Biol. Chem. 280:3621-3627 (2005). In some embodiments, the mutation results in a non-conservative substitution of a residue in a 4HBT domain or a residue affecting substrate specificity. In some embodiments, the mutation is a splice site mutation. In some embodiments, the mutation is a nonsense mutation in which a premature stop codon (TGA, TAA, or TAG) is introduced, resulting in the production of a truncated polypeptide.

SEQ ID NOs:1-4 set forth the nucleotide sequences encoding isoforms 1-4, respectively, and containing exemplary nonsense mutations that result in truncated FATB polypeptides. SEQ ID NO:1 is the nucleotide sequence of isoform 1 having a mutation at position 154, which changes the codon from CAG to TAG. SEQ ID NO:2 is the nucleotide sequence of isoform 2 having a mutation at position 695, which changes the codon from CAG to TAG. SEQ ID NO:3 is the nucleotide sequence of isoform 3 having a mutation at position 276, which changes the codon from TGG to TGA. SEQ ID NO:4 is the nucleotide sequence of isoform 4 having a mutation at position 336, which changes the codon from TGG to TGA.

Two or more different mutant FATB alleles may be combined in a plant by making a genetic cross between mutant lines. For example, a plant having a mutant allele at a FATB locus encoding isoform 1 can be crossed or mated with a second plant having a mutant allele at a FATB locus encoding isoform 2. Seeds produced from the cross are planted and the resulting plants are selfed in order to obtain progeny seeds. These progeny seeds can be screened in order to identify those seeds carrying both mutant alleles. In some embodiments, progeny are selected over multiple generations (e.g., 2 to 5 generations) to obtain plants having mutant alleles at two different FATB loci. Similarly, a plant having mutant alleles at two or more different FATB isoforms can be crossed with a second plant having mutant alleles at two or more different FATB alleles, and progeny seeds can be screened to identify those seeds carrying mutant alleles at four or more different FATB loci. Again, progeny can be selected for multiple generations to obtain the desired plant.

In some embodiments, the Brassica plants described herein that comprise as one or more allele QTL1 and/or QTL2 of the Salomon line further comprise a mutant allele at a FATA2 locus and mutant alleles at two or more (e.g., three or four) different FATB loci can be combined in a plant. For example, a plant having a mutant allele at a FATA2 locus can be crossed or mated with a second plant having mutant alleles at two or more different FATB loci. Seeds produced from the cross are planted and the resulting plants are selfed in order to obtain progeny seeds. These progeny seeds can be screened in order to identify those seeds carrying mutant FATA2 and FATB alleles. Progeny can be selected over multiple generations (e.g., 2 to 5 generations) to obtain plants having a mutant allele at a FATA2 locus and mutant alleles at two or more different FATB loci. As described herein, plants having a mutant allele at a FATA2b locus and mutant alleles at three or four different FATB loci have a low total saturated fatty acid content that is stable over different growing conditions, i.e., is less subject to variation due to warmer or colder temperatures during the growing season. Due to the differing substrate profiles of the FatB and FatA enzymes with respect to 16:0 and 18:0, respectively, plants having mutations in FatA2 and FatB loci exhibit a substantial reduction in amounts of both 16:0 and 18:0 in seed oil. In such embodiments, the plants are not plants of the B. napus Salomon line, the 1764 line, the 15.24 line, or any other plants comprising QTL1 and/or QTL2 of the Salomon line set forth in WO 2011/075716. Plants of such an embodiment may comprise as an allele QTL1 with the proviso they do not comprise as an allele QTL2 of Salomon or, alternatively, such plants may comprise as an allele QTL2 of Salomon with the proviso they do not comprise as an allele QTL1.

The Brassica plants described herein comprising as one or more alleles QTL1 or QTL2 of the Salomon line may further comprise mutant alleles at FATA2 and/or FATB loci and also may include mutant alleles at loci controlling fatty acid desaturase activity such that the oleic acid and linolenic acid levels can be tailored to the end use of the oil. For example, such Brassica plants also can exhibit reduced activity of delta-15 desaturase (also known as FADS), which is involved in the enzymatic conversion of linoleic acid to α-linolenic acid. The gene encoding delta-15 fatty acid desaturase is referred to as fad3 in Brassica and Arabidopsis. Sequences of higher plant fad3 genes are disclosed in Yadav et al., Plant Physiol., 103:467-476 (1993), WO 93/11245, and Arondel et al., Science, 258:1353-1355 (1992). Decreased activity, including absence of detectable activity, of delta-15 desaturase can be achieved by mutagenesis. Decreased activity, including absence of detectable activity, can be inferred from the decreased level of linolenic acid (product) and in some cases, increased level of linoleic acid (the substrate) in the plant compared with a corresponding control plant (e.g., the Brassica line Topas). For example, parent plants can contain the mutation from the APOLLO or STELLAR B. napus variety (both developed at the University of Manitoba, Manitoba, Canada) that confers low linolenic acid. In some embodiments, the parents contain the fad3A and/or fad3B mutation from IMC02 that confers a low linolenic acid phenotype. IMC02 contains a mutation in both the fad3A and fad3B genes. The fad3A gene contains a C to T mutation at position 2565, numbered from the ATG in genomic DNA, resulting in the substitution of a cysteine for arginine at position 275 of the encoded FAD3A polypeptide. The fad3B gene contains a G to A mutation at position 3053, numbered from the ATG in genomic DNA, located in the exon-intron splice site recognition sequence. IMC02 was obtained from a cross of IMC01×Westar. See Example 3 of U.S. Pat. No. 5,750,827. IMC01 was deposited with the ATCC under Accession No. 40579. IMC02 was deposited with the ATCC under Accession No. PTA-6221. In such embodiments, the plants are not plants of the B. napus Salomon line, the 1764 line, the 15.24 line, or any other plants comprising QTL1 and/or QTL2 of the Salomon line set forth in WO 2011/075716. Plants of such an embodiment may comprise as an allele QTL1 with the proviso they do not comprise as an allele QTL2 of Salomon or, alternatively, such plants may comprise as an allele QTL2 of Salomon with the proviso they do not comprise as an allele QTL1.

In some embodiments, the Brassica plants described herein comprising as one or more alleles QTL1 or QTL2 of the Salomon line further comprise a mutant allele at a FATA2 locus and a mutant allele at a FAD3 locus. For example, a Brassica plant can contain a mutant allele at a FATA2 locus and a mutant allele at a FAD3 locus that contains a nucleic acid encoding a FAD3 polypeptide with a cysteine substituted for arginine at position 275 and/or a nucleic acid having a mutation in an exon-intron splice site recognition sequence. A Brassica plant also can contain mutant alleles at two or more different FATB loci (three or four different loci) and a FAD3 locus that contains a nucleic acid encoding a FAD3 polypeptide with a cysteine substituted for arginine at position 275 and/or a nucleic acid having a mutation in an exon-intron splice site recognition sequence. A Brassica plant also contains a mutant allele at a FATA2 locus, mutant alleles at two or more different FATB loci (three or four different loci) and a FAD3 locus that contains a nucleic acid encoding a FAD3 polypeptide with a cysteine substituted for arginine at position 275 and/or a nucleic acid having a mutation in an exon-intron splice site recognition sequence. In such embodiments, the plants are not plants of the B. napus Salomon line, the 1764 line, the 15.24 line, or any other plants comprising QTL1 and/or QTL2 of the Salomon line set forth in WO 2011/075716. Plants of such an embodiment may comprise as an allele QTL1 with the proviso they do not comprise as an allele QTL2 of Salomon or, alternatively, such plants may comprise as an allele QTL2 of Salomon with the proviso they do not comprise as an allele QTL1.

In other embodiments, Brassica plants comprising as one or more alleles QTL1 and/or QTL2 of the Salomon line also can have decreased activity of a delta-12 desaturase, which is involved in the enzymatic conversion of oleic acid to linoleic acid, to confer a mid or high oleic acid content in the seed oil. Brassica plants can exhibit reduced activity of delta-12 desaturase (also known as FAD2) in combination with reduced activity of FATA2 and/or FATB. The sequences for the wild-type fad2 genes from B. napus (termed the D form and the F form) are disclosed in WO 98/56239. A reduction in delta-12 desaturase activity, including absence of detectable activity, can be achieved by mutagenesis. Decreased delta-12 desaturase activity can be inferred from the decreased level of linoleic acid (product) and increased level of oleic acid (substrate) in the plant compared with a corresponding control plant. Non-limiting examples of suitable fad2 mutations include the G to A mutation at nucleotide 316 within the fad2-D gene, which results in the substitution of a lysine residue for glutamic acid in a HECGH (SEQ ID NO:5) motif. Such a mutation is found within the variety IMC129, which has been deposited with the ATCC under Accession No. 40811. Another suitable fad2 mutation can be the T to A mutation at nucleotide 515 of the fad2-F gene, which results in the substitution of a histidine residue for leucine in a KYLNNP (SEQ ID NO:6) motif (amino acid 172 of the Fad2 F polypeptide). Such a mutation is found within the variety Q508. See U.S. Pat. No. 6,342,658. Another example of a fad2 mutation is the G to A mutation at nucleotide 908 of the fad2-F gene, which results in the substitution of a glutamic acid for glycine in the DRDYGILNKV (SEQ ID NO:7) motif (amino acid 303 of the Fad2 F polypeptide). Such a mutation is found within the variety Q4275, which has been deposited with the ATCC under Accession No. 97569. See U.S. Pat. No. 6,342,658. Another example of a suitable fad2 mutation can be the C to T mutation at nucleotide 1001 of the fad2-F gene (as numbered from the ATG), which results in the substitution of an isoleucine for threonine (amino acid 334 of the Fad2 F polypeptide). Such a mutation is found within the high oleic acid variety Q7415. In such embodiments, the plants are not plants of the B. napus Salomon line, the 1764 line, the 15.24 line, or any other plants comprising QTL1 and/or QTL2 of the Salomon line set forth in WO 2011/075716. Plants of such an embodiment may comprise as an allele QTL1 with the proviso they do not comprise as an allele QTL2 of Salomon or, alternatively, such plants may comprise as an allele QTL2 of Salomon with the proviso they do not comprise as an allele QTL1.

Typically, the presence of one of the fad2-D or fad2-F mutations confers a mid-oleic acid phenotype (e.g., 70-80% oleic acid) to the seed oil, while the presence of both fad2-D and fad2-F mutations confers a higher oleic acid phenotype (e.g., >80% oleic acid). For example, Q4275 contains the fad2-D mutation from IMC129 and a fad2-F mutation at amino acid 303. Q508 contains fad2-D mutation from IMC129 and a fad2-F mutation at amino acid 172. Q7415 contains the fad2-D mutation from IMC129 and a fad2-F mutation at amino acid 334. Each of the varieties Q4275, Q508 and Q7415 have a mid-oleic acid phenotype. In contrast, the presence of fad2 mutations in Q4275, Q508, and Q7415 confers a high oleic acid phenotype of greater than 80% oleic acid.

Thus, in some embodiments, the Brassica plants described herein contain as one or more alleles QTL1 or QTL2 of the Salomon line and further comprise a mutant allele at a FATA2 locus (e.g., FATA2b locus) and a mutant allele at a FAD2 locus, with the proviso that the plants do not comprise both QTL1 and QTL2. For example, a Brassica plant can comprise either QTL1 or QTL2, and further comprise a mutant allele at a FATA2 locus and a mutant allele at a FAD2 locus described above. The Brassica plants described herein may also comprise either QTL1 or QTL2, and further comprise mutant alleles at two or more different FATB loci (three or four different loci) and a FAD2 locus described above. The Brassica plants described herein may also comprise either QTL1 or QTL2, and further comprise a mutant allele at a FATA2 locus, mutant alleles at two or more different FATB loci (three or four different loci) and a mutant allele at a FAD2 locus described above. In some embodiments, the Brassica plants described herein may also comprise either QTL1 or QTL2, and further comprise a mutant allele at a FATA2 locus, a mutant allele at a FAD2 locus, and a mutant allele at a FADS locus described above. The Brassica plants described herein may also comprise either QTL1 or QTL2, and further comprise mutant alleles at two or more different FATB loci (three or four different loci), mutant alleles at FAD2 loci, and mutant alleles at FAD3 loci described above. The Brassica plants described herein may also comprise either QTL1 or QTL2, and further comprise a mutant allele at a FATA2 locus, mutant alleles at two or more different FATB loci (three or four different loci), mutant alleles at FAD2 loci, and mutant alleles at FAD3 loci described above. In such embodiments, the plants are not plants of the B. napus Salomon line, the 1764 line, the 15.24 line, or any other plants comprising QTL1 and/or QTL2 of the Salomon line set forth in WO2011/075716.

The plants described herein are non-transgenic to the extent that they are derived by mutagenesis. Transgenic” or “genetically modified organisms” (GMO) as used herein are organisms whose genetic material has been altered using techniques generally known as “recombinant DNA technology.” Recombinant DNA technology is the ability to combine DNA molecules from different sources into one molecule ex vivo (e.g., in a test tube). This terminology generally does not cover organisms whose genetic composition has been altered by conventional cross-breeding or by “mutagenesis” breeding, as these methods predate the discovery of recombinant DNA techniques. See World Health Organization, Biorisk Management Laboratory Biosecurity Guidance, 2006 World Health Organization (WHO/CDS/EPR/2006.6). “Non-transgenic” as used herein refers to plants and food products derived from plants that are not “transgenic” or “genetically modified organisms.”

The plants described herein may be modified and/or selected to display a herbicide tolerance trait. That trait can be introduced by selection with the herbicide for which tolerance is sought, or by transgenic means where the genetic basis for the tolerance has been identified. Accordingly, the plants described herein, or parts thereof such as cells or protoplasts, may display tolerance to a herbicide selected from the group consisting of imidazolinone, dicamba, cyclohexanedione, sulfonylurea, glyphosate, glufosinate, phenoxy proprionic acid, L-phosphinothricin, triazine and benzonitrile. Where the plants have been genetically modified to acquire herbicide tolerance by transgenic means they may be non-transgenic to the extent of all other traits except herbicide tolerance.

### Production of Hybrid Brassica Varieties

Hybrid Brassica varieties can be produced by preventing self-pollination of female parent plants (i.e., seed parents), permitting pollen from male parent plants to fertilize such female parent plants, and allowing F₁ hybrid seeds to form on the female plants. Self-pollination of female plants can be prevented by emasculating the flowers at an early stage of flower development. Alternatively, pollen formation can be prevented on the female parent plants using a form of male sterility. For example, male sterility can be cytoplasmic male sterility (CMS), nuclear male sterility, molecular male sterility (wherein a transgene inhibits microsporogenesis and/or pollen formation), or be produced by self-incompatibility. Female parent plants containing CMS are particularly useful. CMS can be, for example, of the ogu (Ogura), nap, pol, tour, or mur type. See, for example, Pellan-Delourme and Renard, 1987, Proc. 7^{th} Int. Rapeseed Conf, Poznan, Poland, p. 199-203, and Pellan-Delourme and Renard, 1988, Genome 30:234-238, for a description of Ogura type CMS. See Riungu and McVetty, 2003, Can. J. Plant Sci., 83:261-269 for a description of nap, pol, tour, and mur type CMS.

In embodiments in which the female parent plants are CMS, the male parent plants typically contain a fertility restorer gene to ensure that the F₁ hybrids are fertile. For example, when the female parent contains an Ogura type CMS, a male parent is used that contains a fertility restorer gene that can overcome the Ogura type CMS. Non-limiting examples of such fertility restorer genes include the Kosena type fertility restorer gene (U.S. Pat. No. 5,644,066) and Ogura fertility restorer genes (U.S. Pat. Nos. 6,229,072 and 6,392,127). In other embodiments in which the female parents are CMS, male parents can be used that do not contain a fertility restorer. F₁ hybrids produced from such parents are male sterile. Male sterile hybrid seed can be inter-planted with male fertile seed to provide pollen for seed-set on the resulting male sterile plants.

The methods described herein can be used to form single-cross Brassica F₁ hybrids. In such embodiments, the parent plants can be grown as substantially homogeneous adjoining populations to facilitate natural cross-pollination from the male parent plants to the female parent plants. The F₁ seed formed on the female parent plants is selectively harvested by conventional means. One also can grow the two parent plants in bulk and harvest a blend of F₁ hybrid seed formed on the female parent and seed formed on the male parent as the result of self-pollination. Alternatively, three-way crosses can be carried out wherein a single-cross F₁ hybrid is used as a female parent and is crossed with a different male parent that satisfies the fatty acid parameters for the female parent of the first cross. Here, assuming a bulk planting, the overall oleic acid content of the vegetable oil may be reduced over that of a single-cross hybrid; however, the seed yield will be further enhanced in view of the good agronomic performance of both parents when making the second cross. As another alternative, double-cross hybrids can be created wherein the F₁ progeny of two different single-crosses are themselves crossed. Self-incompatibility can be used to particular advantage to prevent self-pollination of female parents when forming a double-cross hybrid.

Hybrids described herein have good agronomic properties and exhibit hybrid vigor, which results in seed yields that exceed that of either parent used in the formation of the F₁ hybrid. For example, yield can be at least 10% (e.g., 10% to about 20%, 10% to about 15%, about 15% to about 20%, about 15% to about 25%, about 20% to about 30%, or about 25% to about 35%) above that of either one or both parents. In some embodiments, the yield exceeds that of open-pollinated spring canola varieties such as 46A65 (Pioneer) or Q2 (University of Alberta), when grown under similar growing conditions. For example, yield can be at least 10% (e.g., 10% to about 15% or about 15% to about 20%) above that of an open-pollinated variety.

Hybrids described herein typically produce seeds having very low levels of glucosinolates (<30 μmol/gram of de-fatted meal at a moisture content of 8.5%). In particular, hybrids can produce seeds having <20 μmol of glucosinolates/gram of de-fatted meal. As such, hybrids can incorporate mutations that confer low glucosinolate levels. See, for example, U.S. Pat. No. 5,866,762. Glucosinolate levels can be determined in accordance with known techniques, including high performance liquid chromatography (HPLC), as described in ISO 9167-1:1992(E), for quantification of total, intact glucosinolates, and gas-liquid chromatography for quantification of trimethylsilyl (TMS) derivatives of extracted and purified desulfoglucosinolates. Both the HPLC and TMS methods for determining glucosinolate levels analyze de-fatted or oil-free meal.

### Canola Oil

Brassica plants disclosed herein are useful for producing canola oils with low or no total saturated fatty acids. For example, oil obtained from seeds of Brassica plants described herein may have a total saturated fatty acid content of about 2.5 to about 5.5%, about 3 to about 5%, about 3 to about 4.5%, about 3.25 to about 3.75%, about 3 to about 3.5%, about 3.4 to about 3.7%, about 3.6 to about 5%, about 4 to about 5.5%, about 4 to about 5%, or about 4.25 to about 5.25%. In some embodiments, an oil has a total saturated fatty acid content of about 4 to about 5.5%, an oleic acid content of about 60 to about 70% (e.g., about 62 to about 68%, about 63 to about 67%, or about 65 to about 66%), and an α-linolenic acid content of about 2.5 to about 5%. In some embodiments, an oil has a total saturated fatty acid content of about 2.5 to about 5.5% (e.g., about 4 to about 5%), an oleic acid content of about 71 to about 80% (e.g., about 72 to about 78%, about 73 to about 75%, about 74 to about 78%, or about 75 to about 80%) and an α-linolenic acid content of about 2 to about 5.0% (e.g., about 2 to about 2.8%, about 2.25 to about 3%, about 2.5 to about 3%, about 3 to about 3.5%, about 3.25 to about 3.75%, about 3.5 to about 4%, about 3.75 to about 4.25%, about 4 to about 4.5%, about 4.25 to about 4.75%, about 4.5 to about 5%). In some embodiments, a canola oil can have a total saturated fatty acid content of about 2.5 to about 5.5%, an oleic acid content of about 78 to about 80%, and an α-linolenic acid content of no more than about 4% (e.g., about 2 to about 4%). In some embodiments, an oil has a total saturated fatty acid content of about 3.5 to about 5.5% (e.g., about 4 to about 5%), an oleic acid content of about 81 to about 90% (e.g., about 82 to about 88% or about 83 to about 87% oleic acid) and an α-linolenic acid content of about 2 to about 5% (e.g., about 2 to about 3% or about 3 to about 5%). In some embodiments, an oil has a total saturated fatty acid content of no more than about 3.7% (e.g., about 3.4 to about 3.7% or about 3.4 to about 3.6%) and an oleic acid content of about 72 to about 75%.

Low saturate oils obtained from seed of Brassica plants described herein can have a palmitic acid content of about 1.5 to about 3.5% (e.g., about 2 to about 3% or about 2.2 to about 2.4%). The stearic acid content of such oils can be about 0.5 to about 2.5% (e.g., about 0.5 to about 0.8%, about 1 to about 2%, or about 1.5 to about 2.5%).

Oils obtained from seed of Brassica plants described herein can have an eicosenoic acid content greater than about 1.6%, e.g., about 1.6 to about 1.9%, about 1.7 to about 2.3%, about 1.8 to about 2.3%, or about 1.9 to about 2.3%, in addition to a low total saturates content.

Oils obtained from seed of Brassica plants described herein can have a linoleic acid content of about 3 to about 20%, e.g., about 3.4 to about 5%, about 3.75 to about 5%, about 8 to about 10%, about 10 to about 12%, about 11 to about 13%, about 13 to about 16%, or about 14 to about 18%, in addition to a low total saturates content.

Oils obtained from seed of Brassica plants described herein have an erucic acid content of less than about 2% (e.g., less than about 1%, about 0.5%, about 0.2%, or about 0.1%) in addition to a low total saturates content.

The fatty acid composition of oil obtained from seed of Brassica plants can be determined by first crushing and extracting oil from seed samples (e.g., bulk seed samples of 10 or more seeds). TAGs in the seed are hydrolyzed to produce free fatty acids, which then can be converted to fatty acid methyl esters and analyzed using techniques known to the skilled artisan, e.g., gas-liquid chromatography (GLC) according to AOCS Procedure Ce 1e-91. Near infrared (NIR) analysis can be performed on whole seed according to AOCS Procedure Am-192 (revised 1999).

Seeds harvested from plants described herein can be used to make a crude canola oil or a refined, bleached, and deodorized (RBD) canola oil with a low or no total saturated fatty acid content. Harvested canola seed can be crushed by techniques known in the art. The seed can be tempered by spraying the seed with water to raise the moisture to, for example, about 8.5%. The tempered seed can be flaked using a smooth roller with, for example, a gap setting of 0.23 to 0.27 mm. Heat may be applied to the flakes to deactivate enzymes, facilitate further cell rupturing, coalesce the oil droplets, or agglomerate protein particles in order to ease the extraction process. Typically, oil is removed from the heated canola flakes by a screw press to press out a major fraction of the oil from the flakes. The resulting press cake contains some residual oil.

Crude oil produced from the pressing operation typically is passed through a settling tank with a slotted wire drainage top to remove the solids expressed out with the oil in the screw pressing operation. The clarified oil can be passed through a plate and frame filter to remove the remaining fine solid particles. Canola press cake produced from the screw pressing operation can be extracted with commercial n-Hexane. The canola oil recovered from the extraction process is combined with the clarified oil from the screw pressing operation, resulting in a blended crude oil.

Free fatty acids and gums typically are removed from the crude oil by adding food grade phosphoric acid and heating the acidified oil in a batch refining tank. The acid serves to convert the non-hydratable phosphatides to a hydratable form, and to chelate minor metals that are present in the crude oil. The phosphatides and the metal salts are removed from the oil along with the soapstock. The oil-acid mixture is subsequently treated with sodium hydroxide solution to neutralize the free fatty acids and the remaining phosphoric acid in the acid-oil mixture. The neutralized free fatty acids, phosphatides, etc. (soapstock) are drained off from the neutralized oil. A water wash may be done to further reduce the soap content of the oil. The oil may be bleached and deodorized before use, if desired, by techniques known in the art.

Oils obtained from the Brassica plant described herein can have increased oxidative stability, which can be measured using, for example, an Oxidative Stability Index Instrument (e.g., from Omnion, Inc., Rockland, Mass.) according to AOCS Official Method Cd 12b-92 (revised 1993). Oxidative stability is often expressed in terms of “AOM” hours.

### Food Compositions

The present disclosure also includes and provides for food compositions containing the oils described above. For example, oils having a low (6% or less) or no (3.5% or less) total saturated fatty acid content in combination with a typical (60-70%), mid (71-80%), or high (>80%) oleic acid content can be used to replace or reduce the amount of saturated fatty acids and hydrogenated oils (e.g., partially hydrogenated oils) in various food products such that the levels of saturated fatty acids and trans fatty acids are reduced in the food products. In particular, canola oils having a low total saturated fatty acid content and a mid or high oleic acid content in combination with a low linolenic acid content can be used to replace or reduce the amount of saturated fats and partially hydrogenated oils in processed or packaged food products, including bakery products such as cookies, muffins, doughnuts, pastries (e.g., toaster pastries), pie fillings, pie crusts, pizza crusts, frostings, breads, biscuits, cakes, breakfast cereals, breakfast bars, puddings, and crackers.

For example, an oil described herein can be used to produce sandwich cookies that contain reduced saturated fatty acids and no or reduced levels of partially hydrogenated oils in the cookie and/or créme filling. In addition to canola oil, such a cookie composition can include, for example, flour, sweetener (e.g., sugar, molasses, honey, high fructose corn syrup, naturally sweet compounds such as those from Stevia rebaudiana plants (stevioside, rebaudioside A, B, C, D, and/or E), artificial sweetener such as sucralose, saccharine, aspartame, or acesulfame potassium, and combinations thereof), eggs, salt, flavorants (e.g., chocolate, vanilla, or lemon), a leavening agent (e.g., sodium bicarbonate or other baking acid such as monocalcium phosphate monohydrate, sodium aluminum sulfate, sodium acid pyrophosphate, sodium aluminum phosphate, dicalcium phosphate, glucano-deltalactone, or potassium hydrogen tartrate, or combinations thereof), and optionally, an emulsifier (e.g., mono- and diglycerides of fatty acids, propylene glycol mono- and di-esters of fatty acids, glycerol-lactose esters of fatty acids, ethoxylated or succinylated mono- and diglycerides, lecithin, diacetyl tartaric acid esters or mono- and diglycerides, sucrose esters of glycerol, and combinations thereof). In addition to canola oil, a créme filling composition can include sweetener (e.g., powdered sugar, granulated sugar, honey, high fructose corn syrup, artificial sweetener, or combinations thereof), flavorant (e.g., vanilla, chocolate, or lemon), salt, and, optionally, emulsifier.

Canola oils (e.g., with low total saturated fatty acid content, low oleic acid, and low linolenic acid content) also are useful for frying applications due to the polyunsaturated content, which is low enough to have improved oxidative stability for frying yet high enough to impart the desired fried flavor to the food being fried. For example, canola oils can be used to produce fried foods such as snack chips (e.g., corn or potato chips), French fries, or other quick serve foods.

Oils described herein also can be used to formulate spray coatings for food products (e.g., cereals or snacks such as crackers). In some embodiments, the spray coating can include other vegetable oils such as sunflower, cottonseed, corn, or soybean oils. A spray coating also can include an antioxidant and/or a seasoning.

Oils described herein also can be used in the manufacturing of dressings, mayonnaises, and sauces to provide a reduction in the total saturated fat content of the product. The low saturate oil can be used as a base oil for creating structured fat solutions such as microwave popcorn solid fats or canola butter formulations.

The invention throughout this disclosure will be further described in the following examples, which do not limit the scope of the invention described in the claims.

### EXAMPLES

In the Tables described herein, the fatty acids are referred to by the length of the carbon chain and number of double bonds within the chain. For example, C140 refers to C14:0 or myristic acid; C160 refers to C16:0 or palmitic acid; C180 refers to C18:0 or stearic acid; C181 refers to C18:1 or oleic acid; C182 refers to C18:2 or linoleic acid; C183 refers to C18:3 or linolenic acid; C200 refers to C20:0 or archidic acid; C201 refers to C20:1 or eicosenoic acid, C220 refers to C22:0 or behenic acid, C221 refers to C22:1 or erucic acid, C240 refers to C24:0 or lignoceric acid, and C241 refers to C24:1 or nervonic acid. “Total Sats” refers to the total of C140, C160, C180, C200, C220, and C240. Representative fatty acid profiles are provided for each of the specified samples.

Unless otherwise indicated, all percentages refer to weight % based on total weight % of fatty acids in the oil.

### Example 1

### Brassica Plant Line 15.24

Plants producing an oil with a high oleic acid and low total saturated fatty acid content were obtained from crosses of plants designated 90A24 and plants designated 90I22. 90A24 plants were obtained from a cross between HIO 11-5, a high oleic acid selection from the IMC 129 lineage (ATCC Deposit No. 40811; U.S. Pat. No. 5,863,589), and LS 6-5, a low saturated fatty acid selection from the IMC 144 lineage (ATCC Deposit No. 40813; U.S. Pat. No. 5,668,299). 90I22 plants were obtained from a cross between LS 4-3, a low saturated fatty acid selection from the IMC 144 lineage (ATCC Deposit No. 40813) and D336, a low I-linolenic acid selection from the IMC 01 lineage (ATCC Deposit No. 40579; U.S. Pat. No. 5,750,827). Table 1 contains the fatty acid profile for the LS6-5, LS4-3, and HIO 11-5 parent lines, as well as IMC 01.

The F₁ generation progeny of crosses between 90A24 and 90I22 were designated 91AS. F₁ 91 AS plants were self-pollinated to produce F₂ seeds, which were harvested and analyzed for fatty acid composition by gas chromatography (GLC). F₂ seeds having a low linolenic acid content and high oleic acid content were planted and self-pollinated to produce F₃ seeds. The fatty acid composition of F₃ seeds was analyzed. F₃ seeds having a high oleic acid and low linolenic acid content were planted to generate F₃ plants, which were selfed to produce F₄ seeds. The fatty acid composition of F₄ seeds was analyzed by GC. F₄ seeds having a high oleic acid and low linolenic acid content were planted to generate F₄ plants, of which 8 plants were self-pollinated to produce F₅ seeds. The fatty acid composition of F₅ seeds was analyzed by GC (Table 2).

F₅ seeds from one of the lines designated 91AS51057 were selected based on a total saturated fatty acid level of 4.99%, with low palmitic acid of 2.64% and stearic acid of 1.33% (Table 2). This line also had a higher eicosenoic acid (C20:1) content of 1.73%. The seeds of this selection (F₅ 91 AS51057) were planted to generate F₅ plants, which were selfed to produce F₆ seeds. F₆ seeds were harvested from three of five selfed plants. The fatty acid composition of F₆ seeds harvested from each of the three plants is shown in Table 3. Selfing and selection within the 91AS51057 line were continued for an additional 5 generations. Table 4 provides the fatty acid composition for field harvested F₁₀ seeds from 22 lines of self-pollinated 91AS51057 plants. The total saturated fatty acid content ranged from 4.38 to 6.28%, oleic acid content ranged from 74.9 to 82.5%, and I-linolenic acid content ranged from 2.1 to 4.8%. The eicosenoic acid content ranged from 1.28 to 2.30%, with most 91AS51057 F₉ plants producing F₁₀ seeds having an eicosenoic acid content from 1.90 to 2.25%. See Table 4. Seed of four individual F₁₀ 91 AS51057 lines (X723868, X723977, X724734, and X724738) were selected and their seeds planted in the field in individual isolation tents. The low total saturate line X724734 was designated as 15.24 based on its nursery field position of range 15, row 24, and used in future crosses to introduce traits for low saturates through the reduction of palmitic and stearic acids. Line 15.24, which was deposited with the ATCC and designated Deposit PTA-11452, also retained the higher level of eicosinoic acid of 2.06% associated with the saturate reduction.

TABLE 1
Seed Fatty Acid Profile of Parental Lines

| Line | C140 | C160 | C161 | C180 | C181 | C182 | C183 | C200 | C201 | C202 | C220 | C221 | C240 | C241 | Total Sats |
|---|---|---|---|---|---|---|---|---|---|---|---|---|---|---|---|
| LS0004-3 | 0 | 3.01 | 0.00 | 1.27 | 66.75 | 20.03 | 6.08 | 0.45 | 1.31 | 0.11 | 0.26 | 0 | 0.14 | 0.12 | 5.12 |
| LS0006-5 | 0 | 3.07 | 0.06 | 1.11 | 64.83 | 22.18 | 6.10 | 0.40 | 1.29 | 0.11 | 0.24 | 0 | 0.13 | 0.13 | 4.94 |
| HIO011-5 | 0 | 3.79 | 0.24 | 1.91 | 78.60 | 7.86 | 4.64 | 0.71 | 1.44 | 0.00 | 0.39 | 0 | 0.23 | 0.00 | 7.04 |
| IMC 01 | 0 | 4.81 | 0.31 | 2.48 | 61.9i | 24.81 | 2.61 | 0.85 | 1.06 | 0.07 | 0.48 | 0 | 0.33 | 0.15 | 9.02 |

TABLE 2
Fatty Acid Composition of Field Harvested F₅ Seed from Self-pollinated Plants

| TRIAL_ID | C140 | C160 | C161 | C180 | C181 | C182 | C183 | C200 | C201 | C202 | C220 | C221 | C240 | C241 | Total Sats |
|---|---|---|---|---|---|---|---|---|---|---|---|---|---|---|---|
| 91AS51023 | 0.05 | 3.32 | 0.18 | 1.03 | 65.59 | 18.89 | 7.95 | 0.58 | 1.46 | 0.07 | 0.43 | 0.03 | 0.21 | 0.21 | 5.62 |
| 91AS51026 | 0.09 | 4.50 | 0.32 | 1.57 | 63.81 | 24.19 | 2.90 | 0.55 | 1.25 | 0.07 | 0.39 | 0.02 | 0.20 | 0.14 | 7.30 |
| 91AS51026 | 0.09 | 4.36 | 0.29 | 1.51 | 63.09 | 25.21 | 3.11 | 0.49 | 1.14 | 0.07 | 0.33 | 0.01 | 0.17 | 0.13 | 6.95 |
| 91AS51028 | 0.06 | 3.91 | 0.25 | 1.35 | 64.68 | 24.32 | 3.08 | 0.46 | 1.19 | 0.05 | 0.31 | 0.03 | 0.16 | 0.15 | 6.27 |
| 91AS51028 | 0.06 | 3.71 | 0.24 | 1.32 | 64.77 | 24.38 | 2.97 | 0.47 | 1.30 | 0.05 | 0.34 | 0.04 | 0.16 | 0.19 | 6.06 |
| 91AS51034 | 0.04 | 2.68 | 0.17 | 1.31 | 74.75 | 11.44 | 5.88 | 0.57 | 1.88 | 0.25 | 0.42 | 0.20 | 0.26 | 0.17 | 5.27 |
| 91AS51044 | 0.02 | 2.66 | 0.17 | 1.35 | 75.19 | 12.23 | 5.20 | 0.54 | 1.81 | 0.12 | 0.34 | 0.04 | 0.18 | 0.16 | 5.08 |
| 91AS51057 | 0.03 | 2.64 | 0.16 | 1.33 | 71.68 | 12.85 | 8.23 | 0.50 | 1.73 | 0.08 | 0.36 | 0.09 | 0.14 | 0.18 | 4.99 |

TABLE 3
Fatty Acid Composition of Field Harvested F₆ Generation Seed of 91AS51057

| Line | C140 | C160 | C161 | C180 | C181 | C182 | C183 | C200 | C201 | C202 | C220 | C221 | C240 | C241 | Total Sats |
|---|---|---|---|---|---|---|---|---|---|---|---|---|---|---|---|
| 91AS51057 | 0.02 | 2.98 | 0.13 | 2.30 | 78.00 | 9.12 | 2.67 | 0.97 | 2.00 | 0.11 | 0.65 | 0.06 | 0.45 | 0.53 | 7.37 |
| 91AS51057 | 0.03 | 2.86 | 0.14 | 1.41 | 73.94 | 12.02 | 5.74 | 0.61 | 1.95 | 0.10 | 0.43 | 0.05 | 0.25 | 0.49 | 5.58 |
| 91AS51057 | 0.02 | 2.89 | 0.13 | 2.07 | 76.29 | 10.06 | 3.35 | 0.92 | 2.17 | 0.13 | 0.65 | 0.06 | 0.49 | 0.76 | 7.05 |

TABLE 4
Fatty Acid Composition of Field Harvest F₁₀ Generation Seed of 91AS51057

| | Sample | | | | | | | | | | | | | | | Total |
|---|---|---|---|---|---|---|---|---|---|---|---|---|---|---|---|---|
| Line | No. | C140 | C160 | C161 | C180 | C181 | C182 | C183 | C200 | C201 | C202 | C220 | C221 | C240 | C241 | Sats |
| 91AS51057 | X723860 | 0.04 | 3.16 | 0.19 | 1.10 | 78.79 | 9.13 | 3.37 | 0.53 | 2.05 | 0.30 | 0.37 | 0.05 | 0.24 | 0.68 | 5.43 |
| 91AS51057 | X723861 | 0.04 | 2.94 | 0.18 | 1.58 | 81.26 | 7.55 | 2.79 | 0.65 | 1.91 | 0.08 | 0.38 | 0.05 | 0.25 | 0.34 | 5.84 |
| 91AS51057 | X723862 | 0.04 | 3.01 | 0.19 | 1.69 | 80.31 | 7.83 | 2.81 | 0.71 | 2.02 | 0.09 | 0.44 | 0.06 | 0.32 | 0.50 | 6.21 |
| 91AS51057 | X723863 | 0.04 | 2.97 | 0.19 | 1.87 | 80.88 | 7.37 | 2.95 | 0.73 | 1.79 | 0.07 | 0.41 | 0.05 | 0.25 | 0.44 | 6.27 |
| 91AS51057 | X723868 | 0.04 | 2.66 | 0.17 | 0.92 | 78.20 | 10.71 | 3.81 | 0.39 | 2.11 | 0.12 | 0.26 | 0.06 | 0.11 | 0.44 | 4.38 |
| 91AS51057 | X723869 | 0.04 | 3.17 | 0.21 | 1.18 | 80.01 | 8.47 | 2.99 | 0.50 | 2.16 | 0.12 | 0.34 | 0.05 | 0.24 | 0.51 | 5.47 |
| 91AS51057 | X723924 | 0.04 | 2.81 | 0.16 | 1.11 | 80.23 | 9.38 | 3.01 | 0.42 | 1.93 | 0.12 | 0.23 | 0.03 | 0.12 | 0.39 | 4.74 |
| 91AS51057 | X723931 | 0.04 | 2.82 | 0.15 | 0.91 | 79.65 | 9.22 | 3.33 | 0.41 | 2.13 | 0.14 | 0.27 | 0.06 | 0.13 | 0.74 | 4.58 |
| 91AS51057 | X723932 | 0.10 | 2.75 | 0.15 | 0.98 | 79.62 | 9.21 | 3.15 | 0.44 | 2.18 | 0.16 | 0.31 | 0.05 | 0.15 | 0.76 | 4.73 |
| 91AS51057 | X723933 | 0.02 | 2.81 | 0.14 | 0.93 | 80.13 | 9.15 | 3.31 | 0.41 | 2.15 | 0.13 | 0.26 | 0.04 | 0.14 | 0.40 | 4.56 |
| 91AS51057 | X723970 | 0.04 | 3.25 | 0.25 | 1.73 | 82.09 | 8.11 | 2.34 | 0.51 | 1.28 | 0.05 | 0.19 | 0.00 | 0.10 | 0.06 | 5.83 |
| 91AS51057 | X723971 | 0.04 | 3.20 | 0.23 | 1.68 | 82.46 | 7.79 | 2.25 | 0.52 | 1.29 | 0.05 | 0.22 | 0.01 | 0.13 | 0.12 | 5.79 |
| 91AS51057 | X723977 | 0.04 | 2.72 | 0.19 | 1.19 | 80.64 | 9.76 | 2.10 | 0.52 | 1.92 | 0.07 | 0.32 | 0.02 | 0.15 | 0.35 | 4.95 |
| 91AS51057 | X723978 | 0.03 | 2.84 | 0.13 | 1.04 | 80.36 | 8.24 | 3.56 | 0.58 | 2.30 | 0.12 | 0.38 | 0.00 | 0.23 | 0.20 | 5.09 |
| 91AS51057 | X723984 | 0.04 | 2.73 | 0.16 | 1.01 | 79.33 | 9.37 | 4.00 | 0.45 | 1.97 | 0.10 | 0.29 | 0.04 | 0.14 | 0.36 | 4.66 |
| 91AS51057 | X724733 | 0.04 | 3.22 | 0.24 | 1.33 | 74.93 | 12.62 | 4.76 | 0.52 | 1.67 | 0.07 | 0.28 | 0.02 | 0.13 | 0.17 | 5.51 |
| 91AS51057 | X724734 | 0.03 | 2.82 | 0.18 | 0.98 | 80.14 | 8.92 | 3.27 | 0.44 | 2.24 | 0.13 | 0.28 | 0.04 | 0.16 | 0.37 | 4.72 |
| 91AS51057 | X724735 | 0.03 | 2.80 | 0.17 | 1.08 | 79.37 | 9.54 | 3.38 | 0.45 | 2.24 | 0.13 | 0.26 | 0.04 | 0.16 | 0.34 | 4.79 |
| 91AS51057 | X724736 | 0.04 | 3.16 | 0.25 | 1.73 | 80.96 | 7.68 | 2.59 | 0.70 | 1.90 | 0.07 | 0.40 | 0.05 | 0.25 | 0.23 | 6.28 |
| 91AS51057 | X724737 | 0.04 | 2.80 | 0.20 | 1.54 | 80.29 | 8.36 | 3.49 | 0.64 | 1.75 | 0.06 | 0.38 | 0.04 | 0.17 | 0.25 | 5.57 |
| 91AS51057 | X724738 | 0.03 | 2.72 | 0.17 | 1.12 | 81.88 | 7.71 | 2.84 | 0.52 | 2.06 | 0.10 | 0.32 | 0.05 | 0.17 | 0.30 | 4.89 |
| 91AS51057 | X724754 | 0.04 | 2.79 | 0.18 | 1.64 | 80.73 | 8.19 | 3.39 | 0.60 | 1.64 | 0.06 | 0.33 | 0.03 | 0.16 | 0.22 | 5.56 |
| AVERAGE | | 0.04 | 2.92 | 0.19 | 1.29 | 80.1 | 8.83 | 3.16 | 0.53 | 1.94 | 0.11 | 0.31 | 0.04 | 0.18 | 0.37 | 5.27 |

### Example 2

### Identification of FatA2 Mutation in 15.24 Plants

Genome mapping, map-based gene cloning, and direct-sequencing strategies were used to identify loci associated with the low total saturated fatty acid phenotype in the 15.24 lines described in Example 1. A DH (doubled haploid) population was developed from a cross between 15.24 and 01OB240, a B line used in the maintenance of cytoplasmic male sterile (CMS) A lines for hybrid production. The two parental lines were screened with 1066 SNP (single nucleotide polymorphism) markers using the MassARRAY platform (Sequenom Inc., San Diego, Calif.) to identify polymorphic SNP markers between the two parents; 179 polymorphic SNP markers were identified.

Single marker correlations between fatty acid components and SNP markers were carried out using the SAS program (SAS Institute 1988). A B. napus genetic linkage map was constructed using the Kosambi function in JoinMap 3.0 (Kyazma). Interval quantitative trait loci (QTL) mapping was done with MapQTL 4.0 (Kyazma). A LOD score >3.0 was considered as threshold to declare the association intervals. For fine QTL mapping, a BC₃S (backcrossing self) population was developed from a cross between 15.24 and 01PRO6RR.001B, a canola R (restorer) line. SNP haplotype blocks and recombinant/crossover events within the identified QTL interval were identified using MS Excel® program.

Comparative genome mapping was performed to locate the identified QTL in B. napus chromosomes and further identify the B. rapa BAC (Bacterial Artificial chromosome) clones encompassing the identified SNP markers and the candidate genes in the identified QTL interval for the low total saturated fatty acid using publicly available Brassica and Arabidosis genome sequences, genes, genetic linkage maps, and other information from the World Wide Web at brassica.bbsrc.ac.uk/ and ncbi.nlm nih gov/.

A total of 148 DH lines were genotyped with 179 polymorphic SNP markers. QTL mapping identified a major QTL interval (5 cM) encompassing 7 SNP markers for saturated fatty acid content (C18:0 and C20:0). Fine mapping using 610 BC₃S₁ lines from a cross between 15.24 and 01PRO6RR.001B, a canola restorer line, identified two SNP markers flanking a 1 cM QTL interval that was associated with the low total saturated fatty acid phenotype. Comparative genomics initially located this QTL on the N3 chromosome using 179 SNP markers and identified a FATA2 candidate in that QTL interval. Subsequent mapping using the Brassica 60K SNP confined the involvement of the FATA2 locus and placed the QTL on chromosome N4 (see Example 14 and FIG. 8).

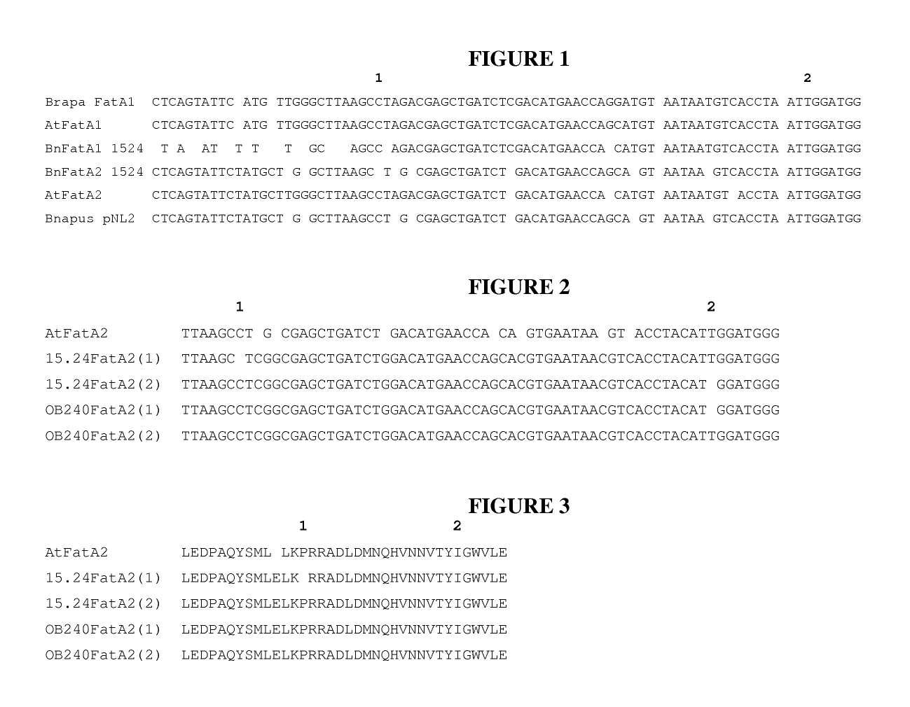

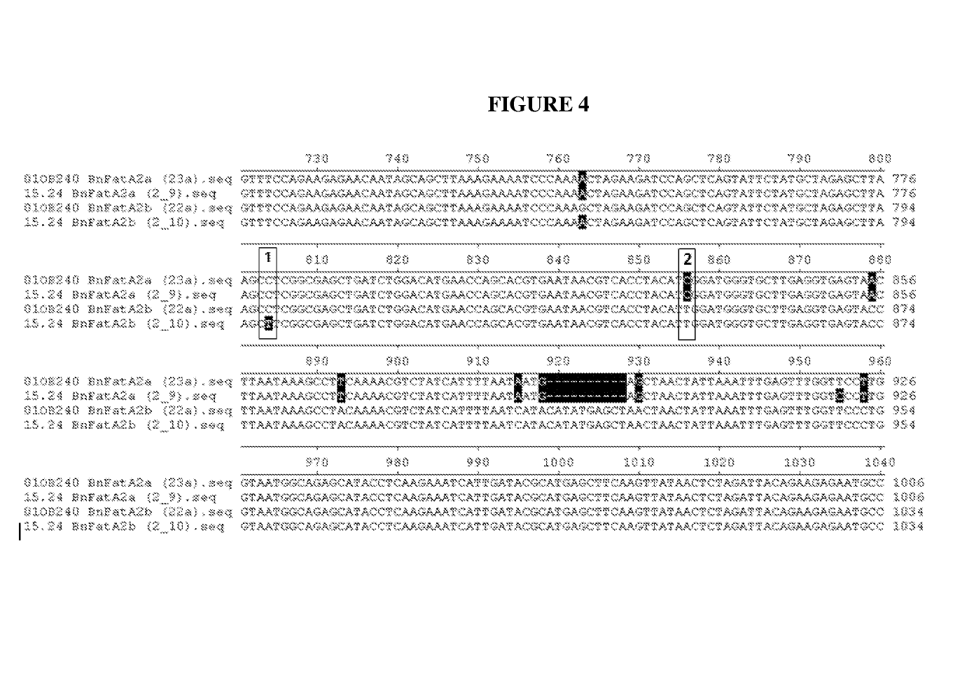

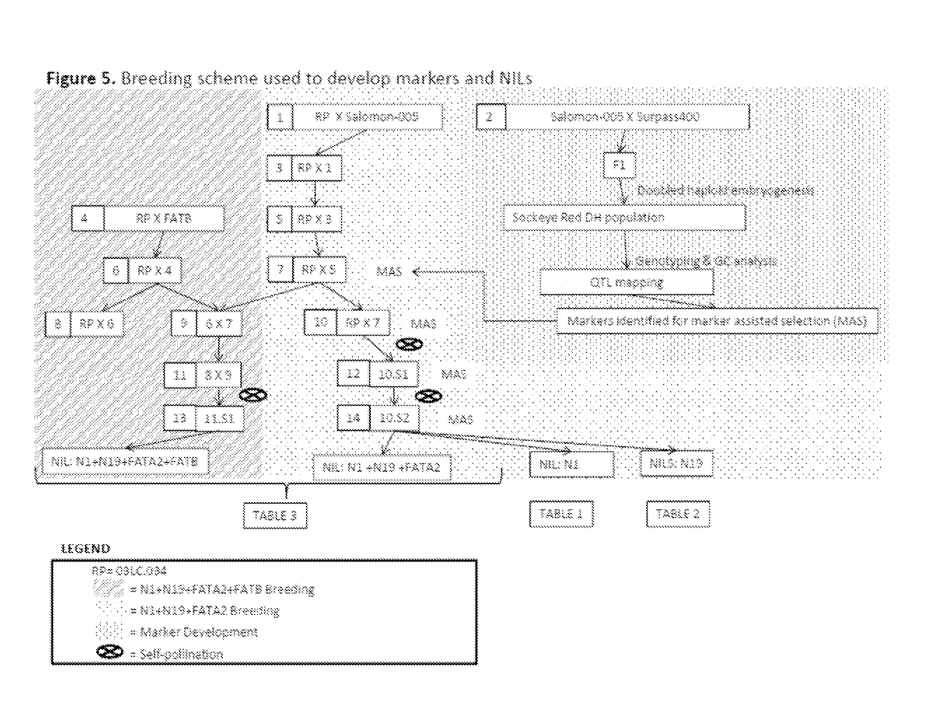

DNA from lines 15.24 and 01OB240 was used as a template to amplify FatA sequences. Resultant sequences were analyzed using BLAST (the Basic Local Alignment Search Tool) and MegAlign and EditSeq programs from DNASTAR/Lasergene 8.0 (DNASTAR, Inc). Isoforms of FatA1 and FatA2 were amplified and a representative sampling is shown in FIG. 1. The BnFatA1 sequence from 15.24 is homologous to the B. rapa FatA1 and A. thaliana FatA1 sequences, while the BnFatA2 sequence from 15.24 is homologous to the AtFatA2 and B. napus pNL2 sequences. Two isoforms (or alleles) of FatA2 were evident in the sequencing results and were named FatA2a and FatA2b. Differences between the sequences of these two isoforms are shown in FIG. 4. FIGS. 1 and 2 show a representative nucleotide (position labeled “2;” only the FatA2b isoform is represented in FIG. 1) where, in that position, FatA2a is a “C” and FatA2b is a “T” (summarized in FIG. 4). The FatA2 sequencing results indicated that, within the FatA2b isoform sequences, 15.24 contained a single nucleotide polymorphism represented by the position labeled “1” in FIGS. 1, 2 and 4. In 15.24, the FatA2b sequences contain a “C” to “T” mutation that was not present in the 01OB240 sequences (“1” in FIGS. 1,2, 4). The nucleotide substitution of position “1” in FIGS. 1 and 2 corresponds to position 942 of the FatA2 coding sequence (numbering based on the A. thaliana sequence set forth in GenBank Accession No. NM_117374.3) and results in the substitution of a leucine residue for proline at position 255 of the encoded protein. See SEQ ID NO:28 and SEQ ID NO:32, which provide representative nucleotide sequences of the B. napus FatA2b gene from 15.24. In FIG. 4, position 798 is marked at the “C” to “T” SNP that correlates with low saturate content in the 15.24 lines. SEQ ID NO:29 contains the amino acid sequence of residues 242 to 277 of a wild-type B. napus FatA2 polypeptide. Position 14 of SEQ ID NO:29 (position 255 in the full-length amino acid sequence) is a leucine in the FatA2 polypeptide from 15.24. SEQ ID NO:30 contains the wild-type Arabidopsis FatA2 polypeptide. SEQ ID NO:31 contains the predicted amino acid sequence of the B. napus FATA2b polypeptide from exons 2-6.

FIG. 3 contains an alignment of the conserved region around position 255 in the Arabidopsis FatA2 protein, and Brassica FatA2 protein from 15.24 and 01OB240. The proline at position 255 is conserved among Brassica, Arabidopsis, B. napus, B. rapa, B. juncea, Zea mays, Sorghum bicolor, Oryza sativa Indica (rice), Triticum aestivum, Glycine max, Jatropha (tree species), Carthamus tinctorius, Cuphea hookeriana, Iris tectorum, Perilla frutescens, Helianthus annuus, Garcinia mangostana, Picea sitchensis, Physcomitrella patens subsp. Patens, Elaeis guineensis, Vitis vinifera, Elaeis oleifera, Camellia oleifera, Arachis hypogaea, Capsicum annuum, Populus trichocarpa, and Diploknema butyracea. Furthermore, many amino acids in the region spanning amino acids 242 to 277 are homologous in both FatA and FatB (see Facciotti and Yuan, Fett/Lipid 100 (1998) 167-172) in Arabidopsis and Brassica.

FIG. 4 shows a portion of representative BnFatA2a and BnFatA2b sequences from 01OB240 and 15.24 germplasm. The positions labeled “1” and “2” correspond to the “1” and “2” positions in FIGS. 1, 2 and 3.

Large scale screening of the parental lines (15.24 and 01OB240) as well as other germplasm populations (including IMC144, IMC129, Q508, and Q7415) indicated the FatA2 SNP was 15.24-specific and was statistically significantly associated with the low total saturated fatty acid phenotype (R-square=0.28 for total saturated content, R-square=0.489 for C18:0; R-square=0.385 for C20:0) and increased eicosenoic acid content (R-square=0.389). The FatA2 SNP1 mutation was not significantly associated with the percent C14:0 and C16:0 content of oil from 15.24 plants. However, it was found that the C18:0 content of oil from 15.24 plants was negatively correlated with C20:1 content (R-square value=−0.61).

### Example 3

### Brassica Line 15.36

Plants producing an oil with a high oleic acid and low total saturated fatty acid content were obtained from crosses of plants from lines A12.20010 and Q508. The A12.20010 line was obtained from a cross of a selection from the IMC144 lineage and a selection from the IMC129 lineage. Line Q508 is a high oleic acid line that contains a mutation in each of the fad2 D and F genes. See Examples 5 and 7 of U.S. Pat. No. 6,342,658.

Plants designated 92EP.1039 were selected on the basis of fatty acid composition from progeny of the A12.20010×Q508 cross. 92EP.1039 plants were crossed with plants of Trojan, a commercially available Canadian spring canola variety. The F₁ generation progeny of 92EP.1039 and Trojan were designated 93PI. F₁ 93 PI plants were self-pollinated to produce F₂ seeds, which were harvested and analyzed for fatty acid composition by GC.

F₂ seeds having a high oleic acid content were selected and planted to obtained F₂ plants. The F₂ plants were self-pollinated to produce F₃ seeds. The fatty acid composition of F₃ seeds was analyzed. Table 5 contains the fatty acid profile of 93PI21 F₃ seeds from 13 different F₂ plants. F₃ 93 PI21 seeds having a low saturated fatty acid content were planted to generate F₃ plants, which were selfed to produce F₄ seeds. The fatty acid composition of F₄ 93 PI21 seeds was analyzed by GC. Table 6 contains the fatty acid profile of F₄ 93 PI21 seeds from thirteen different self-pollinated F₃ plants. The three 93PI21 plants (T7440796, T740797, and T740799) with the lowest total saturated fatty acid content were subjected to additional rounds of selfing and selection for low total saturated fatty acid content for 5 generations. The 93PI2I line T740799 was designated as 93P141003 at the F₄ generation and advanced. Table 7 provides the fatty acid composition for F₈ seeds harvested from 24 self-pollinated F₇ generation 93P141003 plants. The results indicate that total saturated fatty acid content ranged from 4.51 to 6.29%, oleic acid content ranged from 64 to 71%, and I-linolenic acid content ranged from 4.8 to 7.5%. The eicosenoic acid content ranged from 1.51 to 1.99%. The 93P141003 F₈ plant line X727712 was renamed as line 15.36 based on its nursery field position of range 15, row 36, and had a total saturated fatty acid composition of 4.51%, with reduced palmitic acid of 2.65% and stearic acid of 0.94%. Line 15.36, which was deposited with the ATCC and designated deposit PTA-11451, was used in crosses to introduce low saturate traits to other genetic backgrounds.

TABLE 5
Seed Fatty Acid Composition of F₃ Generation of 93PI21

| Line | C140 | C160 | C161 | C180 | C181 | C182 | C183 | C200 | C201 | C202 | C220 | C221 | C240 | C241 | Total Sats |
|---|---|---|---|---|---|---|---|---|---|---|---|---|---|---|---|
| 93PI21 | 0.04 | 3.15 | 0.22 | 1.77 | 80.06 | 6.95 | 4.23 | 0.75 | 1.77 | 0.08 | 0.43 | 0.04 | 0.33 | 0.19 | 6.46 |
| 93PI21 | 0.04 | 3.22 | 0.21 | 1.29 | 79.05 | 7.82 | 4.90 | 0.60 | 1.79 | 0.09 | 0.37 | 0.07 | 0.31 | 0.24 | 5.82 |
| 93PI21 | 0.03 | 3.32 | 0.28 | 1.69 | 77.63 | 8.95 | 4.31 | 0.73 | 1.88 | 0.09 | 0.47 | 0.07 | 0.35 | 0.20 | 6.59 |
| 93PI21 | 0.04 | 3.57 | 0.33 | 1.43 | 81.33 | 6.09 | 3.89 | 0.63 | 1.61 | 0.05 | 0.41 | 0.17 | 0.25 | 0.20 | 6.34 |
| 93PI21 | 0.05 | 3.47 | 0.34 | 1.38 | 80.70 | 6.28 | 4.85 | 0.58 | 1.55 | 0.05 | 0.35 | 0.05 | 0.22 | 0.13 | 6.05 |
| 93PI21 | 0.05 | 3.63 | 0.34 | 1.41 | 80.06 | 6.54 | 4.99 | 0.60 | 1.57 | 0.05 | 0.37 | 0.04 | 0.22 | 0.15 | 6.27 |
| 93PI21 | 0.03 | 3.14 | 0.25 | 1.33 | 77.85 | 8.98 | 4.98 | 0.59 | 1.80 | 0.07 | 0.40 | 0.15 | 0.24 | 0.19 | 5.72 |
| 93PI21 | 0.03 | 3.00 | 0.24 | 1.34 | 77.65 | 8.02 | 6.23 | 0.61 | 1.90 | 0.08 | 0.40 | 0.06 | 0.24 | 0.22 | 5.60 |
| 93PI21 | 0.06 | 3.66 | 0.38 | 1.73 | 77.25 | 8.83 | 4.87 | 0.72 | 1.53 | 0.06 | 0.44 | 0.00 | 0.31 | 0.16 | 6.91 |
| 93PI21 | 0.08 | 4.34 | 0.52 | 2.17 | 77.22 | 6.57 | 4.94 | 0.99 | 1.75 | 0.06 | 0.66 | 0.07 | 0.40 | 0.24 | 8.64 |
| 93PI21 | 0.05 | 3.49 | 0.39 | 1.71 | 85.90 | 2.86 | 2.94 | 0.64 | 1.32 | 0.04 | 0.32 | 0.00 | 0.22 | 0.15 | 6.43 |
| 93PI21 | 0.04 | 3.13 | 0.25 | 1.44 | 80.58 | 6.99 | 4.31 | 0.60 | 1.81 | 0.07 | 0.36 | 0.04 | 0.23 | 0.15 | 5.80 |
| 93PI21 | 0.05 | 4.21 | 0.24 | 1.66 | 73.40 | 14.31 | 2.83 | 0.67 | 1.45 | 0.04 | 0.45 | 0.03 | 0.50 | 0.16 | 7.54 |

TABLE 6
Seed Fatty Acid Composition of Field Grown F₄ Seed Generation of 93PI21

| Line | Sample No. | C140 | C160 | C161 | C180 | C181 | C182 | C183 | C200 | C201 | C202 | C220 | C221 | C240 | C241 | Total Sats |
|---|---|---|---|---|---|---|---|---|---|---|---|---|---|---|---|---|
| 93PI21 | T738147 | 0.03 | 2.78 | 0.15 | 1.60 | 69.57 | 13.82 | 8.81 | 0.62 | 1.77 | 0.08 | 0.37 | 0.04 | 0.14 | 0.22 | 5.54 |
| 93PI21 | T738149 | 0.04 | 2.87 | 0.17 | 1.47 | 71.02 | 11.74 | 9.63 | 0.57 | 1.75 | 0.07 | 0.35 | 0.00 | 0.12 | 0.21 | 5.42 |
| 93PI21 | T738148 | 0.05 | 3.35 | 0.29 | 1.84 | 73.71 | 11.27 | 5.81 | 0.64 | 1.40 | 0.07 | 0.35 | 0.06 | 0.17 | 0.99 | 6.40 |
| 93PI21 | T740387 | 0.04 | 3.28 | 0.22 | 1.68 | 65.96 | 15.57 | 9.38 | 0.62 | 1.89 | 0.14 | 0.46 | 0.06 | 0.29 | 0.41 | 6.36 |
| 93PI21 | T740388 | 0.03 | 3.00 | 0.20 | 1.66 | 71.33 | 11.89 | 8.15 | 0.63 | 1.93 | 0.10 | 0.49 | 0.05 | 0.29 | 0.26 | 6.09 |
| 93PI21 | T740389 | 0.03 | 2.72 | 0.20 | 1.42 | 75.27 | 8.72 | 7.90 | 0.57 | 2.06 | 0.10 | 0.46 | 0.06 | 0.22 | 0.27 | 5.42 |
| 93PI21 | T740749 | 0.03 | 2.86 | 0.18 | 1.31 | 68.64 | 13.27 | 10.44 | 0.50 | 1.90 | 0.09 | 0.34 | 0.06 | 0.16 | 0.22 | 5.21 |
| 93PI21 | T740797 | 0.03 | 2.99 | 0.21 | 1.23 | 72.19 | 10.92 | 9.37 | 0.48 | 1.78 | 0.07 | 0.33 | 0.04 | 0.14 | 0.22 | 5.20 |
| 93PI21 | T740798 | 0.03 | 2.78 | 0.20 | 1.26 | 76.73 | 7.47 | 7.39 | 0.58 | 2.35 | 0.14 | 0.47 | 0.07 | 0.18 | 0.34 | 5.29 |
| 93PI21 | T740799 | 0.03 | 3.03 | 0.22 | 1.19 | 72.63 | 11.46 | 8.18 | 0.49 | 1.76 | 0.11 | 0.37 | 0.05 | 0.17 | 0.34 | 5.27 |
| 93PI21 | T738147 | 0.03 | 2.78 | 0.15 | 1.60 | 69.57 | 13.82 | 8.81 | 0.62 | 1.77 | 0.08 | 0.37 | 0.04 | 0.14 | 0.22 | 5.54 |
| 93PI21 | T7381149 | 0.04 | 2.87 | 0.17 | 1.47 | 71.02 | 11.74 | 9.63 | 0.57 | 1.75 | 0.007 | 0.35 | 0.00 | 0.12 | 0.21 | 5.42 |
| 93PI21 | T738148 | 0.05 | 3.35 | 0.29 | 1.84 | 73.71 | 11.27 | 5.81 | 0.64 | 1.40 | 0.07 | 0.35 | 0.06 | 0.17 | 0.99 | 6.40 |

TABLE 7
Fatty Acid Composition of F₉ Seeds from 93PI41003 Plants in Isolation Tents

| RESCHID | SAMPLE ID | C140 | C160 | C161 | C180 | C181 | C182 | C183 | C200 | C201 | C202 | C220 | C221 | C240 | C241 | Total Sats |
|---|---|---|---|---|---|---|---|---|---|---|---|---|---|---|---|---|
| 93PI41003 | X723830 | 0.04 | 2.99 | 0.23 | 1.21 | 60.88 | 23.17 | 8.30 | 0.53 | 1.67 | 0.12 | 0.33 | 0.04 | 0.18 | 0.33 | 5.26 |
| 93PI41003 | X723846 | 0.03 | 2.73 | 0.22 | 1.22 | 66.06 | 20.77 | 6.22 | 0.45 | 1.57 | 0.09 | 0.26 | 0.03 | 0.15 | 0.22 | 4.83 |
| 93PI41003 | X723847 | 0.04 | 2.89 | 0.20 | 1.18 | 68.08 | 17.98 | 6.16 | 0.54 | 1.89 | 0.10 | 0.33 | 0.03 | 0.23 | 0.35 | 5.21 |
| 93PI41003 | X723848 | 0.03 | 2.80 | 0.21 | 1.23 | 64.93 | 20.91 | 7.09 | 0.47 | 1.58 | 0.08 | 0.27 | 0.02 | 0.15 | 0.22 | 4.95 |
| 93PI41003 | X723882 | 0.06 | 2.84 | 0.20 | 1.38 | 69.81 | 16.49 | 5.47 | 0.58 | 1.94 | 0.10 | 0.39 | 0.06 | 0.27 | 0.41 | 5.53 |
| 93PI41003 | X723883 | 0.04 | 2.87 | 0.19 | 1.35 | 68.41 | 17.07 | 5.98 | 0.60 | 1.95 | 0.14 | 0.42 | 0.06 | 0.31 | 0.61 | 5.59 |
| 93PI41003 | X723916 | 0.04 | 3.12 | 0.17 | 1.43 | 69.74 | 16.59 | 5.19 | 0.63 | 1.99 | 0.09 | 0.41 | 0.04 | 0.31 | 0.25 | 5.93 |
| 93PI41003 | X723917 | 0.02 | 2.51 | 0..20 | 1.02 | 65.86 | 19.54 | 7.61 | 0.41 | 1.77 | 0.11 | 0.29 | 0.04 | 0.11 | 0.53 | 4.35 |
| 93PI41003 | X723918 | 0.03 | 2.48 | 0.17 | 1.20 | 68.96 | 17.58 | 5.99 | 0.52 | 1.94 | 0.09 | 0.33 | 0.04 | 0.19 | 0.49 | 4.74 |
| 93PI41003 | X723919 | 0.03 | 3.12 | 0.18 | 1.10 | 67.25 | 18.48 | 6.46 | 0.48 | 1.90 | 0.11 | 0.32 | 0.04 | 0.23 | 0.31 | 5.27 |
| 93PI41003 | X724063 | 0.04 | 2.73 | 0.19 | 1.18 | 66.70 | 19.56 | 6.43 | 0.50 | 1.80 | 0.09 | 0.29 | 0.02 | 0.18 | 0.28 | 4.92 |
| 93PI41003 | X724064 | 0.04 | 2.71 | 0.21 | 1.22 | 64.00 | 21.73 | 7.06 | 0.45 | 1.60 | 0.08 | 0.27 | 0.04 | 0.15 | 0.44 | 4.83 |
| 93PI41003 | X724077 | 0.03 | 2.60 | 0.16 | 1.16 | 67.89 | 19.14 | 5.78 | 0.52 | 1.87 | 0.09 | 0.32 | 0.04 | 0.19 | 0.20 | 4.82 |
| 93PI41003 | X724091 | 0.03 | 2.72 | 0.18 | 1.27 | 68.62 | 18.11 | 5.76 | 0.57 | 1.93 | 0.10 | 0.34 | 0.00 | 0.18 | 0.19 | 5.11 |
| 93PI41003 | X724092 | 0.03 | 2.65 | 0.19 | 1.11 | 63.98 | 21.64 | 7.13 | 0.45 | 1.81 | 0.10 | 0.28 | 0.03 | 0.16 | 0.44 | 4.69 |
| 93PI41003 | X724093 | 0.03 | 2.57 | 0.19 | 1.21 | 67.35 | 19.67 | 5.77 | 0.47 | 1.80 | 0.09 | 0.29 | 0.04 | 0.18 | 0.36 | 4.74 |
| 93PI41003 | X724412 | 0.03 | 2.65 | 0.18 | 0.94 | 65.27 | 20.41 | 7.54 | 0.44 | 1.71 | 0.09 | 0.26 | 0.04 | 0.18 | 0.26 | 4.51 |
| 93PI41003 | X724416 | 0.04 | 3.02 | 0.22 | 1.19 | 68.18 | 18.57 | 5.49 | 0.54 | 1.67 | 0.08 | 0.34 | 0.05 | 0.29 | 0.33 | 5.41 |
| 93PI41003 | X724417 | 0.04 | 2.72 | 0.23 | 1.05 | 66.68 | 19.31 | 6.59 | 0.47 | 1.93 | 0.11 | 0.31 | 0.05 | 0.17 | 0.35 | 4.75 |
| 93PI41003 | X724420 | 0.03 | 2.81 | 0.19 | 1.22 | 69.48 | 17.31 | 5.43 | 0.57 | 1.71 | 0.09 | 0.36 | 0.05 | 0.36 | 0.42 | 5.34 |
| 93PI41003 | X724421 | 0.03 | 2.86 | 0.20 | 1.14 | 66.28 | 19.70 | 6.81 | 0.49 | 1.61 | 0.08 | 0.28 | 0.05 | 0.21 | 0.26 | 5.01 |
| 93PI41003 | X724422 | 0.04 | 3.18 | 0.21 | 1.04 | 64.87 | 20.88 | 7.02 | 0.50 | 1.51 | 0.07 | 0.30 | 0.05 | 0.15 | 0.18 | 5.20 |
| 93PI41003 | X724423 | 0.03 | 2.88 | 0.17 | 1.15 | 68.48 | 17.75 | 6.50 | 0.51 | 1.69 | 0.08 | 0.30 | 0.05 | 0.20 | 0.20 | 5.08 |
| 93PI41003 | X724611 | 0.04 | 3.28 | 0.25 | 1.64 | 71.09 | 15.28 | 4.78 | 0.64 | 1.73 | 0.08 | 0.39 | 0.05 | 0.31 | 0.45 | 6.29 |

### Example 4

### Cloning of Brassica napus FatB

Cloning of the Brassica napus Fat B gene was initiated by performing polymerase chain reaction (PCR) with primers Fat B1 (5′-ATGAAGGTTAAACCAAACGCTCAGGC-3′; SEQ ID NO:8) and Fat B2 (5′-TGTTCTTCCTCTCACCACTTCAGC-3′; SEQ ID NO:9), respectively, using Westar genomic DNA as template and Taq polymerase (Qiagen). Each 50 TL reaction contained 0.5 TM primers, 1× Qiagen Taq polymerase buffer, 2.5 U Taq polymerase, and 0.2 mM dNTPs. The target was amplified using the following cycling conditions: 1 cycle of 94° C. for 30 seconds; 5 cycles of 94° C. for 10 seconds, 58° C. for 30 seconds, and 72° C. for 1 min. 30 secs; 5 cycles of 94° C. for 10 seconds, 54° C. for 30 seconds, and 72° C. for 1 min. 30 secs; and 24 cycles of 94° C. for 10 seconds, 51° C. for 30 seconds, and 72° C. for 1 min. 45 secs. Aliquots of the PCR reactions were run on an agarose gel and selected bands were excised; DNA was eluted from the bands using the Qiagen Qiaquick kit. The DNA eluate was subjected to a ‘polishing’ reaction to facilitate T/A cloning and then TOPO® T/A cloned using the TOPO® T/A® cloning kit (Invitrogen). Sequences were obtained for the clones then analyzed using BLAST to search for homology. One of the clones appeared to be a FatB.

PCR was repeated using Invitrogen Platinum® Pfx polymerase, its buffer, supplementary MgSO₄ at a final concentration of 2 mM, and IMC201 strain genomic DNA with cycling conditions as follows: 1 cycle of 94° C. for 2 minutes; 5 cycles of 94° C. for 10 seconds, 60° C. for 30 seconds, and 72° C. for 1 min. 20 secs; 5 cycles of 94° C. for 10 seconds, 57° C. for 30 seconds, and 72° C. for 1 min. 30 secs; and 24 cycles of 94° C. for 10 seconds, 54° C. for 30 seconds, and 72° C. for 1 min. 30 secs. The PCR product from this reaction also was Topo®T/A® cloned using the Topo® T/A® cloning system (Invitrogen).

A number of the clones that were sequenced showed homology to Fat B (SEQ ID NOS:10, 11, 12, 13), with 4 distinct isoforms of the gene identified. To obtain the sequence of the start and stop regions of each gene, a ‘walking’ procedure was employed utilizing GenomeWalker3 kits (Clontech), according to manufacturer protocols. Based on the sequence information from the walking procedure, primers corresponding to 5′ UTR and 3′UTR or near-stop codon regions of the FatB genes were designed. PCR was performed using IMC201 genomic DNA as template and two sets of primers in 50 TL reactions containing 1× Platinum® Taq High Fidelity buffer; 2.5 U Platinum® Taq High Fidelity polymerase; 0.2 mM dNTPs; 0.5 TM primers; and 2 mM MgSO₄. Primers for the first reaction were 5′-CTTTGAACGCTCAGCTCCTCAGCC-3′ (SEQ ID NO:14) and 5′-‘AAACGAACCAAAGAACCCATGTTTGC-3’ (SEQ ID NO:15). Primers for the second reaction were 5′-CTTTGAAAGCTCATCTTCCTCGTC-3′ (SEQ ID NO:16) and 5′-GGTTGCAAGGTAGCAGCAGGTACAG-3′ (SEQ ID NO:17). The first reaction was performed under the following cycling conditions: 1 cycle of 94° C. for 2 minutes; 5 cycles of 94° C. for 10 seconds, 56° C. for 40 seconds, and 68° C. for 1 min. 30 secs; 30 cycles of 94° C. for 10 seconds, 53° C. for 30 seconds, and 68° C. for 2 min. The second reaction was performed under the following cycling conditions: 1 cycle of 95° C. for 2 minutes; 5 cycles of 94° C. for 10 seconds, 58° C. for 40 seconds, and 68° C. for 2 min; and 30 cycles of 94° C. for 10 seconds, 55° C. for 30 seconds, and 68° C. for 2 min. Both reaction sets produced bands with an expected size of ˜1.6 Kb.

To clone the DNA, PCR reactions were performed using 1 cycle of 94° C. for 2 minutes, and 35 cycles of 94° C. for 10 seconds, 58° C. for 40 seconds, and 68° C. for 2 min. The resultant bands were gel purified and run over Qiagen Qiex II columns to purify the DNA from the agarose gel. The DNA was Topo®T/A® cloned using the Invitrogen T/A® cloning system. The nucleotide sequences set forth in SEQ ID NOS:18-21 represent full-length (or near full-length) FatB isoforms 1, 2, 3, and 4, respectively.

FatB isoforms 5 and 6 were identified as follows. Primers 5′-ACAGTGGATGATGCTTGACTC-3′ (SEQ ID NO:22) and 5′-TAGTAATATACCTGTAAGTGG-3′ (SEQ ID NO:23) were designed based on FatB sequences from B. napus 01OB240 and used to amplify B. napus genomic DNA from IMC201. The resulting products were cloned and sequenced, and a new FatB partial length isoform was identified. Sequence walking was performed with GenomeWalker3 kits (Clontech). Primers 5′-TACGATGTAGTGTCCCAAGTTGTTG-3′ (SEQ ID NO:24) and 5′-TTTCTGTGGTGTCAGTGTGTCT-3′ (SEQ ID NO:25) were designed based on the sequence obtained through genome walking and used to amplify a contiguous ORF region of the new FatB isoform. PCR products were cloned and sequenced to identify FatB isoforms 5 and 6 (SEQ ID NO:26 and SEQ ID NO:27). The six isoforms have 82 to 95% sequence identity as assessed with the ClustalW algorithm.

### Example 5

### Mutant FatB Genes

A population of B. napus IMC201 seeds was subjected to chemical mutagenesis. The typical fatty acid composition of field grown IMC201 is 3.6% C16:0, 1.8% C18:0, 76% C18:1, 12.5% C18:2, 3% C18:3, 0.7% C20:0, 1.5% C20:1, 0.3% C22:0, 0% C22:1, with total saturates of 6.4%. Prior to mutagenesis, IMC201 seeds were pre-imbibed in 700 gm seed lots by soaking for 15 mM then draining for 5 mM at room temperature. This was repeated four times to soften the seed coat. The pre-imbibed seeds then were treated with 4 mM methyl N-nitrosoguanidine (MNNG) for three hours. Following the treatment with MNNG, seeds were drained of the mutagen and rinsed with water for one hour. After removing the water, the seeds were treated with 52.5 mM ethyl methanesulfonate (EMS) for sixteen hours. Following the treatment with EMS, the seeds were drained of mutagen and rinsed with water for one and half hours. This dual mutagen treatment was lethal to about 50% of the seed population (about the LD₅₀).

Approximately 200,000 treated seeds were planted in standard greenhouse potting soil and placed in an environmentally controlled greenhouse. The plants were grown under sixteen hours of day light. At maturity, M₂ seed was harvested from the plants and bulked together. The M₂ generation was planted and leaf samples from the early, post-cotyledon stage of development from 8 plants were pooled and DNA was extracted from leaves of these plants. The leaf harvest, pooling and DNA extraction was repeated for approximately 32,000 plants, and resulted in approximately forty 96-well blocks containing mutagenized B. napus IMC201 DNA. This grouping of mutagenized DNA is referred to below as the DNA mutagenesis library.

The DNA mutagenesis library was screened to identify stop-codon containing FatB mutants. In general, PCR primers were designed to amplify a region of each FatB isoform. The reaction products were analyzed using temperature gradient capillary electrophoresis on a REVEAL3 instrument (Transgenomics Inc.), which allows PCR reactions containing heterogeneous PCR products to be distinguished from reactions containing only homogeneous products, as would be the case if a single-nucleotide polymorphism (SNP) existed in genomic DNA from chemical mutagenesis and subsequent PCR amplification.

Individual seeds representing the primary hit of each M₂ plant that was the source genomic DNA mix for this primary mutagenesis screen were sampled and genomic DNA was isolated in order to perform the isoform PCR. PCR reactions were performed using B. napus IMC201 genomic DNA in a 30 TL reaction containing 1× Platinum® Taq High Fidelity buffer; 2.0 U Platinum3 Taq High Fidelity polymerase; 0.2 mM dNTPs; 0.5 TM primers; and 2 mM MgSO₄. Cycling conditions were as follows: 1 cycle of 95° C. for 2 minutes followed by 34 cycles of 94° C. for 6 seconds, 64° C. for 40 seconds, and 68° C. for 40 seconds. PCR products were sequenced and the sequences were compared to the wild-type sequence for each isoform.

The sequence comparisons indicated that mutations had been generated and mutant plants obtained for each of isoforms 1, 2, 3 and 4. The mutant sequences are shown in SEQ ID NOS: 1-4. SEQ ID NO:1 contains the nucleotide sequence of isoform 1 having a mutation at position 154, changing the codon from CAG to TAG. SEQ ID NO:2 contains the nucleotide sequence of isoform 2 having a mutation at position 695, changing the codon from CAG to TAG. SEQ ID NO:3 contains the nucleotide sequence of isoform 3 having a mutation at position 276, changing the codon from TGG to TGA. SEQ ID NO:4 contains the nucleotide sequence of isoform 4 having a mutation at position 336, changing the codon from TGG to TGA.

### Example 6

### Brassica napus Plants Carrying Combinations of Mutant Brassica FatB Genes

B. napus plants carrying different combinations of mutants in different FatB isoforms were generated in order to determine the effect of the various mutant Brassica FatB alleles described in Example 5 on the fatty acid composition of B. napus seed oil. Parent plants, each carrying one or more mutations in a different isoform, were crossed in various ways, and progeny were screened by DNA sequence analysis to identify the mutation(s) present, followed by self-pollination and DNA sequence analysis to determine whether the mutations were present in the homozygous or heterozygous state.

Using this process, three Brassica plants were generated that carried mutant alleles of four FatB isoforms. Each of these plants was self-pollinated, harvested and replanted in the greenhouse to create a population of 1,140 plants. All 1,140 plants were screened via DNA sequence analysis to determine whether the mutant alleles were present in the homozygous or heterozygous state at each of the FatB isoform loci. Progeny were identified that were homozygous for the following combinations of mutant FatB isoforms: FatB isoforms 1, 2 and 3; FatB isoforms 1, 2 and 4; FatB isoforms 2, 3 and 4; FatB isoforms 1, 3 and 4; and FatB isoforms 1, 2, 3 and 4.

Plants carrying combinations of mutant FatB isoforms were self-pollinated and seeds were harvested. The resulting seeds were planted in growth chambers under two different temperature regimes, in order to assess the effect of the different combinations of mutant alleles on fatty acid composition. The IMC201 parent was used as a control in both temperature regimes.

The seeds were planted in Premier Pro-Mix BX potting soil (Premier Horticulture, Quebec, Canada) in four inch plastic pots. Planted seeds were watered and stratified at 5° C. for 5 days and germinated at 20° C. day temperature and 17° C. night temperature (20/17) in Conviron ATC60 controlled-environment growth chambers (Controlled Environments, Winnipeg, MB). Each gene combination was randomized and replicated 10 times in each of two separate growth chambers. At flowering, one chamber was reduced to a diurnal temperature cycle of 14° C. day temperature and 11° C. night temperature (14/11) while the other remained at 20/17. The temperature treatments were imposed to identify the effects of temperature on fatty acid composition. Plants were watered five times per week and fertilized bi-weekly using a 20:20:20 (NPK) liquid fertilizer at a rate of 150 ppm. Plants were bagged individually to ensure self-pollination and genetic purity of the seed. Seeds from each plant were harvested at physiological seed maturity. All plants were analyzed using PCR based assays to confirm the presence of the FatB mutant alleles at the expected loci as well as the presence of mutant alleles of fatty acid desaturase genes (mFad3a, mFad3b and mFad2d) from the IMC201 pedigree.

IMC201 was selected from a cross of 91AE.318×IMC02. 91AE.318 is a sister or descendent of IMC129, which is described in U.S. Pat. No. 5,668,299. IMC02 was obtained from a cross of IMC01×Westar. See Example 3 of U.S. Pat. No. 5,750,827. IMC02 contains a mutation in both the fad3A and fad3B genes. The fad3A gene contains a C to T mutation at position 2565 from ATG in genomic DNA, resulting in the substitution of a cysteine for arginine at position 275 of the Fad3A protein. The fad3B gene contains a G to A mutation at position 3053 from ATG in genomic DNA, located in the exon-intron splice site recognition sequence.

A modified method for gas chromatograph determination of fatty acid profile per the American Oil Chemist's Society protocol (AOCS, 2009) was used for sample evaluation. Vials were placed in a Hewlett-Packard 5890 Series II gas chromatograph (Hewlett-Packard, Palo Alto, Calif.) equipped with a fused silica capillary column (5 m×0.180 mm and 0.20 μm film thickness) packed with a polyethylene glycol based DB-Wax® for liquid phase separation (J&W Scientific, Folsom, Calif.). Hydrogen (H²) was used as the carrier gas at a flow rate of 2.5 mL/min and the column temperature was isothermal at 200° C. Seed from each plant was tested via this method in replicates of three.

Fatty acid data from plants grown under the different temperature regimes was analyzed in two ways. First, data was analyzed separately as different environments and then it was pooled and analyzed across environments. Data was analyzed in SAS (SAS Institute, 2003) using proc glm to estimate differences in mean fatty acid values. Table 8 contains the genotype, population size, mean value and standard deviation of palmitic, stearic and total saturated fatty acid of seeds produced by plants carrying various combinations of mutant FatB alleles grown in two environmental growth chambers set at different diurnal temperature regimens (20° C. day/17° C. night; 14° C. day/11° C. night) as discussed above. Genotypes preceded by Iso are mutant allele combinations and the numbers thereafter indicate the specific locus. Means with different letters are significantly different as determined by a Student-Newman-Keuls mean separation test

TABLE 8
Across Environments

| Genotype | N | C16:0 | s.d. | Genotype | n | C18:0 | s.d. | Genotype | n | Total Sats | s.d. |
|---|---|---|---|---|---|---|---|---|---|---|---|
| IMC201 | 16 | 3.795a | 0.424 | Iso 234 | 16 | 1.971a | 0.880 | IMC201 | 16 | 6.757a | 0.925 |
| Iso234 | 16 | 3.273b | 0.368 | IMC201 | 16 | 1.831ab | 0.373 | Iso234 | 16 | 6.542a | 1.549 |
| Iso124 | 9 | 3.135bc | 0.109 | Iso124 | 9 | 1.81ab | 0.195 | Iso124 | 9 | 6.168ab | 0.338 |
| Iso123 | 8 | 2.959c | 0.174 | Iso123 | 8 | 1.628ab | 0.227 | Iso123 | 8 | 5.719bc | 0.376 |
| Iso1234 | 17 | 2.721d | 0.240 | Iso1234 | 17 | 1.520b | 0.310 | Iso1234 | 17 | 5.412c | 0.729 |

PCR screening showed that the mFad2d mutant allele from IMC129 was segregating in all of the FatB mutant combinations. It was found to be absent or heterozygous in 70% of the individuals screened. The effect of this allele was statistically significant for palmitic, stearic and total saturated fatty acid contents (F=11.17, p=0.0011; F=4.43, p=0.0376; F=6.55, p=0.0118, respectively) in analyses comparing means across environments. Therefore, the number of copies of this allele (0, 1 or 2) was included as a covariate in ANOVA mean separation tests. Significant differences were discovered for mean values of seed palmitic and total saturated fatty acid content in analyses using data pooled across environments (Table 9).

All plants carrying mutant FatB alleles showed statistically significant reductions in seed palmitic acid relative to the IMC201 control with the largest reduction in plants carrying all 4 mutant alleles. Significant reductions in total saturated fatty acid were found in seeds produced by plants carrying mutant alleles 1, 2 and 3 (i.e., Iso 123 in Tables 9 and 10) as well as Iso 1234.

Statistically significant differences were discovered for mean stearic acid content when seeds produced in the different chambers under different temperature treatments were analyzed separately (Table 10, means with different letters are significantly different as determined by a Student-Newman-Keuls mean separation test). In the 20/17 environment, Iso 123, Iso 124 and Iso 1234 all showed significant reductions in stearic acid. Only Iso 1234 showed this reduction in the 14/11 environment. Reductions in total saturated fatty acid content for Iso 123, Iso 124 and Iso 1234 were significant in the 20/17 environment and all mutant allele combinations showed significant reductions in the 14/11 environment (Tables 9 and 10). Again, plants carrying all forms of the mutant allele combinations showed significant reductions in palmitic acid when data from environments was analyzed separately.

TABLE 9

| | | | | | | | | | | | |
|---|---|---|---|---|---|---|---|---|---|---|---|
| Genotype | N | C16:0 | s.d. | Genotype | N | C18:0 | s.d. | Genotype | n | Total Sats | s.d. |

20/17 Environment

| | | | | | | | | | | | |
|---|---|---|---|---|---|---|---|---|---|---|---|
| IMC201 | 8 | 3.971a | 0.292 | Iso 234 | 7 | 2.771a | 0.807 | Iso234 | 7 | 8.098a | 1.116 |
| Iso234 | 7 | 3.614b | 0.106 | IMC201 | 8 | 2.158b | 0.203 | IMC201 | 8 | 7.465b | 0.244 |
| Iso124 | 9 | 3.135c | 0.109 | Iso124 | 9 | 1.810c | 0.195 | Iso124 | 9 | 6.168c | 0.338 |
| Iso123 | 4 | 2.979cd | 0.159 | Iso123 | 4 | 1.806c | 0.111 | Iso123 | 4 | 5.988c | 0.256 |
| Iso1234 | 9 | 2.916d | 0.102 | Iso1234 | 9 | 1.749c | 0.187 | Iso1234 | 9 | 5.965c | 0.390 |

14/11 Environment

| | | | | | | | | | | | |
|---|---|---|---|---|---|---|---|---|---|---|---|
| IMC201 | 8 | 3.618a | 0.471 | IMC201 | 8 | 1.504a | 0.195 | IMC201 | 8 | 6.050a | 0.826 |
| Iso234 | 9 | 3.007b | 0.317 | Iso123 | 4 | 1.451a | 0.156 | Iso123 | 4 | 5.450b | 0.268 |
| Iso123 | 4 | 2.939b | 0.210 | Iso234 | 9 | 1.349ab | 0.082 | Iso234 | 9 | 5.331b | 0.305 |
| Iso1234 | 8 | 2.501c | 0.119 | Iso1234 | 8 | 1.262b | 0.197 | Iso1234 | 8 | 4.791c | 0.463 |

The mean content of the three fatty acids reported here were significantly different between the environments (C16:0 F=59.59, p<0.0001; C18:0 F=83.42, p<0.0001; Total Sats F=122.02, p<0.0001). The data indicate that a low temperature environment reduces the amount of these saturated fatty acids in the seed oil.

TABLE 10
Fatty Acid Profile of IMC201 and Plants With Mutant FatB Alleles

| Genotype | Environment | 14:0 | 16:0 | 16:1 | 18:0 | 18:1 | 18:2 | 18:3 | 20:0 | 20:1 | 20:2 | 22:0 | 22:1 | 24:0 | 24:1 | Total Sats |
|---|---|---|---|---|---|---|---|---|---|---|---|---|---|---|---|---|
| IMC201 | High (20/17) | 0.05 | 4.26 | 0.20 | 2.06 | 78.16 | 10.15 | 2.02 | 0.84 | 1.32 | 0.05 | 0.44 | 0.02 | 0.25 | 0.18 | 7.90 |
| IMC201 | High (20/17) | 0.05 | 4.06 | 0.20 | 2.21 | 75.83 | 12.34 | 2.49 | 0.78 | 1.25 | 0.06 | 0.36 | 0.02 | 0.19 | 0.15 | 7.65 |
| IMC201 | High (20/17) | 0.05 | 3.79 | 0.18 | 2.34 | 77.38 | 10.98 | 2.25 | 0.84 | 1.30 | 0.06 | 0.40 | 0.02 | 0.23 | 0.20 | 7.64 |
| IMC201 | High (20/17) | 0.05 | 3.99 | 0.19 | 2.16 | 76.33 | 12.22 | 2.27 | 0.77 | 1.24 | 0.05 | 0.36 | 0.02 | 0.17 | 0.18 | 7.50 |
| IMC201 | High (20/17) | 0.05 | 4.30 | 0.22 | 1.86 | 77.13 | 11.37 | 2.37 | 0.69 | 1.23 | 0.06 | 0.36 | 0.03 | 0.17 | 0.17 | 7.43 |
| IMC201 | High (20/17) | 0.05 | 4.34 | 0.22 | 1.84 | 76.58 | 11.93 | 2.43 | 0.68 | 1.20 | 0.06 | 0.34 | 0.02 | 0.16 | 0.17 | 7.40 |
| IMC201 | High (20/17) | 0.05 | 4.03 | 0.19 | 2.05 | 76.20 | 12.56 | 2.31 | 0.69 | 1.21 | 0.06 | 0.34 | 0.02 | 0.15 | 0.15 | 7.30 |
| IMC201 | High (20/17) | 0.05 | 3.90 | 0.19 | 2.16 | 75.80 | 12.94 | 2.42 | 0.68 | 1.22 | 0.06 | 0.30 | 0.02 | 0.15 | 0.13 | 7.22 |
| IMC201 | High (20/17) | 0.03 | 3.41 | 0.13 | 2.46 | 76.72 | 12.12 | 2.37 | 0.72 | 1.35 | 0.07 | 0.30 | 0.01 | 0.18 | 0.12 | 7.10 |
| IMC201 | Low (14/11) | 0.06 | 4.02 | 0.23 | 1.47 | 74.65 | 13.94 | 3.03 | 0.58 | 1.33 | 0.06 | 0.33 | 0.03 | 0.13 | 0.15 | 6.58 |
| IMC201 | Low (14/11) | 0.05 | 3.97 | 0.22 | 1.49 | 75.43 | 13.43 | 2.74 | 0.58 | 1.36 | 0.06 | 0.34 | 0.03 | 0.11 | 0.18 | 6.54 |
| IMC201 | Low (14/11) | 0.05 | 3.76 | 0.21 | 1.63 | 76.15 | 12.63 | 2.98 | 0.61 | 1.27 | 0.06 | 0.34 | 0.04 | 0.11 | 0.16 | 6.51 |
| IMC201 | Low (14/11) | 0.04 | 3.84 | 0.21 | 1.42 | 75.88 | 12.93 | 2.93 | 0.57 | 1.43 | 0.07 | 0.36 | 0.02 | 0.12 | 0.19 | 6.35 |
| IMC201 | Low (14/11) | 0.04 | 3.66 | 0.20 | 1.59 | 75.94 | 12.96 | 2.98 | 0.55 | 1.32 | 0.08 | 0.34 | 0.05 | 0.10 | 0.20 | 6.28 |
| IMC201 | Low (14/11) | 0.05 | 3.67 | 0.20 | 1.62 | 76.61 | 12.52 | 2.96 | 0.37 | 1.32 | 0.05 | 0.31 | 0.03 | 0.11 | 0.20 | 6.13 |
| IMC201 | Low (14/11) | 0.03 | 3.49 | 0.13 | 1.73 | 74.49 | 14.29 | 3.38 | 0.40 | 1.61 | 0.04 | 0.21 | 0.02 | 0.06 | 0.13 | 5.92 |
| IMC201 | Low (14/11) | 0.02 | 2.53 | 0.16 | 1.09 | 75.76 | 15.24 | 3.20 | 0.14 | 1.29 | 0.08 | 0.24 | 0.02 | 0.07 | 0.16 | 4.08 |
| Iso123 | High (20/17) | 0.04 | 3.20 | 0.26 | 1.82 | 76.55 | 12.40 | 3.05 | 0.68 | 1.21 | 0.06 | 0.35 | 0.02 | 0.17 | 0.18 | 6.26 |
| Iso123 | High (20/17) | 0.03 | 2.85 | 0.27 | 1.97 | 78.31 | 10.98 | 2.78 | 0.76 | 1.17 | 0.04 | 0.38 | 0.04 | 0.25 | 0.15 | 6.25 |
| Iso123 | High (20/17) | 0.04 | 2.96 | 0.24 | 1.95 | 77.09 | 12.09 | 3.06 | 0.68 | 1.15 | 0.06 | 0.33 | 0.02 | 0.16 | 0.18 | 6.10 |
| Iso123 | High (20/17) | 0.04 | 2.82 | 0.32 | 1.75 | 74.68 | 14.64 | 2.96 | 0.69 | 1.17 | 0.05 | 0.38 | 0.01 | 0.25 | 0.26 | 5.92 |
| Iso123 | High (20/17) | 0.04 | 2.94 | 0.27 | 1.70 | 76.32 | 13.20 | 3.21 | 0.57 | 1.11 | 0.06 | 0.29 | 0.01 | 0.13 | 0.16 | 5.67 |
| Iso123 | Low (14/11) | 0.04 | 3.19 | 0.27 | 1.50 | 72.50 | 15.95 | 3.75 | 0.59 | 1.41 | 0.07 | 0.37 | 0.02 | 0.12 | 0.23 | 5.80 |
| Iso123 | Low (14/11) | 0.05 | 2.89 | 0.30 | 1.64 | 75.34 | 13.62 | 3.80 | 0.52 | 1.12 | 0.06 | 0.30 | 0.04 | 0.11 | 0.21 | 5.51 |
| Iso123 | Low (14/11) | 0.03 | 3.00 | 0.24 | 1.29 | 75.38 | 13.96 | 3.40 | 0.53 | 1.41 | 0.06 | 0.35 | 0.03 | 0.11 | 0.21 | 5.32 |
| Iso123 | Low (14/11) | 0.03 | 2.68 | 0.25 | 1.37 | 76.24 | 12.90 | 3.65 | 0.59 | 1.43 | 0.06 | 0.38 | 0.02 | 0.14 | 0.26 | 5.18 |
| Iso124 | High (20/17) | 0.04 | 3.23 | 0.28 | 2.13 | 72.73 | 16.29 | 2.74 | 0.72 | 1.07 | 0.05 | 0.37 | 0.02 | 0.16 | 0.16 | 6.65 |
| Iso124 | High (20/17) | 0.04 | 3.17 | 0.27 | 2.01 | 72.62 | 16.76 | 2.55 | 0.71 | 1.09 | 0.05 | 0.37 | 0.02 | 0.17 | 0.16 | 6.48 |
| Iso124 | High (20/17) | 0.04 | 3.12 | 0.24 | 1.87 | 78.55 | 10.92 | 2.43 | 0.74 | 1.24 | 0.06 | 0.39 | 0.02 | 0.21 | 0.17 | 6.37 |
| Iso124 | High (20/17) | 0.04 | 3.19 | 0.25 | 1.82 | 71.84 | 17.19 | 2.96 | 0.67 | 1.15 | 0.06 | 0.37 | 0.02 | 0.19 | 0.26 | 6.27 |
| Iso124 | High (20/17) | 0.05 | 3.15 | 0.32 | 1.82 | 77.52 | 11.88 | 2.65 | 0.68 | 1.18 | 0.06 | 0.36 | 0.00 | 0.18 | 0.16 | 6.22 |
| Iso124 | High (20/17) | 0.04 | 3.20 | 0.28 | 1.89 | 67.27 | 22.04 | 2.95 | 0.62 | 1.02 | 0.06 | 0.31 | 0.01 | 0.13 | 0.17 | 6.20 |
| Iso124 | High (20/17) | 0.04 | 3.24 | 0.26 | 1.57 | 66.46 | 22.91 | 2.93 | 0.62 | 1.15 | 0.07 | 0.35 | 0.03 | 0.14 | 0.23 | 5.97 |
| Iso124 | High (20/17) | 0.04 | 2.98 | 0.22 | 1.59 | 78.96 | 11.00 | 2.76 | 0.61 | 1.15 | 0.05 | 0.34 | 0.02 | 0.15 | 0.15 | 5.70 |
| Iso124 | High (20/17) | 0.04 | 2.93 | 0.24 | 1.60 | 78.65 | 11.12 | 2.87 | 0.62 | 1.21 | 0.05 | 0.33 | 0.02 | 0.15 | 0.18 | 5.65 |
| Iso124 | Low (14/11) | 0.04 | 2.84 | 0.31 | 1.54 | 73.84 | 15.65 | 3.51 | 0.36 | 1.16 | 0.06 | 0.35 | 0.02 | 0.10 | 0.22 | 5.23 |
| Iso234 | High (20/17) | 0.05 | 3.64 | 0.25 | 3.72 | 69.54 | 16.12 | 2.79 | 1.30 | 1.13 | 0.07 | 0.63 | 0.02 | 0.42 | 0.31 | 9.78 |
| Iso234 | High (20/17) | 0.05 | 3.39 | 0.22 | 3.70 | 67.48 | 18.73 | 3.14 | 1.13 | 1.06 | 0.06 | 0.50 | 0.03 | 0.27 | 0.24 | 9.04 |
| Iso234 | High (20/17) | 0.05 | 3.60 | 0.22 | 3.26 | 70.34 | 17.05 | 2.52 | 1.04 | 1.03 | 0.06 | 0.45 | 0.01 | 0.21 | 0.17 | 8.60 |
| Iso234 | High (20/17) | 0.05 | 3.69 | 0.25 | 2.64 | 70.29 | 17.18 | 3.07 | 0.85 | 1.07 | 0.06 | 0.39 | 0.01 | 0.26 | 0.18 | 7.88 |
| Iso234 | High (20/17) | 0.05 | 3.69 | 0.23 | 2.37 | 72.38 | 15.60 | 2.50 | 0.92 | 1.20 | 0.07 | 0.49 | 0.02 | 0.27 | 0.22 | 7.79 |
| Iso234 | High (20/17) | 0.05 | 3.82 | 0.31 | 1.76 | 74.59 | 14.16 | 2.74 | 0.64 | 1.15 | 0.07 | 0.34 | 0.01 | 0.25 | 0.12 | 6.85 |
| Iso234 | High (20/17) | 0.05 | 3.70 | 0.26 | 1.76 | 76.47 | 12.22 | 2.70 | 0.70 | 1.26 | 0.06 | 0.37 | 0.03 | 0.23 | 0.19 | 6.81 |
| Iso234 | High (20/17) | 0.05 | 3.59 | 0.25 | 1.94 | 70.65 | 18.05 | 2.81 | 0.68 | 1.13 | 0.06 | 0.34 | 0.04 | 0.18 | 0.21 | 6.79 |
| Iso234 | Low (14/11) | 0.06 | 3.71 | 0.32 | 1.27 | 66.07 | 21.66 | 4.24 | 0.35 | 1.32 | 0.07 | 0.43 | 0.06 | 0.16 | 0.29 | 5.98 |
| Iso234 | Low (14/11) | 0.03 | 3.18 | 0.32 | 1.40 | 66.38 | 22.07 | 3.79 | 0.55 | 1.29 | 0.10 | 0.39 | 0.06 | 0.13 | 0.32 | 5.68 |
| Iso234 | Low (14/11) | 0.03 | 3.28 | 0.29 | 1.40 | 66.93 | 23.28 | 2.44 | 0.46 | 1.13 | 0.06 | 0.29 | 0.04 | 0.12 | 0.24 | 5.59 |
| Iso234 | Low (14/11) | 0.04 | 3.13 | 0.28 | 1.43 | 67.90 | 21.10 | 3.53 | 0.52 | 1.30 | 0.08 | 0.34 | 0.02 | 0.11 | 0.23 | 5.57 |
| Iso234 | Low (14/11) | 0.04 | 3.05 | 0.27 | 1.30 | 68.17 | 20.89 | 3.63 | 0.50 | 1.33 | 0.07 | 0.35 | 0.04 | 0.12 | 0.24 | 5.36 |
| Iso234 | Low (14/11) | 0.05 | 3.12 | 0.29 | 1.30 | 66.56 | 22.26 | 3.88 | 0.35 | 1.30 | 0.10 | 0.35 | 0.03 | 0.14 | 0.26 | 5.30 |
| Iso234 | Low (14/11) | 0.02 | 3.12 | 0.30 | 1.33 | 69.56 | 20.66 | 2.59 | 0.33 | 1.28 | 0.08 | 0.34 | 0.04 | 0.11 | 0.24 | 5.25 |
| Iso234 | Low (14/11) | 0.04 | 2.74 | 0.27 | 1.45 | 76.56 | 12.91 | 3.53 | 0.49 | 1.25 | 0.06 | 0.32 | 0.04 | 0.11 | 0.21 | 5.15 |
| Iso234 | Low (14/11) | 0.04 | 2.93 | 0.25 | 1.18 | 70.80 | 18.54 | 3.58 | 0.49 | 1.40 | 0.07 | 0.34 | 0.02 | 0.11 | 0.26 | 5.09 |
| Iso234 | Low (14/11) | 0.03 | 2.52 | 0.38 | 1.35 | 72.27 | 16.85 | 3.81 | 0.54 | 1.30 | 0.06 | 0.40 | 0.00 | 0.15 | 0.33 | 4.99 |
| Iso1234 | High (20/17) | 0.04 | 3.07 | 0.26 | 2.09 | 69.61 | 18.91 | 2.87 | 0.88 | 1.18 | 0.07 | 0.52 | 0.03 | 0.23 | 0.23 | 6.84 |
| Iso1234 | High (20/17) | 0.04 | 2.90 | 0.26 | 1.92 | 68.36 | 20.89 | 3.05 | 0.72 | 1.06 | 0.06 | 0.37 | 0.00 | 0.20 | 0.18 | 6.15 |
| Iso1234 | High (20/17) | 0.04 | 2.92 | 0.26 | 1.75 | 73.39 | 15.92 | 2.90 | 0.75 | 1.17 | 0.06 | 0.42 | 0.03 | 0.20 | 0.21 | 6.07 |
| Iso1234 | High (20/17) | 0.04 | 2.87 | 0.28 | 1.83 | 71.68 | 17.50 | 3.11 | 0.72 | 1.11 | 0.06 | 0.37 | 0.03 | 0.19 | 0.22 | 6.02 |
| Iso1234 | High (20/17) | 0.04 | 3.01 | 0.26 | 1.54 | 71.11 | 18.51 | 2.66 | 0.71 | 1.19 | 0.07 | 0.44 | 0.03 | 0.20 | 0.23 | 5.94 |
| Iso1234 | High (20/17) | 0.04 | 3.01 | 0.29 | 1.57 | 70.56 | 18.63 | 3.40 | 0.62 | 1.12 | 0.06 | 0.34 | 0.02 | 0.16 | 0.19 | 5.74 |
| Iso1234 | High (20/17) | 0.04 | 2.79 | 0.27 | 1.80 | 70.89 | 18.95 | 2.88 | 0.63 | 1.06 | 0.06 | 0.31 | 0.02 | 0.15 | 0.15 | 5.74 |
| Iso1234 | High (20/17) | 0.04 | 2.77 | 0.24 | 1.71 | 72.23 | 17.53 | 2.90 | 0.66 | 1.12 | 0.07 | 0.35 | 0.01 | 0.19 | 0.18 | 5.72 |
| Iso1234 | High (20/17) | 0.04 | 2.89 | 0.28 | 1.53 | 67.27 | 22.56 | 3.11 | 0.57 | 1.03 | 0.07 | 0.31 | 0.01 | 0.14 | 0.19 | 5.47 |
| Iso1234 | Low (14/11) | 0.04 | 2.61 | 0.29 | 1.36 | 68.44 | 20.55 | 3.93 | 0.63 | 1.24 | 0.08 | 0.46 | 0.03 | 0.14 | 0.20 | 5.24 |
| Iso1234 | Low (14/11) | 0.02 | 2.51 | 0.27 | 1.42 | 69.76 | 19.44 | 3.75 | 0.64 | 1.27 | 0.10 | 0.44 | 0.02 | 0.15 | 0.23 | 5.17 |
| Iso1234 | Low (14/11) | 0.03 | 2.54 | 0.26 | 1.33 | 64.33 | 25.15 | 3.73 | 0.56 | 1.22 | 0.09 | 0.40 | 0.01 | 0.12 | 0.24 | 4.97 |
| Iso1234 | Low (14/11) | 0.04 | 2.68 | 0.27 | 1.36 | 70.70 | 18.79 | 3.82 | 0.38 | 1.19 | 0.05 | 0.38 | 0.01 | 0.12 | 0.21 | 4.96 |
| Iso1234 | Low (14/11) | 0.03 | 2.47 | 0.29 | 1.31 | 68.06 | 21.43 | 3.63 | 0.59 | 1.35 | 0.07 | 0.40 | 0.02 | 0.11 | 0.27 | 4.89 |
| Iso1234 | Low (14/11) | 0.03 | 2.53 | 0.29 | 1.29 | 65.90 | 23.37 | 3.93 | 0.53 | 1.30 | 0.08 | 0.39 | 0.02 | 0.10 | 0.24 | 4.86 |
| Iso1234 | Low (14/11) | 0.03 | 2.39 | 0.27 | 1.24 | 70.56 | 18.96 | 3.64 | 0.59 | 1.38 | 0.08 | 0.43 | 0.03 | 0.15 | 0.26 | 4.82 |
| Iso1234 | Low (14/11) | 0.04 | 2.48 | 0.28 | 1.34 | 71.51 | 18.58 | 3.57 | 0.34 | 1.14 | 0.08 | 0.34 | 0.02 | 0.10 | 0.19 | 4.63 |
| Iso1234 | Low (14/11) | 0.02 | 2.27 | 0.14 | 0.76 | 74.15 | 13.60 | 6.46 | 0.32 | 1.67 | 0.12 | 0.23 | 0.04 | 0.07 | 0.16 | 3.66 |

### Example 7

### Brassica Plant Lines 1764, 1975, and 2650

Lines 1764, 1975, and 2650 were selected from the mutagenized population of IMC201 seeds of Example 5 as follows. Three thousand bulk M₂ generation seeds were planted. Upon maturity, M₃ seed (2500 individuals) was harvested from 2500 M₂ plants and analyzed via GC. Table 11 provides the fatty acid profile of seed from three lines identified as having a low total saturates content in seed oil: 1764, 1975, and 2650. M₃ seeds of 1764, 1975, and 2650 were planted (100 per line) and the resulting plants were self-pollinated. M₄ seeds were harvested from the plants and analyzed via GC (see Table 12).

TABLE 11
Fatty acid composition of M₃ generation seed from mutant lines exhibiting reduced saturated fatty acid content

| Line | C140 | C160 | C161 | C180 | C181 | C182 | C183 | C200 | C201 | C202 | C220 | C221 | C240 | C241 | Total Sats |
|---|---|---|---|---|---|---|---|---|---|---|---|---|---|---|---|
| 1764 | 0.05 | 3.30 | 0.31 | 1.65 | 76.30 | 13.40 | 2.00 | 0.668 | 1.46 | 0.06 | 0.38 | 0.02 | 0.28 | 0.15 | 6.32 |
| 1975 | 0.03 | 3.19 | 0.22 | 1.35 | 75.51 | 14.21 | 2.19 | 0.59 | 1.77 | 0.10 | 0.43 | 0.00 | 0.23 | 0.19 | 5.82 |
| 2650 | 0.04 | 3.00 | 0.12 | 3.79 | 77.77 | 8.59 | 2.056 | 1.42 | 1.68 | 0.08 | 0.74 | 0.02 | 0.45 | 0.26 | 9.44 |

TABLE 12
Fatty acid composition of M₄ generation seed from three mutant lines exhibiting reduced saturated fatty acid content

| Line | C140 | C160 | C161 | C180 | C181 | C182 | C183 | C200 | C201 | C202 | C220 | C221 | C240 | C241 | Total sats |
|---|---|---|---|---|---|---|---|---|---|---|---|---|---|---|---|
| 1764-06 | 0.05 | 3.06 | 0.34 | 1.94 | 76.89 | 12.57 | 1.99 | 0.70 | 1.34 | 0.05 | 0.39 | 0.00 | 0.23 | 0.45 | 6.37 |
| 1764-35 | 0.04 | 3.54 | 0.47 | 1.64 | 74.09 | 15.38 | 2.04 | 0.59 | 1.32 | 0.05 | 0.32 | 0.00 | 0.19 | 0.34 | 6.31 |
| 1764-43 | 0.04 | 3.06 | 0.32 | 1.88 | 75.24 | 14.26 | 1.86 | 0.75 | 1.58 | 0.07 | 0.45 | 0.03 | 0.28 | 0.18 | 6.46 |
| 1764-59 | 0.05 | 3.33 | 0.38 | 1.57 | 74.92 | 14.56 | 2.21 | 0.57 | 1.33 | 0.05 | 0.32 | 0.03 | 0.19 | 0.49 | 6.02 |
| 1764-91 | 0.05 | 3.11 | 0.34 | 1.77 | 75.83 | 13.70 | 2.15 | 0.67 | 1.37 | 0.05 | 0.38 | 0.02 | 0.24 | 0.32 | 6.21 |
| 1764-92 | 0.04 | 3.00 | 0.30 | 2.07 | 76.75 | 12.79 | 2.11 | 0.74 | 1.40 | 0.05 | 0.40 | 0.00 | 0.22 | 0.13 | 6.47 |
| 1764-95 | 0.06 | 3.38 | 0.40 | 1.62 | 74.11 | 15.17 | 2.18 | 0.63 | 1.36 | 0.06 | 0.37 | 0.03 | 0.22 | 0.43 | 6.27 |
| 1975-01 | 0.05 | 3.31 | 0.23 | 1.52 | 73.60 | 15.79 | 2.17 | 0.62 | 1.51 | 0.08 | 0.40 | 0.03 | 0.17 | 0.51 | 6.07 |
| 1975-04 | 0.02 | 3.04 | 0.16 | 1.74 | 77.28 | 12.64 | 2.08 | 0.66 | 1.54 | 0.06 | 0.36 | 0.00 | 0.17 | 0.24 | 6.00 |
| 1975-32 | 0.03 | 3.54 | 0.22 | 1.52 | 73.89 | 15.44 | 2.35 | 0.59 | 1.55 | 0.09 | 0.34 | 0.00 | 0.18 | 0.26 | 6.20 |
| 1975-65 | 0.03 | 3.18 | 0.16 | 1.71 | 75.16 | 14.26 | 2.22 | 0.63 | 1.64 | 0.09 | 0.36 | 0.00 | 0.16 | 0.39 | 6.07 |
| 1975-76 | 0.05 | 3.52 | 0.19 | 1.48 | 73.18 | 16.04 | 2.37 | 0.62 | 1.63 | 0.09 | 0.39 | 0.03 | 0.23 | 0.19 | 6.28 |
| 1975-84 | 0.04 | 3.12 | 0.14 | 1.68 | 75.57 | 14.07 | 2.35 | 0.64 | 1.61 | 0.09 | 0.35 | 0.00 | 0.20 | 0.12 | 6.03 |
| 1975-90 | 0.04 | 3.34 | 0.23 | 1.40 | 72.21 | 17.44 | 2.25 | 0.58 | 1.70 | 0.11 | 0.35 | 0.00 | 0.20 | 0.16 | 5.92 |
| 1975-96 | 0.04 | 3.13 | 0.17 | 1.99 | 76.43 | 12.99 | 2.05 | 0.76 | 1.60 | 0.07 | 0.40 | 0.00 | 0.23 | 0.13 | 6.55 |
| 1975-99 | 0.04 | 3.13 | 0.20 | 1.83 | 74.80 | 14.34 | 2.15 | 0.72 | 1.68 | 0.08 | 0.43 | 0.04 | 0.21 | 0.35 | 6.37 |
| 2650-20 | 0.06 | 2.81 | 0.13 | 4.08 | 74.24 | 11.71 | 2.29 | 1.38 | 1.84 | 0.11 | 0.62 | 0.05 | 0.38 | 0.31 | 9.32 |
| 2650-36 | 0.05 | 2.93 | 0.14 | 3.63 | 74.55 | 11.95 | 2.58 | 1.20 | 1.64 | 0.09 | 0.55 | 0.00 | 0.28 | 0.40 | 8.64 |
| 2650-45 | 0.06 | 3.02 | 0.14 | 3.74 | 75.16 | 11.27 | 2.49 | 1.19 | 1.58 | 0.08 | 0.51 | 0.00 | 0.26 | 0.52 | 8.77 |
| IMC02-01 | 0.06 | 3.73 | 0.21 | 2.96 | 70.35 | 18.37 | 1.30 | 0.98 | 1.15 | 0.05 | 0.44 | 0.01 | 0.26 | 0.13 | 8.43 |
| IMC02-02 | 0.05 | 3.64 | 0.23 | 2.85 | 70.86 | 17.82 | 1.28 | 0.98 | 1.21 | 0.05 | 0.48 | 0.02 | 0.29 | 0.26 | 8.27 |
| IMC02-03 | 0.05 | 3.66 | 0.21 | 2.90 | 69.84 | 18.96 | 1.32 | 0.94 | 1.15 | 0.05 | 0.42 | 0.02 | 0.27 | 0.21 | 8.24 |
| IMC02-04 | 0.05 | 3.62 | 0.23 | 3.06 | 68.94 | 19.37 | 1.38 | 1.01 | 1.20 | 0.06 | 0.49 | 0.00 | 0.31 | 0.28 | 8.54 |
| IMC02-05 | 0.04 | 3.62 | 0.24 | 3.13 | 69.27 | 19.33 | 1.34 | 0.96 | 1.13 | 0.06 | 0.41 | 0.01 | 0.25 | 0.20 | 8.42 |
| IMC02-06 | 0.05 | 3.87 | 0.25 | 3.74 | 70.11 | 17.17 | 1.40 | 1.21 | 1.14 | 0.06 | 0.58 | 0.00 | 0.34 | 0.09 | 9.79 |
| IMC02-07 | 0.06 | 3.75 | 0.27 | 2.89 | 66.48 | 22.22 | 1.34 | 0.89 | 1.11 | 0.05 | 0.40 | 0.00 | 0.23 | 0.29 | 8.23 |
| IMC02-08 | 0.06 | 3.71 | 0.25 | 2.83 | 69.87 | 18.87 | 1.25 | 0.95 | 1.16 | 0.05 | 0.43 | 0.00 | 0.27 | 0.30 | 8.26 |
| IMC02-09 | 0.07 | 4.51 | 0.35 | 3.83 | 65.22 | 20.57 | 1.96 | 1.20 | 1.03 | 0.00 | 0.57 | 0.00 | 0.37 | 0.34 | 10.53 |
| IMC02-10 | 0.05 | 3.66 | 0.25 | 2.77 | 68.23 | 20.84 | 1.27 | 0.90 | 1.17 | 0.05 | 0.41 | 0.00 | 0.25 | 0.16 | 8.03 |
| IMC02-11 | 0.05 | 3.79 | 0.23 | 2.95 | 68.43 | 20.15 | 1.32 | 0.98 | 1.15 | 0.06 | 0.46 | 0.00 | 0.29 | 0.13 | 8.52 |
| IMC02-12 | 0.06 | 3.72 | 0.25 | 2.78 | 68.35 | 20.50 | 1.30 | 0.90 | 1.15 | 0.05 | 0.42 | 0.00 | 0.26 | 0.25 | 8.14 |
| IMC02-13 | 0.08 | 3.92 | 0.25 | 2.92 | 67.17 | 21.30 | 1.43 | 0.93 | 1.11 | 0.06 | 0.42 | 0.00 | 0.30 | 0.12 | 8.56 |
| IMC02-14 | 0.05 | 3.64 | 0.23 | 3.09 | 71.73 | 16.73 | 1.36 | 1.05 | 1.19 | 0.05 | 0.51 | 0.00 | 0.28 | 0.09 | 8.62 |
| IMC02-15 | 0.06 | 3.73 | 0.25 | 2.99 | 69.14 | 19.49 | 1.23 | 0.99 | 1.15 | 0.05 | 0.45 | 0.00 | 0.29 | 0.17 | 8.51 |
| IMC02-16 | 0.06 | 3.76 | 0.24 | 2.81 | 69.05 | 19.89 | 1.21 | 0.94 | 1.17 | 0.05 | 0.43 | 0.00 | 0.27 | 0.14 | 8.25 |
| IMC02-17 | 0.05 | 3.63 | 0.25 | 2.61 | 67.52 | 21.91 | 1.33 | 0.83 | 1.12 | 0.06 | 0.39 | 0.00 | 0.21 | 0.10 | 7.72 |
| IMC02-18 | 0.05 | 3.66 | 0.22 | 3.19 | 71.15 | 17.32 | 1.25 | 1.06 | 1.16 | 0.05 | 0.51 | 0.00 | 0.29 | 0.11 | 8.76 |
| IMC02-19 | 0.05 | 3.65 | 0.24 | 3.18 | 68.92 | 19.62 | 1.28 | 1.02 | 1.13 | 0.05 | 0.45 | 0.00 | 0.30 | 0.12 | 8.64 |
| IMC02-20 | 0.05 | 3.71 | 0.26 | 2.79 | 66.85 | 22.13 | 1.55 | 0.87 | 1.10 | 0.06 | 0.41 | 0.00 | 0.22 | 0.00 | 8.05 |
| IMC02Ave | 0.05 | 3.75 | 0.24 | 3.01 | 68.87 | 19.63 | 1.36 | 0.98 | 1.14 | 0.05 | 0.45 | 0.00 | 0.28 | 0.17 | 8.52 |
| IMC201-01 | 0.05 | 4.01 | 0.19 | 2.45 | 77.44 | 10.56 | 2.01 | 0.93 | 1.38 | 0.05 | 0.47 | 0.02 | 0.28 | 0.15 | 8.20 |
| IMC201-02 | 0.05 | 3.94 | 0.18 | 2.44 | 77.52 | 10.55 | 2.09 | 0.92 | 1.38 | 0.05 | 0.46 | 0.02 | 0.26 | 0.15 | 8.07 |
| IMC201-03 | 0.06 | 4.06 | 0.21 | 2.59 | 76.51 | 11.16 | 2.06 | 0.94 | 1.34 | 0.05 | 0.46 | 0.02 | 0.26 | 0.28 | 8.37 |
| IMC201-04 | 0.06 | 4.02 | 0.21 | 2.46 | 76.25 | 11.61 | 2.21 | 0.87 | 1.32 | 0.05 | 0.42 | 0.00 | 0.23 | 0.29 | 8.05 |
| IMC201-05 | 0.05 | 4.10 | 0.20 | 2.56 | 76.42 | 11.35 | 2.07 | 0.93 | 1.34 | 0.05 | 0.46 | 0.02 | 0.28 | 0.15 | 8.39 |
| IMC201-06 | 0.05 | 4.05 | 0.21 | 2.50 | 76.36 | 11.51 | 2.08 | 0.91 | 1.37 | 0.05 | 0.45 | 0.03 | 0.26 | 0.16 | 8.23 |
| IMC201-07 | 0.07 | 4.22 | 0.22 | 2.62 | 75.71 | 11.77 | 2.05 | 0.94 | 1.35 | 0.05 | 0.47 | 0.02 | 0.26 | 0.25 | 8.58 |
| IMC201-08 | 0.05 | 3.64 | 0.18 | 2.63 | 77.81 | 10.20 | 2.02 | 0.96 | 1.47 | 0.06 | 0.47 | 0.02 | 0.31 | 0.17 | 8.07 |
| IMC201-09 | 0.05 | 4.41 | 0.24 | 2.85 | 63.92 | 22.50 | 2.79 | 0.96 | 1.20 | 0.08 | 0.48 | 0.02 | 0.32 | 0.17 | 9.08 |
| IMC201-10 | 0.05 | 4.03 | 0.18 | 2.48 | 77.12 | 10.69 | 2.17 | 0.90 | 1.33 | 0.05 | 0.45 | 0.00 | 0.23 | 0.31 | 8.15 |
| IMC201Ave | 0.06 | 4.05 | 0.20 | 2.56 | 75.51 | 12.19 | 2.16 | 0.93 | 1.35 | 0.05 | 0.46 | 0.02 | 0.27 | 0.21 | 8.32 |
| Westar16-01 | 0.06 | 4.41 | 0.30 | 2.34 | 65.36 | 18.31 | 6.50 | 0.76 | 1.13 | 0.06 | 0.35 | 0.00 | 0.22 | 0.19 | 8.15 |
| Westar16-02 | 0.06 | 4.25 | 0.26 | 2.37 | 67.28 | 16.80 | 6.24 | 0.75 | 1.13 | 0.05 | 0.35 | 0.02 | 0.20 | 0.24 | 7.99 |
| Westar16-03 | 0.06 | 4.20 | 0.26 | 2.46 | 66.06 | 17.62 | 6.71 | 0.76 | 1.13 | 0.06 | 0.37 | 0.00 | 0.20 | 0.11 | 8.05 |
| Westar16-04 | 0.07 | 4.52 | 0.29 | 2.54 | 64.75 | 18.82 | 6.53 | 0.74 | 1.04 | 0.06 | 0.34 | 0.00 | 0.19 | 0.11 | 8.40 |
| Westar16-05 | 0.07 | 4.30 | 0.27 | 2.43 | 65.09 | 18.31 | 6.67 | 0.80 | 1.19 | 0.07 | 0.39 | 0.00 | 0.25 | 0.17 | 8.23 |
| Westar16-06 | 0.08 | 4.54 | 0.30 | 2.39 | 65.63 | 17.74 | 6.44 | 0.81 | 1.15 | 0.06 | 0.39 | 0.00 | 0.25 | 0.21 | 8.46 |
| Westar16-07 | 0.08 | 4.34 | 0.28 | 2.57 | 65.47 | 17.92 | 6.57 | 0.79 | 1.12 | 0.06 | 0.35 | 0.00 | 0.20 | 0.27 | 8.32 |
| Westar16-08 | 0.07 | 4.37 | 0.28 | 2.18 | 64.49 | 19.54 | 6.61 | 0.64 | 1.05 | 0.06 | 0.28 | 0.00 | 0.15 | 0.28 | 7.70 |
| Westar16-09 | 0.08 | 4.65 | 0.29 | 2.35 | 61.81 | 21.30 | 6.72 | 0.72 | 1.21 | 0.08 | 0.33 | 0.00 | 0.20 | 0.27 | 8.33 |
| Westar16-10 | 0.06 | 4.26 | 0.25 | 2.54 | 67.17 | 16.96 | 5.85 | 0.80 | 1.17 | 0.06 | 0.38 | 0.00 | 0.22 | 0.28 | 8.27 |
| Westar16Ave | 0.07 | 4.39 | 0.28 | 2.42 | 65.31 | 18.33 | 6.48 | 0.76 | 1.13 | 0.06 | 0.35 | 0.00 | 0.21 | 0.21 | 8.19 |

### Example 8

### DH Line Salomon

A cross was made between 15.24 (Example 1) and 1764-92-05 (Example 7). A DH population was generated by collecting F₁ microspores from the cross, treating the microspores with colchicine, and propagating them in vitro. Plantlets formed in vitro from the microspores were moved to a greenhouse and inflorescences that formed were self-pollinated. Seed was harvested from the DH₁ plants at maturity and analyzed for fatty acid profile. Seeds from those plants exhibiting reduced saturated fatty acid content were grown in the greenhouse and in the field. Table 13 contains the fatty acid profile of seeds produced by greenhouse-grown plants of a DH₁ population designated Salomon. Table 14 contains the fatty acid profile of seeds from three plants of DH line Salomon-05 grown in the field and re-coded to Salomon-005. The fatty acid profile of IMC111RR, a registered Canadian B. napus variety, is included as a control in Table 14. The field grown seed of individual plants of Salomon 005 had a range of 3.83% to 4.44% total saturates with 2.92% to 3.35% palmitic acid and 0.29% to 0.47% stearic acid. Line Salomon-005 demonstrated the lowest total saturated fatty acid profile of the DH lines in the greenhouse and in the field.

Table 15 contains the fatty acid profile of seeds from individual Salomon-005 plants, progeny of DH line Salomon, as grown in a growth chamber under the conditions described in Example 6. Under the high temperature environment (20/17), selfed plants of Salomon 005 had a total saturated fatty acid range of 4.13% to 4.67% with palmitic acid of 2.55% to 2.70% and stearic acid of 1.05 to 0.78%. Seed from the same Salomon 005 DH1 source when grown in a low temperature environment (14/11) had a total saturates of 3.45% to 3.93% with palmitic acid of 2.25% to 2.39% and stearic acid of 0.57% to 0.85%. The FATA2 mutation from 15.24 when combined with other low saturate mutations such as 1764, 1975, and 2650 can further reduce total saturates through the additive reduction of palmitic and stearic acids.

In the low 14/11 environment, Salomon-005-09 exhibited the lowest palmitic acid content, Salomon-005-05 exhibited the lowest stearic acid content, and Salomon-005-07 exhibited the lowest total saturated fatty acid content. Table 15 also contains the profile of individual plants of 15.24, IMC201, and F6 progeny of 1764-43-06×1975-90-14 (see Example 10). The data indicate that a low temperature environment reduces the amount of saturated fatty acids in the seed oil.

Lines 1764, 1975 and 2650 are also crossed with 15.36 (Example 3) to generate progeny having reduced saturated fatty acid content.

TABLE 13
Seed Fatty acid composition of progeny of DH₁ line Salomon in the greenhouse

| | | | | | | | | | | | | | | | Total |
|---|---|---|---|---|---|---|---|---|---|---|---|---|---|---|---|
| Line | C140 | C160 | C161 | C180 | C181 | C182 | C183 | C200 | C201 | C202 | C220 | C221 | C240 | C241 | Sats |
| Salomon-01 | 0.05 | 3.68 | 0.33 | 2.24 | 72.56 | 16.20 | 2.16 | 0.73 | 1.19 | 0.064 | 0.35 | 0.00 | 0.23 | 0.23 | 7.28 |
| Salomon-02 | 0.04 | 2.66 | 0.17 | 1.60 | 72.33 | 14.38 | 4.28 | 0.67 | 2.05 | 0.16 | 0.30 | 0.00 | 0.24 | 0.13 | 6.49 |
| Salomon-03 | 0.04 | 2.89 | 0.21 | 1.59 | 76.24 | 13.68 | 2.28 | 0.66 | 1.57 | 0.07 | 0.33 | 0.00 | 0.24 | 0.20 | 5.74 |
| Salomon-04 | 0.05 | 3.17 | 0.19 | 1.40 | 79.15 | 9.70 | 3.47 | 0.56 | 1.52 | 0.06 | 0.29 | 0.00 | 0.24 | 0.21 | 5.70 |
| Salomon-05 | 0.03 | 3.19 | 0.16 | 1.22 | 75.41 | 12.99 | 3.58 | 0.57 | 1.92 | 0.14 | 0.33 | 0.00 | 0.30 | 0.16 | 5.65 |
| Salomon-06 | 0.05 | 3.67 | 0.24 | 1.53 | 76.15 | 12.12 | 3.49 | 0.66 | 1.53 | 0.06 | 0.32 | 0.00 | 0.18 | 0.00 | 6.40 |
| Salomon-07 | 0.05 | 4.37 | 0.20 | 0.87 | 77.28 | 10.76 | 3.46 | 0.43 | 1.81 | 0.10 | 0.25 | 0.00 | 0.22 | 0.20 | 6.19 |
| Salomon-08 | 0.05 | 4.19 | 0.25 | 1.29 | 78.05 | 10.35 | 2.99 | 0.59 | 1.65 | 0.08 | 0.34 | 0.00 | 0.19 | 0.00 | 6.64 |
| Average | 0.05 | 3.48 | 0.22 | 1.47 | 75.9 | 12.52 | 3.21 | 0.61 | 1.66 | 0.092 | 0.31 | 0.00 | 0.23 | 0.14 | 6.26 |

TABLE 14
Seed Fatty acid composition of DH₂ line Salomon-005 in the field

| | | | | | | | | | | | | | | | Total |
|---|---|---|---|---|---|---|---|---|---|---|---|---|---|---|---|
| Line | C140 | C160 | C161 | C180 | C181 | C182 | C183 | C200 | C201 | C202 | C220 | C221 | C240 | C241 | Sats |
| Salomon-005 | 0.036 | 2.92 | 0 | 0.29 | 73.16 | 14.02 | 5.83 | 0.21 | 2.57 | 0.13 | 0.27 | 0.05 | 0.11 | 0.406 | 3.83 |
| Salomon-005 | 0.036 | 2.85 | 0 | 0.55 | 74.17 | 13.24 | 5.74 | 0.27 | 2.44 | 0.12 | 0.28 | 0.02 | 0.02 | 0.268 | 3.99 |
| Salomon-005 | 0.043 | 3.35 | 0 | 0.47 | 71.35 | 15.20 | 5.90 | 0.24 | 2.63 | 0.17 | 0.32 | 0.06 | 0.03 | 0.251 | 4.44 |
| Average | 0.038 | 3.04 | 0.0 | 0.44 | 72.89 | 14.15 | 5.82 | 0.24 | 2.55 | 0.14 | 0.29 | 0.04 | 0.05 | 0.308 | 4.09 |
| IMC111RR | 0.08 | 5.06 | 0.41 | 2.07 | 56.80 | 28.40 | 3.87 | 0.83 | 1.44 | 0.14 | 0.50 | 0.00 | 0.23 | 0.162 | 8.77 |
| IMC111RR | 0.09 | 5.38 | 0.50 | 2.09 | 56.61 | 28.38 | 3.50 | 0.81 | 1.41 | 0.13 | 0.50 | 0.01 | 0.53 | 0.083 | 9.40 |
| IMC111RR | 0.21 | 6.15 | 0.50 | 1.46 | 47.82 | 36.03 | 3.38 | 0.71 | 1.24 | 0.14 | 0.56 | 0.00 | 1.43 | 0.369 | 10.52 |

TABLE 15
Seed fatty acid profile of individual DH line Salomon-005 Plants, 15.24, IMC201, and F6 plants in the growth chamber

| Genotype | Environment | 14:0 | 16:0 | 16:1 | 18:0 | 18:1 | 18:2 | 18:3 | 20:0 | 20:1 | 20:2 | 22:0 | 22:1 | 24:0 | 24:1 | Total Sats |
|---|---|---|---|---|---|---|---|---|---|---|---|---|---|---|---|---|
| Salomon-005-01 | High 20/17 | 0.02 | 2.59 | 0.14 | 1.05 | 76.66 | 11.34 | 5.29 | 0.44 | 1.68 | 0.12 | 0.27 | 0.04 | 0.14 | 0.21 | 4.51 |
| Salomon-005-02 | High 20/17 | 0.02 | 2.64 | 0.13 | 0.93 | 76.44 | 12.61 | 4.65 | 0.37 | 1.56 | 0.13 | 0.23 | 0.04 | 0.11 | 0.15 | 4.31 |
| Salomon-005-03 | High 20/17 | 0.02 | 2.63 | 0.12 | 0.83 | 77.05 | 11.95 | 4.82 | 0.34 | 1.62 | 0.12 | 0.23 | 0.04 | 0.10 | 0.13 | 4.15 |
| Salomon-005-04 | High 20/17 | 0.02 | 2.57 | 0.12 | 0.84 | 77.73 | 11.43 | 4.60 | 0.35 | 1.68 | 0.13 | 0.24 | 0.04 | 0.11 | 0.14 | 4.13 |
| Salomon-005-05 | High 20/17 | 0.02 | 2.67 | 0.13 | 1.08 | 75.92 | 12.43 | 4.82 | 0.47 | 1.67 | 0.13 | 0.28 | 0.05 | 0.15 | 0.19 | 4.67 |
| Salomon-005-06 | High 20/17 | 0.02 | 2.56 | 0.13 | 1.03 | 76.63 | 12.18 | 4.84 | 0.40 | 1.56 | 0.12 | 0.25 | 0.04 | 0.11 | 0.13 | 4.37 |
| Salomon-005-07 | High 20/17 | 0.02 | 2.58 | 0.13 | 0.78 | 77.50 | 11.64 | 4.49 | 0.36 | 1.78 | 0.14 | 0.26 | 0.05 | 0.12 | 0.18 | 4.11 |
| Salomon-005-08 | High 20/17 | 0.02 | 2.70 | 0.14 | 0.90 | 76.60 | 11.80 | 4.92 | 0.41 | 1.73 | 0.14 | 0.27 | 0.04 | 0.13 | 0.20 | 4.44 |
| Salomon-005-09 | High 20/17 | 0.02 | 2.58 | 0.12 | 0.88 | 77.75 | 11.62 | 4.50 | 0.34 | 1.61 | 0.12 | 0.22 | 0.04 | 0.10 | 0.10 | 4.14 |
| Salomon-005-10 | High 20/17 | 0.02 | 2.46 | 0.13 | 0.99 | 77.92 | 11.20 | 4.55 | 0.41 | 1.62 | 0.13 | 0.26 | 0.04 | 0.13 | 0.16 | 4.28 |
| Salomon-005-01 | Low 14/11 | 0.02 | 2.27 | 0.12 | 0.68 | 73.66 | 13.53 | 6.86 | 0.32 | 1.83 | 0.13 | 0.25 | 0.06 | 0.06 | 0.21 | 3.59 |
| Salomon-005-02 | Low 14/11 | 0.02 | 2.39 | 0.14 | 0.85 | 74.61 | 13.00 | 6.54 | 0.34 | 1.48 | 0.05 | 0.25 | 0.05 | 0.07 | 0.18 | 3.93 |
| Salomon-005-03 | Low 14/11 | 0.02 | 2.39 | 0.14 | 0.74 | 73.41 | 14.26 | 6.47 | 0.32 | 1.66 | 0.13 | 0.24 | 0.03 | 0.05 | 0.15 | 3.76 |
| Salomon-005-04 | Low 14/11 | 0.02 | 2.37 | 0.15 | 0.68 | 73.55 | 14.02 | 6.52 | 0.31 | 1.71 | 0.12 | 0.24 | 0.05 | 0.06 | 0.19 | 3.69 |
| Salomon-005-05 | Low 14/11 | 0.01 | 2.33 | 0.11 | 0.57 | 72.96 | 15.04 | 6.19 | 0.27 | 1.84 | 0.16 | 0.23 | 0.04 | 0.06 | 0.19 | 3.47 |
| Salomon-005-06 | Low 14/11 | 0.02 | 2.32 | 0.14 | 0.84 | 73.64 | 13.54 | 6.96 | 0.32 | 1.59 | 0.10 | 0.25 | 0.06 | 0.07 | 0.16 | 3.82 |
| Salomon-005-07 | Low 14/11 | 0.02 | 2.31 | 0.12 | 0.60 | 72.14 | 15.60 | 6.54 | 0.25 | 1.78 | 0.14 | 0.21 | 0.05 | 0.06 | 0.17 | 3.45 |
| Salomon-005-08 | Low 14/11 | 0.02 | 2.39 | 0.14 | 0.61 | 72.72 | 14.76 | 6.38 | 0.30 | 1.97 | 0.14 | 0.24 | 0.05 | 0.07 | 0.21 | 3.64 |
| Salomon-005-09 | Low 14/11 | 0.02 | 2.25 | 0.14 | 0.73 | 74.30 | 13.27 | 6.75 | 0.31 | 1.66 | 0.10 | 0.23 | 0.04 | 0.05 | 0.15 | 3.60 |
| Salomon-005-10 | Low 14/11 | 0.03 | 2.30 | 0.14 | 0.81 | 74.10 | 13.40 | 6.91 | 0.13 | 1.60 | 0.05 | 0.24 | 0.06 | 0.06 | 0.18 | 3.57 |
| F6-01 | High 20/17 | 0.03 | 2.60 | 0.14 | 0.97 | 77.08 | 13.84 | 2.51 | 0.44 | 1.57 | 0.09 | 0.30 | 0.04 | 0.17 | 0.23 | 4.51 |
| F6-02 | High 20/17 | 0.03 | 2.66 | 0.16 | 1.08 | 75.93 | 14.82 | 2.56 | 0.46 | 1.55 | 0.09 | 0.29 | 0.03 | 0.14 | 0.18 | 4.68 |
| F6-03 | High 20/17 | 0.02 | 2.54 | 0.12 | 0.97 | 74.35 | 16.41 | 2.44 | 0.45 | 1.85 | 0.13 | 0.31 | 0.04 | 0.15 | 0.22 | 4.44 |
| F6-04 | High 20/17 | 0.03 | 2.59 | 0.16 | 1.17 | 77.17 | 13.62 | 2.55 | 0.50 | 1.48 | 0.08 | 0.29 | 0.03 | 0.15 | 0.20 | 4.72 |
| F6-05 | High 20/17 | 0.03 | 2.39 | 0.12 | 1.24 | 74.19 | 15.98 | 2.97 | 0.50 | 1.77 | 0.12 | 0.31 | 0.04 | 0.16 | 0.20 | 4.62 |
| F6-06 | High 20/17 | 0.03 | 2.46 | 0.12 | 1.30 | 74.78 | 15.28 | 2.97 | 0.53 | 1.72 | 0.11 | 0.32 | 0.05 | 0.14 | 0.21 | 4.77 |
| F6-07 | High 20/17 | 0.03 | 2.59 | 0.17 | 1.23 | 75.88 | 14.86 | 2.49 | 0.52 | 1.45 | 0.08 | 0.34 | 0.03 | 0.18 | 0.16 | 4.88 |
| F6-08 | High 20/17 | 0.03 | 2.43 | 0.13 | 1.35 | 74.57 | 15.91 | 2.65 | 0.53 | 1.59 | 0.11 | 0.31 | 0.03 | 0.19 | 0.16 | 4.84 |
| F6-09 | High 20/17 | 0.03 | 2.58 | 0.18 | 1.27 | 77.36 | 13.34 | 2.44 | 0.54 | 1.46 | 0.08 | 0.34 | 0.03 | 0.18 | 0.19 | 4.94 |
| F6-10 | High 20/17 | 0.03 | 2.31 | 0.12 | 1.28 | 75.12 | 14.90 | 2.99 | 0.53 | 1.84 | 0.12 | 0.33 | 0.04 | 0.17 | 0.23 | 4.65 |
| F6-01 | Low 14/11 | 0.02 | 2.47 | 0.14 | 0.92 | 73.90 | 16.63 | 3.30 | 0.39 | 1.51 | 0.10 | 0.27 | 0.03 | 0.10 | 0.22 | 4.17 |
| F6-02 | Low 14/11 | 0.02 | 2.34 | 0.14 | 0.88 | 75.11 | 15.79 | 3.16 | 0.37 | 1.56 | 0.09 | 0.25 | 0.03 | 0.08 | 0.18 | 3.94 |
| F6-03 | Low 14/11 | 0.02 | 2.38 | 0.12 | 0.91 | 74.76 | 15.89 | 3.28 | 0.37 | 1.57 | 0.11 | 0.28 | 0.03 | 0.09 | 0.19 | 4.04 |
| F6-04 | Low 14/11 | 0.02 | 2.35 | 0.15 | 0.97 | 74.66 | 16.22 | 3.15 | 0.39 | 1.50 | 0.09 | 0.26 | 0.03 | 0.07 | 0.17 | 4.06 |
| F6-05 | Low 14/11 | 0.03 | 2.50 | 0.17 | 0.98 | 74.94 | 15.83 | 3.10 | 0.37 | 1.42 | 0.06 | 0.27 | 0.05 | 0.08 | 0.19 | 4.23 |
| F6-06 | Low 14/11 | 0.02 | 2.45 | 0.14 | 0.91 | 74.36 | 16.44 | 3.10 | 0.36 | 1.52 | 0.07 | 0.27 | 0.06 | 0.08 | 0.20 | 4.10 |
| F6-07 | Low 14/11 | 0.03 | 2.49 | 0.15 | 0.94 | 75.38 | 15.37 | 3.45 | 0.25 | 1.42 | 0.06 | 0.17 | 0.04 | 0.07 | 0.18 | 3.94 |
| F6-08 | Low 14/11 | 0.02 | 2.34 | 0.14 | 0.89 | 74.17 | 16.578 | 3.21 | 0.37 | 1.67 | 0.10 | 0.25 | 0.04 | 0.07 | 0.17 | 3.94 |
| F6-09 | Low 14/11 | 0.03 | 2.69 | 0.23 | 1.10 | 69.80 | 20.52 | 2.73 | 0.46 | 1.59 | 0.13 | 0.32 | 0.08 | 0.12 | 0.23 | 4.71 |
| F6-10 | Low 14/11 | 0.02 | 2.44 | 0.16 | 0.92 | 73.55 | 16.87 | 3.39 | 0.38 | 1.60 | 0.09 | 0.28 | 0.04 | 0.07 | 0.19 | 4.12 |
| IMC201-01 | High 20/17 | 0.05 | 3.79 | 0.18 | 2.34 | 77.38 | 10.98 | 2.25 | 0.84 | 1.30 | 0.06 | 0.40 | 0.02 | 0.23 | 0.20 | 7.64 |
| IMC201-02 | High 20/17 | 0.05 | 4.30 | 0.22 | 1.86 | 77.13 | 11.37 | 2.37 | 0.69 | 1.23 | 0.06 | 0.36 | 0.03 | 0.17 | 0.17 | 7.43 |
| IMC201-04 | High 20/17 | 0.05 | 4.03 | 0.19 | 2.05 | 76.20 | 12.56 | 2.31 | 0.69 | 1.21 | 0.06 | 0.34 | 0.02 | 0.15 | 0.15 | 7.30 |
| IMC201-05 | High 20/17 | 0.05 | 4.34 | 0.22 | 1.84 | 76.58 | 11.93 | 2.43 | 0.68 | 1.20 | 0.06 | 0.34 | 0.02 | 0.16 | 0.17 | 7.40 |
| IMC201-06 | High 20/17 | 0.05 | 4.06 | 0.20 | 2.21 | 75.83 | 12.34 | 2.49 | 0.78 | 1.25 | 0.06 | 0.36 | 0.02 | 0.19 | 0.15 | 7.65 |
| IMC201-07 | High 20/17 | 0.05 | 3.99 | 0.19 | 2.16 | 76.33 | 12.22 | 2.27 | 0.77 | 1.24 | 0.05 | 0.36 | 0.02 | 0.17 | 0.18 | 7.50 |
| IMC201-08 | High 20/17 | 0.05 | 3.90 | 0.19 | 2.16 | 75.80 | 12.94 | 2.42 | 0.68 | 1.22 | 0.06 | 0.30 | 0.02 | 0.15 | 0.13 | 7.22 |
| IMC201-09 | High 20/17 | 0.03 | 3.41 | 0.13 | 2.46 | 76.72 | 12.12 | 2.37 | 0.72 | 1.35 | 0.07 | 0.30 | 0.01 | 0.18 | 0.12 | 7.10 |
| IMC201-10 | High 20/17 | 0.05 | 4.26 | 0.20 | 2.06 | 78.16 | 10.15 | 2.02 | 0.84 | 1.32 | 0.05 | 0.44 | 0.02 | 0.25 | 0.18 | 7.90 |
| IMC201-01 | Low 14/11 | 0.05 | 3.76 | 0.21 | 1.63 | 76.15 | 12.63 | 2.98 | 0.61 | 1.27 | 0.06 | 0.34 | 0.04 | 0.11 | 0.16 | 6.51 |
| IMC201-02 | Low 14/11 | 0.05 | 3.67 | 0.20 | 1.62 | 76.61 | 12.52 | 2.96 | 0.37 | 1.32 | 0.05 | 0.31 | 0.03 | 0.11 | 0.20 | 6.13 |
| IMC201-04 | Low 14/11 | 0.06 | 4.02 | 0.23 | 1.47 | 74.65 | 13.94 | 3.03 | 0.58 | 1.33 | 0.06 | 0.33 | 0.03 | 0.13 | 0.15 | 6.58 |
| IMC201-05 | Low 14/11 | 0.05 | 3.97 | 0.22 | 1.49 | 75.43 | 13.43 | 2.74 | 0.58 | 1.36 | 0.06 | 0.34 | 0.03 | 0.11 | 0.18 | 6.54 |
| IMC201-06 | Low 14/11 | 0.04 | 3.66 | 0.20 | 1.59 | 75.94 | 12.96 | 2.98 | 0.55 | 1.32 | 0.08 | 0.34 | 0.05 | 0.10 | 0.20 | 6.28 |
| IMC201-07 | Low 14/11 | 0.02 | 2.53 | 0.16 | 1.09 | 75.76 | 15.24 | 3.20 | 0.14 | 1.29 | 0.08 | 0.24 | 0.02 | 0.07 | 0.16 | 4.08 |
| IMC201-08 | Low 14/11 | 0.03 | 3.49 | 0.13 | 1.73 | 74.49 | 14.29 | 3.38 | 0.40 | 1.61 | 0.04 | 0.21 | 0.02 | 0.06 | 0.13 | 5.92 |
| IMC201-10 | Low 14/11 | 0.04 | 3.84 | 0.21 | 1.42 | 75.88 | 12.93 | 2.93 | 0.57 | 1.43 | 0.07 | 0.36 | 0.020 | 0.12 | 0.19 | 6.35 |
| 15.24-01 | High 20/17 | 0.03 | 3.14 | 0.12 | 1.12 | 77.45 | 11.38 | 3.87 | 0.46 | 1.71 | 0.13 | 0.28 | 0.04 | 0.14 | 0.14 | 5.17 |
| 15.24-02 | High 20/17 | 0.03 | 3.16 | 0.14 | 1.45 | 76.54 | 11.27 | 4.38 | 0.56 | 1.70 | 0.11 | 0.30 | 0.05 | 0.15 | 0.17 | 5.65 |
| 15.24-03 | High 20/17 | 0.03 | 3.18 | 0.14 | 1.39 | 77.14 | 10.63 | 4.44 | 0.58 | 1.70 | 0.11 | 0.30 | 0.03 | 0.16 | 0.16 | 5.64 |
| 15.24-04 | High 20/17 | 0.02 | 3.25 | 0.12 | 1.11 | 76.16 | 11.90 | 4.40 | 0.48 | 1.79 | 0.14 | 0.28 | 0.04 | 0.13 | 0.17 | 5.28 |
| 15.24-05 | High 20/17 | 0.03 | 3.12 | 0.12 | 1.10 | 77.38 | 11.11 | 4.20 | 0.44 | 1.81 | 0.14 | 0.26 | 0.04 | 0.14 | 0.13 | 5.08 |
| 15.24-06 | High 20/17 | 0.03 | 2.90 | 0.13 | 1.28 | 76.83 | 11.53 | 4.00 | 0.51 | 1.90 | 0.15 | 0.29 | 0.05 | 0.17 | 0.24 | 5.18 |
| 15.24-07 | High 20/17 | 0.02 | 3.19 | 0.13 | 1.28 | 75.24 | 12.39 | 4.88 | 0.49 | 1.70 | 0.14 | 0.27 | 0.03 | 0.12 | 0.13 | 5.37 |
| 15.24-08 | High 20/17 | 0.03 | 3.18 | 0.13 | 1.23 | 76.44 | 11.21 | 4.67 | 0.51 | 1.83 | 0.12 | 0.29 | 0.04 | 0.15 | 0.18 | 5.39 |
| 15.24-09 | High 20/17 | 0.02 | 3.12 | 0.14 | 1.41 | 77.36 | 10.36 | 4.48 | 0.58 | 1.75 | 0.10 | 0.31 | 0.03 | 0.16 | 0.17 | 5.60 |
| 15.24-10 | High 20/17 | 0.04 | 3.18 | 0.14 | 1.43 | 76.19 | 11.33 | 4.71 | 0.56 | 1.67 | 0.11 | 0.29 | 0.05 | 0.14 | 0.16 | 5.64 |
| 15.24-02 | Low 14/11 | 0.04 | 3.09 | 0.15 | 0.64 | 75.62 | 11.81 | 5.84 | 0.37 | 1.76 | 0.11 | 0.27 | 0.07 | 0.09 | 0.16 | 4.49 |
| 15.24-03 | Low 14/11 | 0.03 | 2.71 | 0.12 | 1.04 | 75.80 | 11.73 | 5.74 | 0.28 | 1.95 | 0.07 | 0.20 | 0.04 | 0.11 | 0.19 | 4.36 |
| 15.24-04 | Low 14/11 | 0.02 | 2.85 | 0.11 | 0.97 | 76.63 | 10.60 | 5.58 | 0.45 | 2.00 | 0.12 | 0.33 | 0.06 | 0.08 | 0.22 | 4.69 |
| 15.24-06 | Low 14/11 | 0.02 | 2.86 | 0.13 | 1.07 | 76.75 | 10.47 | 5.70 | 0.44 | 1.88 | 0.10 | 0.30 | 0.04 | 0.09 | 0.15 | 4.78 |
| 15.24-07 | Low 14/11 | 0.03 | 3.05 | 0.14 | 1.22 | 75.85 | 11.13 | 5.99 | 0.48 | 1.47 | 0.11 | 0.29 | 0.04 | 0.07 | 0.14 | 5.14 |
| 15.24-08 | Low 14/11 | 0.02 | 2.98 | 0.13 | 0.97 | 75.51 | 11.78 | 5.72 | 0.39 | 1.84 | 0.11 | 0.27 | 0.04 | 0.08 | 0.15 | 4.71 |
| 15.24-09 | Low 14/11 | 0.02 | 2.98 | 0.13 | 1.00 | 75.11 | 12.02 | 5.81 | 0.42 | 1.81 | 0.12 | 0.28 | 0.04 | 0.08 | 0.19 | 4.78 |
| 15.24-10 | Low 14/11 | 0.01 | 2.96 | 0.12 | 0.89 | 76.55 | 11.00 | 5.53 | 0.40 | 1.85 | 0.12 | 0.32 | 0.03 | 0.08 | 0.14 | 4.66 |

### Example 9

### DH Population Skechers

A DH population designated Skechers was obtained from a cross between 15.24 and 06SE-04GX-33. The 06SE-04GX-33 parent line was selected from progeny of a cross between 04GX-33 and 01NM.304. Line 04GX-33, which has an oleic acid content of about 80% and reduced saturated fatty acid content, was produced by crossing 01NM.304 and a European spring growth habit line ‘Lila’ and developing a DH population from the F₁ cross. Line 01NM.304 was developed from a DH population of an F₁ cross between IMC302 and Surpass 400. 06SE-04GX-33 seeds have a mean C14:0 content of 0.091%, a C16:0 content of 4.47%, a C16:1 content of 0.68%, a C18:0 content of 1.69%, a C18:1 content of 79.52%, a C18:2 content of 6.62%, a C18:3 content of 4.12%, a C20:0 content of 0.63%, a C20:1 content of 1.22%, a C22:0 content of 0.49%, a C22:1 content of 0.0%, a C24:0 content of 0.21%, and a C24:1 content of 0.24%.

This DH population was generated from the cross of 15.24 and 06SE-04GX-33 by collecting microspores, treating the microspores with colchicine, and propagating them in vitro.

Plantlets formed in vitro from the microspores were moved to a greenhouse and inflorescences that formed were self-pollinated. Seed was harvested from the DH₁ plants at maturity and analyzed for fatty acid profile via GC. Table 16 contains the fatty acid profile of seeds produced by plants grown in the greenhouse and in the field of DH lines selected from the Skechers population. The fatty acid profile of IMC111RR is included as a control in Table 16. Skechers-159 and Skechers-339 exhibited a low total saturated fatty acid profile in the greenhouse and in the field (Table 16).

TABLE 16
Fatty acid composition of seed of Skechers 339 and Skechers 159

| | | | | | | | | | | | | | | | Total |
|---|---|---|---|---|---|---|---|---|---|---|---|---|---|---|---|
| Line | C140 | C160 | C161 | C180 | C181 | C182 | C183 | C200 | C201 | C202 | C220 | C221 | C240 | C241 | Sats |
| Greenhouse | | | | | | | | | | | | | | | |
| Skechers-339 | 0.04 | 2.86 | 0.20 | 1.11 | 84.53 | 4.40 | 3.61 | 0.48 | 1.98 | 0.12 | 0.27 | 0.00 | 0.23 | 0.17 | 4.98 |
| Skechers-159 | 0.03 | 2.91 | 0.19 | 1.26 | 84.24 | 4.05 | 3.57 | 0.55 | 1.88 | 0.15 | 0.34 | 0.00 | 0.00 | 0.82 | 5.08 |
| Field | | | | | | | | | | | | | | | |
| Skechers-339 | 0.00 | 2.55 | 0.12 | 0.94 | 82.64 | 5.07 | 5.44 | 0.39 | 2.11 | 0.16 | 0.28 | 0.04 | 0.14 | 0.13 | 4.29 |
| Skechers-339 | 0.00 | 2.80 | 0.16 | 1.22 | 81.55 | 5.57 | 4.89 | 0.50 | 2.25 | 0.21 | 0.52 | 0.00 | 0.19 | 0.16 | 5.22 |
| Skechers-339 | 0.000 | 3.01 | 0.22 | 1.04 | 79.43 | 7.39 | 5.12 | 0.46 | 2.21 | 0.20 | 0.55 | 0.04 | 0.17 | 0.18 | 5.23 |
| Mean | 0.00 | 2.79 | 0.17 | 1.07 | 81.20 | 6.01 | 5.15 | 0.45 | 2.19 | 0.19 | 0.44 | 0.03 | 0.17 | 0.16 | 4.91 |
| Skechers-159 | 0.03 | 2.65 | 0.14 | 1.03 | 83.52 | 5.07 | 5.09 | 0.41 | 2.04 | 0.00 | 0.00 | 0.01 | 0.01 | 0.00 | 4.13 |
| Skechers-159 | 0.03 | 2.60 | 0.15 | 0.97 | 82.93 | 4.80 | 5.52 | 0.39 | 2.16 | 0.13 | 0.33 | 0.00 | 0.00 | 0.01 | 4.32 |
| Skechers-159 | 0.04 | 2.69 | 0.23 | 0.95 | 82.99 | 5.08 | 5.18 | 0.39 | 2.06 | 0.12 | 0.28 | 0.00 | 0.01 | 0.00 | 4.35 |
| Skechers-159 | 0.04 | 2.59 | 0.15 | 0.90 | 80.65 | 5.50 | 5.48 | 0.36 | 2.08 | 0.12 | 2.12 | 0.00 | 0.00 | 0.00 | 6.01 |
| Mean | 0.04 | 2.63 | 0.17 | 0.96 | 82.52 | 5.11 | 5.32 | 0.39 | 2.08 | 0.09 | 0.68 | 0.00 | 0.01 | 0.01 | 4.70 |
| IMC111RR | 0.08 | 5.06 | 0.41 | 2.07 | 56.80 | 28.40 | 3.87 | 0.83 | 1.44 | 0.14 | 0.50 | 0.00 | 0.23 | 0.16 | 8.77 |
| IMC111RR | 0.09 | 5.38 | 0.50 | 2.09 | 56.61 | 28.38 | 3.50 | 0.81 | 1.41 | 0.13 | 0.50 | 0.01 | 0.53 | 0.08 | 9.40 |
| IMC111RR | 0.21 | 6.15 | 0.50 | 1.46 | 47.82 | 36.03 | 3.38 | 0.71 | 1.24 | 0.14 | 0.56 | 0.00 | 1.43 | 0.37 | 10.52 |

### Example 10

### Line 1764-43-06×1975-90-14

A pedigree selection program was carried out with progeny of a cross of 1764-43-06×1975-90-14 over multiple cycles of single plant selections in the greenhouse for low total saturated fatty acid content in seeds. Table 17 contains the seed fatty acid profile of each parent used to make the F₁ cross. Table 18 contains the seed fatty acid profile of selections advanced through the F₆ generation. The mean seed fatty acid profiles of the inbred 01PR06RR.001B and the variety IMC201 are shown for comparison. Additional rounds of self-pollination and selection for low total saturated fatty acids can be performed.

TABLE 17
Fatty acid composition of seed of Lines 1975-90-14 and 1764-43-06

| Line | C140 | C160 | C161 | C180 | C181 | C182 | C183 | C200 | C201 | C202 | C220 | C221 | C240 | C241 | Total Sats |
|---|---|---|---|---|---|---|---|---|---|---|---|---|---|---|---|
| 1975-90-14 | 0.00 | 3.78 | 0.23 | 1.54 | 75.12 | 14.06 | 2.08 | 0.64 | 1.62 | 0.09 | 0.38 | 0.0 | 0.27 | 0.18 | 6.61 |
| 1764-43-06 | 0.039 | 3.28 | 0.31 | 2.40 | 75.45 | 12.97 | 1.96 | 0.90 | 1.54 | 0.08 | 0.48 | 0.0 | 0.42 | 0.17 | 7.52 |

TABLE 18
Seed Fatty acid composition of F2-F6 generations selected in progeny 1764-43-06 x 1975-90-14

| Line | C140 | C160 | C161 | C180 | C181 | C182 | C183 | C200 | C201 | C202 | C220 | C221 | C240 | C241 | Total Sats |
|---|---|---|---|---|---|---|---|---|---|---|---|---|---|---|---|
| F2 seed | | | | | | | | | | | | | | | |
| E626033 | 0.063 | 4.33 | 0.63 | 1.59 | 63.33 | 22.87 | 4.04 | 0.63 | 1.50 | 0.13 | 0.39 | 0.00 | 0.26 | 0.25 | 7.26 |
| E626088 | 0.051 | 3.41 | 0.26 | 1.58 | 72.76 | 16.44 | 2.16 | 0.64 | 1.68 | 0.10 | 0.39 | 0.00 | 0.23 | 0.30 | 6.30 |
| E626134 | 0.042 | 3.40 | 0.23 | 1.66 | 73.61 | 15.71 | 1.96 | 0.70 | 1.69 | 0.09 | 0.42 | 0.03 | 0.26 | 0.20 | 6.48 |
| E626082 | 0.05 | 3.50 | 0.26 | 1.69 | 72.38 | 16.58 | 2.05 | 0.69 | 1.61 | 0.09 | 0.41 | 0.00 | 0.24 | 0.45 | 6.58 |
| 01PR06RR.001B Mean | 0.07 | 4.73 | 0.37 | 2.17 | 66.27 | 21.15 | 2.13 | 0.87 | 1.12 | 0.06 | 0.49 | 0.01 | 0.36 | 0.21 | 8.69 |
| F3 seed | | | | | | | | | | | | | | | |
| E642092 | 0.05 | 3.57 | 0.32 | 1.06 | 60.40 | 27.06 | 4.14 | 0.46 | 1.85 | 0.17 | 0.43 | 0.00 | 0.20 | 0.29 | 5.77 |
| E642105 | 0.03 | 2.98 | 0.16 | 1.67 | 74.64 | 14.52 | 2.39 | 0.67 | 1.88 | 0.10 | 0.39 | 0.04 | 0.28 | 0.24 | 6.02 |
| E641751 | 0.04 | 3.16 | 0.19 | 1.40 | 73.53 | 15.88 | 2.57 | 0.57 | 1.74 | 0.12 | 0.35 | 0.00 | 0.23 | 0.23 | 5.75 |
| E641767 | 0.04 | 2.99 | 0.18 | 1.46 | 72.85 | 16.25 | 2.59 | 0.59 | 1.92 | 0.14 | 0.39 | 0.06 | 0.27 | 0.26 | 5.74 |
| E642058 | 0.02 | 3.56 | 0.31 | 1.26 | 70.79 | 18.60 | 2.65 | 0.51 | 1.59 | 0.12 | 0.30 | 0.00 | 0.18 | 0.11 | 5.84 |
| E642706 | 0.00 | 2.95 | 0.20 | 1.49 | 72.76 | 16.92 | 2.58 | 0.60 | 1.59 | 0.11 | 0.30 | 0.00 | 0.23 | 0.27 | 5.57 |
| E641983 | 0.03 | 3.21 | 0.23 | 1.62 | 71.50 | 17.62 | 2.51 | 0.63 | 1.74 | 0.12 | 0.33 | 0.00 | 0.22 | 0.23 | 6.05 |
| E641989 | 0.0403 | 2.9929 | 0.22 | 1.44 | 73.11 | 16.43 | 2.67 | 0.57 | 1.65 | 0.11 | 0.34 | 0.00 | 0.22 | 0.21 | 5.61 |
| E642042 | 0.0000 | 2.8352 | 0.16 | 1.81 | 75.94 | 13.78 | 2.08 | 0.69 | 1.86 | 0.10 | 0.35 | 0.00 | 0.25 | 0.14 | 5.94 |
| E642071 | 0.0371 | 3.0309 | 0.20 | 1.77 | 72.45 | 16.74 | 2.74 | 0.63 | 1.75 | 0.12 | 0.31 | 0.00 | 0.21 | 0.00 | 6.00 |
| 01PR06RR.001B Mean | 0.0637 | 4.6079 | 0.36 | 1.94 | 66.25 | 21.83 | 2.03 | 0.77 | 1.15 | 0.06 | 0.43 | 0.01 | 0.32 | 0.19 | 8.12 |
| F4 seed | | | | | | | | | | | | | | | |
| F604402 | 0.0266 | 2.4461 | 0.14 | 1.15 | 75.79 | 14.69 | 2.74 | 0.46 | 1.83 | 0.12 | 0.25 | 0.04 | 0.12 | 0.22 | 4.44 |
| F603986 | 0.0183 | 2.323 | 0.13 | 1.32 | 77.47 | 13.68 | 2.57 | 0.51 | 1.37 | 0.07 | 0.32 | 0 | 0 | 0.22 | 4.50 |
| 01PR06RR.001B Mean | 0.0501 | 4.5160 | 0.33 | 1.84 | 66.82 | 21.10 | 2.32 | 0.77 | 1.22 | 0.06 | 0.48 | 0.02 | 0.27 | 0.21 | 7.93 |
| F5 Seed Chamber 15°/12° | | | | | | | | | | | | | | | |
| Seed from F604402: | | | | | | | | | | | | | | | |
| FTF647808 | 0 | 2.45 | 0.2 | 1.16 | 76.27 | 14.48 | 2.97 | 0.45 | 1.39 | 0.06 | 0.26 | 0 | 0.08 | 0.22 | 4.41 |
| FTF647745 | 0 | 2.2 | 0 | 1.20 | 75.65 | 14.88 | 3.56 | 0.46 | 1.47 | 0.08 | 0.27 | 0 | 0 | 0.21 | 4.13 |
| FTF647752 | 0 | 2.41 | 0.15 | 1.21 | 76.42 | 14.52 | 2.86 | 0.43 | 1.42 | 0.09 | 0.23 | 0 | 0.07 | 0.19 | 4.34 |
| FTF647789 | 0 | 2.51 | 0.2 | 1.12 | 74.75 | 16.15 | 2.72 | 0.43 | 1.44 | 0.08 | 0.26 | 0.04 | 0.09 | 0.22 | 4.4 |
| Seed from F603986: | | | | | | | | | | | | | | | |
| FTF647754 | 0 | 2.28 | 0.15 | 1.12 | 77.12 | 13.73 | 3.01 | 0.44 | 1.49 | 0.07 | 0.27 | 0.04 | 0.07 | 0.21 | 4.19 |
| FTF647775 | 0 | 2.28 | 0.16 | 1.15 | 76.91 | 13.82 | 2.96 | 0.47 | 1.54 | 0.07 | 0.31 | 0.04 | 0.08 | 0.22 | 4.28 |
| FTF647804 | 0 | 2.39 | 0.17 | 1.21 | 77.55 | 13.2 | 3.07 | 0.48 | 1.40 | 0.00 | 0.25 | 0 | 0.08 | 0.2 | 4.41 |
| FTF647777 | 0 | 2.25 | 0.17 | 1.17 | 77.39 | 13.63 | 2.83 | 0.46 | 1.46 | 0.06 | 0.27 | 0.03 | 0.07 | 0.21 | 4.22 |
| FTF647778 | 0 | 2.29 | 0 | 1.26 | 77.6 | 13.41 | 2.94 | 0.47 | 1.38 | 0.07 | 0.30 | 0 | 0.08 | 0.21 | 4.39 |
| IMC201 Mean | 0.038 | 3.9 | 0.20 | 1.80 | 77.244 | 11.588 | 2.81 | 0.65 | 1.15 | 0.03 | 0.30 | 0 | 0.11 | 0.2 | 6.80 |
| F6 Seed Chamber 20°/17° | | | | | | | | | | | | | | | |
| Seed from FTF647754: | | | | | | | | | | | | | | | |
| FTG603509 | 0.03 | 2.66 | 0.14 | 1.39 | 76.57 | 14.16 | 2.52 | 0.53 | 1.36 | 0.08 | 0.28 | 0.02 | 0.13 | 0.13 | 5.01 |
| FTG603519 | 0.03 | 2.56 | 0.15 | 1.32 | 76.7 | 14.05 | 2.43 | 0.57 | 1.43 | 0.08 | 0.32 | 0.03 | 0.17 | 0.16 | 4.97 |
| FTG603505 | 0.02 | 2.47 | 0.14 | 1.33 | 79.5 | 11.43 | 2.22 | 0.58 | 1.52 | 0.07 | 0.34 | 0.02 | 0.21 | 0.16 | 4.95 |
| FTG603506 | 0.07 | 3.59 | 0.14 | 2.73 | 68.43 | 19.28 | 3.41 | 0.76 | 0.89 | 0.04 | 0.30 | 0.00 | 0.22 | 0.13 | 7.67 |
| FTG603517 | 0.03 | 2.66 | 0.16 | 1.44 | 76.75 | 13.94 | 2.44 | 0.56 | 1.37 | 0.07 | 0.29 | 0.02 | 0.14 | 0.13 | 5.12 |
| FTG603507 | 0.03 | 2.63 | 0.15 | 1.31 | 76.59 | 14.18 | 2.5 | 0.53 | 1.39 | 0.05 | 0.29 | 0.02 | 0.16 | 0.18 | 4.94 |
| FTG603508 | 0.03 | 2.51 | 0.12 | 1.38 | 75.88 | 14.61 | 2.58 | 0.56 | 1.56 | 0.09 | 0.33 | 0.03 | 0.16 | 0.15 | 4.97 |
| FTG603515 | 0.03 | 2.74 | 0.13 | 1.33 | 75.67 | 14.91 | 2.71 | 0.49 | 1.36 | 0.08 | 0.25 | 0.02 | 0.12 | 0.14 | 4.97 |
| FTG603516 | 0.03 | 2.65 | 0.13 | 1.41 | 76.32 | 14.16 | 2.37 | 0.6 | 1.54 | 0.09 | 0.34 | 0.03 | 0.18 | 0.16 | 5.21 |
| FTG603520 | 0.03 | 2.72 | 0.14 | 1.42 | 75.61 | 14.9 | 2.37 | 0.57 | 1.49 | 0.09 | 0.32 | 0.03 | 0.16 | 0.15 | 5.23 |

### Example 11

### Seed Fatty Acid Profiles for Field-Grown Plants

Plants of 15.24, Salomon-03, Salomon-05, Salomon-07, and F6 selected line described in Example 10, Skechers-159 and Skecher-339 were grown in field plots in Aberdeen, SK, Canada. At maturity, seeds from each line were harvested and fatty acid content determined by GC analysis. The ranges of palmitic, stearic, oleic, linoleic, and linolenic acid content, and the range of total saturated fatty acids are shown in Table 19. The ranges for seed of line Q2 and Pioneer® variety 46A65 are shown for comparison.

TABLE 19
Fatty Acid Profiles for Field-Grown Plants

| Genotype | C16:0 | C18:0 | C18:1 | C18:2 | C18:3 | Total Sats |
|---|---|---|---|---|---|---|
| 46A65 | 3.37-4.12 | 1.53-2.29 | 64.85-71.46 | 13.57-19.16 | 5.06-7.95 | 6.24-7.52 |
| Q2 | 3.53-4.10 | 1.46-2.10 | 63.03-70.49 | 13.79-19.44 | 6.15-10.28 | 6.14-7.62 |
| Salomon-07 | 3.44-4.20 | 0.71-0.81 | 73.68-76.74 | 11.76-13.24 | 3.66-4.08 | 4.96-5.97 |
| Salomon-05 | 3.02-3.34 | 0.95-1.11 | 72.74-74.51 | 13.70-15.94 | 3.40-4.69 | 4.34-5.22 |
| 15.24 | 2.77-3.19 | 0.95-1.06 | 77.16-77.95 | 10.76-12.02 | 3.42-3.68 | 4.53-5.36 |
| Selection from | 2.42-2.73 | 0.97-1.29 | 71.07-73.56 | 15.65-18.80 | 2.75-2.91 | 4.21-5.19 |
| 1764-43-06 x | | | | | | |
| 1975-90-14 | | | | | | |
| Salomon-03 | 2.24-2.51 | 1.08-1.36 | 72.20-76.70 | 14.15-18.15 | 2.03-2.71 | 4.38-4.81 |
| Skechers-339 | 2.38-2.84 | 0.91-1.28 | 79.93-86.50 | 3.95-4.90 | 3.23-4.90 | 4.03-5.23 |
| Skechers-159 | 2.37-3.75 | 0.91-1.26 | 83.97-86.45 | 3.49-4.80 | 4.11-4.47 | 4.11-4.47 |

### Example 12

### Radiation Mutagenesis (RMU) of 15.24 Germplasm

About 30 grams (8000 seeds) of M₀ seeds from an individual selected from the DH population of 15.24×01OB240 on the basis of low total saturates (see Example 2) were mutagenized using cesium irradiation at 45 krad. About 1500 of the mutagenized seeds were planted in the greenhouse immediately after irradiation, about 500 of them developed into plants to produce M₁ seeds. About 840 M₁ seeds were planted and M₂ seed was harvested. M₂ seed was planted along with F₁ progeny plants of a cross of 15.24×01OB240 (designated control 1; M₀ seed) were also planted. The fatty acid composition of M₃ seeds produced by individual M₂ plants and control plants was analyzed by GC. The results are shown in Table 20 under the M₂ heading. The individual M₂ plant producing M₃ seeds with the lowest total saturates was 08AP-RMU-tray 3-18, which had 5.28% total saturates compared to 6.48% for control-1. The individual M₂ plant producing M₃ seeds with the lowest 16:0 was 08AP-RMU-tray 13-25, which had 2.55% 16:0 compared with 3.19% for control-1. The individual M₂ plant producing M₃ seeds with the lowest 18:0 was 08AP-RMU-tray 10-34, which had an 18:0 content of 0.93% compared with 1.7% for control-1. M₃ seed used to generate fatty acid profiles shown in Table 20 was planted from these three lines in the greenhouse.

M₄ plants derived from M₃ seed with low total saturates, 16:0, and 18:0, respectively, from each of the three groups were selected for use in crosses. Line M4-L1601-12 had a total saturates content of 5.28% in the M₃ generation and was selected from the 08AP-RMU-tray 3-18 lineage. A cross was made between plants of line M4-L1601-12 and a line containing the homozygous mutant alleles of Isoforms 1, 2, 3, 4 of FatB (described in Example 6). Seed fatty acid profiles from F₂ seeds for two F₁ individuals are shown in Table 20. Plants of lines M4-Lsat1-23 and M4-L1601-22 were crossed, and the fatty acid profile for seeds produced on an F₁ individual designated 09AP-RMU-003-06 are shown in Table 20. M4-Lsat1-23 and M4-L1601-22 were selected from the M3 generation with total saturate of 5.02% and 16:0 of 2.43%. Plants of lines M4-L1601-12×M4-D60-2-01 were crossed, and the fatty acid profile for seeds produced on an F₁ individual designated 09AP-RMU-012-2 are shown in Table 20. M4-L1601-12×M4-D60-2-01 were selected from the M3 generation with total saturates of 5.28% and 18:0 of 0.88%, respectively. Seeds from F₁ plants with low total saturated fatty acid content, low 16:0, and low 18:0 were grown for further pedigree selection breeding. Some plants were self-pollinated and used to generate DH populations for further selection. It is expected that total saturated fatty acid content in seeds produced on F₂ plants and on progeny of the DH populations will be lower than that in seeds produced on F₁ plants, due to genetic segregation for homozygosity for mutant alleles at loci that confer the low total saturates phenotype.

TABLE 20

| | | | | | | | | | | | | | | | Total |
|---|---|---|---|---|---|---|---|---|---|---|---|---|---|---|---|
| Identifer | C140 | C160 | C161 | C180 | C181 | C182 | C183 | C200 | C201 | C202 | C220 | C221 | C240 | C241 | Sats |
| 15.24 x 01OB240 | 0.04 | 3.19 | 0.14 | 1.7 | 78.32 | 10.51 | 2.23 | 0.73 | 2.03 | 0.13 | 0.41 | 0.05 | 0.39 | 0.13 | 6.45 |
| (control 1) | | | | | | | | | | | | | | | |
| M₂ | | | | | | | | | | | | | | | |
| 08AP-RMU-tray13-25 | 0.03 | 2.55 | 0.1 | 1.68 | 78.86 | 10.86 | 2.14 | 0.7 | 2.16 | 0.14 | 0.35 | 0.01 | 0.3 | 0.13 | 5.61 |
| 08AP-RMU-tray10-34 | 0.02 | 3.08 | 0.02 | 0.93 | 79.4 | 10.51 | 2.24 | 0.61 | 2.11 | 0.15 | 0.4 | 0.05 | 0.34 | 0.14 | 5.39 |
| 08AP-RMU-tray3-18 | 0.02 | 2.96 | 0.1 | 1.16 | 80.49 | 9.68 | 2.07 | 0.56 | 2.12 | 0.14 | 0.35 | 0.04 | 0.23 | 0.08 | 5.28 |
| M₄ | | | | | | | | | | | | | | | |
| M4-L1601-12 | 0 | 3.26 | 0 | 1.76 | 72.64 | 15.82 | 2.95 | 0.76 | 2.13 | 0.2 | 0.49 | 0 | 0 | 0 | 6.26 |
| Salomon-05 | 0 | 2.49 | 0.11 | 1.67 | 77.95 | 10.05 | 4.25 | 0.64 | 1.93 | 0.12 | 0.32 | 0.05 | 0.23 | 0.2 | 5.58 |
| (control 2) | | | | | | | | | | | | | | | |
| F1 of RMU mutants x | | | | | | | | | | | | | | | |
| RMU mutants | | | | | | | | | | | | | | | |
| 09AP-RMU-003-06 | 0.03 | 2.66 | 0.06 | 1.47 | 79.34 | 10.6 | 2.11 | 0.67 | 2.06 | 0.15 | 0.36 | 0.07 | 0.25 | 0.2 | 5.42 |
| [M4-Lsat1-23 X M4- | | | | | | | | | | | | | | | |
| L1601-22] | | | | | | | | | | | | | | | |
| 09AP-RMU-012-2 | 0.03 | 2.79 | 0.11 | 1.44 | 77.36 | 12.4 | 2.36 | 0.64 | 1.93 | 0.14 | 0.38 | 0.05 | 0.21 | 0.15 | 5.5 |
| [M4-L1601-12 X M4- | | | | | | | | | | | | | | | |
| D60-2-01] | | | | | | | | | | | | | | | |
| F1 of RMU mutants x | | | | | | | | | | | | | | | |
| mutant FatB 1, 2, 3, 4 | | | | | | | | | | | | | | | |
| 09AP-RMU-008-07 | 0.04 | 2.98 | 0.18 | 1.74 | 72.18 | 17.38 | 2.37 | 0.7 | 1.44 | 0.11 | 0.37 | 0.04 | 0.26 | 0.22 | 6.09 |
| [M4-L1601-12 X | | | | | | | | | | | | | | | |
| Iso1234 | | | | | | | | | | | | | | | |
| 09AP-RMU-008-05 | 0.02 | 2.74 | 0.17 | 2.08 | 74.02 | 15.78 | 2.14 | 0.74 | 1.41 | 0.11 | 0.35 | 0.02 | 0.24 | 0.2 | 6.17 |
| [M4-L1601-12 X | | | | | | | | | | | | | | | |
| Iso1234 | | | | | | | | | | | | | | | |

### Example 13

### Development of Hybrid Canola Producing Reduced Saturated Fat Seed Oil

A hybrid canola variety yielding seeds with a total saturated fatty acid content of less than 6% was produced by introducing genes from the low saturate line 15.24 into a commercially grown hybrid, Victory® v1035. Hybrid v1035 has an average oleic acid content of 65%. Plants of the line 15.24, and the inbreds 01PR06RR.001B and 95CB504, were planted in a greenhouse. Inbred 01PR06RR.001B is the male parent of v1035. Inbred 95CB504 is the B line female parent of v1035. Plants of 010PR06RR.001B and 15.24 were cross pollinated in the greenhouse as were 95CB504 and 15.24, as shown in Table 21.

TABLE 21

| Female | x | Male |
|---|---|---|
| 01PR06RR.001B (R-line) | | 15.24 |
| 95CB504 (B-line) | | 15.24 |

F₁ progeny from the cross of 95CB504 and 15.24 were backcrossed to 95CB04 to produce BC₁—B progeny, which were selfed (BC₁S). Plants with low total saturates were selected from the BC₁—B selfed progeny, and backcrossed to 95CB504 to produce BC₂—B progeny. F₁ progeny from the cross of 01PR06RR.001B and 15.24 were backcrossed to 01PR06RR.001B to produce BC₁—R progeny, which were selfed. Plants with low total saturates were selected from the BC₁—R selfed progeny, and backcrossed to 01PR06RR.001B to produce BC₂—R progeny. Backcrossing, selection, and self-pollination of the BC-B and BC-R progeny were continued for multiple generations. The 95CB504 male sterile A line, 000A05, was converted to a low saturated phenotype in parallel with the conversion of the 95CB504 B line.

Hybrid seed was generated by hand, using BC₁S₃ generation plants of the 95CB504 B line as the female parent and BC₁S₃ generation plants of the 01PR06RR.001B R line as the male parent. The hybrid seed was grown at 5 locations×4 replications in Western Canada. In the trial plot locations, some individual plants were bagged for self-pollination (5 locations×2 reps) and seeds harvested at maturity. The remaining plants were not bagged (5 locations×4 reps) and seeds were harvested in bulk. As such, the bulk samples had some level of out crossing with non-low saturate fatty acid lines in adjacent plots. Seeds from the individual and bulk samples were analyzed for fatty acid content. Seeds from control plants of line Q2, hybrid v1035 and commercial variety 46A65 were also harvested individually and in bulk.

Table 22 shows the fatty acid profile of the individually bagged samples and bulked samples for hybrid 1524 and controls. The results indicate that seed produced by Hybrid 1524 has a statistically significant decrease in 16:0 content and 18:0 content relative to the controls, and a statistically significant increase in 20:1 content relative to controls. In addition, seeds produced by Hybrid 1524 have a statistically significant decrease in total saturated fatty acid content relative to controls. The total saturated fatty acid content for individually bagged plants is about 5.7%, or about 0.8% less than the parent hybrid which lacks the FatA2 mutation contributed by line 15.24. The total saturated fatty acid content for bulk seed is about 5.9%, or more than 0.9% less than the parent hybrid which lacks the FatA2 mutation contributed by line 15.24.

TABLE 22
Seed Fatty Acid Profile

| Mean C16:0 | N | Line | Mean C18:0 | N | Line |
|---|---|---|---|---|---|
| 3.902 a | 11 | Q2 | 1.903 a | 16 | V1035Bulk |
| 3.876 a | 16 | Q2Bulk | 1.899 a | 16 | Q2Bulk |
| 3.675 b | 16 | 46A65Bulk | 1.887 a | 16 | 46A65Bulk |
| 3.669 b | 16 | V1035Bulk | 1.803 ab | 9 | 46A65 |
| 3.594 bc | 9 | 46A65 | 1.765 b | 11 | Q2 |
| 3.513 cd | 10 | V1035 | 1.744 b | 10 | V1035 |
| 3.414 de | 16 | H1524Bulk | 1.405 c | 16 | H1524Bulk |
| 3.344 e | 10 | H1524 | 1.283 d | 10 | H1524 |
| Mean C20:1 | N | Line | Mean Total Sats | N | Line |
| 1.660 a | 10 | H1524 | 6.986 a | 16 | Q2Bulk |
| 1.599 a | 16 | H1524Bulk | 6.875 a | 11 | Q2 |
| 1.421 b | 10 | V1035 | 6.859 a | 16 | V1035Bulk |
| 1.398 b | 16 | Q2Bulk | 6.776 ab | 16 | 46A65Bulk |
| 1.336 b | 16 | V1035Bulk | 6.601 b | 9 | 46A65 |
| 1.332 b | 16 | 46A65Bulk | 6.568 b | 10 | V1035 |
| 1.331 b | 9 | 46A65 | 5.911 c | 16 | H1524Bulk |
| 1.265 b | 11 | Q2 | 5.704 d | 10 | H1524 |

Another hybrid canola variety yielding seeds with a low total saturated fatty acid content is produced by introducing genes from the low saturate line Skechers-339 into a commercially grown hybrid, using the backcrossing and selection program described above for v1035.

Another hybrid canola variety yielding seeds with a low total saturated fatty acid content is produced by crossing F₆ progeny of a cross of 1764-43-06×1975-90-14, selected for low total saturates, with the parent inbreds of a commercially grown hybrid. An A line, a B line and an R line are selected for low total saturates, using backcrossing and selection as described above for v1035.

Another hybrid canola variety yielding seeds with a low total saturated fatty acid content is produced by crossing Salomon-05, with the parent inbreds of a commercially grown hybrid. An A line, a B line and an R line are selected for low total saturates, using backcrossing and selection as described above for v1035.

Another hybrid canola variety yielding seeds with a low total saturated fatty acid content is produced by crossing Iso1234 with the parent inbreds of hybrid 1524. An A line, a B line and an R line are selected for low total saturates, using backcrossing and selection as described above for v1035. The resulting hybrid, designated Hybrid A2-1234, carries a mutant FatA2 allele and mutant FatB alleles at isoforms 1, 2, 3, and 4.

Another hybrid canola variety yielding seeds with a low total saturated fatty acid content is produced by crossing a variety homozygous for a mutant Fad2 allele and a mutant Fad3 allele with the parent inbreds of Hybrid A2-1234. An A line, a B line and an R line are selected for low total saturates, using backcrossing and selection as described above for v1035. The resulting hybrid carries a mutant FatA2 allele, mutant FatB alleles at isoforms 1, 2, 3, and 4, a mutant Fad2 allele, and a mutant Fad3 allele.

Another hybrid canola variety yielding seeds with a low total saturated fatty acid content is produced by introducing genes from the low saturate line 15.36 into a commercially grown hybrid, using the backcrossing and selection program described above for v1035.

### Example 14

### Identification of Loci Contributing to the Low Saturated Fatty Acid Phenotype of Salomon

A mapping study was conducted to further examine the loci contributing to the low saturated fatty acid phenotype of Salomon (see Example 8). For the study, a mapping population was created to further elucidate the genetic basis of the low saturated fatty acid phenotype by crossing Salomon with Surpass 400. The F₁ microspores resulting from the cross were subjected to the Double Haploid (DH) process. Microspores were treated with colchicine, embryos were regenerated in vitro, and plantlets transferred to a greenhouse for self-pollination. The resulting DH population was designated Sockeye Red. See FIG. 5.

Near-isogenic lines (NILs): Molecular markers identified from QTL mapping in the Sockeye Red DH population were utilized for marker assisted selection (MAS) to introgress the QTL's associated with the low saturated fatty acid phenotype into an elite parent line. In addition, the FATA2 mutation described in PCT/US2010/061226 (published as WO 2011/075716) was also introgressed using a molecular marker developed specifically for that mutation into progeny of. Salomon was crossed with an elite breeding line (03LC.034), which is low in linolenic acid (C18:3). The resulting F1 generation was backcrossed to 03LC.034 three times (generations 1,3,5,7 in FIG. 5). The BC3 was self-pollinated twice to create the BC3S2 seeds during which marker assisted individual plant selections were made to create lines homozygous for QTLs at loci on the B. napus N1 and N19 chromosomes (see FIGS. 6 and 7) as well as to create individuals homozygous for all three of the genetic factors (N1/N19/FATA2) from Salomon (generations 10, 12, 14 in FIG. 5). In an attempt to further reduce the total saturated fatty acid, a cross was made between a BC2 generation individual selection carrying the Salomon genetics (N1/N19/FATA2) with a BC1 individual carrying mutations in four FATB isoforms (generation 9 in FIG. 5). This was crossed again with a BC2 individual carrying all four FATB isoforms and selfed (generations 11, 13 in FIG. 5). An individual homozygous for N1/N19/FATA2/FATB1/FATB4 was identified and self-pollinated in generation 13.

14.1 Genotyping and QTL Mapping

207 lines from Sockeye Red DH population were genotyped on the Illumina (San Diego, Calif.) Brassica 60K Infinium array at DNA Landmarks (Quebec, Canada). The Brassica napus genetic linkage map was constructed using the Kosambi function in JoinMap 3.0 (Van Ooijen et al., 2001). The QTL mapping was performed using the single marker approach (Whitaker, Thompson and Visscher 1996) as well as Haley-Knott Regression (Haley and Knott, 1992) in R/qtl using 1 cM steps. The significance of each QTL was determined based on significance thresholds made from 1000 permutations (Churchill and Doerge, 1994).

14.2 Next-Generation Sequencing and Alignment

Genomic DNA was isolated from leaf tissue using DNeasy Maxi Kit (Qiagen) following standard protocol. Quality and concentration of isolated DNA was measured via spectrophotometry on a NanoDrop 8000 (Thermo Scientific). Isolated genomic DNA from IMC201 (182.1 ng/ul), Salomon (107.4 ng/ul) and Surpass 400 (83.7 ng/ul) was prepared for sequencing by Global Biologics (Columbia, Mo.) using Illumina TruSeq DNA library preparation following standard protocol. Library preparation yielded libraries with the following insert sizes as determined using an Agilent Bioanalyzer: IMC201=367 bp, Salomon=367 bp, Surpass 400=347 bp. DNA libraries were sequenced by the BioMedical Genomics Center at the University of Minnesota (St. Paul, Minn.) on an Illumina HiSeq 2000 to generate 100 bp, paired-end reads. Sequencing yielded 36.5 Gb for IMC201, 30.41 Gb for Salomon, and 36.49 Gb for Surpass 400.

Mapping of the genomic sequencing data (fastq files) to a Brassica napus reference genome (Version 1.0; 19 linkage groups of B. napus genotype DH12075, CanSeq Consortium) was performed using SeqMan NGen v4 (DNAStar, Madison, Wis.). Assembly type chosen was “template assembly—normal workflows” and default setting were used for mapping and SNP calling. These include:

| | |
|---|---|
| format: BAM | SplitTemplateContigs: false |
| merSize: 21 | FilterDeepLayout: true |
| merSkipQuery: 2 | repeatCnt: 100 |
| LayoutType: once | autoTrim: true |
| minMatchPercent: 93 | snp: true |
| gapPenalty: 30 | snpMethod: diploid |
| mismatchPenalty: 20 | snp_minPctToScore: 0.05 |
| matchScore: 10 | snp_minProbNonrefToCall: 0.1 |
| MaxGap: 6 | snp_minVariantDepthToScore: 2 |
| minAlignedLength: 35 | snp_minWeight: 5 |

The SNP report created by SeqMan NGen was exported to ArrayStar v4 (DNAStar, Madison, Wis.) for further filtering. A high-quality SNP list was generated requiring that SNP calls have a quality call score ≧30 (Phred scale), SNP %≧5, depth ≧5, probability that the base is different than the reference base (“p not ref”) ≧90, and be unique to Salomon, i.e., no SNP included in the report was found in IMC201 or Surpass400 at the same position. SNP selection was made for the QTL intervals on linkage groups N1 and N19.

14.3 Fatty Acid Profile of Sockeye Red DH Lines

The fatty acid profile of seeds from individual DH plants were analyzed using gas chromatography (GLC). Seeds were crushed, and lipids were extracted using an alkaline extraction method employing potassium hydroxide/methanol to form the methyl esters followed by, sodium chloride, iso-octane partitioning. The sample was centrifuged and the top layer was used for GC analysis. Least Squares Means (LSMEANS) for each DH line were determined using the GLM procedure in the SAS software package (SAS Institute, 2004) and tests of significance were determined by Student-Newman-Keuls multiple comparison test. The resulting least squares means (LSMEANS) for the full fatty acid profile of Salomon, Surpass 400, and 209 lines from the Sockeye Red DH population are presented in Table 23a. The Pearson correlation coefficients for the fatty acid profile of 207 Sockeye DH lines is provided in Table 23b. The Pearson correlation data in Table 23b, which provides the correlation coefficients between each of the fatty acids shown, indicates there is: a weak and/or very weak correlation between C16:0 and C18:0, 18:1, C18:2 and C18:3; a moderate correlation between C16:0 and total saturated fatty acids (TOTSATS); a strong correlation between C16:0 and C14:0. In addition, there is a strong correlation between C18:0 and C20:0, and a strong correlation between C18:0 and TOTSATS.

TABLE 23a
LSMEANS for the full fatty acid profile of Salomon, Surpass 400,
and 207 lines from the Sockeye Red DH population

| | C14:0 | C16:0 | C16:1 | C18:0 | C18:1 | C18:2 | C18:3 | C20:0 | C20:1 |
|---|---|---|---|---|---|---|---|---|---|
| Salomon-005 | 0.03 | 2.72 | 0.14 | 1.31 | 81.31 | 8.49 | 2.37 | 0.56 | 2.17 |
| Surpass400 | 0.07 | 4.67 | 0.26 | 3.20 | 64.34 | 19.80 | 5.30 | 0.66 | 0.93 |
| SR01 | 0.03 | 3.04 | 0.18 | 1.92 | 65.60 | 20.63 | 5.47 | 0.67 | 1.53 |
| SR02 | 0.04 | 3.47 | 0.23 | 1.94 | 69.07 | 17.12 | 5.43 | 0.63 | 1.20 |
| SR03 | 0.04 | 3.47 | 0.15 | 1.69 | 77.40 | 9.50 | 4.17 | 0.66 | 1.89 |
| SR04 | 0.06 | 3.87 | 0.21 | 1.93 | 77.79 | 8.81 | 4.57 | 0.58 | 1.42 |
| SR05 | 0.05 | 3.26 | 0.23 | 2.09 | 68.89 | 18.13 | 5.35 | 0.48 | 0.92 |
| SR06 | 0.05 | 4.13 | 0.19 | 1.71 | 72.33 | 14.79 | 4.76 | 0.44 | 1.05 |
| SR07 | 0.04 | 2.90 | 0.15 | 3.14 | 68.55 | 19.41 | 3.05 | 0.70 | 1.35 |
| SR08 | 0.05 | 3.41 | 0.24 | 2.98 | 70.05 | 17.46 | 3.14 | 0.79 | 1.02 |
| SR09 | 0.05 | 3.74 | 0.16 | 2.68 | 67.92 | 16.55 | 5.65 | 0.85 | 1.37 |
| SR10 | 0.05 | 3.83 | 0.25 | 2.33 | 78.63 | 7.48 | 4.53 | 0.78 | 1.22 |
| SR100 | 0.04 | 2.87 | 0.17 | 3.11 | 69.92 | 16.71 | 3.85 | 0.87 | 1.49 |
| SR101 | 0.05 | 3.33 | 0.18 | 1.22 | 69.39 | 17.28 | 5.77 | 0.48 | 1.46 |
| SR102 | 0.04 | 3.42 | 0.25 | 1.64 | 71.60 | 17.30 | 3.39 | 0.54 | 1.06 |
| SR103 | 0.05 | 3.82 | 0.21 | 2.70 | 71.81 | 15.10 | 3.43 | 0.84 | 1.13 |
| SR104 | 0.04 | 3.28 | 0.13 | 1.44 | 74.42 | 13.15 | 4.26 | 0.58 | 1.73 |
| SR105 | 0.03 | 2.93 | 0.20 | 3.43 | 78.26 | 7.82 | 3.44 | 1.07 | 1.55 |
| SR106 | 0.02 | 2.91 | 0.13 | 1.47 | 80.93 | 8.57 | 2.81 | 0.56 | 1.72 |
| SR107 | 0.03 | 2.73 | 0.11 | 1.04 | 69.08 | 20.29 | 4.02 | 0.38 | 1.63 |
| SR108 | 0.06 | 3.68 | 0.25 | 1.64 | 79.48 | 8.70 | 4.16 | 0.46 | 1.04 |
| SR109 | 0.05 | 3.79 | 0.23 | 2.37 | 74.22 | 13.16 | 3.92 | 0.61 | 0.99 |
| SR11 | 0.03 | 2.54 | 0.14 | 1.74 | 78.98 | 10.07 | 3.50 | 0.60 | 1.61 |
| SR110 | 0.05 | 4.15 | 0.19 | 1.64 | 70.16 | 18.37 | 3.43 | 0.45 | 1.04 |
| SR111 | 0.05 | 4.24 | 0.19 | 1.30 | 69.85 | 18.97 | 3.64 | 0.34 | 0.95 |
| SR112 | 0.05 | 3.21 | 0.21 | 1.56 | 80.12 | 8.48 | 4.06 | 0.47 | 1.21 |
| SR113 | 0.05 | 3.47 | 0.21 | 1.65 | 79.14 | 8.28 | 4.88 | 0.45 | 1.24 |
| SR114 | 0.05 | 4.21 | 0.23 | 2.32 | 70.63 | 15.93 | 4.76 | 0.50 | 0.86 |
| SR115 | 0.05 | 3.18 | 0.17 | 1.69 | 73.51 | 14.31 | 4.03 | 0.62 | 1.48 |
| SR116 | 0.06 | 3.08 | 0.18 | 1.44 | 81.08 | 8.85 | 2.47 | 0.57 | 1.44 |
| SR117 | 0.04 | 3.19 | 0.14 | 1.59 | 69.77 | 19.55 | 3.15 | 0.43 | 1.52 |
| SR118 | 0.03 | 3.32 | 0.14 | 1.52 | 67.72 | 19.19 | 5.36 | 0.43 | 1.45 |
| SR119 | 0.06 | 3.45 | 0.25 | 2.44 | 79.02 | 8.51 | 3.52 | 0.65 | 1.23 |
| SR12 | 0.04 | 3.72 | 0.14 | 1.92 | 70.21 | 15.10 | 4.96 | 0.76 | 2.00 |
| SR120 | 0.04 | 3.40 | 0.23 | 2.28 | 68.73 | 18.42 | 4.18 | 0.71 | 1.11 |
| SR121 | 0.04 | 2.86 | 0.27 | 2.41 | 81.17 | 8.25 | 2.22 | 0.75 | 1.17 |
| SR122 | 0.03 | 2.60 | 0.15 | 2.20 | 79.04 | 7.63 | 4.97 | 0.80 | 1.67 |
| SR123 | 0.05 | 3.70 | 0.18 | 1.54 | 79.49 | 8.58 | 3.48 | 0.65 | 1.48 |
| SR124 | 0.05 | 3.18 | 0.22 | 2.53 | 72.60 | 14.99 | 3.72 | 0.78 | 1.10 |
| SR125 | 0.03 | 2.75 | 0.13 | 2.94 | 72.43 | 14.45 | 4.74 | 0.64 | 1.22 |
| SR126 | 0.06 | 3.59 | 0.23 | 1.73 | 76.63 | 11.17 | 4.46 | 0.46 | 1.07 |
| SR127 | 0.04 | 3.47 | 0.19 | 1.60 | 70.71 | 17.10 | 4.84 | 0.45 | 1.09 |
| SR128 | 0.04 | 3.68 | 0.26 | 2.72 | 71.12 | 15.47 | 3.67 | 0.83 | 1.21 |
| SR13 | 0.04 | 3.23 | 0.25 | 2.41 | 69.96 | 17.20 | 4.10 | 0.74 | 1.14 |
| SR131 | 0.05 | 3.45 | 0.25 | 3.50 | 69.55 | 17.88 | 2.91 | 0.74 | 0.92 |
| SR132 | 0.04 | 3.10 | 0.14 | 1.86 | 79.68 | 7.59 | 4.37 | 0.65 | 1.66 |
| SR133 | 0.05 | 3.16 | 0.25 | 2.38 | 79.18 | 9.45 | 2.49 | 0.79 | 1.38 |
| SR134 | 0.06 | 3.83 | 0.23 | 2.01 | 71.46 | 16.81 | 3.13 | 0.61 | 1.14 |
| SR135 | 0.04 | 3.24 | 0.18 | 1.39 | 79.33 | 9.04 | 4.14 | 0.52 | 1.38 |
| SR136 | 0.05 | 3.88 | 0.14 | 1.37 | 67.93 | 21.03 | 3.56 | 0.35 | 1.15 |
| SR137 | 0.03 | 3.15 | 0.15 | 1.79 | 74.52 | 15.52 | 2.32 | 0.45 | 1.40 |
| SR138 | 0.04 | 3.07 | 0.12 | 2.35 | 71.49 | 17.15 | 3.34 | 0.53 | 1.33 |
| SR139 | 0.04 | 2.99 | 0.13 | 1.64 | 79.71 | 10.34 | 2.65 | 0.42 | 1.50 |
| SR14 | 0.05 | 3.32 | 0.21 | 2.65 | 71.66 | 16.09 | 2.98 | 0.87 | 1.26 |
| SR140 | 0.06 | 3.35 | 0.27 | 2.08 | 79.01 | 8.71 | 4.69 | 0.47 | 0.91 |
| SR141 | 0.03 | 2.72 | 0.15 | 1.99 | 68.12 | 20.40 | 3.76 | 0.61 | 1.41 |
| SR142 | 0.04 | 2.79 | 0.14 | 2.73 | 80.39 | 6.96 | 3.36 | 0.90 | 1.73 |
| SR143 | 0.04 | 3.64 | 0.23 | 1.87 | 78.58 | 10.10 | 3.07 | 0.59 | 1.16 |
| SR144 | 0.03 | 3.24 | 0.14 | 1.05 | 64.62 | 20.55 | 7.24 | 0.40 | 1.81 |
| SR145 | 0.05 | 3.65 | 0.21 | 1.78 | 68.59 | 19.82 | 3.00 | 0.64 | 1.38 |
| SR146 | 0.06 | 4.48 | 0.24 | 2.63 | 68.27 | 17.32 | 4.81 | 0.60 | 0.96 |
| SR147 | 0.06 | 3.58 | 0.26 | 2.29 | 70.76 | 17.51 | 3.24 | 0.57 | 1.09 |
| SR148 | 0.04 | 3.42 | 0.27 | 2.09 | 68.27 | 19.83 | 3.53 | 0.65 | 1.09 |
| SR149 | 0.04 | 3.37 | 0.22 | 2.11 | 71.27 | 16.04 | 4.34 | 0.68 | 1.11 |
| SR15 | 0.05 | 3.94 | 0.16 | 1.42 | 78.01 | 10.72 | 2.84 | 0.47 | 1.57 |
| SR150 | 0.05 | 3.64 | 0.18 | 1.14 | 79.12 | 7.30 | 5.87 | 0.46 | 1.51 |
| SR151 | 0.06 | 4.20 | 0.16 | 1.82 | 73.22 | 13.90 | 3.54 | 0.69 | 1.46 |
| SR152 | 0.04 | 3.29 | 0.21 | 2.58 | 73.46 | 15.32 | 3.09 | 0.54 | 0.90 |
| SR153 | 0.06 | 3.44 | 0.16 | 2.13 | 73.95 | 14.22 | 3.81 | 0.52 | 1.10 |
| SR154 | 0.04 | 2.87 | 0.14 | 1.49 | 79.40 | 8.56 | 4.27 | 0.56 | 1.86 |
| SR155 | 0.04 | 2.95 | 0.18 | 1.56 | 81.18 | 8.61 | 2.91 | 0.56 | 1.29 |
| SR156 | 0.05 | 3.77 | 0.16 | 1.38 | 71.65 | 17.31 | 2.99 | 0.53 | 1.39 |
| SR157 | 0.04 | 3.22 | 0.27 | 1.93 | 79.91 | 9.31 | 3.32 | 0.52 | 0.93 |
| SR158 | 0.05 | 3.84 | 0.15 | 1.63 | 79.23 | 9.97 | 3.09 | 0.40 | 1.11 |
| SR159 | 0.05 | 3.35 | 0.23 | 2.88 | 77.40 | 8.57 | 3.91 | 0.94 | 1.52 |
| SR16 | 0.07 | 3.79 | 0.25 | 3.02 | 69.54 | 16.34 | 4.57 | 0.70 | 1.03 |
| SR160 | 0.04 | 3.32 | 0.24 | 2.04 | 70.68 | 18.42 | 2.63 | 0.64 | 1.16 |
| SR161 | 0.09 | 4.58 | 0.26 | 1.92 | 68.28 | 18.16 | 4.59 | 0.47 | 1.05 |
| SR162 | 0.04 | 3.37 | 0.15 | 1.96 | 69.35 | 17.28 | 5.04 | 0.50 | 1.58 |
| SR163 | 0.06 | 3.85 | 0.28 | 3.38 | 74.32 | 12.65 | 2.45 | 0.99 | 1.06 |
| SR164 | 0.04 | 3.12 | 0.19 | 2.43 | 68.70 | 18.44 | 3.96 | 0.71 | 1.42 |
| SR165 | 0.07 | 4.38 | 0.34 | 3.70 | 77.77 | 7.63 | 3.14 | 0.97 | 1.13 |
| SR166 | 0.05 | 3.38 | 0.21 | 2.79 | 69.41 | 19.37 | 2.78 | 0.54 | 0.92 |
| SR167 | 0.07 | 4.08 | 0.44 | 3.17 | 67.87 | 19.51 | 2.55 | 0.72 | 0.91 |
| SR168 | 0.04 | 3.12 | 0.18 | 1.55 | 83.14 | 6.25 | 2.74 | 0.63 | 1.52 |
| SR169 | 0.03 | 3.46 | 0.22 | 1.97 | 66.26 | 19.78 | 5.16 | 0.66 | 1.49 |
| SR17 | 0.05 | 3.25 | 0.18 | 1.09 | 70.05 | 16.69 | 6.19 | 0.43 | 1.34 |
| SR170 | 0.03 | 2.89 | 0.11 | 1.38 | 81.38 | 8.46 | 2.59 | 0.55 | 1.77 |
| SR171 | 0.05 | 4.05 | 0.23 | 1.91 | 68.91 | 16.80 | 5.36 | 0.64 | 1.24 |
| SR172 | 0.05 | 3.90 | 0.23 | 2.68 | 73.10 | 13.48 | 3.71 | 0.83 | 1.13 |
| SR173 | 0.06 | 4.19 | 0.16 | 1.67 | 71.20 | 17.21 | 3.00 | 0.55 | 1.22 |
| SR174 | 0.05 | 3.67 | 0.20 | 1.20 | 79.73 | 8.07 | 4.54 | 0.47 | 1.37 |
| SR175 | 0.03 | 3.41 | 0.24 | 2.22 | 72.07 | 15.56 | 3.85 | 0.67 | 1.12 |
| SR176 | 0.02 | 3.04 | 0.20 | 2.31 | 68.21 | 17.93 | 5.26 | 0.74 | 1.45 |
| SR177 | 0.04 | 3.26 | 0.18 | 1.26 | 69.70 | 16.89 | 5.98 | 0.47 | 1.39 |
| SR178 | 0.05 | 3.68 | 0.13 | 1.55 | 67.29 | 19.85 | 4.83 | 0.45 | 1.50 |
| SR179 | 0.07 | 3.84 | 0.24 | 1.75 | 77.99 | 9.18 | 4.50 | 0.47 | 1.20 |
| SR18 | 0.05 | 3.33 | 0.20 | 1.18 | 78.00 | 8.66 | 5.78 | 0.48 | 1.48 |
| SR180 | 0.04 | 3.31 | 0.16 | 1.73 | 72.45 | 15.67 | 4.58 | 0.42 | 1.10 |
| SR181 | 0.05 | 3.59 | 0.24 | 3.63 | 79.07 | 6.09 | 3.79 | 1.13 | 1.31 |
| SR182 | 0.04 | 3.33 | 0.25 | 2.14 | 69.06 | 19.43 | 3.97 | 0.46 | 0.82 |
| SR183 | 0.04 | 3.16 | 0.14 | 2.42 | 72.21 | 15.67 | 3.07 | 0.77 | 1.60 |
| SR184 | 0.05 | 3.47 | 0.22 | 2.12 | 79.81 | 7.99 | 3.58 | 0.72 | 1.25 |
| SR185 | 0.04 | 2.78 | 0.17 | 2.01 | 67.92 | 18.87 | 5.26 | 0.66 | 1.39 |
| SR186 | 0.11 | 3.75 | 0.38 | 2.55 | 74.99 | 11.78 | 4.05 | 0.62 | 0.94 |
| SR187 | 0.06 | 3.75 | 0.35 | 2.01 | 67.48 | 18.83 | 4.64 | 0.69 | 1.22 |
| SR188 | 0.05 | 3.77 | 0.16 | 1.15 | 78.64 | 8.61 | 5.02 | 0.45 | 1.43 |
| SR189 | 0.05 | 4.15 | 0.16 | 1.46 | 72.49 | 14.02 | 4.77 | 0.60 | 1.43 |
| SR19 | 0.06 | 4.02 | 0.16 | 2.27 | 70.73 | 16.26 | 4.15 | 0.58 | 1.14 |
| SR190 | 0.04 | 3.55 | 0.25 | 2.60 | 73.64 | 15.66 | 2.27 | 0.57 | 0.88 |
| SR191 | 0.04 | 3.22 | 0.12 | 1.35 | 72.33 | 17.18 | 2.62 | 0.49 | 1.82 |
| SR192 | 0.07 | 3.91 | 0.31 | 2.49 | 77.47 | 10.02 | 2.36 | 0.89 | 1.44 |
| SR193 | 0.06 | 4.26 | 0.20 | 1.92 | 73.10 | 14.21 | 3.61 | 0.61 | 1.23 |
| SR194 | 0.04 | 2.80 | 0.15 | 2.35 | 68.64 | 18.56 | 4.13 | 0.75 | 1.58 |
| SR195 | 0.06 | 4.31 | 0.19 | 2.00 | 71.98 | 13.60 | 5.01 | 0.69 | 1.25 |
| SR196 | 0.04 | 3.06 | 0.18 | 1.74 | 80.23 | 7.71 | 5.00 | 0.47 | 1.08 |
| SR197 | 0.04 | 2.91 | 0.13 | 1.30 | 68.86 | 18.04 | 5.78 | 0.46 | 1.71 |
| SR198 | 0.05 | 3.33 | 0.25 | 1.86 | 70.60 | 18.87 | 2.92 | 0.48 | 1.01 |
| SR199 | 0.04 | 3.11 | 0.22 | 2.56 | 80.30 | 7.79 | 3.80 | 0.62 | 0.95 |
| SR20 | 0.06 | 4.36 | 0.21 | 2.54 | 71.48 | 14.53 | 4.05 | 0.68 | 1.27 |
| SR200 | 0.05 | 3.43 | 0.20 | 1.70 | 68.31 | 17.38 | 6.20 | 0.60 | 1.27 |
| SR201 | 0.03 | 2.81 | 0.13 | 1.50 | 79.33 | 9.75 | 3.19 | 0.59 | 1.77 |
| SR202 | 0.06 | 3.49 | 0.37 | 2.26 | 74.42 | 10.61 | 5.34 | 0.72 | 1.32 |
| SR203 | 0.05 | 3.30 | 0.30 | 1.81 | 77.64 | 8.47 | 5.71 | 0.66 | 1.27 |
| SR204 | 0.04 | 3.82 | 0.26 | 2.07 | 76.67 | 11.07 | 3.51 | 0.67 | 1.12 |
| SR205 | 0.05 | 2.98 | 0.18 | 3.11 | 70.69 | 15.57 | 4.76 | 0.65 | 1.17 |
| SR206 | 0.03 | 3.08 | 0.12 | 1.16 | 68.32 | 18.99 | 4.80 | 0.49 | 2.03 |
| SR207 | 0.05 | 3.34 | 0.24 | 2.04 | 78.32 | 10.36 | 2.80 | 0.70 | 1.25 |
| SR208 | 0.03 | 2.46 | 0.17 | 1.61 | 77.68 | 9.64 | 5.16 | 0.59 | 1.81 |
| SR209 | 0.05 | 3.34 | 0.17 | 1.83 | 71.64 | 16.96 | 2.68 | 0.69 | 1.60 |
| SR21 | 0.05 | 4.05 | 0.20 | 1.83 | 79.59 | 7.78 | 4.10 | 0.53 | 1.23 |
| SR22 | 0.04 | 3.43 | 0.24 | 2.01 | 65.37 | 22.48 | 3.88 | 0.64 | 1.10 |
| SR23 | 0.05 | 3.21 | 0.15 | 1.39 | 69.90 | 16.86 | 6.47 | 0.39 | 1.08 |
| SR24 | 0.05 | 3.92 | 0.16 | 1.45 | 73.01 | 13.70 | 4.97 | 0.54 | 1.41 |
| SR25 | 0.05 | 3.66 | 0.17 | 1.99 | 81.01 | 7.67 | 3.20 | 0.52 | 1.15 |
| SR26 | 0.06 | 3.96 | 0.23 | 2.63 | 78.71 | 7.83 | 3.91 | 0.68 | 1.14 |
| SR27 | 0.05 | 3.66 | 0.24 | 1.83 | 64.50 | 20.53 | 6.44 | 0.60 | 1.22 |
| SR28 | 0.03 | 3.04 | 0.15 | 2.34 | 78.96 | 8.12 | 4.36 | 0.75 | 1.39 |
| SR29 | 0.05 | 3.58 | 0.23 | 2.82 | 79.58 | 7.29 | 4.35 | 0.62 | 0.95 |
| SR30 | 0.05 | 3.92 | 0.18 | 1.44 | 80.07 | 7.60 | 4.57 | 0.45 | 1.17 |
| SR31 | 0.05 | 3.12 | 0.15 | 1.40 | 68.67 | 20.20 | 4.32 | 0.38 | 1.19 |
| SR32 | 0.06 | 4.31 | 0.18 | 1.53 | 68.16 | 16.63 | 6.35 | 0.58 | 1.34 |
| SR33 | 0.07 | 3.65 | 0.18 | 1.64 | 74.90 | 11.31 | 5.97 | 0.42 | 1.20 |
| SR34 | 0.05 | 3.38 | 0.23 | 1.80 | 78.57 | 8.63 | 4.44 | 0.67 | 1.31 |
| SR35 | 0.04 | 3.19 | 0.21 | 2.10 | 70.33 | 16.34 | 5.31 | 0.64 | 1.08 |
| SR36 | 0.04 | 3.20 | 0.17 | 2.32 | 78.24 | 7.78 | 4.25 | 0.89 | 1.93 |
| SR37 | 0.05 | 3.46 | 0.25 | 2.77 | 70.11 | 16.22 | 4.83 | 0.64 | 0.97 |
| SR38 | 0.03 | 2.85 | 0.13 | 1.55 | 69.67 | 19.58 | 3.03 | 0.55 | 1.69 |
| SR39 | 0.06 | 4.14 | 0.20 | 2.90 | 69.83 | 16.15 | 4.45 | 0.64 | 0.94 |
| SR40 | 0.04 | 3.42 | 0.21 | 2.43 | 73.10 | 15.37 | 2.63 | 0.75 | 1.19 |
| SR41 | 0.05 | 3.20 | 0.23 | 2.59 | 79.79 | 8.83 | 3.26 | 0.57 | 0.91 |
| SR42 | 0.04 | 3.47 | 0.18 | 1.22 | 72.09 | 15.26 | 5.25 | 0.46 | 1.35 |
| SR43 | 0.06 | 3.82 | 0.20 | 1.76 | 79.76 | 7.99 | 4.15 | 0.48 | 1.15 |
| SR44 | 0.04 | 3.34 | 0.26 | 1.85 | 74.97 | 13.60 | 4.00 | 0.42 | 0.97 |
| SR45 | 0.05 | 3.63 | 0.21 | 1.60 | 70.12 | 19.10 | 3.31 | 0.42 | 1.03 |
| SR46 | 0.06 | 3.47 | 0.24 | 2.33 | 69.54 | 15.97 | 5.71 | 0.69 | 1.16 |
| SR47 | 0.04 | 3.43 | 0.12 | 1.58 | 72.72 | 16.27 | 2.73 | 0.57 | 1.67 |
| SR48 | 0.03 | 3.20 | 0.25 | 2.15 | 78.02 | 9.64 | 4.22 | 0.69 | 1.11 |
| SR49 | 0.05 | 3.90 | 0.22 | 1.83 | 78.32 | 8.65 | 4.23 | 0.68 | 1.29 |
| SR50 | 0.05 | 3.86 | 0.19 | 1.45 | 79.47 | 7.22 | 4.84 | 0.59 | 1.48 |
| SR51 | 0.07 | 3.90 | 0.21 | 2.74 | 77.18 | 9.13 | 4.21 | 0.68 | 1.11 |
| SR52 | 0.05 | 3.02 | 0.15 | 1.58 | 80.88 | 9.56 | 2.70 | 0.39 | 1.13 |
| SR53 | 0.05 | 4.03 | 0.22 | 1.51 | 78.14 | 9.04 | 5.22 | 0.43 | 0.97 |
| SR54 | 0.02 | 3.09 | 0.16 | 2.26 | 79.69 | 8.72 | 3.53 | 0.62 | 1.23 |
| SR55 | 0.03 | 3.00 | 0.14 | 3.04 | 78.20 | 7.47 | 5.47 | 0.67 | 1.31 |
| SR56 | 0.05 | 3.39 | 0.17 | 1.62 | 71.77 | 15.45 | 5.25 | 0.46 | 1.15 |
| SR57 | 0.05 | 3.23 | 0.16 | 1.48 | 66.78 | 21.00 | 4.64 | 0.51 | 1.30 |
| SR58 | 0.03 | 2.43 | 0.15 | 1.75 | 77.41 | 9.34 | 5.97 | 0.62 | 1.52 |
| SR59 | 0.05 | 3.93 | 0.21 | 1.54 | 80.05 | 7.88 | 4.36 | 0.45 | 1.02 |
| SR60 | 0.03 | 2.91 | 0.13 | 1.65 | 70.29 | 16.49 | 5.52 | 0.58 | 1.55 |
| SR61 | 0.03 | 3.21 | 0.15 | 2.30 | 69.61 | 18.63 | 2.47 | 0.78 | 1.67 |
| SR62 | 0.04 | 2.93 | 0.13 | 1.26 | 70.74 | 16.63 | 5.29 | 0.48 | 1.69 |
| SR63 | 0.06 | 4.33 | 0.19 | 1.61 | 69.83 | 18.22 | 3.08 | 0.58 | 1.22 |
| SR64 | 0.07 | 4.07 | 0.22 | 2.05 | 68.34 | 19.81 | 2.86 | 0.53 | 1.23 |
| SR65 | 0.03 | 2.87 | 0.17 | 2.21 | 74.96 | 12.49 | 3.75 | 0.71 | 1.66 |
| SR66 | 0.04 | 3.13 | 0.15 | 2.69 | 77.31 | 10.84 | 3.33 | 0.59 | 1.28 |
| SR67 | 0.04 | 3.44 | 0.18 | 1.26 | 68.93 | 20.11 | 3.29 | 0.47 | 1.44 |
| SR68 | 0.04 | 3.43 | 0.24 | 3.05 | 70.73 | 15.22 | 4.33 | 0.92 | 1.13 |
| SR69 | 0.05 | 3.79 | 0.22 | 2.76 | 74.00 | 12.65 | 3.69 | 0.83 | 1.10 |
| SR70 | 0.05 | 3.59 | 0.24 | 2.51 | 80.20 | 7.30 | 3.96 | 0.63 | 0.94 |
| SR71 | 0.07 | 4.32 | 0.29 | 3.10 | 73.16 | 12.87 | 3.44 | 0.81 | 1.11 |
| SR72 | 0.04 | 3.55 | 0.21 | 0.93 | 65.58 | 20.69 | 6.68 | 0.36 | 1.26 |
| SR73 | 0.03 | 3.17 | 0.15 | 2.14 | 65.79 | 20.20 | 6.19 | 0.54 | 1.17 |
| SR74 | 0.04 | 2.95 | 0.15 | 2.15 | 80.16 | 7.65 | 4.58 | 0.55 | 1.15 |
| SR75 | 0.05 | 3.52 | 0.18 | 1.53 | 69.88 | 17.63 | 4.37 | 0.58 | 1.33 |
| SR76 | 0.06 | 4.03 | 0.19 | 1.96 | 72.99 | 15.20 | 3.37 | 0.56 | 1.03 |
| SR77 | 0.05 | 3.28 | 0.24 | 1.90 | 67.79 | 18.18 | 5.77 | 0.62 | 1.28 |
| SR78 | 0.04 | 3.71 | 0.19 | 2.04 | 80.14 | 9.03 | 2.79 | 0.51 | 1.02 |
| SR79 | 0.04 | 3.16 | 0.21 | 2.76 | 72.69 | 14.76 | 3.01 | 0.93 | 1.21 |
| SR80 | 0.03 | 3.13 | 0.16 | 1.25 | 65.47 | 23.07 | 3.96 | 0.49 | 1.57 |
| SR81 | 0.05 | 4.27 | 0.22 | 2.04 | 69.44 | 18.88 | 3.01 | 0.53 | 0.93 |
| SR82 | 0.04 | 3.60 | 0.26 | 1.90 | 66.55 | 21.89 | 3.30 | 0.61 | 1.05 |
| SR83 | 0.05 | 4.18 | 0.26 | 2.29 | 70.20 | 17.12 | 4.08 | 0.51 | 0.79 |
| SR84 | 0.06 | 3.78 | 0.23 | 2.60 | 73.10 | 15.96 | 2.41 | 0.55 | 0.85 |
| SR85 | 0.05 | 4.64 | 0.29 | 2.70 | 69.07 | 16.27 | 3.78 | 0.86 | 1.23 |
| SR86 | 0.05 | 3.38 | 0.15 | 1.44 | 64.75 | 21.57 | 5.35 | 0.53 | 1.80 |
| SR87 | 0.04 | 3.23 | 0.14 | 1.74 | 67.98 | 20.06 | 4.31 | 0.42 | 1.38 |
| SR88 | 0.04 | 3.78 | 0.28 | 1.93 | 68.17 | 19.90 | 3.30 | 0.63 | 1.10 |
| SR89 | 0.05 | 3.84 | 0.21 | 2.50 | 71.49 | 16.92 | 2.82 | 0.56 | 0.98 |
| SR90 | 0.04 | 3.40 | 0.18 | 1.48 | 72.79 | 15.19 | 4.13 | 0.54 | 1.36 |
| SR91 | 0.05 | 4.19 | 0.23 | 1.43 | 69.60 | 16.39 | 5.48 | 0.54 | 1.26 |
| SR92 | 0.02 | 2.74 | 0.16 | 1.40 | 76.70 | 9.99 | 6.31 | 0.51 | 1.41 |
| SR93 | 0.05 | 3.88 | 0.19 | 1.42 | 79.29 | 9.73 | 2.68 | 0.55 | 1.42 |
| SR94 | 0.05 | 3.82 | 0.23 | 2.13 | 67.31 | 20.89 | 3.49 | 0.51 | 0.94 |
| SR95 | 0.04 | 3.31 | 0.21 | 3.09 | 73.12 | 13.80 | 4.12 | 0.67 | 0.95 |
| SR96 | 0.04 | 3.03 | 0.21 | 1.44 | 81.47 | 8.09 | 2.96 | 0.56 | 1.37 |
| SR97 | 0.04 | 2.67 | 0.18 | 2.25 | 80.35 | 8.29 | 2.87 | 0.77 | 1.60 |
| SR98 | 0.08 | 3.96 | 0.37 | 3.53 | 78.30 | 8.81 | 1.97 | 0.99 | 1.02 |
| SR99 | 0.04 | 2.51 | 0.13 | 1.93 | 73.93 | 14.81 | 3.74 | 0.62 | 1.48 |

| | C20:2 | C22:0 | C22:1 | C24:0 | C24:1 | TOTAL SATS |
|---|---|---|---|---|---|---|
| Salomon-005 | 0.12 | 0.33 | 0.05 | 0.26 | 0.13 | 5.21 |
| Surpass400 | 0.07 | 0.23 | 0.02 | 0.26 | 0.21 | 9.07 |
| SR01 | 0.11 | 0.36 | 0.04 | 0.20 | 0.22 | 6.21 |
| SR02 | 0.08 | 0.30 | 0.03 | 0.26 | 0.20 | 6.64 |
| SR03 | 0.12 | 0.36 | 0.05 | 0.29 | 0.20 | 6.51 |
| SR04 | 0.11 | 0.23 | 0.04 | 0.22 | 0.16 | 6.88 |
| SR05 | 0.08 | 0.18 | 0.02 | 0.17 | 0.16 | 6.22 |
| SR06 | 0.08 | 0.17 | 0.03 | 0.15 | 0.14 | 6.64 |
| SR07 | 0.12 | 0.25 | 0.03 | 0.20 | 0.15 | 7.23 |
| SR08 | 0.06 | 0.33 | 0.02 | 0.29 | 0.15 | 7.84 |
| SR09 | 0.08 | 0.41 | 0.05 | 0.34 | 0.16 | 8.06 |
| SR10 | 0.05 | 0.37 | 0.03 | 0.26 | 0.19 | 7.62 |
| SR100 | 0.10 | 0.36 | 0.03 | 0.34 | 0.14 | 7.59 |
| SR101 | 0.10 | 0.28 | 0.05 | 0.24 | 0.16 | 5.59 |
| SR102 | 0.07 | 0.27 | 0.02 | 0.20 | 0.20 | 6.11 |
| SR103 | 0.06 | 0.38 | 0.02 | 0.30 | 0.16 | 8.08 |
| SR104 | 0.13 | 0.32 | 0.05 | 0.28 | 0.20 | 5.92 |
| SR105 | 0.07 | 0.51 | 0.04 | 0.36 | 0.29 | 8.33 |
| SR106 | 0.12 | 0.27 | 0.04 | 0.27 | 0.17 | 5.51 |
| SR107 | 0.14 | 0.21 | 0.04 | 0.12 | 0.17 | 4.50 |
| SR108 | 0.08 | 0.18 | 0.02 | 0.18 | 0.08 | 6.19 |
| SR109 | 0.06 | 0.23 | 0.02 | 0.17 | 0.16 | 7.23 |
| SR11 | 0.10 | 0.31 | 0.04 | 0.21 | 0.15 | 5.42 |
| SR110 | 0.08 | 0.18 | 0.02 | 0.12 | 0.11 | 6.60 |
| SR111 | 0.08 | 0.14 | 0.02 | 0.07 | 0.14 | 6.16 |
| SR112 | 0.08 | 0.21 | 0.03 | 0.16 | 0.17 | 5.65 |
| SR113 | 0.07 | 0.19 | 0.02 | 0.23 | 0.11 | 6.04 |
| SR114 | 0.06 | 0.18 | 0.02 | 0.13 | 0.14 | 7.38 |
| SR115 | 0.10 | 0.30 | 0.05 | 0.36 | 0.16 | 6.18 |
| SR116 | 0.08 | 0.29 | 0.04 | 0.28 | 0.13 | 5.73 |
| SR117 | 0.15 | 0.17 | 0.03 | 0.17 | 0.10 | 5.59 |
| SR118 | 0.16 | 0.18 | 0.04 | 0.17 | 0.28 | 5.66 |
| SR119 | 0.09 | 0.25 | 0.05 | 0.28 | 0.19 | 7.14 |
| SR12 | 0.15 | 0.39 | 0.06 | 0.35 | 0.20 | 7.18 |
| SR120 | 0.07 | 0.34 | 0.03 | 0.27 | 0.20 | 7.03 |
| SR121 | 0.04 | 0.35 | 0.03 | 0.33 | 0.13 | 6.74 |
| SR122 | 0.08 | 0.41 | 0.03 | 0.25 | 0.13 | 6.29 |
| SR123 | 0.08 | 0.37 | 0.04 | 0.23 | 0.15 | 6.53 |
| SR124 | 0.06 | 0.34 | 0.02 | 0.25 | 0.16 | 7.13 |
| SR125 | 0.09 | 0.22 | 0.03 | 0.18 | 0.16 | 6.76 |
| SR126 | 0.09 | 0.19 | 0.03 | 0.15 | 0.15 | 6.18 |
| SR127 | 0.08 | 0.19 | 0.02 | 0.12 | 0.11 | 5.86 |
| SR128 | 0.08 | 0.36 | 0.03 | 0.35 | 0.19 | 7.98 |
| SR13 | 0.09 | 0.34 | 0.05 | 0.27 | 0.17 | 7.05 |
| SR131 | 0.07 | 0.24 | 0.02 | 0.22 | 0.20 | 8.22 |
| SR132 | 0.11 | 0.30 | 0.05 | 0.25 | 0.20 | 6.20 |
| SR133 | 0.06 | 0.37 | 0.03 | 0.28 | 0.14 | 7.02 |
| SR134 | 0.06 | 0.28 | 0.02 | 0.23 | 0.12 | 7.02 |
| SR135 | 0.08 | 0.28 | 0.03 | 0.19 | 0.16 | 5.67 |
| SR136 | 0.09 | 0.15 | 0.02 | 0.17 | 0.10 | 5.97 |
| SR137 | 0.12 | 0.17 | 0.03 | 0.19 | 0.14 | 5.78 |
| SR138 | 0.10 | 0.20 | 0.02 | 0.17 | 0.10 | 6.35 |
| SR139 | 0.13 | 0.18 | 0.05 | 0.13 | 0.11 | 5.39 |
| SR14 | 0.07 | 0.39 | 0.03 | 0.26 | 0.17 | 7.53 |
| SR140 | 0.05 | 0.18 | 0.01 | 0.14 | 0.08 | 6.28 |
| SR141 | 0.11 | 0.28 | 0.03 | 0.23 | 0.17 | 5.86 |
| SR142 | 0.07 | 0.40 | 0.03 | 0.29 | 0.18 | 7.15 |
| SR143 | 0.06 | 0.26 | 0.02 | 0.21 | 0.17 | 6.62 |
| SR144 | 0.18 | 0.23 | 0.05 | 0.23 | 0.22 | 5.19 |
| SR145 | 0.10 | 0.34 | 0.04 | 0.29 | 0.12 | 6.75 |
| SR146 | 0.08 | 0.22 | 0.02 | 0.20 | 0.11 | 8.19 |
| SR147 | 0.10 | 0.23 | 0.03 | 0.20 | 0.12 | 6.91 |
| SR148 | 0.08 | 0.30 | 0.03 | 0.23 | 0.17 | 6.73 |
| SR149 | 0.06 | 0.31 | 0.03 | 0.28 | 0.14 | 6.78 |
| SR15 | 0.12 | 0.22 | 0.03 | 0.29 | 0.15 | 6.39 |
| SR150 | 0.08 | 0.27 | 0.03 | 0.16 | 0.17 | 5.73 |
| SR151 | 0.08 | 0.37 | 0.03 | 0.35 | 0.11 | 7.48 |
| SR152 | 0.07 | 0.18 | 0.01 | 0.17 | 0.13 | 6.80 |
| SR153 | 0.08 | 0.18 | 0.05 | 0.20 | 0.10 | 6.52 |
| SR154 | 0.13 | 0.29 | 0.04 | 0.24 | 0.10 | 5.50 |
| SR155 | 0.07 | 0.27 | 0.04 | 0.21 | 0.12 | 5.59 |
| SR156 | 0.09 | 0.28 | 0.04 | 0.20 | 0.18 | 6.19 |
| SR157 | 0.06 | 0.21 | 0.02 | 0.12 | 0.14 | 6.04 |
| SR158 | 0.08 | 0.17 | 0.03 | 0.17 | 0.10 | 6.25 |
| SR159 | 0.09 | 0.45 | 0.07 | 0.39 | 0.18 | 8.06 |
| SR16 | 0.07 | 0.25 | 0.01 | 0.24 | 0.15 | 8.06 |
| SR160 | 0.08 | 0.30 | 0.03 | 0.23 | 0.19 | 6.57 |
| SR161 | 0.09 | 0.18 | 0.04 | 0.18 | 0.11 | 7.42 |
| SR162 | 0.16 | 0.19 | 0.04 | 0.15 | 0.18 | 6.22 |
| SR163 | 0.05 | 0.42 | 0.03 | 0.35 | 0.11 | 9.05 |
| SR164 | 0.12 | 0.31 | 0.06 | 0.33 | 0.16 | 6.95 |
| SR165 | 0.06 | 0.37 | 0.01 | 0.28 | 0.16 | 9.77 |
| SR166 | 0.07 | 0.20 | 0.02 | 0.18 | 0.10 | 7.13 |
| SR167 | 0.11 | 0.24 | 0.02 | 0.22 | 0.12 | 8.49 |
| SR168 | 0.09 | 0.34 | 0.05 | 0.23 | 0.13 | 5.90 |
| SR169 | 0.12 | 0.37 | 0.04 | 0.25 | 0.19 | 6.74 |
| SR17 | 0.09 | 0.24 | 0.04 | 0.19 | 0.15 | 5.25 |
| SR170 | 0.11 | 0.29 | 0.04 | 0.22 | 0.17 | 5.36 |
| SR171 | 0.08 | 0.31 | 0.05 | 0.22 | 0.16 | 7.19 |
| SR172 | 0.05 | 0.40 | 0.04 | 0.29 | 0.12 | 8.15 |
| SR173 | 0.08 | 0.28 | 0.02 | 0.25 | 0.12 | 6.99 |
| SR174 | 0.09 | 0.26 | 0.03 | 0.17 | 0.14 | 5.82 |
| SR175 | 0.06 | 0.30 | 0.03 | 0.29 | 0.16 | 6.91 |
| SR176 | 0.09 | 0.32 | 0.03 | 0.24 | 0.16 | 6.67 |
| SR177 | 0.12 | 0.25 | 0.04 | 0.21 | 0.20 | 5.49 |
| SR178 | 0.12 | 0.21 | 0.04 | 0.20 | 0.13 | 6.13 |
| SR179 | 0.12 | 0.19 | 0.12 | 0.20 | 0.15 | 6.51 |
| SR18 | 0.08 | 0.28 | 0.04 | 0.25 | 0.17 | 5.57 |
| SR180 | 0.08 | 0.16 | 0.02 | 0.16 | 0.10 | 5.82 |
| SR181 | 0.05 | 0.46 | 0.03 | 0.41 | 0.18 | 9.26 |
| SR182 | 0.06 | 0.16 | 0.01 | 0.11 | 0.15 | 6.24 |
| SR183 | 0.10 | 0.35 | 0.03 | 0.34 | 0.10 | 7.08 |
| SR184 | 0.06 | 0.35 | 0.03 | 0.21 | 0.15 | 6.91 |
| SR185 | 0.10 | 0.34 | 0.05 | 0.23 | 0.17 | 6.06 |
| SR186 | 0.08 | 0.26 | 0.05 | 0.28 | 0.18 | 7.56 |
| SR187 | 0.09 | 0.39 | 0.03 | 0.26 | 0.22 | 7.16 |
| SR188 | 0.08 | 0.25 | 0.04 | 0.22 | 0.12 | 5.89 |
| SR189 | 0.09 | 0.33 | 0.04 | 0.23 | 0.19 | 6.81 |
| SR19 | 0.08 | 0.21 | 0.02 | 0.16 | 0.15 | 7.30 |
| SR190 | 0.06 | 0.19 | 0.02 | 0.17 | 0.11 | 7.12 |
| SR191 | 0.16 | 0.25 | 0.04 | 0.23 | 0.15 | 5.59 |
| SR192 | 0.07 | 0.40 | 0.03 | 0.36 | 0.17 | 8.12 |
| SR193 | 0.08 | 0.26 | 0.03 | 0.25 | 0.17 | 7.36 |
| SR194 | 0.11 | 0.37 | 0.04 | 0.31 | 0.17 | 6.61 |
| SR195 | 0.07 | 0.32 | 0.04 | 0.30 | 0.20 | 7.66 |
| SR196 | 0.06 | 0.18 | 0.02 | 0.12 | 0.11 | 5.62 |
| SR197 | 0.15 | 0.22 | 0.05 | 0.21 | 0.15 | 5.13 |
| SR198 | 0.08 | 0.20 | 0.05 | 0.15 | 0.15 | 6.08 |
| SR199 | 0.06 | 0.21 | 0.05 | 0.16 | 0.12 | 6.71 |
| SR20 | 0.09 | 0.26 | 0.03 | 0.22 | 0.23 | 8.12 |
| SR200 | 0.09 | 0.32 | 0.04 | 0.22 | 0.18 | 6.32 |
| SR201 | 0.11 | 0.31 | 0.05 | 0.27 | 0.17 | 5.50 |
| SR202 | 0.06 | 0.35 | 0.03 | 0.25 | 0.74 | 7.12 |
| SR203 | 0.07 | 0.35 | 0.04 | 0.20 | 0.14 | 6.37 |
| SR204 | 0.06 | 0.30 | 0.04 | 0.20 | 0.18 | 7.09 |
| SR205 | 0.09 | 0.26 | 0.04 | 0.34 | 0.12 | 7.39 |
| SR206 | 0.18 | 0.32 | 0.06 | 0.29 | 0.14 | 5.37 |
| SR207 | 0.06 | 0.34 | 0.05 | 0.31 | 0.15 | 6.77 |
| SR208 | 0.11 | 0.34 | 0.04 | 0.25 | 0.12 | 5.28 |
| SR209 | 0.11 | 0.37 | 0.08 | 0.34 | 0.16 | 6.61 |
| SR21 | 0.07 | 0.22 | 0.02 | 0.20 | 0.12 | 6.89 |
| SR22 | 0.08 | 0.31 | 0.03 | 0.20 | 0.20 | 6.63 |
| SR23 | 0.09 | 0.16 | 0.02 | 0.11 | 0.14 | 5.29 |
| SR24 | 0.07 | 0.29 | 0.03 | 0.25 | 0.14 | 6.50 |
| SR25 | 0.06 | 0.20 | 0.02 | 0.20 | 0.09 | 6.62 |
| SR26 | 0.08 | 0.28 | 0.04 | 0.29 | 0.17 | 7.91 |
| SR27 | 0.08 | 0.32 | 0.04 | 0.28 | 0.22 | 6.74 |
| SR28 | 0.07 | 0.36 | 0.03 | 0.25 | 0.16 | 6.78 |
| SR29 | 0.04 | 0.22 | 0.02 | 0.16 | 0.09 | 7.46 |
| SR30 | 0.08 | 0.19 | 0.02 | 0.14 | 0.11 | 6.19 |
| SR31 | 0.10 | 0.15 | 0.03 | 0.12 | 0.12 | 5.21 |
| SR32 | 0.10 | 0.33 | 0.04 | 0.22 | 0.19 | 7.03 |
| SR33 | 0.09 | 0.18 | 0.03 | 0.21 | 0.14 | 6.17 |
| SR34 | 0.06 | 0.38 | 0.04 | 0.28 | 0.16 | 6.56 |
| SR35 | 0.08 | 0.29 | 0.02 | 0.21 | 0.15 | 6.47 |
| SR36 | 0.09 | 0.47 | 0.05 | 0.38 | 0.19 | 7.29 |
| SR37 | 0.06 | 0.23 | 0.02 | 0.28 | 0.11 | 7.42 |
| SR38 | 0.14 | 0.27 | 0.04 | 0.29 | 0.18 | 5.55 |
| SR39 | 0.07 | 0.24 | 0.03 | 0.22 | 0.12 | 8.20 |
| SR40 | 0.07 | 0.33 | 0.03 | 0.31 | 0.14 | 7.27 |
| SR41 | 0.05 | 0.20 | 0.02 | 0.18 | 0.12 | 6.79 |
| SR42 | 0.09 | 0.25 | 0.03 | 0.20 | 0.13 | 5.62 |
| SR43 | 0.08 | 0.21 | 0.03 | 0.21 | 0.11 | 6.54 |
| SR44 | 0.07 | 0.16 | 0.02 | 0.13 | 0.17 | 5.95 |
| SR45 | 0.09 | 0.17 | 0.03 | 0.12 | 0.13 | 5.99 |
| SR46 | 0.06 | 0.31 | 0.03 | 0.29 | 0.15 | 7.15 |
| SR47 | 0.13 | 0.30 | 0.04 | 0.21 | 0.21 | 6.12 |
| SR48 | 0.04 | 0.34 | 0.02 | 0.16 | 0.13 | 6.57 |
| SR49 | 0.06 | 0.38 | 0.03 | 0.22 | 0.14 | 7.06 |
| SR50 | 0.08 | 0.33 | 0.04 | 0.21 | 0.18 | 6.51 |
| SR51 | 0.07 | 0.27 | 0.03 | 0.25 | 0.16 | 7.91 |
| SR52 | 0.09 | 0.15 | 0.05 | 0.15 | 0.11 | 5.33 |
| SR53 | 0.07 | 0.17 | 0.01 | 0.09 | 0.07 | 6.27 |
| SR54 | 0.07 | 0.25 | 0.02 | 0.20 | 0.14 | 6.44 |
| SR55 | 0.07 | 0.23 | 0.03 | 0.22 | 0.11 | 7.20 |
| SR56 | 0.10 | 0.20 | 0.03 | 0.23 | 0.13 | 5.95 |
| SR57 | 0.11 | 0.25 | 0.04 | 0.23 | 0.22 | 5.74 |
| SR58 | 0.08 | 0.33 | 0.03 | 0.19 | 0.12 | 5.35 |
| SR59 | 0.08 | 0.17 | 0.03 | 0.11 | 0.13 | 6.24 |
| SR60 | 0.11 | 0.28 | 0.03 | 0.24 | 0.18 | 5.70 |
| SR61 | 0.13 | 0.38 | 0.05 | 0.30 | 0.28 | 7.01 |
| SR62 | 0.13 | 0.25 | 0.04 | 0.25 | 0.14 | 5.21 |
| SR63 | 0.09 | 0.28 | 0.05 | 0.28 | 0.18 | 7.14 |
| SR64 | 0.10 | 0.21 | 0.04 | 0.33 | 0.14 | 7.26 |
| SR65 | 0.11 | 0.31 | 0.05 | 0.29 | 0.38 | 6.42 |
| SR66 | 0.11 | 0.20 | 0.02 | 0.16 | 0.14 | 6.81 |
| SR67 | 0.13 | 0.26 | 0.04 | 0.22 | 0.19 | 5.68 |
| SR68 | 0.07 | 0.41 | 0.02 | 0.25 | 0.15 | 8.10 |
| SR69 | 0.06 | 0.37 | 0.03 | 0.29 | 0.17 | 8.09 |
| SR70 | 0.04 | 0.23 | 0.02 | 0.16 | 0.12 | 7.17 |
| SR71 | 0.06 | 0.30 | 0.02 | 0.33 | 0.12 | 8.93 |
| SR72 | 0.11 | 0.20 | 0.05 | 0.16 | 0.18 | 5.24 |
| SR73 | 0.10 | 0.22 | 0.02 | 0.14 | 0.14 | 6.24 |
| SR74 | 0.07 | 0.22 | 0.04 | 0.17 | 0.13 | 6.06 |
| SR75 | 0.09 | 0.32 | 0.04 | 0.29 | 0.19 | 6.30 |
| SR76 | 0.07 | 0.21 | 0.02 | 0.15 | 0.15 | 6.96 |
| SR77 | 0.09 | 0.33 | 0.04 | 0.25 | 0.20 | 6.42 |
| SR78 | 0.05 | 0.21 | 0.02 | 0.15 | 0.11 | 6.65 |
| SR79 | 0.07 | 0.45 | 0.06 | 0.40 | 0.25 | 7.74 |
| SR80 | 0.15 | 0.29 | 0.06 | 0.19 | 0.20 | 5.37 |
| SR81 | 0.08 | 0.22 | 0.02 | 0.13 | 0.19 | 7.24 |
| SR82 | 0.07 | 0.31 | 0.03 | 0.20 | 0.21 | 6.65 |
| SR83 | 0.05 | 0.18 | 0.01 | 0.12 | 0.17 | 7.32 |
| SR84 | 0.05 | 0.18 | 0.02 | 0.10 | 0.13 | 7.26 |
| SR85 | 0.09 | 0.38 | 0.02 | 0.37 | 0.23 | 9.01 |
| SR86 | 0.18 | 0.30 | 0.05 | 0.28 | 0.18 | 5.99 |
| SR87 | 0.15 | 0.17 | 0.03 | 0.24 | 0.11 | 5.84 |
| SR88 | 0.08 | 0.30 | 0.03 | 0.26 | 0.21 | 6.94 |
| SR89 | 0.07 | 0.20 | 0.01 | 0.20 | 0.13 | 7.36 |
| SR90 | 0.08 | 0.28 | 0.04 | 0.28 | 0.20 | 6.03 |
| SR91 | 0.08 | 0.33 | 0.03 | 0.18 | 0.22 | 6.72 |
| SR92 | 0.10 | 0.29 | 0.04 | 0.16 | 0.18 | 5.13 |
| SR93 | 0.07 | 0.29 | 0.04 | 0.25 | 0.14 | 6.44 |
| SR94 | 0.08 | 0.20 | 0.03 | 0.16 | 0.16 | 6.87 |
| SR95 | 0.06 | 0.24 | 0.02 | 0.23 | 0.14 | 7.58 |
| SR96 | 0.07 | 0.29 | 0.03 | 0.24 | 0.19 | 5.59 |
| SR97 | 0.09 | 0.35 | 0.06 | 0.35 | 0.15 | 6.42 |
| SR98 | 0.06 | 0.40 | 0.03 | 0.36 | 0.14 | 9.31 |
| SR99 | 0.09 | 0.29 | 0.03 | 0.27 | 0.15 | 5.66 |

TABLE 23b
Pearson Correlation Coefficients, N = 207
Prob > |r| under H0: Rho = 0

| | C14:0 | C16:0 | C16:1 | C18:0 | C18:1 | C18:2 | C18:3 | C20:0 | C20:1 |
|---|---|---|---|---|---|---|---|---|---|
| C14:0 | 1 | 0.70162 | 0.54851 | 0.22355 | 0.0106 | −0.06955 | −0.08687 | 0.0519 | −0.44712 |
| | | <.0001 | <.0001 | 0.0012 | 0.8795 | 0.3194 | 0.2133 | 0.4577 | <.0001 |
| C16:0 | 0.70162 | 1 | 0.43505 | 0.09372 | −0.14566 | 0.07803 | −0.05032 | −0.01339 | −0.45138 |
| | <.0001 | | <.0001 | 0.1792 | 0.0362 | 0.2638 | 0.4715 | 0.8481 | <.0001 |
| C16:1 | 0.54851 | 0.43505 | 1 | 0.4634 | −0.00143 | −0.05856 | −0.14183 | 0.35005 | −0.60908 |
| | <.0001 | <.0001 | | <.0001 | 0.9837 | 0.402 | 0.0415 | <.0001 | <.0001 |
| C18:0 | 0.22355 | 0.09372 | 0.4634 | 1 | 0.01709 | −0.09611 | −0.3107 | 0.75625 | −0.38885 |
| | 0.0012 | 0.1792 | <.0001 | | 0.8069 | 0.1683 | <.0001 | <.0001 | <.0001 |
| C18:1 | 0.0106 | −0.14566 | −0.00143 | 0.01709 | 1 | −0.9676 | −0.28335 | 0.09383 | 0.06349 |
| | 0.8795 | 0.0362 | 0.9837 | 0.8069 | | <.0001 | <.0001 | 0.1787 | 0.3634 |
| C18:2 | −0.06955 | 0.07803 | −0.05856 | −0.09611 | −0.9676 | 1 | 0.10847 | −0.20029 | −0.07547 |
| | 0.3194 | 0.2638 | 0.402 | 0.1683 | <.0001 | | 0.1198 | 0.0038 | 0.2798 |
| C18:3 | −0.08687 | −0.05032 | −0.14183 | −0.3107 | −0.28335 | 0.10847 | 1 | −0.25085 | 0.12901 |
| | 0.2133 | 0.4715 | 0.0415 | <.0001 | <.0001 | 0.1198 | | 0.0003 | 0.0639 |
| C20:0 | 0.0519 | −0.01339 | 0.35005 | 0.75625 | 0.09383 | −0.20029 | −0.25085 | 1 | 0.0803 |
| | 0.4577 | 0.8481 | <.0001 | <.0001 | 0.1787 | 0.0038 | 0.0003 | | 0.2501 |
| C20:1 | −0.44712 | −0.45138 | −0.60908 | −0.38885 | 0.06349 | −0.07547 | 0.12901 | 0.0803 | 1 |
| | <.0001 | <.0001 | <.0001 | <.0001 | 0.3634 | 0.2798 | 0.0639 | 0.2501 | |
| C20:2 | −0.31159 | −0.29126 | −0.55856 | −0.46592 | −0.36455 | 0.39258 | 0.19101 | −0.32714 | 0.70965 |
| | <.0001 | <.0001 | <.0001 | <.0001 | <.0001 | <.0001 | 0.0058 | <.0001 | <.0001 |
| C22:0 | −0.10425 | −0.11896 | 0.15042 | 0.31775 | 0.05824 | −0.15925 | −0.0645 | 0.8436 | 0.42211 |
| | 0.1349 | 0.0878 | 0.0305 | <.0001 | 0.4045 | 0.0219 | 0.3558 | <.0001 | <.0001 |
| C22:1 | −0.09649 | −0.24488 | −0.29163 | −0.27587 | −0.01843 | 0.00616 | 0.09763 | 0.04089 | 0.57946 |
| | 0.1666 | 0.0004 | <.0001 | <.0001 | 0.7921 | 0.9298 | 0.1617 | 0.5586 | <.0001 |
| C24:0 | 0.07602 | −0.05931 | 0.10062 | 0.36898 | −0.00515 | −0.07664 | −0.16702 | 0.74112 | 0.40791 |
| | 0.2763 | 0.3959 | 0.1492 | <.0001 | 0.9413 | 0.2724 | 0.0162 | <.0001 | <.0001 |
| C24:1 | −0.10744 | −0.03897 | 0.16831 | 0.01226 | −0.17914 | 0.11097 | 0.13951 | 0.24178 | 0.22866 |
| | 0.1234 | 0.5772 | 0.0153 | 0.8609 | 0.0098 | 0.1114 | 0.045 | 0.0004 | 0.0009 |
| TOTAL | 0.50393 | 0.54341 | 0.58881 | 0.85308 | −0.04141 | −0.07387 | −0.27894 | 0.75466 | −0.39455 |
| SATS | <.0001 | <.0001 | <.0001 | <.0001 | 0.5535 | 0.2902 | <.0001 | <.0001 | <.0001 |

| | C20:2 | C22:0 | C22:1 | C24:0 | C24:1 | TOTAL SATS |
|---|---|---|---|---|---|---|
| C14:0 | −0.31159 | −0.10425 | −0.09649 | 0.07602 | −0.10744 | 0.50393 |
| | <.0001 | 0.1349 | 0.1666 | 0.2763 | 0.1234 | <.0001 |
| C16:0 | −0.29126 | −0.11896 | −0.24488 | −0.05931 | −0.03897 | 0.54341 |
| | <.0001 | 0.0878 | 0.0004 | 0.3959 | 0.5772 | <.0001 |
| C16:1 | −0.55856 | 0.15042 | −0.29163 | 0.10062 | 0.16831 | 0.58881 |
| | <.0001 | 0.0305 | <.0001 | 0.1492 | 0.0153 | <.0001 |
| C18:0 | −0.46592 | 0.31775 | −0.27587 | 0.36898 | 0.01226 | 0.85308 |
| | <.0001 | <.0001 | <.0001 | <.0001 | 0.8609 | <.0001 |
| C18:1 | −0.36455 | 0.05824 | −0.01843 | −0.00515 | −0.17914 | −0.04141 |
| | <.0001 | 0.4045 | 0.7921 | 0.9413 | 0.0098 | 0.5535 |
| C18:2 | 0.39258 | −0.15925 | 0.00616 | −0.07664 | 0.11097 | −0.07387 |
| | <.0001 | 0.0219 | 0.9298 | 0.2724 | 0.1114 | 0.2902 |
| C18:3 | 0.19101 | −0.0645 | 0.09763 | −0.16702 | 0.13951 | −0.27894 |
| | 0.0058 | 0.3558 | 0.1617 | 0.0162 | 0.045 | <.0001 |
| C20:0 | −0.32714 | 0.8436 | 0.04089 | 0.74112 | 0.24178 | 0.75466 |
| | <.0001 | <.0001 | 0.5586 | <.0001 | 0.0004 | <.0001 |
| C20:1 | 0.70965 | 0.42211 | 0.57946 | 0.40791 | 0.22866 | −0.39455 |
| | <.0001 | <.0001 | <.0001 | <.0001 | 0.0009 | <.0001 |
| C20:2 | 1 | −0.09292 | 0.52251 | 0.04744 | 0.12912 | −0.49687 |
| | | 0.183 | <.0001 | 0.4973 | 0.0637 | <.0001 |
| C22:0 | −0.09292 | 1 | 0.28934 | 0.78175 | 0.34771 | 0.41565 |
| | 0.183 | | <.0001 | <.0001 | <.0001 | <.0001 |
| C22:1 | 0.52251 | 0.28934 | 1 | 0.37021 | 0.19306 | −0.23744 |
| | <.0001 | <.0001 | | <.0001 | 0.0053 | 0.0006 |
| C24:0 | 0.04744 | 0.78175 | 0.37021 | 1 | 0.24026 | 0.46135 |
| | 0.4973 | <.0001 | <.0001 | | 0.0005 | <.0001 |
| C24:1 | 0.12912 | 0.34771 | 0.19306 | 0.24026 | 1 | 0.07225 |
| | 0.0637 | <.0001 | 0.0053 | 0.0005 | | 0.3009 |
| TOTAL | −0.49687 | 0.41565 | −0.23744 | 0.46135 | 0.07225 | 1 |
| SATS | <.0001 | <.0001 | 0.0006 | <.0001 | 0.3009 | |

14.4 Identification and Mapping of Loci Contributing to the Reduced C16:0 Content

QTL mapping found three loci to be associated with the reduced low saturated fatty acid phenotype in the Sockeye Red population. Two of those loci were previously unrecognized. One of the previously unidentified loci (QTL1) mapped to chromosome NO1 (Table 24, FIG. 6) and the other (QTL2) to chromosome N19 (Table 25, FIG. 7). QTL1 on chromosome N1 explained up to 35% of the variation in C16:0 while QTL2 on chromosome N19 explained up to 34% of the variation in C16:0. The third locus, which was previously mapped to the N3 chromosome, was found to map to chromosome N04 and explained 46% of the variation in C18:0 (Table 26, FIG. 8).

TABLE 24
SNPs associated with QTL1 contributing to the reduced C16:0 content phenotype inherited
from Salomon.

| | Position in | Position in | | | | | |
|---|---|---|---|---|---|---|---|
| | B. rapa | B. napus | Salomon | Surpass | | | |
| SNP | (Chiifu-401) | (DH12075) | allele | 400 allele | Flanking sequence | R-value | R2 |
| A01_20393111 | 20393111 | 20280290 | A | G | TTCGATTCATCACAATGGAGTACT | 0.53 | 0.28 |
| | | | | | ACCTGA[A/G]GAATGTCAGCGGG | | |
| | | | | | AAGTTGCCAGCCGTGTC | | |
| A01_20909816 | 20909816 | 20809964 | A | G | TTGCTGCAAACTGGACATGTTTTG | 0.58 | 0.34 |
| | | | | | GTTCCT[A/G]GTCATTGACACAA | | |
| | | | | | GTTTCTCAAGGTCAATG | | |
| A01_21103014 | 21103014 | na | A | C | CTTGATGAAGACTAAACAATTAGT | 0.59 | 0.35 |
| | | | | | ATTTGT[A/C]TCATCCTAGASAG | | |
| | | | | | TAGTAATCTGCARACTY | | |
| A01_21106413 | 21106413 | na | G | A | AATTAGCTGTAAAAGCTGTAAAAA | 0.59 | 0.35 |
| | | | | | CCTATG[A/G]AATAGGGAGATAA | | |
| | | | | | TATTTGATTATTTAAAC | | |
| A01_21219526 | 21219526 | 21094358 | G | A | GAGTGTTTTATCTATTTATGAGAT | 0.57 | 0.33 |
| | | | | | ATATAA[A/G]TTAAGAATCTATG | | |
| | | | | | AGATATATAAGTTAATA | | |
| A01_21393260 | 21393260 | 21381690 | G | A | TTCATTCATGACATTATCCACAAA | 0.57 | 0.32 |
| | | | | | GCTTTT[A/G]TTGATCATCAGCA | | |
| | | | | | ATGGAAGGAGGCCAAAT | | |
| A01_21519780 | 21519780 | 21514264 | G | A | GCATATCAAGGCGATCATCACGGG | 0.54 | 0.30 |
| | | | | | GCAAGA[A/G]GTTTTGATTCTGA | | |
| | | | | | ATTCGAAGGATCCTTCG | | |
| A01_21605865 | 21605865 | 21604285 | G | A | GTGGAAAATATGATAAAATAAGAA | 0.54 | 0.29 |
| | | | | | AAAAAA[A/G]TTTAGCACGTCTA | | |
| | | | | | AAGTCAAGTGAATGATA | | |
| A01_21651835 | 21651835 | 21636899 | G | A | TAATCCTASTAGTAGTAGTAACCA | 0.54 | 0.29 |
| | | | | | ATAGAA[A/G]ACCGTATAAATAT | | |
| | | | | | ATGAAACGAGGATTATA | | |
| A01_21776155 | 21776155 | 21764485 | C | A | GCACTGAAMTTGGCTTGCAACCCA | 0.52 | 0.27 |
| | | | | | GTAGGC[A/C]TGTGTCTTCAAGA | | |
| | | | | | ATATTTCGATTGTGTTG | | |
| A01_21855770 | 21855770 | 21832963 | G | A | ATGGTTWTAAAATATARKTGTCTG | 0.52 | 0.28 |
| | | | | | GTGTAT[A/G]TCAGTTACGTTTT | | |
| | | | | | AGCATTTAGACAAAACT | | |
| A01_22014179 | 22014179 | 22001815 | A | G | CAGTGAAGCAATATGCAAGAGAAG | 0.49 | 0.24 |
| | | | | | ATTCCG[A/G]AGAARCAGCAAAG | | |
| | | | | | GCGGTGGCAGACMGGAT | | |
| A01_22016353 | 22016353 | 22003987 | A | G | GCCTCCTATTCTTTATGTTCTTCA | 0.49 | 0.24 |
| | | | | | TTACTT[A/G]GGAATGAARCCGT | | |
| | | | | | GGCTATGTTACCGTGAC | | |
| A01_22022023 | 22022023 | 22014059 | A | G | AGCTTCTTTGACATCTTCTCCAGC | 0.49 | 0.24 |
| | | | | | TTTCTT[A/G]MACTCTTCTTGTT | | |
| | | | | | TWCTAGCTTCGTCAGTG | | |
| A01_22681496 | 22681496 | 22242804 | G | A | CAATTCTGAATAAATGAGTTTATA | 0.48 | 0.23 |
| | | | | | AGTATT[A/G]TGAGCTGATAAAT | | |
| | | | | | GTTTGATTAGCTGTTGC | | |
| A01_22768523 | 22768523 | 22311096 | A | G | CACCACAAGCCAATCTCTTCTTRA | 0.45 | 0.20 |
| | | | | | TGTGAG[A/G]AATGAGTGTGATC | | |
| | | | | | TCAAAGCTCATCAAGCG | | |
| A01_22777288 | 22777288 | 22321005 | A | G | GAGGAGATGCCAAGTGATTAGGTT | 0.45 | 0.20 |
| | | | | | CTGTTG[A/G]TATGCAAAGAGAG | | |
| | | | | | AAACGAGGGGGAAGTGA | | |
| A01_23095567 | 23095567 | 22597454 | G | A | TACGGAGAACTTGCAATTGGGAGA | 0.45 | 0.20 |
| | | | | | AGACAG[A/G]TTCAGAGAGATGT | | |
| | | | | | TAGGGCAAGAGATTGAG | | |
| A01_23097693 | 23097693 | 22599580 | A | C | TAGAATGAAATCTATAAAAGAATC | 0.44 | 0.20 |
| | | | | | ACTTAA[A/C]AATAGGCAAATAT | | |
| | | | | | TATTTTTTAYTGCTATC | | |

The columns of the table are:
SNP name,B. rapa location, B. napus location, flanking sequence, Pearson correlation coefficient and R² values between SNP markers on N1 and C16:0 content in the Sockeye Red DH population.

TABLE 25
SNPs associated with QTL2 allele(s) contributing to the reduced C16:0 content
phenotype inherited from Salomon.

| | Position in | Position in | | Surpass | | | |
|---|---|---|---|---|---|---|---|
| | B. oleracea | B. napus | Salomon | 400 | | | |
| SNP | (TO1000) | (DH12075) | allele | allele | Flanking sequence | R-value | R2 |
| 19436_1-p236134 | 13851710 | 14188467 | C | A | AAAAATCTTCATAAGCTC | 0.58 | 0.33 |
| | | | | | TTCGGGTAAAGC[A/C]A | | |
| | | | | | AACCATGGCCTCTGATTT | | |
| | | | | | TGACTGCTACT | | |
| 22835_1-p434032 | 14338666 | 14766331 | C | A | GAATTCCAAAAGTATATA | 0.58 | 0.34 |
| | | | | | ATATTTCTCAAC[A/C]G | | |
| | | | | | GTTCACAAACTCATTTAT | | |
| | | | | | CTATATGTAGA | | |
| 16547_1-p315514 | NA | 14934986 | A | G | ATTATCGCTATAACTTAA | 0.58 | 0.34 |
| | | | | | CATGGTATCAGA[A/G]C | | |
| | | | | | CTTTTTAGACCATCAGTT | | |
| | | | | | CCTAGGATCAT | | |
| 20836_1-p261578 | 15987481 | na | A | G | ACTCCCTCCATCTTAAGC | 0.55 | 0.30 |
| | | | | | TTTCCATTCTAT[A/G]C | | |
| | | | | | AGAACACTCAAAACGAAC | | |
| | | | | | AAATACAAACT | | |
| 15847_1-p265134 | 16796332 | 17339410 | A | G | GGGTTACTAACCATTTCT | 0.53 | 0.28 |
| | | | | | GCGTTCACAAAG[A/G]T | | |
| | | | | | CACCAGCTTCAACGCGTT | | |
| | | | | | TACGCCCATTA | | |
| 15847_1-p264646 | 16796820 | 17339897 | A | G | AAACACAACCCCGGAAGG | 0.54 | 0.29 |
| | | | | | ATTGCCTTCCCA[A/G]A | | |
| | | | | | AGATGMGGTCCTTGCTTG | | |
| | | | | | AAAAAGTGGAT | | |
| 18100_1-p194352 | 16993809 | 17588687 | G | A | TTTCCATTGCACAGCAGT | 0.53 | 0.28 |
| | | | | | AATAAACCATAA[A/G]G | | |
| | | | | | GTACGTACCCTTATCATG | | |
| | | | | | TTTTCCTTGAG | | |
| 18100_1-p271298 | 17070755 | na | G | A | CTTCTTCGAGCAAAATCA | 0.53 | 0.29 |
| | | | | | AAGAAGATCCCA[A/G]A | | |
| | | | | | AACTAGSTGCAAAACAAT | | |
| | | | | | TGATTGGGAAC | | |
| 18100_1-p544438 | 17343895 | 17940483 | A | C | ATTGTTTTGCATCTATTG | 0.54 | 0.29 |
| | | | | | TTGAATATAAAA[A/C]C | | |
| | | | | | CTCAAAACYCACTTTCTC | | |
| | | | | | TGCAAGGTTAA | | |
| 18100_1-p568863 | 17368320 | 17949506 | C | A | AAAAAAACAAACAAACCA | 0.53 | 0.28 |
| | | | | | AACTAGTCAAAC[A/C]A | | |
| | | | | | GACTACCAAAATTGTATG | | |
| | | | | | CCCTATGCAGC | | |
| 18100_1-p750941 | 17550398 | 18167872 | A | C | AAATAAAAATATTWAGCG | 0.53 | 0.28 |
| | | | | | ATATAWTAGART[A/C]T | | |
| | | | | | AAAGCTAATATAAGGGTA | | |
| | | | | | TAAATTTGATA | | |

The columns of the table are:
SNP name, B. oleracea location, B. napus location, flanking sequence, Pearson correlation coefficient and R² values between SNP markers on chromosome N19 and C16:0 content in the Sockeye Red DH population.

TABLE 26
SNPs flanking the FATA2 allele(s) contributing to the reduced C18:0 content
phenotype inherited from Salomon.

| | Position | Position in | | | | | |
|---|---|---|---|---|---|---|---|
| | in B. rapa | B. napus | Salomon | Surpass | | | |
| SNP | (Chiifu-401) | (DH12075) | allele | 400 allele | Flanking sequences | R-value | R² |
| A04_3263085 | 3263085 | 3170762 | A | C | GTTCGAGATTTGTGTAAGTTGAGGA | 0.65 | 0.42 |
| | | | | | AATGG[A/C]GGCTACAGATGCATC | | |
| | | | | | AGTRATATCTAYCCC | | |
| A04_3589494 | 3589494 | 3505048 | A | G | CAGATACATCAAATTYATAAACCTA | 0.64 | 0.41 |
| | | | | | TCCCT[A/G]AACCCCTAGCTAAGA | | |
| | | | | | ACTCGAATAGTTCAG | | |
| A04_3750975 | 3750975 | 3653112 | A | G | TTGAGTAACGAATGCGCCACCCATG | 0.65 | 0.42 |
| | | | | | CTGTA[A/G]ATATAGATGCTACGT | | |
| | | | | | AGGATCATTGTAAGA | | |
| A04_4891169 | 4891169 | 5310615 | G | A | GTATTYTTAGGGTTAGATTTTGGAA | 0.67 | 0.45 |
| | | | | | ARCTT[A/G]GTTTGCAATCACTGG | | |
| | | | | | TTGTTGGTTCCATGA | | |
| A04_5270881 | 5270881 | 5713624 | A | G | GATAAAGAAGGTGRTGATGATGGGA | 0.68 | 0.46 |
| | | | | | GCAGC[A/G]ATGAAAGTGGTAGCA | | |
| | | | | | TTCGGTCAGAAAGTA | | |
| FatA2 | 5529590 | 6082512 | T | C | CAGCTCAGTATTCTATGCTAGAGCT | | |
| | | | | | TAAGC[T/C]TCGGCGAGCTGATCT | | |
| | | | | | GGACATGAACCAGCA | | |
| A04_2411039 | 2411039 | 6128312 | G | A | TAACCGAAAATCTGAAACAAAAACA | 0.68 | 0.46 |
| | | | | | TATTC[A/G]CAATATCCAAAAAAT | | |
| | | | | | ACCTAATGACAAAAT | | |
| A04_5873190 | 5873190 | na | A | C | AATTTGGACGGAACCGAAACCGAAA | 0.67 | 0.44 |
| | | | | | ACCAA[A/C]CCAGTTCGYAAAACC | | |
| | | | | | GAACTTAGATTAGTA | | |
| A04_6050616 | 6050616 | 7707855 | G | A | AAGCTTGCTAGGAAAAAACATCGAT | 0.69 | 0.47 |
| | | | | | CGATT[A/G]TCGTATAGATAAAYC | | |
| | | | | | GATTGACTTTCTGAA | | |
| A04_6264043 | 6264043 | 7932174 | A | G | TGGCTTTATTCATTTGTTTTGTTCG | 0.68 | 0.47 |
| | | | | | AGGAC[A/G]AACAAAGGTTCAAGC | | |
| | | | | | CGGCGAAAGTTAATA | | |
| A04_6646210 | 6646210 | 8366049 | C | A | AAAGCATCTGAAATTACAAACACTG | 0.67 | 0.45 |
| | | | | | ACCAG[A/C]AAATTATTTTTCAGG | | |
| | | | | | CAGTTCTGCTAGAAA | | |
| A04_3229572 | 3229572 | 8456270 | A | G | TCCTCCTCCTCCTCCTTCGCTGGGG | 0.67 | 0.45 |
| | | | | | GAAAC[A/G]CCGGTTATCGATCTA | | |
| | | | | | CAAACARTTTCCGGC | | |
| A04_6749114 | 6749114 | na | C | A | GTAAATATTGTCATCAGCAGAAATT | 0.68 | 0.46 |
| | | | | | TTTCA[A/C]GTTTTAGGATTCTTG | | |
| | | | | | TTTTTTTGTTTGTGT | | |
| A04_6980459 | 6980459 | 8736901 | G | A | YACCTATTGRTTASWTAAAAGAAGC | 0.68 | 0.46 |
| | | | | | AATGC[A/G]TGAACATCTTCAATT | | |
| | | | | | TTTGTGTAGACAAAA | | |
| A04_7471870 | 7471870 | 9311436 | A | G | GAAGAATTGTAAACTTYTGWGAGAA | 0.65 | 0.43 |
| | | | | | ATTAG[A/G]TTAGTTATCGTCAAT | | |
| | | | | | ATTGAAACTTGTCCA | | |
| A04_7588820 | 7588820 | 9456872 | A | G | TGTGTAACCAACAACCCAGAGTATC | 0.65 | 0.43 |
| | | | | | TAAGT[A/G]TTTATACCTGAACAA | | |
| | | | | | TATACCAGACTGAAA | | |
| A04_7883120 | 7883120 | 9775987 | G | A | GAGAAGGCCACTTTTGTTATCCTTT | 0.65 | 0.42 |
| | | | | | AGGGT[A/G]TCAACCCACGTGGGT | | |
| | | | | | CGAAAAGTCATAAAT | | |
| A04_8116942 | 8116942 | 9985687 | C | A | GTSTACTACKCCTTCCTTTAAGAAG | 0.65 | 0.42 |
| | | | | | AGTGG[A/C]AGTAAGAAAAACAGC | | |
| | | | | | AGAAGTAGACAGGGT | | |

The columns of the table are:
SNP name, B. rapa location, B. napus location, flanking sequence, Pearson correlation coefficient and R² values between SNP markers on chromosome N4 and C18:0 content in the Sockeye Red DH population.

A detailed genotype of the Salomon QTLs on N1 and N19 was created using the sequence alignment data. Single-nucleotide polymorphism (SNP) selections were made across the genomic intervals identified through marker and phenotype analysis of the NILs. Each selected SNP were required to be unique to Salomon, i.e., having a different genotype to both Surpass 400 and IMC201, which serves as evidence that the mutations were created through the mutagenesis process. Tables 27 and 28 list the selected SNPs for the N1 and N19 QTLs, respectively. Included is the position of the SNP (according to the sequence of the reference linkage group), the reference genotype, the Salomon genotype and flanking sequence 30 bp upstream and downstream from the SNP site.

TABLE 27
B. napus position relative to the DH12075 reference genome, wild-type allele, Salomon
allele, flanking sequence and sequence ID number of SNPs identified in QTL1 on N1.

| Position in | | | | |
|---|---|---|---|---|
| DH12075 | Wild-type | Salomon | | Sequence ID |
| (Base Pairs) | allele | allele | Flanking Sequence | Number |
| 20772548 | C | T | ACATATAGCCAATGGCTCCAACTCTCCTCT[C/T]CCTATATCA | 53 |
| | | | CCATTAGTAGACTCACAATCT | |
| 20780679 | C | T | ATGGTCATGGTTCTCTCATTTGGTAAACAT[C/T]TTGTTCCTC | 54 |
| | | | ATAAATCATAATGATTCCCTC | |
| 20843387 | C | T | AGCGTCATTGGAGTTATGGAATACAGAAAC[C/T]CACTTGTCG | 55 |
| | | | TAATCCTGATTCACTAGAAGC | |
| 20874199 | C | T | CGGGATCTGCATCCACCGCCGCTAGAGATT[C/T]CGGTTCTGA | 56 |
| | | | TGCTCCACCAAGGGTTGGTTT | |
| 20874571 | C | T | GAGACCACAAGGGGTTGGAATCAAGATGGG[C/T]TGTTATGGA | 57 |
| | | | GCAAGCCCGTGTCGAGTACTT | |
| 20924967 | C | T | AAACCGGATTTATTAAAGACACAAACCTAA[C/T]CTCCAGATG | 58 |
| | | | AGAGGTGCAATACACATATGG | |
| 20979545 | C | T | TGGCTGAACGAAAACACAATGCACCAATGT[C/T]GATCATTCA | 59 |
| | | | CTACAGAAGATAATTGATATC | |
| 21000713 | C | T | GCCACACCTTTGCTTCACTTGGGTCCGCCC[C/T]TGTCTATAA | 60 |
| | | | CTGAATCCCCAACATGAGACA | |
| 21057761 | C | T | ACCAACAAATCAGAACTAATATTGAATTGT[C/T]CAAAGGTAG | 61 |
| | | | AAGTCATCAATCAAGATATGA | |
| 21080816 | C | T | TAGTCTATGAACCACTCAAACCCTTAACCC[C/T]TAGTGGCTT | 62 |
| | | | TGCTTTTCCTTATTCGGACTT | |
| 21126589 | C | T | AAATGAAGGGTGAAAATGTAATAAATAAAT[C/T]TCTTATATA | 63 |
| | | | CTAAAGCACAAGTCACTCTAC | |
| 21175577 | C | T | CAATCTTGAAAACCCTTTGTCTACTTGCGC[C/T]ACAAGGAAT | 64 |
| | | | ACAATGCTTTGCTTTTGTTTT | |
| 21244175 | A | G | CAACCACACTCTATTTTTCACTCTAAAATA[A/G]AGTTTAGAG | 65 |
| | | | TAAAAATGCTCCAATAAGACT | |
| 21273898 | G | A | AACCAATATGAGAGAACCAACTCCTAAAAA[G/A]AATCCTCGA | 66 |
| | | | AAACTAACGAGCGGGCTGATT | |
| 21301953 | C | T | ATACAGTAAACATATGCGTTCACATGGCCA[C/T]TGCCAAGTT | 67 |
| | | | AATGAAGGAATTACGTACTCT | |
| 21342623 | C | T | AACATTTCCAAAATGCGGTTAAAATGTTTA[C/T]CACCAAATA | 68 |
| | | | TTTAGTATATTTTAACATGAA | |
| 21378815 | C | T | GATGAGATACAACGCTTCCGTGATCGAGCT[C/T]GGGAGCGAA | 69 |
| | | | GACACCGCGCTGATGCGGTGT | |
| 21425310 | G | A | CGTCGGAGACAATATCAACGCCTGAACTCT[G/A]CAAATCAAA | 70 |
| | | | ACCATAACATAAGAAACAATC | |
| 21491979 | C | A | GGGCAACCCAACACTTATTTCCAATGTTAT[C/A]TTTCTTCAT | 71 |
| | | | TTTCAATACCCTGCCCCACTT | |
| 21549878 | G | A | TAACTCCATTGAAAGTGCCACTAGCCCAGT[G/A]CTAAAATAC | 72 |
| | | | CTTGCCATTAGTCCTAATCAA | |
| 21597845 | C | T | AAATGCAATAGTTAAATTGACTTTTCTGAC[C/T]GATGATAAT | 73 |
| | | | TAAATGTGAAAAAAACACTGT | |
| 21621627 | C | T | CAAGTACATCGCTCGAAACTCCTCGTTGGT[C/T[AGATCAGCG | 74 |
| | | | AACCGGGTCAACCCGAGTTCG | |
| 21648874 | C | T | TATAGCATTGCTAAATTTAAATTCTATTTT[C/T]CGGTAAGAG | 75 |
| | | | ATTCTTTGTTTCACCGGGAGA | |
| 21700869 | C | T | AACATACATTCTCTTAATGATTGATTGTTC[C/T]CTATAGTAT | 76 |
| | | | ATGGTTAGAAGTGTTGATATG | |
| 21740913 | G | C | AAGCCCTGCCTTCTATGTTACCAAAGCCTG[G/C]TTATCAGTT | 77 |
| | | | TGACTAACTGGGATGGTACAT | |
| 21793927 | C | T | CGTTTCAATCAGACAAAGTTGCATTTTTTT[C/T]TTCATGAGT | 78 |
| | | | AGTTTACACTTTGCACGCCGT | |
| 21825553 | C | T | TTTGATTTTCTTAAAGAACTTGAGACAATC[C/T]TTAGATAAA | 79 |
| | | | ACTTTCTTCAAACCTCATCAC | |
| 21856527 | G | A | TGATTGACAGTGGAAGGCATTATGAAGGAC[G/A]TACGTTCGT | 80 |
| | | | CTACGTTGATGCACCAAGTCA | |
| 21899956 | G | A | AGGGAAGGAGTAAAAACAGGCAAATCTATA[G/A]TATAATGTT | 81 |
| | | | ATTGACTAACTTATTATTACA | |
| 21938801 | T | C | AAGACCTGACTATACAATCTTTGGTTTTTA[T/C]CTAATGCAC | 82 |
| | | | AACTAGCACAAGCATTCATGT | |
| 21980398 | C | T | CCGCCGCTTCTCCGCCTCCGGATCCGACGA[C/T]GGACGAAGT | 83 |
| | | | GAATGAGTCGTTGCGGAGACT | |
| 22001149 | C | T | AAAATTATTCTTTCAATGTATCTTATTTTG[C/T]TTAATCATT | 84 |
| | | | ATTATTTTGAAAATATGTTAT | |
| 22060515 | G | A | TGTTTGTTATGTAACTGCAGAAAACATCAT[G/A]ACAATCGTA | 85 |
| | | | TTCAAATTGTAAGCAAAGGAA | |
| 22100267 | G | A | TCTTGTATTTGAAGTTGGAGATTTCGTTTA[G/A]GCATATCTT | 86 |
| | | | ACACAGGATAGGATGCCAGCT | |
| 22144311 | T | C | CACTTCTCTGTATTTTTCTTCTTTTCTGTG[T/C]AGTTTGGCT | 87 |
| | | | CCTATCATTAATGAAAACTCT | |
| 22180149 | C | T | ACCATTGAAAATTCAAATGAAAATTCAAAT[C/T]GTGTTATAG | 88 |
| | | | AGGGAGAGAGAGAGAGAGAGG | |
| 22217506 | C | T | TATTAAACTTATAAAAACTATTAAAACCAT[C/T]AAAAATCTA | 89 |
| | | | TAAACTATCTATATAAACATA | |
| 22258914 | G | A | AAAACTAGATAAATATATTTTTAAAGTTAT[G/A]TGTTGTAAC | 90 |
| | | | AAAGTTATTTATTGACCCAAA | |
| 22260507 | G | A | ATAAAAAATTATGTTTGAAACTATTTTTCA[G/A]TTTTTTAAT | 91 |
| | | | ATATTTTTTAAGTATTTATTT | |
| 22299725 | C | T | AGAGGAAACATAAACAAGAAACCAAATCCA[C/T]AATATAGCA | 92 |
| | | | TTTCTACTATTTTCAAACTCA | |
| 22347689 | G | A | TGAGTGTCATTTCTTAGGTGTCATTTTCAC[G/A]AGCTCTCTC | 93 |
| | | | ACAACAAAATTTTAAAAATCC | |
| 22379370 | C | T | AACGTCGACGGCTCTTGTTCTCTCGTCTCC[C/T]TTTCTCCGG | 94 |
| | | | AAGAGAACCATCAAAACAAGA | |
| 22420077 | G | A | TCACGGGCCTTACTGAGTCGTATCAACTCT[G/A]TTTGGACTC | 95 |
| | | | AACAAAGAAACAAAAGCTTGA | |
| 22456310 | G | A | AGTCTGAATAACAGTATTCTCCTGGCGAGT[G/A]AACGGCTGT | 96 |
| | | | TTCAAGTATCCAGACCTATGA | |
| 22498876 | C | T | TAAAACGTGAGAGCTCATAGCAAAAAATCA[C/T]TTTGCAAAT | 97 |
| | | | AATTGTATAATAAATATTTTT | |
| 22543194 | T | C | TTTGACTTATACAAATATTTTGCATGCTAG[T/C]CGCTATTTA | 98 |
| | | | ATTTTTGTTTACCGGATATTT | |
| 22580394 | C | T | TGATCTTAGCGACGACGATTAGTGTTTACT[C/T]TCTTTAATG | 99 |
| | | | CCTAATAAAGCGTCCCTAACA | |
| 22621466 | G | A | CATTCGACCCATCTCGAAGCCCATTCCCGA[G/A]CCACTCTCT | 100 |
| | | | CGTAAGCATAATCCCGTGTTC | |
| 22659331 | G | A | AGTTTGGGTTTTTGGATAAAAATCTTATAT[G/A]TTAAAACAT | 101 |
| | | | AAGTCATGACTTCTTTCATGT | |
| 22702378 | C | T | AATTTTGATAATGTTTTAATTTTCCAATTG[C/T]CCCCAAAAA | 102 |
| | | | CATTCCAGTTATATAGTTTGT | |
| 22739470 | G | A | CGCGCGGGACAAGCCGGCTGTGACCCGCTC[G/A]ATGACTAAC | 103 |
| | | | CCGCCGTGGTACGGGATGGAA | |
| 22780181 | C | T | AGATGCATATTATCGTACAAGAACAATAAA[C/T]TTCCCGCCA | 104 |
| | | | TTTTTGAGAAAAATGGCATGT | |

TABLE 28
B. napus position relative to the DH12075 reference genome, wild-type allele, Salomon
allele, flanking sequence and sequence ID number of SNPs identified in QTL2 on N19.

| Position in | | | | |
|---|---|---|---|---|
| DH12075 | Wild-type | Salomon | | Sequence |
| (base pairs) | allele | allele | Flanking Sequence | ID |
| 11538807 | C | T | CACCACGTTAAAATAGTTTGTTGCAAAAAA[C/T]CACTTGT | 105 |
| | | | AACAGTTGCAAAAAACCACTTGT | |
| 11763228 | C | T | GTCTCGAGATAGTCGACGCCTTCAGCTTGT[C/T]TGTTGCC | 106 |
| | | | TCTGGAAACAACTCTAGCTCTAT | |
| 11855685 | C | T | GCCAGTATATAAAGATTCCTAGGCGAGAAG[C/T]ATGGGGA | 107 |
| | | | GGACTTTTCTCAGAGCAAACTTA | |
| 12010676 | C | T | ACGTCTTCTCCGACCATAACATTGTACCTG[C/T]CAAAATA | 108 |
| | | | GACAGTTTGTAGATTAACTGTTT | |
| 12205222 | C | T | GCCGGGTTGGTACACCATCACCGTCACCCG[C/T]CGTCCAC | 109 |
| | | | CTCTGTCTCATCCTCGTAACCAA | |
| 12219881 | C | T | GGATGGGACAGATGAGAGGTTGAAATCGCG[C/T]CTGGTTG | 110 |
| | | | TC TATGGAAATAAACAAGTCGAG | |
| 12355162 | C | T | GAAACCCTGTAACCAATCGGCTCTGTTTCC[C/T]CGATAAT | |
| | | | CTATCGTCTGTGTAACTCACCGG | 111 |
| 12378335 | C | T | TTTTTTTTTTGGCAACTATTTTTATTTTCT[C/T]AATTTCT | 112 |
| | | | GTTTCCATAAATAAAATATGACG | |
| 12507143 | C | T | ATGCGACCTTGTTAGGGAACTTGTCGATGG[C/T]GTATTGG | 113 |
| | | | GATCAGGATGATCCGTACGAGAT | |
| 12615691 | C | T | TTATGTATCTATAAAATGACCAAGACTAAT[C/T]TTAAAAA | 114 |
| | | | TGAAGTGAAAGCTACATATAATT | |
| 12847514 | C | T | TATATGAATCTAAATTACCTACGACCGTCT[C/T]CATCGCT | 115 |
| | | | GTGCACATCAAAACTATATAACC | |
| 12979251 | C | T | TCAAACCACAGGAGTCAGCCAATATAGCAG[C/T]AGAGTCT | 116 |
| | | | ACAGAGCCTCTAATCCTAACCGT | |
| 13003942 | C | T | AAGAAAGGAGATCGTCGTAGGAACGCTGAG[C/T]GTAACAC | 117 |
| | | | ACAAAGAAAGCGTGTGTAACGCA | |
| 13008581 | G | A | CATTTCTAGAGATTCAAAATTATATTTTGG[G/A]TTTTATT | 118 |
| | | | GAGATGATTTAGGAGTTTGATGC | |
| 13207412 | C | T | ACTTCTTCTACAGCGAAGTCGCTCGCATCG[C/T]ACATGAT | 119 |
| | | | TTCGAAAGGAAGAGTCCAGTCTG | |
| 13364132 | C | T | ACCCGAAAGAATTTGAATAAGAAGCTGGTT[C/T]TGTATTG | 120 |
| | | | GGTTTGTATGTTTTGCGATATAC | |
| 13429175 | C | T | CGAGCAAACTGATCCATGACTTTTAGGACC[C/T]CACGCAA | 121 |
| | | | ATATCTACTGGTTCTGTCTGTAA | |
| 13429687 | C | T | TTCAATTTCTTCCGGAAGAATCATCTTCAG[C/T]CTTCGCA | 122 |
| | | | CTAATATCTTGGAGATTACCTTA | |
| 13460532 | C | T | GGAAGGTGGAAAAGGTTTTCTCAAGCATAT[C/T]CTTATCC | 123 |
| | | | GTTATATCCTCACCACACAGTTT | |
| 13475876 | C | | AATCAAAAGTCTACCTTCTTAGCTAAGAAA[C/T]TAGATAT | 124 |
| | T | | CACGTGTGATAGCAAAAACAAAA | |
| 13504886 | C | T | GTTAGTATTCCTTACGTCCCAATGCTTACT[C/T]CAACTTG | 125 |
| | | | CATTTCTCTTGTACTTAAGATCC | |
| 13704881 | C | T | TCTCTTTGTTCTTATCCAGGTTCCACTGCT[C/T]TTGCATT | 126 |
| | | | TCAACCAATTCTTGGAAGGTGGA | |
| 13925427 | C | T | TCCAGCATCCTGCAAGAACAACGTAGAGAC[C/T]TCCACAC | 127 |
| | | | CACAGTGGACTAACCTATCACAT | |
| 14046125 | A | G | AACCATATAGATTGTGTTATAAGTCTTTCT[A/G[AATGGTT | 128 |
| | | | ATAAACTCTTATAAATGATTAAA | |
| 14135213 | T | A | TAATAATCTAGATGCTCAAAATTACAATTA[T/A]AAATCTA | 129 |
| | | | AATTTGTTTAGTTATTTTCTGTA | |
| 14377562 | G | A | AATTTCGAGAAAATCTTCACGGACCAGAAA[G/A]TTATGGA | 130 |
| | | | TTTTACAAACTGGAGCTTCTCCA | |
| 14776751 | C | T | TTCTCATTAAACAAAGAAAAATGGCAATCT[C/T]TTTTCTG | 131 |
| | | | TGTCTCTTTCTCATCACCTTTGC | |
| 14801661 | C | T | GGCTGAACCAGAACATTTATCTACTGAAGG[C/T]AGAGCAT | 132 |
| | | | ATTTTTGAAAATATAGTTTATAA | |
| 15173478 | C | T | TTGAGCATGAGAGATAACTGGCTGGAGTGC[C/T]TCTTTGA | 133 |
| | | | GCCTGCCCGTAAGAAGCTGGGAG | |
| 15235513 | C | T | TTTTATTGAAGTGCATTTATCCAAAATTTC[C/T]CCCTAAA | 134 |
| | | | ATGTATTCCCTTAGTTTCACAAA | |
| 15387929 | C | T | TCCATTCCCAAGACTAAGGAGCTCATTCAT[C/T]ACATTAG | 135 |
| | | | ATTGTGTCCTATCAGCTATATCA | |
| 15399385 | C | A | GTTGGCAGCGAGGCGCGGTCTCACGCTCTA[C/A]TATCTCC | 136 |
| | | | TTGCGAAAGGGCTCCAGCTCGTC | |
| 15547466 | C | T | TTTGTAAAATAAATCATGTTTTTCATGAAT[C/T]TTTTTTA | 137 |
| | | | AAGAGAATATGTATTTAATCAAT | |
| 15623646 | C | T | CCAGATTTCCCAATTCCAAGTTTGTCTTTT[C/T]ATGTAAA | 138 |
| | | | TTCTTCGGCAAATACAGGTATGT | |
| 15629066 | C | T | AATGAATCTTCCTGCCGCTCCCTCTGTGAT[C/T]CAGTAGA | 139 |
| | | | ACACTCGTCACAACCTCAAAATA | |
| 15684032 | C | T | TCTTACTATTACTAAACCTTGTCCCCAAAA[C/T]CCCACCC | 140 |
| | | | TTCAACTCTAAACCTTAAGTCTA | |
| 15741164 | C | T | AGTCACCAAGCTCGGTCGTCTCGTTCAGAG[C/T]GGTAAAA | 141 |
| | | | TCACGCAGCTAGAGCATATCTAT | |
| 15768411 | C | T | ATACAGAGGCGATGAATGCGAAAGTGGATA[C/T]AGAGGTG | 142 |
| | | | GAGACTGTGGTGACGATAGATAC | |
| 15898184 | C | T | CACCTCCGCCGTGTGTCGATACCATGAACA[C/T]TCAACCT | 143 |
| | | | CCGCCTGTCTTCACCTCCACTAC | |
| 15943625 | C | T | AGACATCCATGACGATTCCTCGAAGGCAAA[C/T]TCACACA | 144 |
| | | | CGCTTCTGCTAGCTGTTGTAGCG | |
| 15988083 | C | T | CCTTTCCAACACTCCATCAGAAGTACTCCT[C/T]CAACTTA | 145 |
| | | | ATCTTGTACATACCAGTTTATCT | |
| 16211916 | C | T | CTTCCTTTCAACACTACTCGTCGTTTCTGT[C/T]TCCTTTG | 146 |
| | | | AGATTGACTTTAGATCATCTTCT | |
| 16238183 | C | G | ATTGAGATATAAATATTATAATAATATATA[C/T]CTTAAAT | 147 |
| | | | AGCGAGCTCAATAAATTTTATTT | |
| 16293509 | C | T | GTTCGTCACACCCAGCAATGAGCAAGAAAC[C/T]AAGGATA | 148 |
| | | | CTGCGAAAATCCAAGGCCGGAGT | |
| 16468313 | C | T | TGCCAACCTCAAATCTCAACTTTAATAATC[C/T]TTTTATA | 149 |
| | | | TCTCTTTACAAATATCCACCCAA | |
| 16698792 | C | T | ACAACACATTAACAAAAAAAATGTCATTCG[C/T]TTCACTC | 150 |
| | | | TTGTATGCATTCTTCTTGATCTT | |
| 16765722 | C | T | AAAGAATCAAACTGTAGGAATTTATAATTG[C/T]CCTTTGC | 151 |
| | | | AAGTTTTTTTTTTGTAACTGAGC | |
| 16787306 | C | T | TGATATACCGAAAAATCAAACAAGCAGCGT[C/T]CATTGTT | |
| | | | GCAGAACAAGTAGCGTACATATT | 152 |
| 17041989 | C | T | TTATCTTATTAGAACTGATTTTAGTTTCTT[C/T]TTTCATT | |
| | | | CTAGGATTTAATTAATGACATAA | 153 |
| 17052864 | C | T | AGTTCTGCTTCACCAATACCTCCATAAGCT[C/T]CATCCAC | |
| | | | TCAGGCCACGGATGCACCAACTC | 154 |
| 17111885 | C | | AGTTAAAAAAAAATCAATCTTGTTTCATTT[C/T]TATTAAT | |
| | | T | TGTTGAGACGCCAATAATTTTAT | 155 |
| 17219357 | C | | TGTTCCAATATATAAGATGTTCTCATCTTT[C/T]TATGTAA | 156 |
| | | T | TTTTAAGTTTATCAAAAACTGTG | |
| 17443797 | C | T | GAGTCGCTCGCACAGATCTTTGTTTTTATC[C/T]TGAGTTC | 157 |
| | | | CTCTTTGCTCGGAGTTTCTCTCA | |
| 17636667 | C | T | CCAAAACTGAAAAGGAAAGAATGATCTACG[C/T]TGCATCA | 158 |
| | | | GAAGACGACTCCATGGCCGGAGA | |
| 17893475 | C | T | TTAACATAAAGAAATTATTACAATGATAAA[C/T]ATTATAC | 159 |
| | | | ATAGATTTTTTAGACGACTAACT | |
| 17924151 | C | T | CTCTAAATGTAGAGTGCTTGGCGACATATC[C/T]AACGGAG | 160 |
| | | | GCTCTTCTCTCGAAATCATCAAA | |
| 18164787 | C | T | AAAATGTAATCTTTCCCACTCTAAAACTCT[C/T]CAACCTC | 161 |
| | | | TCTCTAATCTCTTTGAACATCAA | |
| 18172630 | C | T | TTCATGTGCTAAGCAGTTATATATTATTAT[C/T]ATATATT | 162 |
| | | | ATTATTACAATAATAAGATGATA | |

14.5 Fine Mapping within the N1 QTL and N19 QTL

In order to more narrowly define the causal genomic interval, fine-mapping was performed using NILs heterozygous at molecular marker loci shown in FIGS. 6 and 7 (spanning the N1 and N19 QTLs) were self-pollinated. The resulting progeny were grown to prepare the plants of generation 1, which were genotyped with KASP SNP genotyping assays (LCG Limited, Teddington, Middlesex, UK, see also www.kbioscience.co.uk) developed using the sequence information in Tables 27 and 28 to identify recombination events occurring within the QTL region. Plants in which such events occurred were identified were grown to maturity and their seed fatty acid profiles were determined via GLC. Pearson correlation coefficients between marker loci and saturated fatty acids were estimated so that the candidate QTL region narrowed as a function of decreasing correlation around a peak. In generation 1, a total of 1,147 plants were genotyped at loci within the N1 QTL from which 145 individuals were selected for seed production and fatty acid analysis. In addition, a total of 1,000 plants were genotyped at loci within the N19 QTL from which 55 were selected for seed production and fatty acid analysis. The results of these analyses guided re-planting of populations from selected individuals of generation 1 (i.e. those carrying rare recombination events) to form the plants of generation 2. From generation 2, a total of 1,024 plants were genotyped at loci within the N1 QTL, from which 65 individuals were selected for seed production and fatty acid analysis. In addition, a total of 928 plants were genotyped at loci within the N19 QTL, from which 79 were selected for seed production and fatty acid analysis. The results of those two generations of fine-mapping are shown in Tables 29-32, which are each divided into two subparts, A and B. Part A of the tables gives the Pearson correlation coefficients between marker loci and saturated fatty acids. The location number column gives the physical location of the loci in the DH12075 reference genome. Part B of the tables provides a comparison of the mean fatty acid values of seeds of plants carrying Salomon and 03LC.034 alleles.

The results provided in Table 29, Part A provide the Pearson correlation coefficients between saturated fatty acids and marker loci for QTL1 on N1 (n=145). Part B of Table 29 provides a comparison of the mean fatty acid values of plants carrying Salomon and 03LC.034 alleles spanning positions 20924967-22780181 of the DH12075 reference genome.

TABLE 29
Table 29A

| | | Total |
|---|---|---|
| Location | C16:0 | Saturates |
| 20924967 | −0.744 | −0.605 |
| 20979545 | −0.812 | −0.692 |
| 21126589 | −0.799 | −0.671 |
| 21342623 | −0.768 | −0.653 |
| 21491979 | −0.696 | −0.620 |
| 21740913 | −0.625 | −0.560 |
| 21793927 | −0.624 | −0.550 |
| 21825553 | −0.622 | −0.548 |
| 21980398 | −0.570 | −0.530 |
| 22060515 | −0.510 | −0.485 |
| 22100267 | −0.515 | −0.476 |
| 22144311 | −0.500 | −0.474 |
| 22180149 | −0.477 | −0.425 |
| 22299725 | −0.408 | −0.401 |
| 22347689 | −0.372 | −0.366 |
| 22420077 | −0.340 | −0.357 |
| 22543194 | −0.284 | −0.350 |
| 22659331 | −0.190 | −0.274 |
| 22780181 | −0.205 | −0.282 |

Table 29B

| | Mean C16:0 | Mean Total |
|---|---|---|
| Salomon allele | 4.17 | 8.49 |
| 03LC.034 allele | 5.09 | 9.42 |

A further refinement of the results provided in Table 29 is set forth in Table 30. The results in Table 30 Part A provide the Pearson correlation coefficients between saturated fatty acids and marker loci on smaller portion of N1 (n=65). Part B of Table 30 provides a comparison of the mean fatty acid values of plants carrying Salomon and 03LC.034 alleles spanning positions 20772548-21342623 of the DH12075 reference genome.

TABLE 30
Table 30A

| | | Total |
|---|---|---|
| Location | C16:0 | Saturates |
| 20772548 | 0 | 0 |
| 20874571 | −0.64 | −0.61 |
| 20924967 | −0.66 | −0.75 |
| 20943214 | −0.66 | −0.75 |
| 20979545 | −0.53 | −0.67 |
| 21080816 | −0.08 | −0.15 |
| 21126589 | 0.13 | −0.12 |
| 21301953 | 0.30 | −0.05 |
| 21342623 | 0.31 | −0.04 |

Table 30B

| | Mean C16:0 | Mean Total |
|---|---|---|
| Salomon allele | 4.17 | 7.92 |
| 03LC.034 allele | 5.01 | 9.42 |

The results provided in Table 31. Part A provide the Pearson correlation coefficients between saturated fatty acids and marker loci for QTL2 on N19 (n=55). Part B of Table 31 provides a comparison of the mean fatty acid values of plants carrying Salomon and 03LC.034 alleles spanning positions 13003942-15547466 of the DH12075 reference genome.

TABLE 31
Table 31A

| | | Total |
|---|---|---|
| Location | C16:0 | Saturates |
| 13003942 | −0.937 | −0.332 |
| 13008581 | −0.939 | −0.336 |
| 13364132 | −0.939 | −0.336 |
| 13429175 | −0.939 | −0.336 |
| 13429687 | −0.939 | −0.336 |
| 13504886 | −0.939 | −0.336 |
| 13704881 | −0.911 | −0.299 |
| 13925427 | −0.889 | −0.283 |
| 14046125 | −0.889 | −0.283 |
| 14135213 | −0.856 | −0.248 |
| 14377562 | −0.838 | −0.230 |
| 14776751 | −0.740 | −0.182 |
| 15173478 | −0.323 | −0.319 |
| 15235513 | −0.323 | −0.319 |
| 15387929 | −0.322 | −0.340 |
| 15547466 | −0.215 | −0.293 |

Table 31B

| | Mean C16:0 | Mean Total |
|---|---|---|
| Salomon allele | 4.12 | 9.20 |
| 03LC.034 allele | 4.95 | 9.60 |

Table 32 provides a further refinement of the results provided in Table 31. The results in Table 32 Part A provide the Pearson correlation coefficients between saturated fatty acids and marker loci on a smaller portion of N19 (n=79). Part B of Table 32 provides a comparison of mean fatty acid values of plants carrying Salomon and 03LC.034 alleles spanning positions 11538807-13704881 of the DH12075 reference genome.

TABLE 32
Table 32A

| | | Total |
|---|---|---|
| Location | C16:0 | Saturates |
| 11538807 | −0.682 | −0.457 |
| 12010676 | −0.683 | −0.464 |
| 12507143 | −0.733 | −0.442 |
| 12847514 | −0.733 | −0.442 |
| 13003942 | −0.687 | −0.425 |
| 13008581 | −0.684 | −0.423 |
| 13364132 | −0.652 | −0.404 |
| 13429175 | −0.653 | −0.406 |
| 13429687 | −0.647 | −0.406 |
| 13504886 | −0.641 | −0.415 |
| 13704881 | −0.199 | −0.193 |

Table 32B

| | Mean C16:0 | Mean Total |
|---|---|---|
| Salomon allele | 4.08 | 8.05 |
| 03LC.034 allele | 4.92 | 8.60 |

Assessments of the contribution various portions of the genomic region associated with QTL1 on N1 make toward fatty acid profile of Salomon was conducted using five plants from generation 2 having different haplotypes at QTL1. The results shown in table 33 indicate that the region including N1_20772548, N1_20874571, N1_20924967, N1_20943214, N1_20979545, and N1_21057761 (e.g., the region from positions 20874571 to 21057761) significantly correlate with the reduced 16:0 fatty acid content in the seeds of Salomon.

TABLE 33

| | Haplotype | 1 | 2 | 3 | 4 | 5 |
|---|---|---|---|---|---|---|
| Allele at | N1_20772548 | wt | wt | wt | wt | wt |
| Physical | N1_20874571 | mut | het | het | wt | wt |
| Location | N1_20924967 | mut | het | het | wt | wt |
| | N1_20943214 | mut | het | het | wt | wt |
| | N1_20979545 | mut | het | het | wt | wt |
| | N1_21057761 | wt | wt | het | wt | wt |
| | N1_21126589 | wt | wt | het | wt | wt |
| | N1_21301953 | wt | wt | het | wt | het |
| | N1_21342623 | wt | wt | het | wt | het |
| | N | 5 | 14 | 18 | 4 | 2 |
| Mean Fatty | C14:0 | 0.06 | 0.07 | 0.07 | 0.08 | 0.09 |
| Acid (%) | C16:0 | 4.14 | 4.54 | 4.60 | 5.01 | 5.22 |
| | C16:1 | 0.30 | 0.29 | 0.29 | 0.29 | 0.31 |
| | C18:0 | 2.20 | 2.40 | 2.50 | 2.91 | 2.73 |
| | C18:1 | 58.54 | 58.80 | 58.14 | 58.81 | 56.18 |
| | C18:2 | 27.52 | 26.73 | 26.94 | 25.45 | 28.43 |
| | C18:3 | 4.86 | 4.79 | 5.00 | 4.89 | 4.77 |
| | C20:0 | 0.61 | 0.64 | 0.67 | 0.79 | 0.63 |
| | C20:1 | 0.94 | 0.91 | 0.91 | 0.91 | 0.81 |
| | C20:2 | 0.08 | 0.07 | 0.08 | 0.07 | 0.08 |
| | C22:0 | 0.28 | 0.30 | 0.30 | 0.35 | 0.25 |
| | C22:1 | 0.02 | 0.02 | 0.01 | 0.01 | 0.01 |
| | C24:0 | 0.19 | 0.20 | 0.22 | 0.28 | 0.19 |
| | C24:1 | 0.26 | 0.23 | 0.29 | 0.17 | 0.30 |
| | Total Sats | 7.49 | 8.15 | 8.35 | 9.42 | 9.09 |

wt = wild type SNP allele
het = heterozygote (both wild type and mutant SNP alleles)
mut = mutant SNP allele

As with QTL1, assessments of the contribution various genomic regions of QTL2 on N19 make toward to the fatty acid profile of Salomon was conducted. The analysis, which employed seven plants from generation 2 having different haplotypes at QTL2 on N9 is shown in table 34. The analysis indicates that the region including N19_11538807, N19_12010676, N19_12507143, N19_12847514, and N19_13003942 (e.g., the region between 11538807 and 13003942) significantly correlates with the reduced 16:0 fatty acid content in the seeds of Salomon.

TABLE 34

| | Haplotype | 1 | 2 | 3 | 4 | 5 | 6 | 7 |
|---|---|---|---|---|---|---|---|---|
| Allele at | N19_11538807 | wt | mut | het | het | wt | het | het |
| Physical | N19_12010676 | wt | mut | het | het | wt | het | het |
| Location | N19_12507143 | wt | mut | het | wt | het | het | mut |
| | N19_12847514 | wt | mut | het | wt | het | het | mut |
| | N19_13003942 | wt | mut | het | wt | het | wt | mut |
| | N19_13008581 | wt | mut | het | wt | het | wt | mut |
| | N19_13364132 | wt | mut | het | wt | het | wt | mut |
| | N | 4 | 12 | 15 | 3 | 3 | 2 | 2 |
| Mean Fatty | C14_0 | 0.08 | 0.05 | 0.06 | 0.08 | 0.07 | 0.07 | 0.05 |
| Acid (%) | C16_0 | 4.92 | 4.08 | 4.54 | 4.68 | 4.56 | 4.51 | 4.10 |
| | C16_1 | 0.28 | 0.16 | 0.24 | 0.29 | 0.26 | 0.25 | 0.18 |
| | C18_0 | 2.57 | 2.74 | 2.70 | 2.80 | 2.98 | 3.00 | 3.20 |
| | C18_1 | 57.42 | 57.31 | 58.37 | 58.83 | 59.39 | 58.38 | 59.66 |
| | C18_2 | 27.26 | 27.72 | 26.57 | 26.20 | 25.74 | 26.53 | 25.41 |
| | C18_3 | 5.24 | 5.27 | 5.03 | 4.96 | 4.79 | 5.02 | 4.84 |
| | C20_0 | 0.60 | 0.66 | 0.67 | 0.61 | 0.61 | 0.61 | 0.64 |
| | C20_1 | 0.88 | 1.15 | 1.01 | 0.84 | 0.91 | 0.89 | 1.08 |
| | C20_2 | 0.08 | 0.11 | 0.09 | 0.08 | 0.09 | 0.08 | 0.11 |
| | C22_0 | 0.24 | 0.31 | 0.29 | 0.23 | 0.22 | 0.23 | 0.24 |
| | C22_1 | 0.01 | 0.01 | 0.01 | 0.01 | 0.00 | 0.00 | 0.00 |
| | C24_0 | 0.19 | 0.21 | 0.19 | 0.16 | 0.15 | 0.18 | 0.21 |
| | C24_1 | 0.22 | 0.21 | 0.22 | 0.23 | 0.23 | 0.24 | 0.30 |
| | TOTSATS | 8.60 | 8.05 | 8.46 | 8.55 | 8.59 | 8.61 | 8.43 |

wt = wild type SNP allele
het = heterozygote (both wild type and mutant SNP alleles)
mut = mutant SNP allele

### Example 15

### Introduction of OTLs Associated with Low C16:0 Content into Other Lines

To assess the impact of the identified loci on plant oil production, NILs for N1 and N19 were developed as outlined in FIG. 5 in the back ground of Recurrent Parent (RP) 03LC.034. By isolating each genetic locus in NILs developed in a common background line the contribution of each locus to the phenotype can be examined. The use of NILs in that fashion eliminates the confounding effects of the other potentially functional loci. NILs having the isolated N1 and N19 loci, and those loci in combination with the QTL identified on the N4 chromosome (FATA2) are presented in FIG. 5. In addition, the three QTL identified in Salomon (i.e., the loci on chromosomes N1, N19 and N4) were incorporated into a single NIL along with FATB mutant alleles resulting in a reduction in that enzymatic activity (see generation 15 in FIG. 5).

For fatty acid profile analysis each NIL was grown in two environments that varied by diurnal temperature (20/17° C. day/night; 15/12° C.). The data provided show that the entire fatty acid profile, with the exception of C22:1 and C24:1, differ between at least one of the NILs and the wild-type control. It also demonstrates an improvement in the reduction of Total Saturated Fat by 1.06% with the addition of FATB mutant alleles.

The fatty acid profile in Table 35, which demonstrates the isolated effect of the N1 QTL on the fatty acid profile, was developed from NILs bearing the N1 locus of Salomon grown in replicate under greenhouse conditions. The introduction of the N1 QTL locus from Salomon significantly lowers C16:0, C18:0, C18:1, C20:0, C20:1, C22:0, C24:0, C24:1 and total saturated fatty acids (Total Sats) relative to wild the type wild type control line 03LC.034. In addition, C18:2 was increased by 3.47% in NILs carrying the introgression relative to wild-type.

The data in Table 36 demonstrates the effects of the isolated N19 locus on the fatty acid profile of 03LC.034. Those data indicate that the N19 locus of Salomon significantly lower the C16:0 and total saturated fatty acids (Total Sats) in lines carrying the genomic introgression from Salomon in both a homozygous and heterozygous genotypic state relative to wild-type control lines. In addition, C20:1 was increased in NILs carrying the N19 locus relative to wild-type.

Finally, the data in Table 37 demonstrates the effect of combining all three QTL originating from Salomon, N1, N19 and N4 (FATA2) in a single NIL having B. napus 03LC.034 as the background. In addition, that combination of loci was introduced into a single NIL having a 03LC.034 background along with mutant alleles of FATB isoforms 1 and 4.

TABLE 35
Mean fatty acid values of lines carrying mutant introgression spanning physical
positions 20874310-22782616 on chromosome N1 relative to wild-type lines

| | | Mean Fatty Acid | | | | | | | |
|---|---|---|---|---|---|---|---|---|---|
| Genotype | N | C14:0 | C16:0 | C16:1 | C18:0 | C18:1 | C18:2 | C18:3 | C20:0 |
| Wild type (line 03LC.034) | 6 | 0.06 | 5.07 | 0.31 | 3.75 | 65.86 | 18.50 | 3.32 | 1.05 |
| (20874310-22782616) | | | | | | | | | |
| Heterozygote (20874310-22782616) | n/a | n/a | n/a | n/a | n/a | n/a | n/a | n/a | n/a |
| Mutant (20874310-22782616) | 49 | 0.05 | 4.45 | 0.32 | 3.09 | 64.52 | 21.97 | 3.37 | 0.73 |
| Different at P < 0.05 | | | * | | * | * | * | | * |

| | | Mean Fatty Acid | | | | | | |
|---|---|---|---|---|---|---|---|---|
| | | | | | | | | Total |
| Genotype | N | C20:1 | C20:2 | C22:0 | C22:1 | C24:0 | C24:1 | Sats |
| Wild type (line 03LC.034) | 6 | 0.99 | 0.06 | 0.39 | 0.00 | 0.38 | 0.27 | 10.70 |
| (20874310-22782616) | | | | | | | | |
| Heterozygote (20874310-22782616) | n/a | n/a | n/a | n/a | n/a | n/a | n/a | n/a |
| Mutant (20874310-22782616) | 49 | 0.80 | 0.05 | 0.25 | 0.00 | 0.23 | 0.17 | 8.80 |
| Different at P < 0.05 | | * | | * | | * | * | * |

TABLE 36
Mean fatty acid values of lines carrying mutant introgression spanning physical
positions 14188467-14907742 on chromosome N1 relative to heterozygous and wild-type lines

| | | Mean Fatty Acid | | | | | | | |
|---|---|---|---|---|---|---|---|---|---|
| Genotype | N | C14:0 | C16:0 | C16:1 | C18:0 | C18:1 | C18:2 | C18:3 | C20:0 |
| Wild type (line 03LC.034) | 5 | 0.05 A | 4.83 A | 0.24 A | 3.17 A | 65.36 A | 20.09 A | 3.13 A | 0.85 A |
| (p14188467-14907742) | | | | | | | | | |
| Heterozygote | 10 | 0.09 A | 4.35 B | 0.28 A | 3.10 A | 64.96 A | 20.97 A | 3.06 A | 0.85 A |
| (p14188467-14907742) | | | | | | | | | |
| Mutant (p14188467-14907742) | 9 | 0.07 A | 3.91 C | 0.21 A | 3.10 A | 65.81 A | 20.70 A | 2.98 A | 0.81 A |
| Significant at P < 0.05 | | | * | | | | | | |

| | | Mean Fatty Acid | | | | | | |
|---|---|---|---|---|---|---|---|---|
| | | | | | | | | Total |
| Genotype | N | C20:1 | C20:2 | C22:0 | C22:1 | C24:0 | C24:1 | Sats |
| Wild type (line 03LC.034) | 5 | 1.00 A | 0.12 A | 0.37 A | 0.02 A | 0.67 A | 0.26 A | 9.94 A |
| (p14188467-14907742) | | | | | | | | |
| Heterozygote | 10 | 1.17 B | 0.10 A | 0.35 A | 0.02 A | 0.37 A | 0.27 A | 9.11 B |
| (p14188467-14907742) | | | | | | | | |
| Mutant (p14188467-14907742) | 9 | 1.31 C | 0.09 A | 0.30 A | 0.02 A | 0.26 A | 0.35 A | 8.45 C |
| Significant at P < 0.05 | | * | | | | | | * |

Means with the same letter are not significantly different.

TABLE 37
Mean fatty acid values of lines carrying mutant introgressions derived from
Salomon (N1/N19/FATA2) and Salomon + FATB (N1/N19/FATA2/FATB1/FATB4)
mutations relative to wild-type (10H3627) in: A) 15/12° C. (day/night);
B) 20/17° C. and C) the mean estimate across treatments

| A) | | Mean Fatty Acid | | | | | | | |
|---|---|---|---|---|---|---|---|---|---|
| Line | N | C14:0 | C16:0 | C16:1 | C18:0 | C18:1 | C18:2 | C18:3 | C20:0 |
| 10H3627 | 6 | 0.05 A | 4.52 A | 0.31 A | 2.22 A | 63.29 A | 23.27 A | 3.95 A | 0.70 A |
| 03LC.034/N1 + N19 + | 5 | 0.02 B | 3.24 B | 0.17 B | 1.44 B | 60.26 B | 27.82 B | 4.45 B | 0.54 B |
| FATA2 | | | | | | | | | |
| 03LC.034/N1 + N19 + | 4 | 0.01 B | 2.47 C | 0.18 B | 1.44 B | 61.93 A | 27.08 B | 4.77 C | 0.40 C |
| FATA2 + FATB1:4 | | | | | | | | | |

| | | Mean Fatty Acid | | | | | | |
|---|---|---|---|---|---|---|---|---|
| A) | | | | | | | | Total |
| Line | N | C20:1 | C20:2 | C22:0 | C22:1 | C24:0 | C24:1 | Sats |
| 10H3627 | 6 | 0.95 A | 0.07 A | 0.34 A | 0.00 A | 0.14 A | 0.19 A | 7.97 A |
| 03LC.034/N1 + N19 + | 5 | 1.20 B | 0.15 B | 0.30 B | 0.01 AB | 0.08 AB | 0.31 A | 5.63 B |
| FATA2 | | | | | | | | |
| 03LC.034/N1 + N19 + | 4 | 1.08 C | 0.15 B | 0.17 C | 0.04 B | 0.00 B | 0.30 A | 4.49 C |
| FATA2 + FATB1:4 | | | | | | | | |

| B) | | Mean Fatty Acid | | | | | | | |
|---|---|---|---|---|---|---|---|---|---|
| Line | N | C14:0 | C16:0 | C16:1 | C18:0 | C18:1 | C18:2 | C18:3 | C20:0 |
| 10H3627 | 8 | 0.06 A | 4.59 A | 0.29 A | 2.0 A | 65.05 A | 21.99 A | 3.80 A | 0.61 A |
| 03LC.034/N1 + N19 + | 5 | 0.01 B | 3.42 B | 0.14 B | 1.46 B | 63.75 A | 24.56 B | 3.97 A | 0.55 B |
| FATA2 | | | | | | | | | |
| 03LC.034/N1 + N19 + | 5 | 0.01 B | 2.74 C | 0.15 B | 1.52 B | 64.68 A | 24.73 B | 4.10 A | 0.40 C |
| FATA2 + FATB1:4 | | | | | | | | | |

| | | Mean Fatty Acid | | | | | | |
|---|---|---|---|---|---|---|---|---|
| B) | | | | | | | | Total |
| Line | N | C20:1 | C20:2 | C22:0 | C22:1 | C24:0 | C24:1 | Sats |
| 10H3627 | 8 | 0.88 A | 0.06 A | 0.26 A | 0.00 A | 0.15 A | 0.23 A | 7.70 A |
| 03LC.034/N1 + N19 + | 5 | 1.31 C | 0.15 B | 0.28 A | 0.03 B | 0.19 A | 0.18 A | 5.92 B |
| FATA2 | | | | | | | | |
| 03LC.034/N1 + N19 + | 5 | 1.12 B | 0.14 B | 0.16 B | 0.00 A | 0.08 B | 0.18 A | 4.91 C |
| FATA2 + FATB1:4 | | | | | | | | |

| C) | | Mean Fatty Acid | | | | | | | |
|---|---|---|---|---|---|---|---|---|---|
| Line | N | C14:0 | C16:0 | C16:1 | C18:0 | C18:1 | C18:2 | C18:3 | C20:0 |
| 10H3627 | 14 | 0.06 A | 4.56 A | 0.30 A | 2.11 A | 64.30 A | 22.54 A | 3.87 A | 0.65 A |
| 03LC.034/N1 + N19 + | 10 | 0.02 B | 3.33 B | 0.16 B | 1.45 B | 62.00 B | 26.19 B | 4.21 AB | 0.55 B |
| FATA2 | | | | | | | | | |
| 03LC.034/N1 + N19 + | 9 | 0.02 B | 2.62 C | 0.16 B | 1.48 B | 63.46 AB | 25.78 B | 4.40 B | 0.40 C |
| FATA2 + FATB1:4 | | | | | | | | | |

| | | Mean Fatty Acid | | | | | | |
|---|---|---|---|---|---|---|---|---|
| C) | | | | | | | | Total |
| Line | N | C20:1 | C20:2 | C22:0 | C22:1 | C24:0 | C24:1 | Sats |
| 10H3627 | 14 | 0.91 A | 0.06 A | 0.29 A | 0.00 A | 0.14 A | 0.21 A | 7.81 A |
| 03LC.034/N1 + N19 + | 10 | 1.25 C | 0.15 B | 0.29 A | 0.02 A | 0.14 A | 0.25 A | 5.78 B |
| FATA2 | | | | | | | | |
| 03LC.034/N1 + N19 + | 9 | 1.10 C | 0.14 B | 0.16 B | 0.02 A | 0.04 B | 0.23 A | 4.72 C |
| FATA2 + FATB1:4 | | | | | | | | |

### Other Embodiments

It is to be understood that, while the invention has been described throughout this disclosure in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1-28. (canceled)

29. A Brassica plant that is non-transgenic or a Brassica plant that is free of transgenes other than those for herbicide tolerance, or part thereof, comprising a nucleic acid sequence having greater than 90%, 95%, 97.5%, 98%, 99%, 99.9%, 99.99%, or 99.999% identity, or having 100% identity to all of, comprising all of, or a part comprising greater than 20, 30, 40, 50 or 60 contiguous nucleotides of, the genomic sequences between the chromosome N1 (QTL1) SNP markers at positions 20772548 and 22780181, 20843387 and 21080816, or 20874571 and 20979545 of the B. napus Salomon line ATCC deposit designation PTA-11453; said plant further comprising mutant alleles at two or more, three or more, or four or more, different fatty acyl ACP thioesterase B (FATB) loci, wherein each said mutant allele results in the production of a FATB polypeptide having reduced thioesterase activity relative to a corresponding wild-type FATB polypeptide.

30. The Brassica plant, or a part thereof, of claim 29, wherein at least one of said mutant alleles comprises a nucleic acid encoding a truncated FATB polypeptide.

31. The Brassica plant, or a part thereof, of claim 29, wherein at least one of said mutant alleles comprises a nucleic acid encoding a FATB polypeptide having a deletion of a helix/4-stranded sheet (4HBT) domain or a portion thereof.

32. The Brassica plant, or a part thereof, of claim 29, wherein at least one of said mutant alleles comprises a nucleic acid encoding a FATB polypeptide having a non-conservative substitution of a residue affecting substrate specificity.

33. The Brassica plant, or a part thereof, of claim 29, wherein at least one of said mutant alleles comprises a nucleic acid encoding a FATB polypeptide having a non-conservative substitution of a residue affecting catalytic activity.

34. The Brassica plant, or a part thereof, of claim 29, wherein said plant produces seeds yielding an oil having a total saturates content of about 2.5% to 5.5%.

35. The Brassica plant, or a part thereof, of claim 34, said oil further having an oleic acid content of about 78% to 80%, a linoleic acid content of about 8% to 10%, and an α-linolenic acid content of about 2% to 4%.

36. The Brassica plant, or a part thereof, of claim 29, wherein said plant produces seeds yielding an oil having a palmitic acid content of about 1.5% to 3.5%.

37. The Brassica plant, or a part thereof, of claim 29, wherein said plant produces seeds yielding an oil having a stearic acid content of about 0.5% to 2.5%.

38. The Brassica plant, or a part thereof of claim 29, said plant further comprising a mutant allele at a delta-12 fatty acid desaturase (FAD2) locus, said mutant allele comprising a nucleic acid encoding a FAD2 polypeptide having a lysine substituted for glutamic acid in a His-Glu-Cys-Gly-His motif.

39. The Brassica plant, or a part thereof, of claim 38, said plant further comprising a mutant allele at a different FAD2 locus, said mutant allele comprising a nucleic acid encoding a FAD2 polypeptide having a glutamic acid substituted for glycine in the DRDYGILNKV motif or a histidine substituted for leucine in a KYLNNP motif.

40. The Brassica plant, or a part thereof, of claim 29, said plant further comprising a mutant allele at a FAD2 locus, said mutant allele comprising a nucleic acid encoding a FAD2 polypeptide having a glutamic acid substituted for glycine in the DRDYGILNKV motif or a histidine substituted for leucine in a KYLNNP motif.

41-92. (canceled)


SEQUENCE LISTING (162 sequences)

SEQ ID NO: 1 - DNA, 1557 bp, Brassica napus
atggtggctacttgcgctacgtcgtcgttttttcatgttccatcttcttcctcgcttgatactaatgggaaggggaacagagttgggtctactaattttgctggacttaactcaacgccaagctctgggaggatgaaggttaagccaaacgcttaggctccacccaagatcaacgggaagaaagctaacttgcctggctctgtagagatatcaaagtctgacaacgagacttcgcaacccgcacacgcaccgaggacgtttatcaaccagctacctgactggagcatgcttcttgctgccataacaactattttcttagcggcggagaaacagtggatgatgcttgactggaaacctaggcgttctgatatgattatggatcctttcggtttagggagaatcgttcaggatggtcttgtgttccgtcagaatttttccattaggtcttatgagataggtgctgatcgctctgcgtctatagaaactgtcatgaatcatttacaggtactgctttgattgtggttacactcacatgttgtcccaatagatatatgctcatgacaagctcttatgctaatgacaggaaacggcgcttaatcatgtgaagtctgccggactgctggaaaatgggtttgggtccactcctgagatgtttaagaagaatttgatatgggtcgttgctcgtatgcaggttgtcgttgataaatatcctacttggtaagccattgttagtcttagcacttgacttaaaatcattttgcatattacagtgtgcgtagatcatttgcttattcaaatatctgactcacaggggagatgttgtggaagtggatacttgggttagtcagtctggaaagaatggtatgcgtcgtgattggctagttcgggattgcaatactggagaaattgtaacgcgagcatcaaggtcagagttcttatattttggtttactccagctattatcgttttgctctctgtttgtattgtttcctctgccattagtttgataattgagtctttatagttgtatatgtatggcaattttcttctttttgcagtttgtgggtgatgatgaataaactcacaaggagattgtcaaagattcctgaagaggttcgaggggaaatagagccttattttgtgaactctgatcctgtcattgccgaagacagcagaaagttaacaaaacttgatgacaagactgctgactatgttcgttctggtctcactgtaagtaccttacctttcgacaagcctgtcaaaactcttgaggttctaatggtttggtaatgaacttttttttggcagccgaggtggagtgacttggatgttaaccagcatgttaacaatgtaaagtacattgggtggatactggagagtgctccagcagggatgctggagagtcagaagctgaaaagcatgactctggagtatcgcagggagtgcgggagagacagtgtgcttcagtctctcaccgcagtctctggatgtgatgtcggtaacctcgggacagccggggaagtggagtgtcagcatttgcttcgactccaggatgga

SEQ ID NO: 2 - DNA, 1572 bp, Brassica napus
atggtggccacctcagctacatcctcattcttccctctcccatcttcccccctcgaccccaccgcaaaaaccaacaaagtcaccacctccaccaacttctccggcctcacacccacgccgaactccgccaggatgaaggttaaaccaaacgctcaggccccacccaagatcaacggcaagagagtcggcctccctggctcggtggagatcttgaagcctgatagcgagacttcgcaaccagcaccgaggacgttcatcaaccagctgcctgactggagcatgctcctcgccgccatcacgaccgtcttcttggcggctgagaagcagtggatgatgctcgactggaaaccgaggcgttctgacgtgattatggatccgtttgggttagggaggatcgttcaggatgggcttgtgttccgtcagaatttttctattcggtcttatgagataggtgctgatcgctctgcgtctatagaaacggttatgaatcatttacaggtactgattatgattatgattgtagtcgcttgttgttactggacaaacttaaatatgtattgctcttatggttgtgataggaaacggcactcaaccatgttaagactgctgggctgcttggagatgggtttggttctactcctgagatggttaagaagaacttgatatgggttgttactcgtatgtaggttgtcgttgataaatatcctacttggtaagctattctcaaacaactctgagaatcactgcttcctttgtgagtcatttgcttattcaaatatctgcctcataggggagatgttgtggaagtagatacatgggtgagccagtctggaaagaacggtatgcgtcgtgattggcttgttcgggatggcaatactggagagattttaacaagagcatcaaggttagattttattttttggtttacttgggttagatatctgataattgagttataatcatctccgtgttgtgtaaactattctttttgcagtgtgtgggtgatgatgaataaactgacaagaagattatcaaagattcctgaagaggttcgaggggagatagagccttactttgttaactcagacccagtccttgccgaggacagcagaaagttaacaaaacttgatgacaaaactgctgtctatgttcgttctggtctcactgtaagtacaaatacttcactctatgtttcaacaaagcctgtaaatttttgagtctcttacaggtttggtaatgaactttttgcagccgcgttggagtgacttggatgttaaccagcacgttaacaatgtgaagtacatcgggtggatactggagagtgctccagtggggatgatggagagtcagaagctgaaaagcatgactctggagtatcgcagggagtgtgggagagacagtgtgctccagtccctcaccgcggtttcgggctgcgatatcggtagcctcgggacagccggtgaagtggaatgtcagcatctgctcagactccaggatggagccgaagtggtgagaggaagaacagagtggagttccaaaacatcaacaacaacttgggacatcacaccgtga

SEQ ID NO: 3 - DNA, 1500 bp, Brassica napus
atggtggccacctcagctacatcctcattcttccctctcccatcttcccccctcgaccccaccgcaaaaaccaacaaagtcaccacctccaccaacttctccggcctcacacccacgccgaactccgccaggatgaaggttaaaccaaacgctcaggccccacccaagatcaacggcaagagagtcggcctccctggctcggtggagatcttgaagcctgatagcgagacttcgcaaccagcaccgaggacgttcatcaaccagctgcctgactgaagcatgctcctcgccgccatcacgaccgtcttcttggcggctgagaagcagtggatgatgctcgactggaaaccgaggcgttctgacgtgattatggatccgtttgggttagggaggatcgttcaggatgggcttgtgttccgtcagaatttttctattcggtcttatgagataggtgctgatcgctctgcgtctatagaaacggttatgaatcatttacaggtactgattatgattatgattgtagtcgcttgttgttactggacaaacttaaatatgtattgctcttatggttgtgataggaaacggcactcaaccatgttaagactgctgggctgcttggagatgggtttggttctactcctgagatggttaagaagaacttgatatgggttgttactcgtatgcaggttgtcgttgataaatatcctacttggtaagctattctcaaacaactctgagaatcactgcttcctttgtgagtcatttgcttattcaaatatctgcctcataggggagatgttgtggaagtagatacatgggtgagccagtctggaaagaacggtatgcgtcgtgattggcttgttcgggatggcaatactggagagattttaacaagagcatcaaggttagattttattttttggtttacttgggttagatatctgataattgagttataatcatctccgtgttgtgtaaactattctttttgcagtgtgtgggtgatgatgaataaactgacaagaagattatcaaagattcctgaagaggttcgaggggagatagagccttactttgttaactcagacccagtccttgccgaggacagcagaaagttaacaaaacttgatgacaaaactgctgtctatgttcgttctggtctcactgtaagtacaaatacttcactctatgtttcaacaaagcctgtaaatttttgagtctcttacaggtttggtaatgaactttttgcagccgcgttggagtgacttggatgttaaccagcacgttaacaatgtgaagtacatcgggtggatactggagagtgctccagtggggatgatggagagtcagaagctgaaaagcatgactctggagtatcgcagggagtgtgggagagacagtgtgctccagtccctcaccgcggtttcgggctgcgatatcggtagcctcgggacagccggtgaagtggaatgtcagcatctgctcagactccaggatgga

SEQ ID NO: 4 - DNA, 1664 bp, Brassica napus
atggtggctacttccgctacgtcgtcgttttttcatgttccatcttcctcctctcttgatactaatgggaaggggaacagagttgcgtccacgaacttcgctggacttaactcaacgccaagctctgggaggatgaaggttaaaccaaacgctcaggctccacccaagatcaacgggaagaaagctaacttgcctggttctgcagagatatcaaagtctgacaacgagacttcgcaacccgcacccgcaccgaggacgtttatcaaccagctgcctgactggagcatgcttctcgctgccataacaactattttcttagcggctgagaaacagtgaatgatgcttgactggaaacccaggcgttctgatatgataatggatcctttcggtttagggagaatcgttcaggatggtcttgtgtttcgtcagaatttctccattaggtcttatgagataggtgctgatcgctctgcgtctatagaaactgttatgaatcatttacaggtaggtactactttgattgttatcacacttgtcactggacacccaatagatatatatgctcatgacaagctcttatgctaatgacaggaaacggccctaaaccatgtgaagtctgccggactgctggaaaatgggtttggttctactcccgagatgtttaagaagaacttgatatgggtcgttgctcgtatgcaggttgtcgttgataaatatcctacttggtaagccattgtcagtcttaccacttaacttaaaatcattatgcatattacagtttgcatagatcattacttattcaaatatctgactaacaggggagatgttgtggaagtggatacatgggttagtcagtccggaaagaatggtatgcgtcgtgattggctggttcgggattgcaatactggagaaattgtaacgcgagcatcaaggtcagagttcttatgttttggtttactgactccagctattatcattttgctctctgtttgtattgtttgctctgccattaatatgataatagagactttatagttgtatatgtatggcaattttcttctttttgcagtttgtgggtgatgatgaataaactgacaaggagattgtcaaagattcctgaagaggttcgtggggaaatagagccttattttgtgaactctgatcctgtcattgccgaagacagcagaaagttaacaaaactggatgacaagactgctgactatgttcgttcgggtctcactgtaagtaccctacctttcaacaagcctttaaaactcttgaggttctaatggtttggtaataaacttttttttcagccgagttggagtgacttagatgttaaccagcatgttaacaatgtaaagtacattgggtggatactggagagtgctccagcagggatgctggagagtcagaagctgaaaagcatgactctggagtatcgcagggagtgcgggagagacagtgtgcttcagtctctcaccgcggtctctggatgtgatgtcggtaacctcgggacagccggggaagtggagtgtcagcatttgcttcgtctccaggatggagctgaagtggtgagaggaagaacagctgaagtggtgagaggaagaacagagtggagttccaagatagaagcaacaacttgggacactgctacatcgtaa

SEQ ID NO: 5 - protein, 5 aa, Artificial Sequence
  Amino acid motif
HECGH

SEQ ID NO: 6 - protein, 6 aa, Artificial Sequence
  Amino acid motif
KYLNNP

SEQ ID NO: 7 - protein, 10 aa, Artificial Sequence
  Amino acid motif
DRDYGILNKV

SEQ ID NO: 8 - DNA, 26 bp, Artificial Sequence
  Primer
atgaaggttaaaccaaacgctcaggc

SEQ ID NO: 9 - DNA, 24 bp, Artificial Sequence
  Primer
tgttcttcctctcaccacttcagc

SEQ ID NO: 10 - DNA, 1399 bp, Brassica napus
tccacccaagatcaacgggaagaaagctaacttgcctggctctgtagagatatcaaagtctgacaacgagacttcgcaacccgcacacgcaccgaggacgtttatcaaccagctacctgactggagcatgcttcttgctgccataacaactattttcttagcggcggagaaacagtggatgatgcttgactggaaacctaggcgttctgatatgattatggatcctttcggtttagggagaatcgttcaggatggtcttgtgttccgtcagaatttttccattaggtcttatgagataggtgctgatcgctctgcgtctatagaaactgtcatgaatcatttacaggtactgctttgattgtggttacactcacatgttgtcccaatagatatatgctcatgacaagctcttatgctaatgacaggaaacggcgcttaatcatgtgaagtctgccggactgctggaaaatgggtttgggtccactcctgagatgtttaagaagaatttgatatgggtcgttgctcgtatgcaggttgtcgttgataaatatcctacttggtaagccattgttagtcttagcacttgacttaaaatcattttgcatattacagtgtgcgtagatcatttgcttattcaaatatctgactcacaggggagatgttgtggaagtggatacttgggttagtcagtctggaaagaatggtatgcgtcgtgattggctagttcgggattgcaatactggagaaattgtaacgcgagcatcaaggtcagagttcttatattttggtttactccagctattatcgttttgctctctgtttgtattgtttcctctgccattagtttgataattgagtctttatagttgtatatgtatggcaattttcttctttttgcagtttgtgggtgatgatgaataaactcacaaggagattgtcaaagattcctgaagaggttcgaggggaaatagagccttattttgtgaactctgatcctgtcattgccgaagacagcagaaagttaacaaaacttgatgacaagactgctgactatgttcgttctggtctcactgtaagtaccttacctttcgacaagcctgtcaaaactcttgaggttctaatggtttggtaatgaacttttttttggcagccgaggtggagtgacttggatgttaaccagcatgttaacaatgtaaagtacattgggtggatactggagagtgctccagcagggatgctggagagtcagaagctgaaaagcatgactctggagtatcgcagggagtgcgggagagacagtgtgcttcagtctctcaccgcagtctctggatgtgatgtcggtaacctcgggacagccggggaagtggagtgtcagcatttgcttcgactccaggatgga

SEQ ID NO: 11 - DNA, 1330 bp, Brassica napus
cccacccaagatcaacggcaagagagtcggtctcccttctggctcggtgaagcctgataacgagacgtcctcacagcatcccgcagcaccgaggacgttcatcaaccagctgcctgactggagcatgcttcttgctgcaataacaaccgtcttcttggcggctgagaagcagtggatgatgcttgactggaaaccgaggcgctctgacgtgattatggatccgtttgggttagggaggatcgttcaggatgggcttgtgttccgtcagaatttctctattcggtcttatgagataggtgctgatcgctctgcgtctatagaaacggttatgaatcatttacaggtactgattatgattatgattatgattgtagttgcttgttgttactggacaaagttaatatgtattgctgttatggttatgataggaaacggcactcaaccatgttaagactgctggactgcttggagatgggtttggttctactcctgagatggttaagaagaacttgatttgggttgttactcgtatgcaggttgtcgttgataaatatcctacttggtaagctattctcaagcaaccctgagaatcactgcttcctttgtcatttgcttattcaaatatctgtctcacaggggagatgttgtggaagtagatacatgggtgagccagtctggaaagaacggtatgcgtcgtgattggctagttcgagatggcaatactggagaaattttaacaagagcatcaaggttagatttttatttatcggttaggtatctgaaaatttgagttactaatgcaaaatattatttttgcagtgtgtgggtgatgatgaataaactgacaagaagattatcaaagattcctgaagaggttcgaggggagatagagccttactttgttaattcagacccagtccttgctgaggacagcagaaagttaactaaacttgatgacaagactgctgactatgttcgttctggtctcactgtaagtatgcatactttctctatgtttcatcaaagcctgtaaacttctgagattcttacagtttttatttggtaatttaaacttttgcagccgcgttggagtgacttggatgttaaccagcacgttaacaatgtgaagtacatcgggtggatactggagagtgcacctgtggggatgatggagagtcagaagctgaaaagcatgactctggagtatcgcagggagtgcgggagggacagtgtgcttcagtccctcaccgcggtttcgggctgcgatgttggtagtcttgggacagctggtgaagtggaatgtcagcacctgctccgtctccaggatgga

SEQ ID NO: 12 - DNA, 1342 bp, Brassica napus
cccacccaagatcaacggcaagagagtcggcctccctggctcggtggagatcttgaagcctgatagcgagacttcgcaaccagcaccgaggacgttcatcaaccagctgcctgactggagcatgctcctcgccgccatcacgaccgtcttcttggcggctgagaagcagtggatgatgctcgactggaaaccgaggcgttctgacgtgattatggatccgtttgggttagggaggatcgttcaggatgggcttgtgttccgtcagaatttttctattcggtcttatgagataggtgctgatcgctctgcgtctatagaaacggttatgaatcatttacaggtactgattatgattatgattgtagtcgcttgttgttactggacaaacttaaatatgtattgctcttatggttgtgataggaaacggcactcaaccatgttaagactgctgggctgcttggagatgggtttggttctactcctgagatggttaagaagaacttgatatgggttgttactcgtatgcaggttgtcgttgataaatatcctacttggtaagctattctcaaacaactctgagaatcactgcttcctttgtgagtcatttgcttattcaaatatctgcctcataggggagatgttgtggaagtagatacatgggtgagccagtctggaaagaacggtatgcgtcgtgattggcttgttcgggatggcaatactggagagattttaacaagagcatcaaggttagattttattttttggtttacttgggttagatatctgataattgagttataatcatctccgtgttgtgtaaactattctttttgcagtgtgtgggtgatgatgaataaactgacaagaagattatcaaagattcctgaagaggttcgaggggagatagagccttactttgttaactcagacccagtccttgccgaggacagcagaaagttaacaaaacttgatgacaaaactgctgtctatgttcgttctggtctcactgtaagtacaaatacttcactctatgtttcaacaaagcctgtaaatttttgagtctcttacaggtttggtaatgaactttttgcagccgcgttggagtgacttggatgttaaccagcacgttaacaatgtgaagtacatcgggtggatactggagagtgctccagtggggatgatggagagtcagaagctgaaaagcatgactctggagtatcgcagggagtgtgggagagacagtgtgctccagtccctcaccgcggtttcgggctgcgatatcggtagcctcgggacagccggtgaagtggaatgtcagcatctgctcagactccaggatgga

SEQ ID NO: 13 - DNA, 1407 bp, Brassica napus
tccacccaagatcaacgggaagaaagctaacttgcctggttctgcagagatatcaaagtctgacaacgagacttcgcaacccgcacccgcaccgaggacgtttatcaaccagctgcctgactggagcatgcttctcgctgccataacaactattttcttagcggctgagaaacagtggatgatgcttgactggaaacccaggcgttctgatatgataatggatcctttcggtttagggagaatcgttcaggatggtcttgtgtttcgtcagaatttctccattaggtcttatgagataggtgctgatcgctctgcgtctatagaaactgttatgaatcatttacaggtaggtactactttgattgttatcacacttgtcactggacacccaatagatatatatgctcatgacaagctcttatgctaatgacaggaaacggccctaaaccatgtgaagtctgccggactgctggaaaatgggtttggttctactcccgagatgtttaagaagaacttgatatgggtcgttgctcgtatgcaggttgtcgttgataaatatcctacttggtaagccattgtcagtcttaccacttaacttaaaatcattatgcatattacagtttgcatagatcattacttattcaaatatctgactaacaggggagatgttgtggaagtggatacatgggttagtcagtccggaaagaatggtatgcgtcgtgattggctggttcgggattgcaatactggagaaattgtaacgcgagcatcaaggtcagagttcttatgttttggtttactgactccagctattatcattttgctctctgtttgtattgtttgctctgccattaatatgataatagagactttatagttgtatatgtatggcaattttcttctttttgcagtttgtgggtgatgatgaataaactgacaaggagattgtcaaagattcctgaagaggttcgtggggaaatagagccttattttgtgaactctgatcctgtcattgccgaagacagcagaaagttaacaaaactggatgacaagactgctgactatgttcgttcgggtctcactgtaagtaccctacctttcaacaagcctttaaaactcttgaggttctaatggtttggtaataaacttttttttcagccgagttggagtgacttagatgttaaccagcatgttaacaatgtaaagtacattgggtggatactggagagtgctccagcagggatgctggagagtcagaagctgaaaagcatgactctggagtatcgcagggagtgcgggagagacagtgtgcttcagtctctcaccgcggtctctggatgtgatgtcggtaacctcgggacagccggggaagtggagtgtcagcatttgcttcgtctccaggatgga

SEQ ID NO: 14 - DNA, 24 bp, Artificial Sequence
  Primer
ctttgaacgctcagctcctcagcc

SEQ ID NO: 15 - DNA, 26 bp, Artificial Sequence
  Primer
aaacgaaccaaagaacccatgtttgc

SEQ ID NO: 16 - DNA, 24 bp, Artificial Sequence
  Primer
ctttgaaagctcatcttcctcgtc

SEQ ID NO: 17 - DNA, 25 bp, Artificial Sequence
  Primer
ggttgcaaggtagcagcaggtacag

SEQ ID NO: 18 - DNA, 1557 bp, Brassica napus
atggtggctacttgcgctacgtcgtcgttttttcatgttccatcttcttcctcgcttgatactaatgggaaggggaacagagttgggtctactaattttgctggacttaactcaacgccaagctctgggaggatgaaggttaagccaaacgctcaggctccacccaagatcaacgggaagaaagctaacttgcctggctctgtagagatatcaaagtctgacaacgagacttcgcaacccgcacacgcaccgaggacgtttatcaaccagctacctgactggagcatgcttcttgctgccataacaactattttcttagcggcggagaaacagtggatgatgcttgactggaaacctaggcgttctgatatgattatggatcctttcggtttagggagaatcgttcaggatggtcttgtgttccgtcagaatttttccattaggtcttatgagataggtgctgatcgctctgcgtctatagaaactgtcatgaatcatttacaggtactgctttgattgtggttacactcacatgttgtcccaatagatatatgctcatgacaagctcttatgctaatgacaggaaacggcgcttaatcatgtgaagtctgccggactgctggaaaatgggtttgggtccactcctgagatgtttaagaagaatttgatatgggtcgttgctcgtatgcaggttgtcgttgataaatatcctacttggtaagccattgttagtcttagcacttgacttaaaatcattttgcatattacagtgtgcgtagatcatttgcttattcaaatatctgactcacaggggagatgttgtggaagtggatacttgggttagtcagtctggaaagaatggtatgcgtcgtgattggctagttcgggattgcaatactggagaaattgtaacgcgagcatcaaggtcagagttcttatattttggtttactccagctattatcgttttgctctctgtttgtattgtttcctctgccattagtttgataattgagtctttatagttgtatatgtatggcaattttcttctttttgcagtttgtgggtgatgatgaataaactcacaaggagattgtcaaagattcctgaagaggttcgaggggaaatagagccttattttgtgaactctgatcctgtcattgccgaagacagcagaaagttaacaaaacttgatgacaagactgctgactatgttcgttctggtctcactgtaagtaccttacctttcgacaagcctgtcaaaactcttgaggttctaatggtttggtaatgaacttttttttggcagccgaggtggagtgacttggatgttaaccagcatgttaacaatgtaaagtacattgggtggatactggagagtgctccagcagggatgctggagagtcagaagctgaaaagcatgactctggagtatcgcagggagtgcgggagagacagtgtgcttcagtctctcaccgcagtctctggatgtgatgtcggtaacctcgggacagccggggaagtggagtgtcagcatttgcttcgactccaggatgga

SEQ ID NO: 19 - DNA, 1563 bp, Brassica napus
atggtggccacctcagctacatcctcattcttccctctcccatctttccccctcgaccccaccgcaaaaaccaacaaagtcaccacctccaccaacttctccggcctctcccccactccaaactcctccggcaggatgaaggttaaaccaaacgctcaggccccacccaagatcaacggcaagagagtcggtctcccttctggctcggtgaagcctgataacgagacgtcctcacagcatcccgcagcaccgaggacgttcatcaaccagctgcctgactggagcatgcttcttgctgcaataacaaccgtcttcttggcggctgagaagcagtggatgatgcttgactggaaaccgaggcgctctgacgtgattatggatccgtttgggttagggaggatcgttcaggatgggcttgtgttccgtcagaatttctctattcggtcttatgagataggtgctgatcgctctgcgtctatagaaacggttatgaatcatttacaggtactgattatgattatgattatgattgtagttgcttgttgttactggacaaagttaatatgtattgctgttatggttatgataggaaacggcactcaaccatgttaagactgctggactgcttggagatgggtttggttctactcctgagatggttaagaagaacttgatttgggttgttactcgtatgcaggttgtcgttgataaatatcctacttggtaagctattctcaagcaaccctgagaatcactgcttcctttgtcatttgcttattcaaatatctgtctcacaggggagatgttgtggaagtagatacatgggtgagccagtctggaaagaacggtatgcgtcgtgattggctagttcgagatggcaatactggagaaattttaacaagagcatcaaggttagatttttatttatcggttaggtatctgaaaatttgagttactaatgcaaaatattatttttgcagtgtgtgggtgatgatgaataaactgacaagaagattatcaaagattcctgaagaggttcgaggggagatagagccttactttgttaattcagacccagtccttgctgaggacagcagaaagttaactaaacttgatgacaagactgctgactatgttcgttctggtctcactgtaagtatgcatactttctctatgtttcatcaaagcctgtaaacttctgagattcttacagtttttatttggtaatttaaacttttgcagccgcgttggagtgacttggatgttaaccagcacgttaacaatgtgaagtacatcgggtggatactggagagtgcacctgtggggatgatggagagtcagaagctgaaaagcatgactctggagtatcgcagggagtgcgggagggacagtgtgcttcagtccctcaccgcggtttcgggctgcgatgttggtagtcttgggacagctggtgaagtggaatgtcagcacctgctccgtctccaggatggagctgaagtggtgagaggaagaacagagtggagttccaaaacatcaacaacaacttgggacattacaccgtga

SEQ ID NO: 20 - DNA, 1572 bp, Brassica napus
atggtggccacctcagctacatcctcattcttccctctcccatcttcccccctcgaccccaccgcaaaaaccaacaaagtcaccacctccaccaacttctccggcctcacacccacgccgaactccgccaggatgaaggttaaaccaaacgctcaggccccacccaagatcaacggcaagagagtcggcctccctggctcggtggagatcttgaagcctgatagcgagacttcgcaaccagcaccgaggacgttcatcaaccagctgcctgactggagcatgctcctcgccgccatcacgaccgtcttcttggcggctgagaagcagtggatgatgctcgactggaaaccgaggcgttctgacgtgattatggatccgtttgggttagggaggatcgttcaggatgggcttgtgttccgtcagaatttttctattcggtcttatgagataggtgctgatcgctctgcgtctatagaaacggttatgaatcatttacaggtactgattatgattatgattgtagtcgcttgttgttactggacaaacttaaatatgtattgctcttatggttgtgataggaaacggcactcaaccatgttaagactgctgggctgcttggagatgggtttggttctactcctgagatggttaagaagaacttgatatgggttgttactcgtatgcaggttgtcgttgataaatatcctacttggtaagctattctcaaacaactctgagaatcactgcttcctttgtgagtcatttgcttattcaaatatctgcctcataggggagatgttgtggaagtagatacatgggtgagccagtctggaaagaacggtatgcgtcgtgattggcttgttcgggatggcaatactggagagattttaacaagagcatcaaggttagattttattttttggtttacttgggttagatatctgataattgagttataatcatctccgtgttgtgtaaactattctttttgcagtgtgtgggtgatgatgaataaactgacaagaagattatcaaagattcctgaagaggttcgaggggagatagagccttactttgttaactcagacccagtccttgccgaggacagcagaaagttaacaaaacttgatgacaaaactgctgtctatgttcgttctggtctcactgtaagtacaaatacttcactctatgtttcaacaaagcctgtaaatttttgagtctcttacaggtttggtaatgaactttttgcagccgcgttggagtgacttggatgttaaccagcacgttaacaatgtgaagtacatcgggtggatactggagagtgctccagtggggatgatggagagtcagaagctgaaaagcatgactctggagtatcgcagggagtgtgggagagacagtgtgctccagtccctcaccgcggtttcgggctgcgatatcggtagcctcgggacagccggtgaagtggaatgtcagcatctgctcagactccaggatggagccgaagtggtgagaggaagaacagagtggagttccaaaacatcaacaacaacttgggacatcacaccgtga

SEQ ID NO: 21 - DNA, 1664 bp, Brassica napus
atggtggctacttccgctacgtcgtcgttttttcatgttccatcttcctcctctcttgatactaatgggaaggggaacagagttgcgtccacgaacttcgctggacttaactcaacgccaagctctgggaggatgaaggttaaaccaaacgctcaggctccacccaagatcaacgggaagaaagctaacttgcctggttctgcagagatatcaaagtctgacaacgagacttcgcaacccgcacccgcaccgaggacgtttatcaaccagctgcctgactggagcatgcttctcgctgccataacaactattttcttagcggctgagaaacagtggatgatgcttgactggaaacccaggcgttctgatatgataatggatcctttcggtttagggagaatcgttcaggatggtcttgtgtttcgtcagaatttctccattaggtcttatgagataggtgctgatcgctctgcgtctatagaaactgttatgaatcatttacaggtaggtactactttgattgttatcacacttgtcactggacacccaatagatatatatgctcatgacaagctcttatgctaatgacaggaaacggccctaaaccatgtgaagtctgccggactgctggaaaatgggtttggttctactcccgagatgtttaagaagaacttgatatgggtcgttgctcgtatgcaggttgtcgttgataaatatcctacttggtaagccattgtcagtcttaccacttaacttaaaatcattatgcatattacagtttgcatagatcattacttattcaaatatctgactaacaggggagatgttgtggaagtggatacatgggttagtcagtccggaaagaatggtatgcgtcgtgattggctggttcgggattgcaatactggagaaattgtaacgcgagcatcaaggtcagagttcttatgttttggtttactgactccagctattatcattttgctctctgtttgtattgtttgctctgccattaatatgataatagagactttatagttgtatatgtatggcaattttcttctttttgcagtttgtgggtgatgatgaataaactgacaaggagattgtcaaagattcctgaagaggttcgtggggaaatagagccttattttgtgaactctgatcctgtcattgccgaagacagcagaaagttaacaaaactggatgacaagactgctgactatgttcgttcgggtctcactgtaagtaccctacctttcaacaagcctttaaaactcttgaggttctaatggtttggtaataaacttttttttcagccgagttggagtgacttagatgttaaccagcatgttaacaatgtaaagtacattgggtggatactggagagtgctccagcagggatgctggagagtcagaagctgaaaagcatgactctggagtatcgcagggagtgcgggagagacagtgtgcttcagtctctcaccgcggtctctggatgtgatgtcggtaacctcgggacagccggggaagtggagtgtcagcatttgcttcgtctccaggatggagctgaagtggtgagaggaagaacagctgaagtggtgagaggaagaacagagtggagttccaagatagaagcaacaacttgggacactgctacatcgtaa

SEQ ID NO: 22 - DNA, 21 bp, Artificial Sequence
  Primer
acagtggatgatgcttgactc

SEQ ID NO: 23 - DNA, 21 bp, Artificial Sequence
  Primer
tagtaatatacctgtaagtgg

SEQ ID NO: 24 - DNA, 25 bp, Artificial Sequence
  Primer
tacgatgtagtgtcccaagttgttg

SEQ ID NO: 25 - DNA, 22 bp, Artificial Sequence
  Primer
tttctgtggtgtcagtgtgtct

SEQ ID NO: 26 - DNA, 1714 bp, Brassica napus
atggtggccacctctgctacatcctcattcttccctctcccatcttcctctctcgaccccaatggcaaaaccaacaaagccacctccaccaacttctccggactcaaccccacaccaaactcttccggcaggttaaaggtcaaaccaaacgctcaggctccatccaagatcaacggcaagaaagtctccttgccaggctcagtacacatcgtaaagactgataataaccacgatctctcgcaacaaaacgcacccagaacgttcatcaaccagctacctgactggagcatgcttctcgccgccatcacaacggtcttcttagcagctgagaagcagtggatgatgcttgatactaaaccgagacgctccgacatgattatggatccgtttgggttagggagaatcgttcaggatgggcttgtgtaccgtcagaatttcgatatcaggtcttatgaaataggtgctgatcgctctgcatctatagaaactgtcatgaatcacttacaggtatattacaatcacactcgtttgatactatagcttgacccgcactgatgttggtttttatatttttataaattgtttagtgacatatagatataggttatttagatatttctaggttcctacgaacctacccggactcaaaccctgtccgtaaaattgagtttaattttaaaccaaaaaaatccgatacccgaaaaaaccgatctgtatctaactcttgtcctcatgacaggaaacggctctcaaccatgtgaagtctgcaggactgctgggagatgggtttggttctacacctgagatggttaagaagaacttgatatgggttgttactcgtatgcaggttgtagttgataaatatcctacttggtaagctctcttgccacttaaccttaaacaatatgcatgaatcatttgcttattcaaatgtctgtttcaccaggggagatgttgttgaagtagatacatgggtcagtaagtctgggaagaatggtatgcgtcgtgattggctagttcgtgattgcaatactggagaaatcttaacacgcgcatcaaggttagctttattttgtttttgtttactccagctattatctgattattgagttataaccatctctatgttacaaaacagtgtgtgggtgatgatgaataaactgacaaggagattatcaaagcttcctgaagaggttcgaggggaaatagagccttactttgtgaactctgacccaatccttgccgaggacagcagaaagttaacaaagctagatgacaagactgctgactatgttcgctctggtctcaccgtaagtataaatattcaactctttatcttttagcgtgtaaaactcttgagagattcttatgagtttggtgatgaacttttgcagccgagatggagtgacttggatgttaaccagcatgttaacaacgtgaagtacattggttggatactcgagagtgctccagtagagatgatggagaagcataagctgaaaagcatgactctggagtataggagggaatgcgggagagacagtgtgcttcagtctctcaccgcggtttcgggatgcgatgttggtagcctcgggacagctggtgaagtggaatgtcagcatttgcttcgacaccaggatggagctgaagtggtgaagggacgaacagtgtggagttcgaaaacaccatcaacaacttgggacactacatcgta

SEQ ID NO: 27 - DNA, 1891 bp, Brassica napus
atggtggccacctctgctacatcctcattcttccctctcccatcttcctctctcgaccctaatggcaaaaccaacaaactcacctccaccaacttctctggactcaaccccataccaaactcttccggcaggttaaaggtcaaaccaaacgcccaagctccatccaagatcaacggcaataatgtctccttgccaggctcagtacacatcgtaaagactgataataaccacgatctctcgcaacaacacgcacccagaacgttcatcaaccagctacctgactggagcatgcttctcgccgccatcacaacggtcttcttagctgctgagaaacagtggatgatgcttgactcgaaaccgaggcgttctgatatgattatggatccgttcgggttagggaggatcgttcaggatgggcttgtgtaccgtcagaacttcgatatcaggtcttatgaaataggtgctgatcgctctgcgtctatagaaacagtcatgaaccacttacaggtatattacaatcacactcgattgatactagagcttgacatgttggtttttatctttttataaattgtttagtgacattttcaaacatatagatataggttatttagatatttctaggttcctacaaacctacccagactcaaaccccgtccggaaatttataatattaataccgaacagagttttattttaaaccaaaaaatcagttgacccgcacgggatgttggtttttatctattttatacattgtttaaggacatttttaaacatataaatataggttatttagatatttctaggttcctacgaacctacccggaaatttataatacccgaacatagtttaatttttaaaccaaaaaatccaatacccgaaaaaaccaatctgtgatatgcatgatctaactcttgtcctcgtgacaggaaacggctctcaaccatgtgaagtctgctggactgctgggagatgggtttggttctacccctgagatggttaagaagaacttgatatgggtcgttactcgtatgcaggttgtcgttgataaatatcctacttggtaagccctcttagcacttaaccttaaaacaatatgcatgaatcatttgcttattcaaatgtctgcttcaccaggggagatgttgttgaagtagatacatgggttagtaagtctgggaagartggtatgcgtcgtgattggcttgttcgggattgtaatactggagaaattttaacaagagcatcaaggttagcttctttttgtttactccagctattatctgattattgagttataaccatctctgtgttgcaaaacagtgtgtgggtgatgatgaataaagtgacaaggagattatcaaagcttcctgaagaggttcgaggggaaatagagccttactttgtgaactctgaccctatccttgccgaggacagcagaaagttaacaaaactagatgagaagactgctgactatgttcgctctggtctcaccgtaagtataaatatttgtttttatctttcagcaagtgagattctgatgggtttggtgattatctaacttttgcagccgagatggagtgacttggatgttaaccagcatgttaacaacgtgaagtacattggttggatactcgagagtgctccagtggagatgatggagaagcataagctgaaaagcatgactctggagtataggagggaatgcgggagagacagtgtgcttcagtctctcaccgcggtttcgggttgcgatgttggtagcctcgggacagctggtgaagtggaatgtcagcatttgcttcgactccaggatggagctgaagtggtgaagggacgaacagtgtggagttccaaaacaccatcaacaacttgggacactacatcgta

SEQ ID NO: 28 - DNA, 1164 bp, Brassica napus
aagtgtggattctcgacggatggatttgccacaacactcaccatgaggaaattgcatctcatatgggtcactgcaagaatgcacattgagatctacaagtacccagcttggtattttcttttcttaggcttctttgactagttgacactttagaggtcggagtttgtaaacctcagagctttttattacttggttaacaggagtgatgttgttgagatagagacatggtgccagagtgaaggaaggattggaacgagacgtgattggattctaagggactctgctacaaatgaagttattgggcgtgctacaaggtttgccaaaaacagatttgttactactattcataaattcatttttttatctgccttcaatcaatataataatgcaaatcactgacattagtcgcacaacagtaactcccatatacgttgcttatttagttataaagacttatgcatattctggaacctgagcttgtttttgtttgacaaatgttacatgggtcttacagcaagtgggtgatgatgaaccaagacacaaggcggcttcaaagagttacagatgaagttcgggacgagtacttggttttctgtcctcgagaacccaggtgaagaagaatcatcatgcttcccttataattgctagttaaacagttaatatttaagcatgtggatctcaacctgttgtcctctgtatttctcgtagactagcgtttccagaagagaacaatagcagcttaaagaaaatcccaaaactagaagatccagctcagtattctatgctagagcttaagcttcggcgagctgatctggacatgaaccagcacgtgaataacgtcacctacattggatgggtgcttgaggtgagtaccttaataaagcctacaaaacgtctatcattttaatcatacatatgagctaactaactattaaatttgagtttggttccctggtaatggcagagcatacctcaagaaatcattgatacgcatgagcttcaagttataactctagattacagaagagaatgccagcaagatgacattgtagattcactcaccacctctgaaatccctgacgacccgatctcaaagcttaccgggaccaacggatctgccacgtcaagcatacaaggacacaatgagagccagttcttgcatat

SEQ ID NO: 29 - protein, 36 aa, Artificial Sequence
  Amino acid motif
LEDPAQYSMLELKPRRADLDMNQHVNNVTYIGWVLE

SEQ ID NO: 30 - protein, 367 aa, Arabidopsis thaliana
MLKLSCNVTDHIHNLFSNSRRIFVPVHRQTRPISCFQLKKEPLRAILSADHGNSSVRVADTVSGTSPADRLRFGRLMEDGFSYKEKFIVRSYEVGINKTATIETIANLLQEVACNHVQNVGFSTDGFATTLTMRKLHLIWVTARMHIEIYKYPAWSDVVEIETWCQSEGRIGTRRDWILKDCATGEVIGRATSKWVMMNQDTRRLQRVTDEVRDEYLVFCPPEPRLAFPEENNSSLKKIPKLEDPAQYSMLGLKPRRADLDMNQHVNNVTYIGWVLESIPQEIIDTHELKVITLDYRRECQQDDIVDSLTTSETPNEVVSKLTGTNGSTTSSKREHNESHFLHILRLSENGQEINRGRTQWRKKSSR

SEQ ID NO: 31 - protein, 223 aa, Brassica napus
GFSTDGFATTLTMRKLHLIWVTARMHIEIYKYPAWSDVVEIETWCQSEGRIGTRRDWILRDSATNEVIGRATSKWVMMNQDTRRLQRVTDEVRDEYLVFCPREPRLAFPEENNSSLKKIPKLEDPAQYSMLELKPRRADLDMNQHVNNVTYIGWVLESIPQEIIDTHELQVITLDYRRECQQDDIVDSLTTSEIPDDPISKLTGTNGSATSSIQGHNESQFLH

SEQ ID NO: 32 - DNA, 1163 bp, Brassica napus
aagtgtggattctcgacggatggatttgccacaacactcaccatgaggaaattgcatctcatatgggtcactgcaagaatgcacattgagatctacaagtacccagcttggtattttcttttcttaggcttctttgactagttgacactttagaggtcggagtttgtaaacctcagagctttttattacttggttaacaggagtgatgttgttgagatagagacatggtgccagagtgaaggaaggattggaacgagacgtgattggattctaagggactctgctacaaatgaagttattgggcgtgctacaaggtttgccaaaaacagatttgttactactattcataaattcatttttttatctgccttcaatcaatataataatgcaaatcactgacattagtcgcacaacagtaactcccatatacgttgcttatttagttataaagacttatgcatattctggaacctgagcttgtttttgtttgacaaatgttacatgggtcttacagcaagtgggtgatgatgaaccaagacacaaggcggcttcaaagagttacagatgaagttcgggacgagtacttggttttctgtcctcgagaacccaggtgaagaagagtcatcatgcttcccttataattgctagttaaacagttaatatttaagcatgtggatctcaacctgttgttctctgtatttctcgtagactagcgtttccagaagagaacaatagcagcttaaagaaaatcccaaaactagaagatccagctcagtattctatgctagagcttaagcttcggcgagctgatctggacatgaaccagcacgtgaataacgtcacctacattggatgggtgcttgaggtgagtaccttaataaagcctacaaaacgtctatcattttaatcatacatatgagctaactaactattaaatttgagtttggttccctggtaatggcagagcatacctcaagaaatcattgatacgcatgagcttcaagttataactctagattacagaagagaatgccagcaagatgacattgtagattcactcaccacctctgaaatccctgacgacccgatctcaaagcttaccgggaccaacggatctgccacgtcaagcatacaaggacacaatgagagccagttcttgcata

SEQ ID NO: 33 - DNA, 86 bp, Brassica rapa
ctcagtattcgatgattgggcttaagcctagacgagctgatctcgacatgaaccaggatgtcaataatgtcacctatattggatgg

SEQ ID NO: 34 - DNA, 86 bp, Arabidopsis thaliana
ctcagtattcaatgattgggcttaagcctagacgagctgatctcgacatgaaccagcatgtcaataatgtcacctatattggatgg

SEQ ID NO: 35 - DNA, 83 bp, Brassica napus
tttataatcatgtttctttgcagccaagacgagctgatctcgacatgaaccatcatgtcaataatgtcacctatattggatgg

SEQ ID NO: 36 - DNA, 86 bp, Arabidopsis thaliana
ctcagtattctatgcttgggcttaagcctagacgagctgatcttgacatgaaccaacatgtgaataatgttacctacattggatgg

SEQ ID NO: 37 - DNA, 86 bp, Brassica napus
ctcagtattctatgctagagcttaagcctcggcgagctgatctggacatgaaccagcacgtgaataacgtcacctacattggatgg

SEQ ID NO: 38 - DNA, 66 bp, Brassica napus
ttaagcctcggcgagctgatctggacatgaaccagcacgtgaataacgtcacctacatcggatggg

SEQ ID NO: 39 - DNA, 86 bp, Brassica napus
ctcagtattctatgctagagcttaagcttcggcgagctgatctggacatgaaccagcacgtgaataacgtcacctacattggatgg

SEQ ID NO: 40 - DNA, 66 bp, Brassica napus
ttaagcttcggcgagctgatctggacatgaaccagcacgtgaataacgtcacctacattggatggg

SEQ ID NO: 41 - DNA, 66 bp, Arabidopsis thaliana
ttaagcctagacgagctgatcttgacatgaaccaacatgtgaataatgttacctacattggatggg

SEQ ID NO: 42 - DNA, 66 bp, Brassica napus; identical to SEQ ID NO: 38

SEQ ID NO: 43 - DNA, 66 bp, Brassica napus
ttaagcctcggcgagctgatctggacatgaaccagcacgtgaataacgtcacctacattggatggg

SEQ ID NO: 44 - DNA, 310 bp, Brassica napus
gtttccagaagagaacaatagcagcttaaagaaaatcccaaaactagaagatccagctcagtattctatgctagagcttaagcctcggcgagctgatctggacatgaaccagcacgtgaataacgtcacctacatcggatgggtgcttgaggtgagtaacttaataaagccttcaaaacgtctatcattttaataatgagctaactattaaatttgagtttggttccttggtaatggcagagcatacctcaagaaatcattgatacgcatgagcttcaagttataactctagattacagaagagaatgcc

SEQ ID NO: 45 - DNA, 320 bp, Brassica napus
gtttccagaagagaacaatagcagcttaaagaaaatcccaaagctagaagatccagctcagtattctatgctagagcttaagcctcggcgagctgatctggacatgaaccagcacgtgaataacgtcacctacattggatgggtgcttgaggtgagtaccttaataaagcctacaaaacgtctatcattttaatcatacatatgagctaactaactattaaatttgagtttggttccctggtaatggcagagcatacctcaagaaatcattgatacgcatgagcttcaagttataactctagattacagaagagaatgcc

SEQ ID NO: 46 - DNA, 310 bp, Brassica napus
gtttccagaagagaacaatagcagcttaaagaaaatcccaaaactagaagatccagctcagtattctatgctagagcttaagcctcggcgagctgatctggacatgaaccagcacgtgaataacgtcacctacatcggatgggtgcttgaggtgagtaacttaataaagccttcaaaacgtctatcattttaataatgagctaactattaaatttgagtttggtcccttggtaatggcagagcatacctcaagaaatcattgatacgcatgagcttcaagttataactctagattacagaagagaatgcc

SEQ ID NO: 47 - DNA, 320 bp, Brassica napus
gtttccagaagagaacaatagcagcttaaagaaaatcccaaaactagaagatccagctcagtattctatgctagagcttaagcttcggcgagctgatctggacatgaaccagcacgtgaataacgtcacctacattggatgggtgcttgaggtgagtaccttaataaagcctacaaaacgtctatcattttaatcatacatatgagctaactaactattaaatttgagtttggttccctggtaatggcagagcatacctcaagaaatcattgatacgcatgagcttcaagttataactctagattacagaagagaatgcc

SEQ ID NO: 48 - protein, 36 aa, Arabidopsis thaliana
LEDPAQYSMLGLKPRRADLDMNQHVNNVTYIGWVLE

SEQ ID NO: 49 - protein, 36 aa, Brassica napus
LEDPAQYSMLELKLRRADLDMNQHVNNVTYIGWVLE

SEQ ID NO: 50 - protein, 36 aa, Brassica napus; identical to SEQ ID NO: 29

SEQ ID NO: 51 - protein, 36 aa, Brassica napus; identical to SEQ ID NO: 29

SEQ ID NO: 52 - protein, 36 aa, Brassica napus; identical to SEQ ID NO: 29

SEQ ID NO: 53 - DNA, 61 bp, Brassica napus
acatatagccaatggctccaactctcctctycctatatcaccattagtagactcacaatct

SEQ ID NO: 54 - DNA, 61 bp, Brassica napus
atggtcatggttctctcatttggtaaacatyttgttcctcataaatcataatgattccctc

SEQ ID NO: 55 - DNA, 61 bp, Brassica napus
agcgtcattggagttatggaatacagaaacycacttgtcgtaatcctgattcactagaagc

SEQ ID NO: 56 - DNA, 61 bp, Brassica napus
cgggatctgcatccaccgccgctagagattycggttctgatgctccaccaagggttggttt

SEQ ID NO: 57 - DNA, 61 bp, Brassica napus
gagaccacaaggggttggaatcaagatgggytgttatggagcaagcccgtgtcgagtactt

SEQ ID NO: 58 - DNA, 61 bp, Brassica napus
aaaccggatttattaaagacacaaacctaayctccagatgagaggtgcaatacacatatgg

SEQ ID NO: 59 - DNA, 61 bp, Brassica napus
tggctgaacgaaaacacaatgcaccaatgtygatcattcactacagaagataattgatatc

SEQ ID NO: 60 - DNA, 61 bp, Brassica napus
gccacacctttgcttcacttgggtccgcccytgtctataactgaatccccaacatgagaca

SEQ ID NO: 61 - DNA, 61 bp, Brassica napus
accaacaaatcagaactaatattgaattgtycaaaggtagaagtcatcaatcaagatatga

SEQ ID NO: 62 - DNA, 61 bp, Brassica napus
tagtctatgaaccactcaaacccttaacccytagtggctttgcttttccttattcggactt

SEQ ID NO: 63 - DNA, 61 bp, Brassica napus
aaatgaagggtgaaaatgtaataaataaatytcttatatactaaagcacaagtcactctac

SEQ ID NO: 64 - DNA, 61 bp, Brassica napus
caatcttgaaaaccctttgtctacttgcgcyacaaggaatacaatgctttgcttttgtttt

SEQ ID NO: 65 - DNA, 61 bp, Brassica napus
caaccacactctatttttcactctaaaataragtttagagtaaaaatgctccaataagact

SEQ ID NO: 66 - DNA, 61 bp, Brassica napus
aaccaatatgagagaaccaactcctaaaaaraatcctcgaaaactaacgagcgggctgatt

SEQ ID NO: 67 - DNA, 61 bp, Brassica napus
atacagtaaacatatgcgttcacatggccaytgccaagttaatgaaggaattacgtactct

SEQ ID NO: 68 - DNA, 61 bp, Brassica napus
aacatttccaaaatgcggttaaaatgtttaycaccaaatatttagtatattttaacatgaa

SEQ ID NO: 69 - DNA, 61 bp, Brassica napus
gatgagatacaacgcttccgtgatcgagctygggagcgaagacaccgcgctgatgcggtgt

SEQ ID NO: 70 - DNA, 61 bp, Brassica napus
cgtcggagacaatatcaacgcctgaactctrcaaatcaaaaccataacataagaaacaatc

SEQ ID NO: 71 - DNA, 61 bp, Brassica napus
gggcaacccaacacttatttccaatgttatmtttcttcattttcaataccctgccccactt

SEQ ID NO: 72 - DNA, 61 bp, Brassica napus
taactccattgaaagtgccactagcccagtrctaaaataccttgccattagtcctaatcaa

SEQ ID NO: 73 - DNA, 61 bp, Brassica napus
aaatgcaatagttaaattgacttttctgacygatgataattaaatgtgaaaaaaacactgt

SEQ ID NO: 74 - DNA, 61 bp, Brassica napus
caagtacatcgctcgaaactcctcgttggtyagatcagcgaaccgggtcaacccgagttcg

SEQ ID NO: 75 - DNA, 61 bp, Brassica napus
tatagcattgctaaatttaaattctattttycggtaagagattctttgtttcaccgggaga

SEQ ID NO: 76 - DNA, 61 bp, Brassica napus
aacatacattctcttaatgattgattgttcyctatagtatatggttagaagtgttgatatg

SEQ ID NO: 77 - DNA, 61 bp, Brassica napus
aagccctgccttctatgttaccaaagcctgsttatcagtttgactaactgggatggtacat

SEQ ID NO: 78 - DNA, 61 bp, Brassica napus
cgtttcaatcagacaaagttgcatttttttyttcatgagtagtttacactttgcacgccgt

SEQ ID NO: 79 - DNA, 61 bp, Brassica napus
tttgattttcttaaagaacttgagacaatcyttagataaaactttcttcaaacctcatcac

SEQ ID NO: 80 - DNA, 61 bp, Brassica napus
tgattgacagtggaaggcattatgaaggacrtacgttcgtctacgttgatgcaccaagtca

SEQ ID NO: 81 - DNA, 61 bp, Brassica napus
agggaaggagtaaaaacaggcaaatctatartataatgttattgactaacttattattaca

SEQ ID NO: 82 - DNA, 61 bp, Brassica napus
aagacctgactatacaatctttggtttttayctaatgcacaactagcacaagcattcatgt

SEQ ID NO: 83 - DNA, 61 bp, Brassica napus
ccgccgcttctccgcctccggatccgacgayggacgaagtgaatgagtcgttgcggagact

SEQ ID NO: 84 - DNA, 61 bp, Brassica napus
aaaattattctttcaatgtatcttattttgyttaatcattattattttgaaaatatgttat

SEQ ID NO: 85 - DNA, 61 bp, Brassica napus
tgtttgttatgtaactgcagaaaacatcatracaatcgtattcaaattgtaagcaaaggaa

SEQ ID NO: 86 - DNA, 61 bp, Brassica napus
tcttgtatttgaagttggagatttcgtttargcatatcttacacaggataggatgccagct

SEQ ID NO: 87 - DNA, 61 bp, Brassica napus
cacttctctgtatttttcttcttttctgtgyagtttggctcctatcattaatgaaaactct

SEQ ID NO: 88 - DNA, 61 bp, Brassica napus
accattgaaaattcaaatgaaaattcaaatygtgttatagagggagagagagagagagagg

SEQ ID NO: 89 - DNA, 61 bp, Brassica napus
tattaaacttataaaaactattaaaaccatyaaaaatctataaactatctatataaacata

SEQ ID NO: 90 - DNA, 61 bp, Brassica napus
aaaactagataaatatatttttaaagttatrtgttgtaacaaagttatttattgacccaaa

SEQ ID NO: 91 - DNA, 61 bp, Brassica napus
ataaaaaattatgtttgaaactatttttcarttttttaatatattttttaagtatttattt

SEQ ID NO: 92 - DNA, 61 bp, Brassica napus
agaggaaacataaacaagaaaccaaatccayaatatagcatttctactattttcaaactca

SEQ ID NO: 93 - DNA, 61 bp, Brassica napus
tgagtgtcatttcttaggtgtcattttcacragctctctcacaacaaaattttaaaaatcc

SEQ ID NO: 94 - DNA, 61 bp, Brassica napus
aacgtcgacggctcttgttctctcgtctccytttctccggaagagaaccatcaaaacaaga

SEQ ID NO: 95 - DNA, 61 bp, Brassica napus
tcacgggccttactgagtcgtatcaactctrtttggactcaacaaagaaacaaaagcttga

SEQ ID NO: 96 - DNA, 61 bp, Brassica napus
agtctgaataacagtattctcctggcgagtraacggctgtttcaagtatccagacctatga

SEQ ID NO: 97 - DNA, 61 bp, Brassica napus
taaaacgtgagagctcatagcaaaaaatcaytttgcaaataattgtataataaatattttt

SEQ ID NO: 98 - DNA, 61 bp, Brassica napus
tttgacttatacaaatattttgcatgctagycgctatttaatttttgtttaccggatattt

SEQ ID NO: 99 - DNA, 61 bp, Brassica napus
tgatcttagcgacgacgattagtgtttactytctttaatgcctaataaagcgtccctaaca

SEQ ID NO: 100 - DNA, 61 bp, Brassica napus
cattcgacccatctcgaagcccattcccgarccactctctcgtaagcataatcccgtgttc

SEQ ID NO: 101 - DNA, 61 bp, Brassica napus
agtttgggtttttggataaaaatcttatatrttaaaacataagtcatgacttctttcatgt

SEQ ID NO: 102 - DNA, 61 bp, Brassica napus
aattttgataatgttttaattttccaattgyccccaaaaacattccagttatatagtttgt

SEQ ID NO: 103 - DNA, 61 bp, Brassica napus
cgcgcgggacaagccggctgtgacccgctcratgactaacccgccgtggtacgggatggaa

SEQ ID NO: 104 - DNA, 61 bp, Brassica napus
agatgcatattatcgtacaagaacaataaayttcccgccatttttgagaaaaatggcatgt

SEQ ID NO: 105 - DNA, 61 bp, Brassica napus
caccacgttaaaatagtttgttgcaaaaaaycacttgtaacagttgcaaaaaaccacttgt

SEQ ID NO: 106 - DNA, 61 bp, Brassica napus
gtctcgagatagtcgacgccttcagcttgtytgttgcctctggaaacaactctagctctat

SEQ ID NO: 107 - DNA, 61 bp, Brassica napus
gccagtatataaagattcctaggcgagaagyatggggaggacttttctcagagcaaactta

SEQ ID NO: 108 - DNA, 61 bp, Brassica napus
acgtcttctccgaccataacattgtacctgycaaaatagacagtttgtagattaactgttt

SEQ ID NO: 109 - DNA, 61 bp, Brassica napus
gccgggttggtacaccatcaccgtcacccgycgtccacctctgtctcatcctcgtaaccaa

SEQ ID NO: 110 - DNA, 61 bp, Brassica napus
ggatgggacagatgagaggttgaaatcgcgyctggttgtctatggaaataaacaagtcgag

SEQ ID NO: 111 - DNA, 61 bp, Brassica napus
gaaaccctgtaaccaatcggctctgtttccycgataatctatcgtctgtgtaactcaccgg

SEQ ID NO: 112 - DNA, 61 bp, Brassica napus
ttttttttttggcaactatttttattttctyaatttctgtttccataaataaaatatgacg

SEQ ID NO: 113 - DNA, 61 bp, Brassica napus
atgcgaccttgttagggaacttgtcgatggygtattgggatcaggatgatccgtacgagat

SEQ ID NO: 114 - DNA, 61 bp, Brassica napus
ttatgtatctataaaatgaccaagactaatyttaaaaatgaagtgaaagctacatataatt

SEQ ID NO: 115 - DNA, 61 bp, Brassica napus
tatatgaatctaaattacctacgaccgtctycatcgctgtgcacatcaaaactatataacc

SEQ ID NO: 116 - DNA, 61 bp, Brassica napus
tcaaaccacaggagtcagccaatatagcagyagagtctacagagcctctaatcctaaccgt

SEQ ID NO: 117 - DNA, 61 bp, Brassica napus
aagaaaggagatcgtcgtaggaacgctgagygtaacacacaaagaaagcgtgtgtaacgca

SEQ ID NO: 118 - DNA, 61 bp, Brassica napus
catttctagagattcaaaattatattttggrttttattgagatgatttaggagtttgatgc

SEQ ID NO: 119 - DNA, 61 bp, Brassica napus
acttcttctacagcgaagtcgctcgcatcgyacatgatttcgaaaggaagagtccagtctg

SEQ ID NO: 120 - DNA, 61 bp, Brassica napus
acccgaaagaatttgaataagaagctggttytgtattgggtttgtatgttttgcgatatac

SEQ ID NO: 121 - DNA, 61 bp, Brassica napus
cgagcaaactgatccatgacttttaggaccycacgcaaatatctactggttctgtctgtaa

SEQ ID NO: 122 - DNA, 61 bp, Brassica napus
ttcaatttcttccggaagaatcatcttcagycttcgcactaatatcttggagattacctta

SEQ ID NO: 123 - DNA, 61 bp, Brassica napus
ggaaggtggaaaaggttttctcaagcatatycttatccgttatatcctcaccacacagttt

SEQ ID NO: 124 - DNA, 61 bp, Brassica napus
aatcaaaagtctaccttcttagctaagaaaytagatatcacgtgtgatagcaaaaacaaaa

SEQ ID NO: 125 - DNA, 61 bp, Brassica napus
gttagtattccttacgtcccaatgcttactycaacttgcatttctcttgtacttaagatcc

SEQ ID NO: 126 - DNA, 61 bp, Brassica napus
tctctttgttcttatccaggttccactgctyttgcatttcaaccaattcttggaaggtgga

SEQ ID NO: 127 - DNA, 61 bp, Brassica napus
tccagcatcctgcaagaacaacgtagagacytccacaccacagtggactaacctatcacat

SEQ ID NO: 128 - DNA, 61 bp, Brassica napus
aaccatatagattgtgttataagtctttctraatggttataaactcttataaatgattaaa

SEQ ID NO: 129 - DNA, 61 bp, Brassica napus
taataatctagatgctcaaaattacaattawaaatctaaatttgtttagttattttctgta

SEQ ID NO: 130 - DNA, 61 bp, Brassica napus
aatttcgagaaaatcttcacggaccagaaarttatggattttacaaactggagcttctcca

SEQ ID NO: 131 - DNA, 61 bp, Brassica napus
ttctcattaaacaaagaaaaatggcaatctyttttctgtgtctctttctcatcacctttgc

SEQ ID NO: 132 - DNA, 61 bp, Brassica napus
ggctgaaccagaacatttatctactgaaggyagagcatatttttgaaaatatagtttataa

SEQ ID NO: 133 - DNA, 61 bp, Brassica napus
ttgagcatgagagataactggctggagtgcytctttgagcctgcccgtaagaagctgggag

SEQ ID NO: 134 - DNA, 61 bp, Brassica napus
ttttattgaagtgcatttatccaaaatttcyccctaaaatgtattcccttagtttcacaaa

SEQ ID NO: 135 - DNA, 61 bp, Brassica napus
tccattcccaagactaaggagctcattcatyacattagattgtgtcctatcagctatatca

SEQ ID NO: 136 - DNA, 61 bp, Brassica napus
gttggcagcgaggcgcggtctcacgctctamtatctccttgcgaaagggctccagctcgtc

SEQ ID NO: 137 - DNA, 61 bp, Brassica napus
tttgtaaaataaatcatgtttttcatgaatyttttttaaagagaatatgtatttaatcaat

SEQ ID NO: 138 - DNA, 61 bp, Brassica napus
ccagatttcccaattccaagtttgtcttttyatgtaaattcttcggcaaatacaggtatgt

SEQ ID NO: 139 - DNA, 61 bp, Brassica napus
aatgaatcttcctgccgctccctctgtgatycagtagaacactcgtcacaacctcaaaata

SEQ ID NO: 140 - DNA, 61 bp, Brassica napus
tcttactattactaaaccttgtccccaaaaycccacccttcaactctaaaccttaagtcta

SEQ ID NO: 141 - DNA, 61 bp, Brassica napus
agtcaccaagctcggtcgtctcgttcagagyggtaaaatcacgcagctagagcatatctat

SEQ ID NO: 142 - DNA, 61 bp, Brassica napus
atacagaggcgatgaatgcgaaagtggatayagaggtggagactgtggtgacgatagatac

SEQ ID NO: 143 - DNA, 61 bp, Brassica napus
cacctccgccgtgtgtcgataccatgaacaytcaacctccgcctgtcttcacctccactac

SEQ ID NO: 144 - DNA, 61 bp, Brassica napus
agacatccatgacgattcctcgaaggcaaaytcacacacgcttctgctagctgttgtagcg

SEQ ID NO: 145 - DNA, 61 bp, Brassica napus
cctttccaacactccatcagaagtactcctycaacttaatcttgtacataccagtttatct

SEQ ID NO: 146 - DNA, 61 bp, Brassica napus
cttcctttcaacactactcgtcgtttctgtytcctttgagattgactttagatcatcttct

SEQ ID NO: 147 - DNA, 61 bp, Brassica napus
attgagatataaatattataataatatataycttaaatagcgagctcaataaattttattt

SEQ ID NO: 148 - DNA, 61 bp, Brassica napus
gttcgtcacacccagcaatgagcaagaaacyaaggatactgcgaaaatccaaggccggagt

SEQ ID NO: 149 - DNA, 61 bp, Brassica napus
tgccaacctcaaatctcaactttaataatcyttttatatctctttacaaatatccacccaa

SEQ ID NO: 150 - DNA, 61 bp, Brassica napus
acaacacattaacaaaaaaaatgtcattcgyttcactcttgtatgcattcttcttgatctt

SEQ ID NO: 151 - DNA, 61 bp, Brassica napus
aaagaatcaaactgtaggaatttataattgycctttgcaagttttttttttgtaactgagc

SEQ ID NO: 152 - DNA, 61 bp, Brassica napus
tgatataccgaaaaatcaaacaagcagcgtycattgttgcagaacaagtagcgtacatatt

SEQ ID NO: 153 - DNA, 61 bp, Brassica napus
ttatcttattagaactgattttagtttcttytttcattctaggatttaattaatgacataa

SEQ ID NO: 154 - DNA, 61 bp, Brassica napus
agttctgcttcaccaatacctccataagctycatccactcaggccacggatgcaccaactc

SEQ ID NO: 155 - DNA, 61 bp, Brassica napus
agttaaaaaaaaatcaatcttgtttcatttytattaattgttgagacgccaataattttat

SEQ ID NO: 156 - DNA, 61 bp, Brassica napus
tgttccaatatataagatgttctcatctttytatgtaattttaagtttatcaaaaactgtg

SEQ ID NO: 157 - DNA, 61 bp, Brassica napus
gagtcgctcgcacagatctttgtttttatcytgagttcctctttgctcggagtttctctca

SEQ ID NO: 158 - DNA, 61 bp, Brassica napus
ccaaaactgaaaaggaaagaatgatctacgytgcatcagaagacgactccatggccggaga

SEQ ID NO: 159 - DNA, 61 bp, Brassica napus
ttaacataaagaaattattacaatgataaayattatacatagattttttagacgactaact

SEQ ID NO: 160 - DNA, 61 bp, Brassica napus
ctctaaatgtagagtgcttggcgacatatcyaacggaggctcttctctcgaaatcatcaaa

SEQ ID NO: 161 - DNA, 61 bp, Brassica napus
aaaatgtaatctttcccactctaaaactctycaacctctctctaatctctttgaacatcaa

SEQ ID NO: 162 - DNA, 61 bp, Brassica napus
ttcatgtgctaagcagttatatattattatyatatattattattacaataataagatgata